(19) United States
(12) Patent Application Publication
(10) Pub. No.: US 2026/0355191 A1
(43) Pub. Date: Nov. 12, 2026

(54) PAN-RAS INHIBITOR COMPOUND

(71) Applicant: ADLAI NORTYE BIOPHARMA CO., LTD., Hangzhou (CN)

(72) Inventors: Xiangyu FU, Hangzhou (CN); Meng LV, Hangzhou (CN); Shuaishuai LIU, Hangzhou (CN); Zhiyong YU, Hangzhou (CN)

(73) Assignee: ADLAI NORTYE BIOPHARMA CO., LTD., Hangzhou (CN)

(21) Appl. No.: 19/694,151

(22) Filed: Jun. 1, 2026

Related U.S. Application Data
(63) Continuation of application No. PCT/CN2025/086875, filed on Apr. 2, 2025.

(30) Foreign Application Priority Data
Apr. 3, 2024 (CN) 202410399678.5
May 20, 2024 (CN) 202410627425.9
Sep. 12, 2024 (CN) 202411280320.7
Dec. 6, 2024 (CN) 202411794717.8

(51) Int. Cl. A61K 31/504; C07D 513/22

(52) U.S. Cl. CPC A61K 31/504; C07D 513/22

(57) ABSTRACT

The disclosure relates to a compound having pan-RAS inhibitory effect represented by formula (A), or its isotopic derivatives, stereoisomers or pharmaceutically acceptable salts thereof, and a pharmaceutical composition comprising the compound, as well as use of the compound of formula (A) for preventing and/or treating cancer, tumors, inflammatory diseases, autoimmune diseases or immune-mediated diseases.

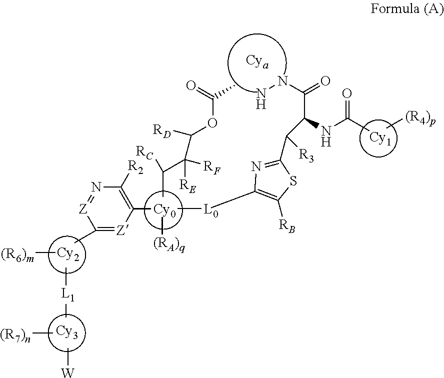

### CROSS REFERENCE TO RELATED APPLICATIONS

The application is a continuation of International Application No. PCT/CN2025/086875, filed on Apr. 2, 2025, which claims priority to and benefit of Chinese Patent Application No. 202410399678.5, filed on Apr. 3, 2024; Chinese Patent Application No. 202410627425.9, filed on May 20, 2024; Chinese Patent Application No. 202411280320.7, filed on Sep. 12, 2024, and Chinese Patent Application No. 202411794717.8, filed on Dec. 6, 2024. All the above-referenced applications are incorporated into the present application by reference herein in their entirety.

### TECHNICAL FIELD

The disclosure relates to a compound, in particular to a highly active pan-RAS inhibitor and its use.

### BACKGROUND TECHNIQUE

RAS is one of the most frequently mutated genes in human tumors, and its mutation occurs in about 30% of tumor patients, among which KRAS accounts for about 85% of RAS mutations. KRAS mutations exist in 88% of pancreatic cancer, 50% of colorectal adenocarcinoma, and 32% of lung adenocarcinoma. The development of targeted KRAS inhibitors has great clinical significance and value.

KRAS is a membrane-bound protein with GTPase activity, which cycles between a GDP-bound inactive conformation and a GTP-bound active conformation through nucleotide exchange, performing the function of a “molecular switch”. KRAS in the GTP-bound state can activate multiple downstream signaling pathways including RAF-MEK-ERK and PI3K-AKT, and regulate life processes such as cell growth, proliferation, differentiation and apoptosis.

KRAS mutations (such as G12C, G12D, G12V, G13D, etc.) will affect the hydrolysis of GTP mediated by GTPase activating proteins (GAPs), increase KRAS in the GTP-bound activated state, and over-activate downstream signaling pathways, eventually lead to the occurrence and development of tumors. However, since the KRAS protein lacks a corresponding hydrophobic pocket suitable for drug binding, and its affinity with GTP and GDP is at the picomolar level (~20 pM), it is very difficult to develop inhibitors that competitively bind to KRAS. KRAS has been considered an undruggable target for the past decades.

In May 2021, AMG510 was approved by the FDA for the treatment of locally advanced or metastatic non-small cell lung cancer carrying the KRAS^{G12C} mutation, breaking the history of KRAS being “undruggable”. However, the G12C mutation only accounts for a small portion of KRAS mutations. For mutations at other sites of KRAS, there is still a lack of satisfactory and effective inhibitor compounds, and a large number of clinical needs have not been met. Therefore, the development of effective pan− RAS inhibitor compounds is a need in the art.

### Contents of the Disclosure

The disclosure provides a pan-KRAS inhibitor. Such structures are different from existing KRAS^{G12C} inhibitors that act through covalent binding, but instead mediate the formation of a ternary complex between ubiquitous intracellular chaperones (such as Cyclophilin A) and KRAS proteins. The formation of the ternary complex can block the combination of KRAS and its downstream effector molecules (such as RAF) through steric blockage, inhibit the activation of MAPK and PI3K-AKT signaling pathways, thereby inhibiting the occurrence and development of tumors, and play a role in the treatment of tumors and other diseases.

In one aspect, the disclosure provides a compound of formula (A), or an isotopic derivative thereof, a stereoisomer thereof, or a pharmaceutically acceptable salt thereof:

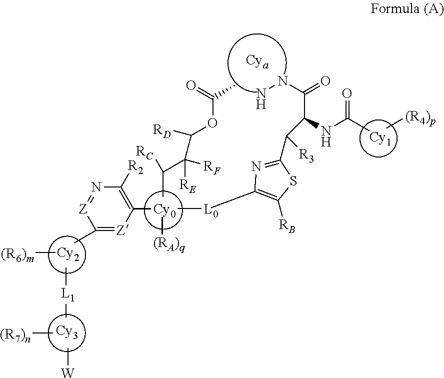

wherein:
Cya represents

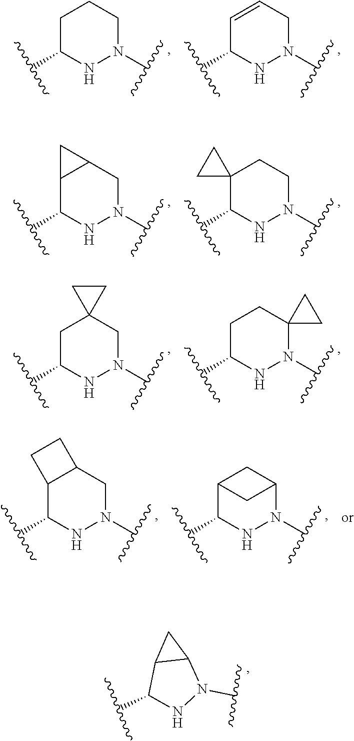

which may optionally be substituted with 0, 1, 2, or 3 substituents selected from halogen or C₁-C₃ alkyl;
R₂ represents halogen, cyano, C₁-C₆ alkyl, —(C₀-C₆ alkylene)-(C₃-C₈ cycloalkyl), or —(C₀-C₆ alkylene)-(4-8-membered heterocycloalkyl), which may optionally be substituted with 0, 1, or 2 substituents selected from: —ORa, —SRa, or —NRaRa′;
R₃ represents hydrogen, hydroxy, —(C₁-C₃ alkylene)-ORa, or C₁-C₃ alkyl;
Cy₁ represents a C₃-C₁₀ cycloalkyl or a 4-10-membered heterocycloalkyl, and Cy₁ may be a mono ring, spiro ring, bridged ring, or fused ring;
R₄ each independently represents hydrogen, halogen, oxo, C₁-C₆ alkyl, —(C₀-C₆ alkylene)(C₃-C₆) cycloalkyl, —(C₀-C₆ alkylene)(4-8-membered) heterocycloalky, —(C₀-C₆ alkylene)ORa, —(C₀-C₆ alkylene)SRa, —(C₀-C₆ alkylene)NRaRa′, —(C₀-C₆ alkylene)CORa, —(C₀-C₆ alkylene)COORa, —(C₀-C₆ alkylene)CONRaRa′, —(C₀-C₆ alkylene) NRaCORa′, —(C₀-C₆ alkylene) OCONRaRa′, —(C₀-C₆ alkylene) NRaCONRaRa′, —(C₀-C₆ alkylene)SORa, —(C₀-C₆ alkylene)S(O)₂Ra, —(C₀-C₆ alkylene)NRaS(O)₂Ra′, —(C₀-C₆ alkylene)CN, —(C₀-C₆ alkylene)(C₆-C₁₀ aryl) or —(C₀-C₆ alkylene)(5-12 membered heteraryl); wherein the R₄ substituents on two carbon atoms of Cy₁, together with the carbon atoms to which they are attached and the atoms between said two carbon atoms, may form a 3-8 membered ring, which may optionally contain 0, 1, 2, or 3 heteroatoms selected from N, O, or S; alternatively, the two R₄ substituents on the same carbon atom of Cy₁, together with the carbon atom to which they are attached, may form a 3-8 membered ring, which may optionally contain 0, 1, 2, or 3 heteroatoms selected from N, O, or S;
optionally, R₃ and R₄ may together form a ring;
Cy₂ represents a 5-membered heteroaryl;
Cy₃ represents a C₃-C₁₂ cycloalkyl or a 4-12-membered heterocycloalkyl, which may be a mono ring, spiro ring, bridged ring, or fused ring;
R₆ and R₇ are each independently selected from: hydrogen, halogen, oxo, —ORa, —NRaRa′, cyano, C₁-C₆ alkyl, C₃-C₈ cycloalkyl, or 4-8-membered heterocycloalkyl; each of the above-mentioned C₁-C₆ alkyl, C₃-C₈ cycloalkyl, and 4-8-membered heterocycloalkyl may be independently substituted with 0, 1, 2, 3, or 4 substituents selected from halogen, oxo, —ORa, —SRa, —NRaRa′, cyano, C₁-C₆ alkyl, C₃-C₈ cycloalkyl, or 4-8-membered heterocycloalkyl;
W is hydrogen or represented by formula (II), wherein * represents the site where W is attached to Cy₃;

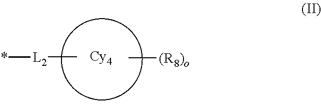

Cy₄ is selected from C₃-C₁₂ cycloalkyl or 4-12-membered heterocycloalkyl, which may be a mono ring, spiro ring, bridged ring, or fused ring;
R₈ is each independently selected from: hydrogen, halogen, oxo, —ORa, —NRaRa′, cyano, or C₁-C₆ alkyl, wherein the C₁-C₆ alkyl may be substituted with 0, 1, 2, 3, or 4 substituents selected from halogen, oxo, —ORa, —NRaRa′, or cyano;
L₁ and L₂ are each independently a single bond or a —(C₁-C₆) alkylene- group, wherein any methylene group of the —(C₁-C₆) alkylene- may be replaced by a carbonyl, NRa, O, or S; the —(C₁-C₆) alkylene- may be substituted with 0, 1, 2, 3, or 4 substituents selected from halogen or C₁-C₃ alkyl; and two substituents on the same carbon atom may together with the carbon atom to which they are attached form a 3-6-membered ring, which optionally contains 0, 1, 2, or 3 heteroatoms selected from N, O, or S;
Cy₀ represents a 5-12-membered aromatic ring or heteroaromatic ring;
R_{A} is each independently selected from H, halogen, CN, C₁-C₆ alkyl, —(C₀-C₆ alkylene)-(C₃-C₈ cycloalkyl), —(C₀-C₆ alkylene)-(4-8-membered heterocycloalkyl), —(C₀-C₆ alkylene)-ORa, —(C₀-C₆ alkylene)-SRa, or —(C₀-C₆ alkylene)-NRaRa′; and, optionally, two R_{A} groups on adjacent or non-adjacent atoms of Cy₀ together with the ring atoms of Cy₀ may form a 6-10-membered ring, which may be further substituted with 0, 1, 2, 3, or 4 substituents selected from halogen, C₁-C₃ alkyl, —ORa, —SRa, or —NRaRa′;
L₀ is selected from a single bond, —(C₁-C₆) alkylene-, or —(C₂-C₆) alkenylene-, wherein any methylene group in the —(C₁-C₆) alkylene- or —(C₂-C₆) alkenylene- may be replaced by a carbonyl, NRa, O, or S; and the —(C₁-C₆) alkylene- or —(C₂-C₆) alkenylene- is optionally substituted with 0, 1, 2, 3, or 4 substituents selected from halogen or C₁-C₃ alkyl;
each R_{B} is independently selected from H, halogen, C₁-C₆ alkyl, —(C₀-C₆ alkylene)-ORa, —(C₀-C₆ alkylene)-SRa, or —(C₀-C₆ alkylene)-NRaRa′; and optionally, R_{B} may form a ring with a substituent R₄ on Cy₁;
R_{C} is selected from H, halogen, C₁-C₆ alkyl, —(C₀-C₆ alkylene)-ORa, —(C₀-C₆ alkylene)-SRa, or —(C₀-C₆ alkylene)-NRaRa′;
R_{D} is selected from H, C₁-C₆ alkyl, —(C₁-C₆ alkylene)-ORa, —(C₁-C₆ alkylene)-SRa, or —(C₁-C₆ alkylene)-NRaRa′;
each of R_{E} and R_{F} is independently selected from H, C₁-C₆ alkyl, halogen, —(C₀-C₆ alkylene)-ORa, —(C₀-C₆ alkylene)-SRa, or —(C₀-C₆ alkylene)-NRaRa′; and optionally, R_{E} and R_{F}, together with the carbon atom to which they are attached, may form a 3-6-membered ring, which may additionally contain 0, 1, or 2 heteroatoms selected from N, O, or S;
Z represents N or CRs; Z′ represents N or CR₅′; wherein R₅ and R₅′ are each independently selected from hydrogen, halogen, C₁-C₆ alkyl, —(C₀-C₆ alkylene)-(C₃-C₈ cycloalkyl), or —(C₀-C₆ alkylene)-CN;
wherein m, n, o, p, and q are each independently selected from 0, 1, 2, 3, or 4;
Ra, Ra′ are each independently selected from hydrogen, C₁-C₆ alkyl, C₃-C₈ cycloalkyl, or a 4-8-membered heterocycloalkyl; when Ra and Ra′ are attached to the same nitrogen atom, Ra and Ra′ together with the nitrogen atom to which they are attached may form a 4-8-membered ring, which may optionally contain 0, 1, 2, or 3 heteroatoms selected from N, O, or S;
the alkyl, cycloalkyl, heterocycloalkyl, and alkylene groups may each independently be substituted by 0, 1, 2, 3, 4, 5, or 6 halogen atoms.

In some embodiments, the compound of formula (A), or an isotopic derivative, stereoisomer, or a pharmaceutically acceptable salt thereof, has a structure represented by formula (A′):

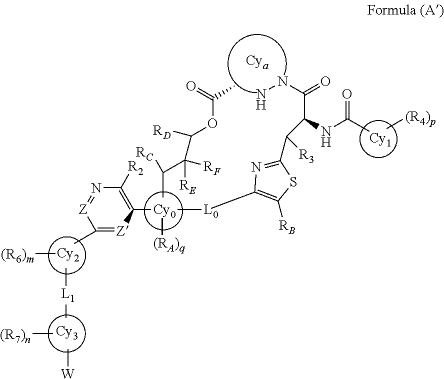

wherein R_{A}, R_{B}, R_{C}, R_{D}, R_{E}, R_{F}, Z, Z′, R₂, R₃, R₄, R₆, R₇, Cya, Cy₀, Cy₁, Cy₂, Cy₃, L₀, L₁, W, m, n, p, q are as defined in formula (A).

In some embodiments, R_{B} is H.

In some embodiments, R_{C}, R_{D} are H.

In some embodiments, Z represents CR₅ and Z′ represents CR₅′.

In some embodiments, L₀ is ethenylene, a (C₁-C₃) alkylene, or a single bond; more preferably, L₀ is a single bond.

In some embodiments, R_{E} and R_{F} are each independently selected from H and C₁-C₆ alkyl, and optionally, R_{E} and R_{F} together with the carbon atom to which they are attached form a 3- to 6-membered ring, which may optionally contain 0, 1, or 2 heteroatoms selected from N, O, and S; more preferably, R_{E} and R_{F} are each independently selected from C₁-C₃ alkyl.

In one aspect, the disclosure provides a compound, or an isotopic derivative, stereoisomer, or a pharmaceutically acceptable salt thereof, having the structure of formula (B):

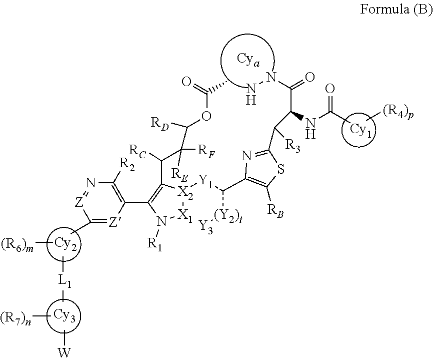

wherein:
--- represents a single bond or a double bond;
X₁, X₂ each independently represents C or N;
Y₁, Y₂, Y₃ each independently represent no bond formed or a single bond, CR_{A}, N, NR₁′, O, S, wherein, R₁′ represents C₁-C₆ alkyl, —(C₀-C₆ alkylene)-(C₃-C₈ cycloalkyl), —(C₀-C₆ alkylene)-(4-8-membered heterocycloalkyl), —(C₁-C₆ alkylene)-ORa, —(C₁-C₆ alkylene)-SRa or —(C₁-C₆ alkylene)-NRaRa′; wherein, when Y₃ is CR_{A}, R₁ and R_{A} substituent on Y₃, together with the N to which R₁ is connected, X₁, and C atoms, may optionally form a 6- to 10-membered ring, which may further be substituted with 0, 1, 2, 3, or 4 substituents selected from halogen, C₁-C₃ alkyl, —ORa, —SRa or —NRaRa′;
R₁ represents C₁-C₆ alkyl, —(C₀-C₆ alkylene)-(C₃-C₈ cycloalkyl), —(C₀-C₆ alkylene)-(4-8-membered heterocycloalky), —(C₁-C₆ alkylene)-ORa, —(C₁-C₆ alkylene)-SRa or —(C₁-C₆ alkylene)-NRaRa′;
t is selected from 0, 1, 2, or 3;
R_{A}, R_{B}, R_{C}, R_{D}, R_{E}, R_{F}, Z, Z′, R₂, R₃, R₄, R₆, R₇, Cya, Cy₁, Cy₂, Cy₃, L₁, W, m, n, p are as defined in any of the preceding embodiments.

In some embodiments, the compound represented by formula (B), or an isotopic derivative, stereoisomer, or a pharmaceutically acceptable salt thereof, has the structure of formula (B′):

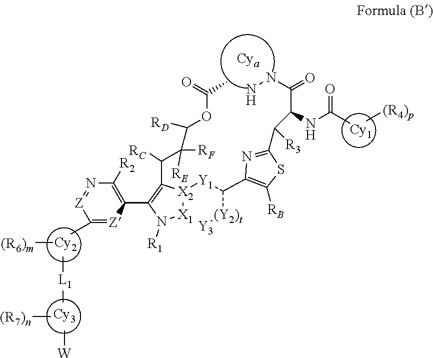

wherein: ---, X₁, X₂, Y₁, Y₂, Y₃, R_{A}, R_{B}, R_{C}, R_{D}, R_{E}, R_{F}, Z, Z′, R₁, R₂, R₃, R₄, R₆, R₇, Cya, Cy₁, Cy₂, Cy₃, L₁, W, m, n, p, t are as defined in formula (B).

In some embodiments, X₁, X₂ are each independently C.

In some embodiments,

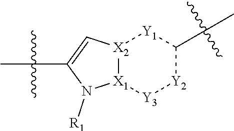

is selected from

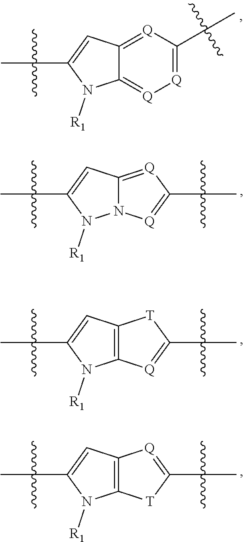

wherein, Q represents CR_{A} or N, T represents NR₁′, O or S; and optionally, when Y₃ is CR_{A}, R₁ may, together with the substituent R_{A} on Y₃ and the ring atoms of Cy₀, form a 6-10-membered ring, which 6-10-membered ring may be further substituted with 0, 1, 2, 3, or 4 substituents independently selected from halogen, C₁-C₃ alkyl, —ORa, —SRa or —NRaRa′.

In some embodiments,

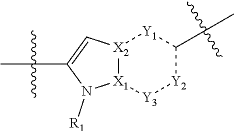

represents

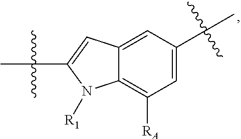

and optionally, R₁ may, together with the R_{A} substituent of Y₃ and the N to which R₁ is connected, X₁, and C atoms, form a 6- to 10-membered ring, which may further be substituted with 0, 1, 2, 3, or 4 substituents selected from halogen, C₁-C₃ alkyl, and —OH.

In some embodiments,

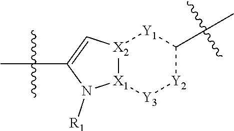

represents

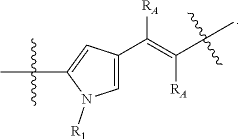

In some embodiments, R_{A} is each independently selected from H, halogen, CN, or C₁-C₃ alkyl; more preferably, R_{A} is each independently H or F.

In one aspect, the disclosure provides a compound having the structure of formula (I), or an isotopic derivative, stereoisomer, or a pharmaceutically acceptable salt thereof:

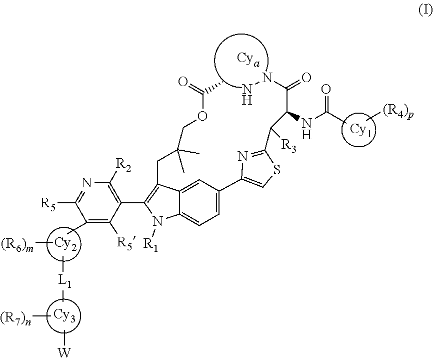

wherein:
R₁ represents C₁-C₆ alkyl, —(C₀-C₆ alkylene)-(C₃-C₈ cycloalkyl), —(C₀-C₆ alkylene)-(4-8-membered heterocycloalkyl), —(C₁-C₆ alkylene)-ORa, —(C₁-C₆ alkylene)-SRa or —(C₁-C₆ alkylene)-NRaRa′;
R₂ represents halogen, cyano, C₁-C₆ alkyl, —(C₀-C₆ alkylene)-(C₃-C₈ cycloalkyl), or —(C₀-C₆ alkylene)-(4-8-membered heterocycloalkyl), which may optionally be substituted with 0, 1, or 2 substituents selected from: —ORa, —SRa, or —NRaRa′;
R₃ represents hydrogen, hydroxy, —(C₁-C₃ alkylene)-ORₐ, or C₁-C₃ alkyl;
Cy₁ represents C₃-C₁₀ cycloalkyl or 4-10-membered heterocycloalkyl, and Cy₁ may be a mono ring, spiro ring, bridged ring, or fused ring;
R₄ each independently represents hydrogen, halogen, oxo, C₁-C₆ alkyl, —(C₀-C₆ alkylene)(C₃-C₆) cycloalkyl, —(C₀-C₆ alkylene)(4-8-membered) heterocycloalkyl, —(C₀-C₆ alkylene)ORa, —(C₀-C₆ alkylene)SRa, —(C₀-C₆ alkylene)NRaRa′, —(C₀-C₆ alkylene)CORa, —(C₀-C₆ alkylene)COORa, —(C₀-C₆ alkylene)CONRaRa′, —(C₀-C₆ alkylene) NRaCORa′, —(C₀-C₆ alkylene) OCONRaRa′, —(C₀-C₆ alkylene) NRaCONRaRa′, —(C₀-C₆ alkylene)SORa, —(C₀-C₆ alkylene)S(O)₂Ra, —(C₀-C₆ alkylene)NRaS(O)₂Ra′, —(C₀-C₆ alkylene)CN, —(C₀-C₆ alkylene) (C₆-C₁₀ aryl) or —(C₀-C₆ alkylene)(5-12-membered heteroaryl); wherein the R₄ substituents on two carbon atoms of Cy₁, together with the carbon atoms to which they are attached and the atoms between said two carbon atoms, may form a 3-8 membered ring, which may optionally contain 0, 1, 2, or 3 heteroatoms selected from N, O, or S; alternatively, the two R₄ substituents on the same carbon atom of Cy₁, together with the carbon atom to which they are attached, may form a 3-8 membered ring, which may optionally contain 0, 1, 2, or 3 heteroatoms selected from N, O, or S;
R₅, R₅′ each independently represents hydrogen, halogen, C₁-C₆ alkyl, —(C₀-C₆ alkylene)(C₃-C₈) cycloalkyl or —(C₀-C₆ alkylene)CN;
Cy₂ represents a 5-membered heteroaryl;
Cy₃ represents C₃-C₁₂ cycloalkyl or 4-12-membered heterocycloalkyl, which may be a mono ring, spiro ring, bridged ring, or fused ring;
R₆ and R₇ are each independently selected from: hydrogen, halogen, oxo, —ORa, —NRaRa′, cyano, C₁-C₆ alkyl, C₃-C₈ cycloalkyl or 4-8-membered heterocycloalkyl; each of the above-mentioned C₁-C₆ alkyl, C₃-C₈ cycloalkyl, 4-8-membered heterocycloalkyl may be independently substituted with 0, 1, 2, 3, or 4 substituents selected from halogen, oxo, —ORa, —SRa, —NRaRa′, cyano, C₁-C₆ alkyl, C₃-C₈ cycloalkyl or 4-8-membered heterocycloalky;
W is H or represented by formula (II), wherein * represents the site where W is attached to Cy₃:

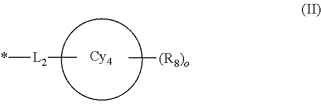

Cy₄ is selected from C₃-C₁₂ cycloalkyl or 4-12-membered heterocycloalkyl, which may be a mono ring, spiro ring, bridged ring, or fused ring;
R₈ is each independently selected from: hydrogen, halogen, oxo, —ORa, —NRaRa′, cyano, C₁-C₆ alkyl, wherein the C₁-C₆ alkyl may be substituted with 0, 1, 2, 3, or 4 substituents selected from halogen, oxo, —ORa, —NRaRa′, or cyano;
L₁, L₂ each independently represents a single bond or a —(C₁-C₆) alkylene- group, wherein any methylene group of the —(C₁-C₆) alkylene- may be replaced by a carbonyl, NRa, O or S; the —(C₁-C₆) alkylene- may be substituted with 0, 1, 2, 3, or 4 substituents selected from halogen or C₁-C₃ alkyl; and two substituents on the same carbon atom may together with the carbon atom to which they are attached form a 3-6-membered ring, which optionally contains 0, 1, 2, or 3 heteroatoms selected from N, O, or S;
wherein m, n, o, p, and q are each independently selected from 0, 1, 2, 3, or 4;
Ra and Ra′ are each independently selected from hydrogen, C₁-C₆ alkyl, C₃-C₈ cycloalkyl, or a 4-8-membered heterocycloalkyl; when Ra and Ra′ are attached to the same nitrogen atom, Ra and Ra′ together with the nitrogen atom to which they are attached may form a 4-8-membered ring, which may optionally contain 0, 1, 2, or 3 heteroatoms selected from N, O, or S;
the alkyl, cycloalkyl, heterocycloalkyl, and alkylene groups may each independently be substituted by 0, 1, 2, 3, 4, 5, or 6 halogen atoms.

In some embodiments, the compound represented by formula (I), or an isotopic derivative, stereoisomer, or a pharmaceutically acceptable salt thereof, has the structure of formula (I′):

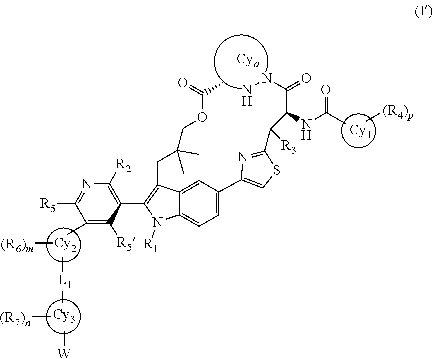

wherein: R₁, R₂, R₃, R₄, R₅, R₅′, R₆, R₇, Cya, Cy₁, Cy₂, Cy₃, L₁, W, m, n, p are as defined in (I).

In some embodiments of the disclosure, Cya represents

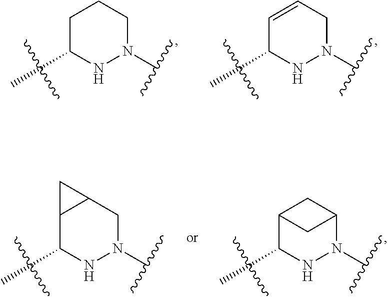

or which is optionally substituted with 0, 1, 2, or 3 substituents selected from halogen or C₁-C₃ alkyl.

In some preferable embodiments of the disclosure, Cya represents

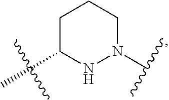 [SMILES: *N1CCC[C@@H](C)N1]

which is optionally substituted with 0, 1, 2, or 3 substituents selected from halogen or C₁-C₃ alkyl; more preferably, Cya represents

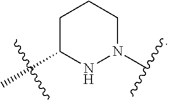 [SMILES: *N1CCC[C@@H](C)N1]

In some embodiments of the disclosure, Cya represents

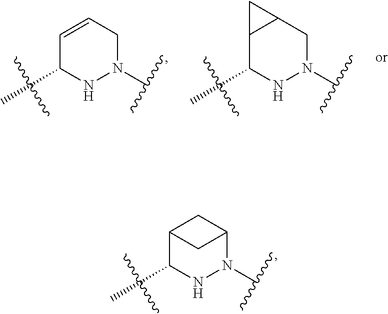

which is optionally substituted with 0, 1, 2, or 3 substituents selected from halogen or C₁-C₃ alkyl; preferably, Cya represents

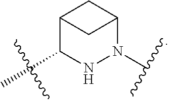 [SMILES: *N1N[C@H](C)C2CC1C2]

In some embodiments of the disclosure, W is represented by (II).

In some embodiments of the disclosure, L₁, L₂ each independently represent a single bond or a —(C₁-C₆)alkylene- group, wherein any methylene group of the —(C₁-C₆)alkylene- may optionally be replaced by a carbonyl, NRa, O, or S, and the —(C₁-C₆)alkylene- group may be substituted with 0, 1, 2, 3, or 4 C₁-C₃ alkyl substituents, and two substituents on the same carbon atom may together form a 3- to 6-membered ring with the carbon atom to which they are attached, wherein the 3- to 6-membered ring optionally contains 0, 1, 2, or 3 heteroatoms selected from N, O, or S.

In some embodiments of the disclosure, R₁ represents a C₁-C₆ alkyl group, a —(C₀-C₆ alkylene)-(C₃-C₈ cycloalkyl) group, a —(C₀-C₆ alkylene)-(4-8-membered heterocycloalkyl) group, a halogen-substituted C₁-C₆ alkyl group, a halogen-substituted —(C₀-C₆ alkylene)-(C₃-C₈ cycloalkyl) group, or a halogen-substituted —(C₀-C₆ alkylene)-(4-8-membered heterocycloalkyl) group; preferably, represents C₁-C₆ alkyl, —(C₀-C₆ alkylene)-(C₃-C₈ cycloalkyl), —(C₀-C₆ alkylene)-(4-8-membered heterocycloalkyl), halogen-substituted C₁-C₆ alkyl, halogen-substituted —(C₀-C₆ alkylene)-(C₃-C₈ cycloalkyl), halogen-substituted —(C₀-C₆ alkylene)-(4-8-membered heterocycloalkyl) ∘

In some embodiments of the disclosure, R₁ represents C₁-C₆ alkyl, —(C₀-C₆ alkylene)-(C₃-C₈ cycloalkyl), —(C₀-C₆ alkylene)-(4-8-membered heterocycloalkyl) ∘

In some embodiments of the disclosure, R₁ represents C₁-C₆ alkyl or halogen-substituted C₁-C₆ alkyl; more preferably, R₁ represents ethyl or —CH₂CF₃ ∘

In some embodiments of the disclosure, R₂ represents a C₁-C₆ alkyl group optionally substituted with 0 or 1 C₁-C₃ alkoxy group; preferably, R₂ represents 1-methoxyethyl; more preferably, R₂ represents

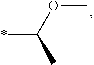

wherein, * represents the site where R₂ is attached to the corresponding position in formula.

In some embodiments of the disclosure, R₃ is preferably H.

In some embodiments of the disclosure, Cy₁ represents a C₃-C₈ cycloalkyl group or a 4-8 membered heterocycloalkyl group; preferably, Cy₁ represents a C₃-C₈ cycloalkyl group; more preferably, Cy₁ represents a cyclopropyl group.

In some embodiments of the disclosure, R₄ each independently represents hydrogen, halogen, or a C₁-C₃ alkyl group.

In some embodiments of the disclosure, the structure of -Cy₁-(R₄)p is selected from the following:

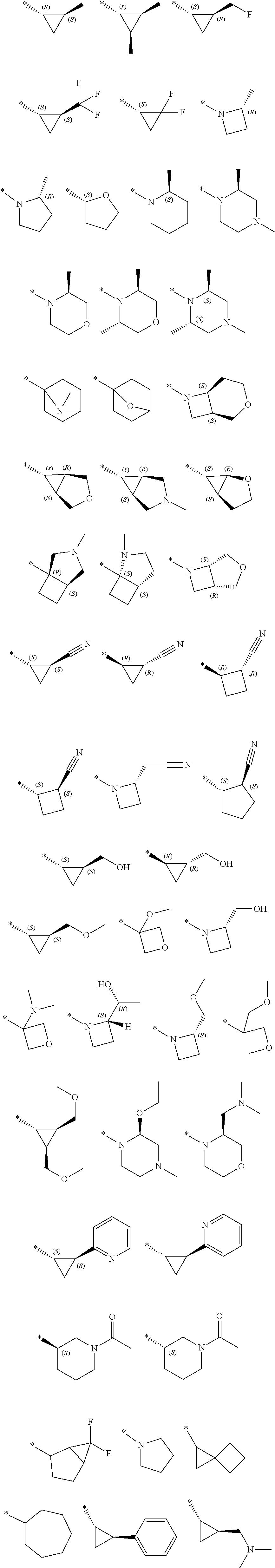

wherein, * represents the site where -Cy₁-(R₄)p is attached to the corresponding position in formula.

In some preferable embodiments of the disclosure,

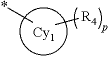

represents

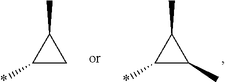

wherein, * represents the site where Cy₁ is attached to the corresponding position in formula.

In some embodiments of the disclosure, R₅, R₅′ are each independently selected from hydrogen, halogen, or C₁-C₆ alkyl; preferably, R₅ and R₅′ are hydrogen.

In some embodiments of the disclosure,

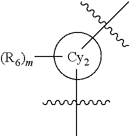

is selected from

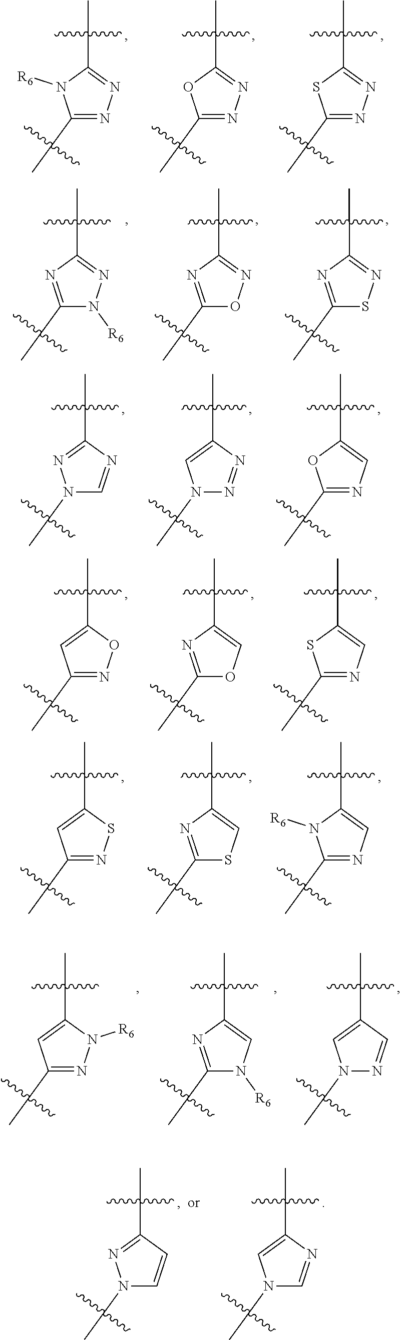

In some embodiments of the disclosure, Cy₂ is not pyrazole or isoxazole.

In some embodiments of the disclosure, Cy₂ is not

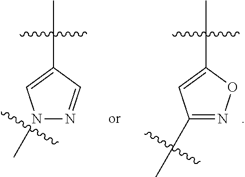

In some embodiments of the disclosure,

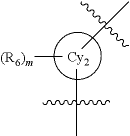

is not

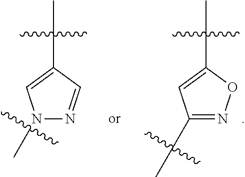

In some embodiments of the disclosure,

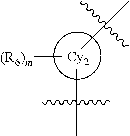

is selected from

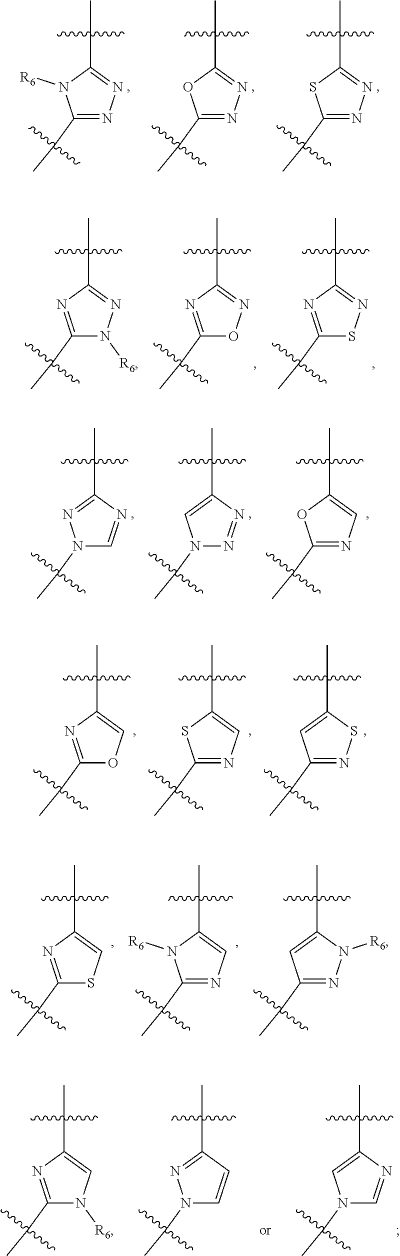

preferably,

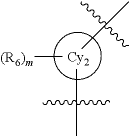

is selected from

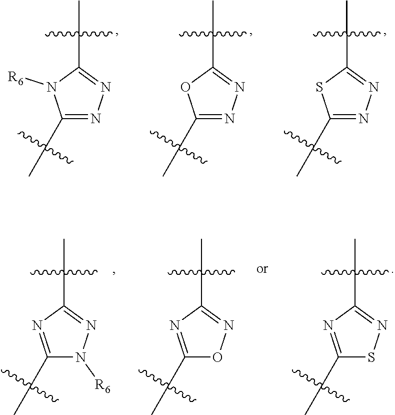

In some embodiments of the disclosure, Cy₃ represents a 4- to 12-membered heterocycloalkyl, which may be a mono ring, spiro ring, bridged ring, or fused ring; preferably, Cy₃ represents a 4- to 8-membered heterocycloalkyl, which may be a mono ring, spiro ring, bridged ring, or fused ring.

In some embodiments of the disclosure, Cy₃ represents

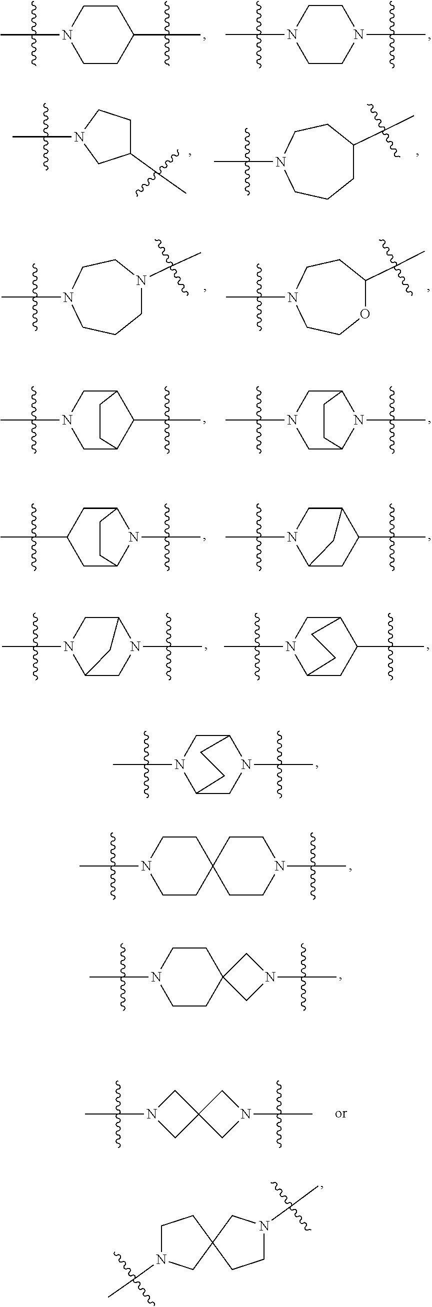

and Cy₃ may have n substituents selected from R₇; more preferably, Cy₃ represents

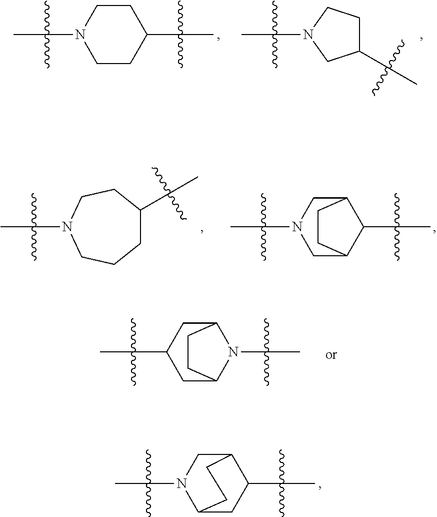

and Cy₃ may have n substituents selected from R₇.

In some embodiments of the disclosure, R₆ is each independently selected from hydrogen, C₁-C₆ alkyl, C₃-C₈ cycloalkyl, or 4- to 8-membered heterocycloalkyl, each of said C₁-C₆ alkyl, C₃-C₈ cycloalkyl, and 4- to 8-membered heterocycloalkyl being independently optionally substituted with 0, 1 or 2 substituents selected from halogen, oxo, —ORa, —NRaRa′, cyano, C₁-C₆ alkyl, C₃-C₈ cycloalkyl, or 4- to 8-membered heterocycloalkyl; preferably, R₆ is each independently selected from hydrogen, C₁-C₆ alkyl or C₃-C₈ cycloalkyl, each of said C₁-C₆ alkyl and C₃-C₈ cycloalkyl being independently optionally substituted with 0, 1 or 2 substituents selected from halogen, C₁-C₆ alkyl or C₃-C₆ cycloalkyl; more preferably, R₆ is each independently selected from hydrogen or methyl.

In some embodiments of the disclosure, R₇ is each independently selected from: hydrogen, halogen, oxo, —ORa, —NRaRa′, cyano, or C₁-C₆ alkyl, wherein each of said C₁-C₆ alkyl is independently optionally substituted with 0, 1, or 2 substituents selected from halogen, oxo, —ORa, —NRaRa′, or cyano; preferably, R₇ is each independently selected from hydrogen, halogen, oxo, hydroxy, cyano, or C₁-C₆ alkyl.

In some embodiments of the disclosure, L₁ represents a single bond, —CH₂—, —O—, or —NH—.

In some embodiments of the disclosure,

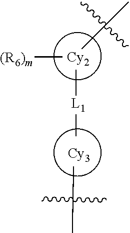

represents

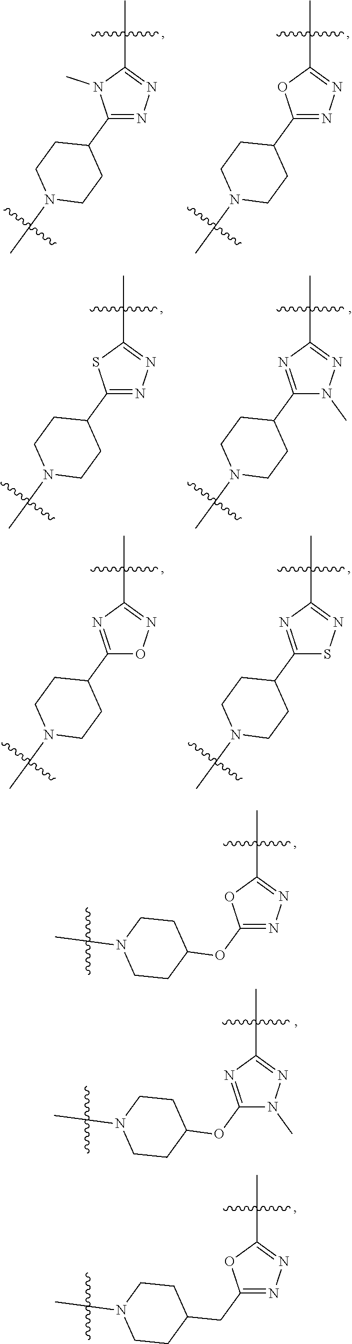

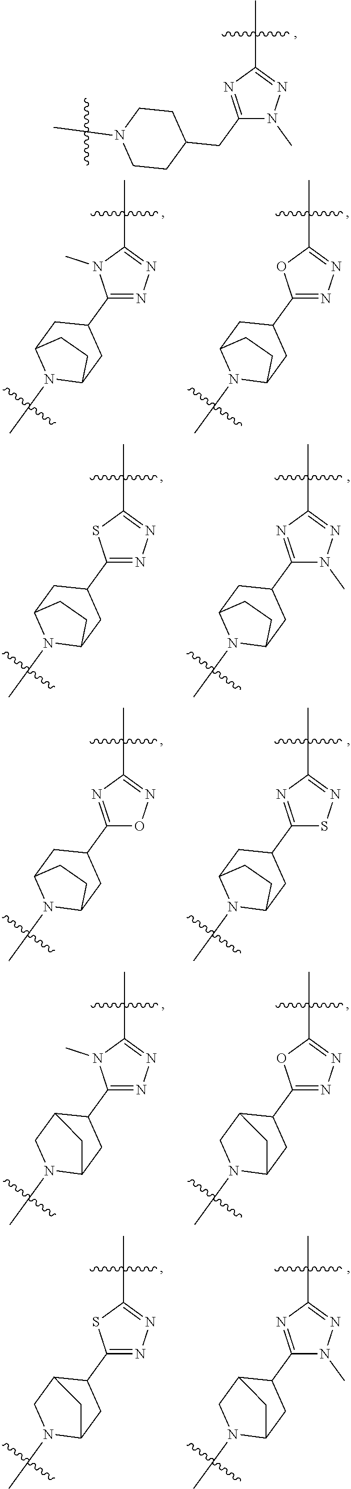

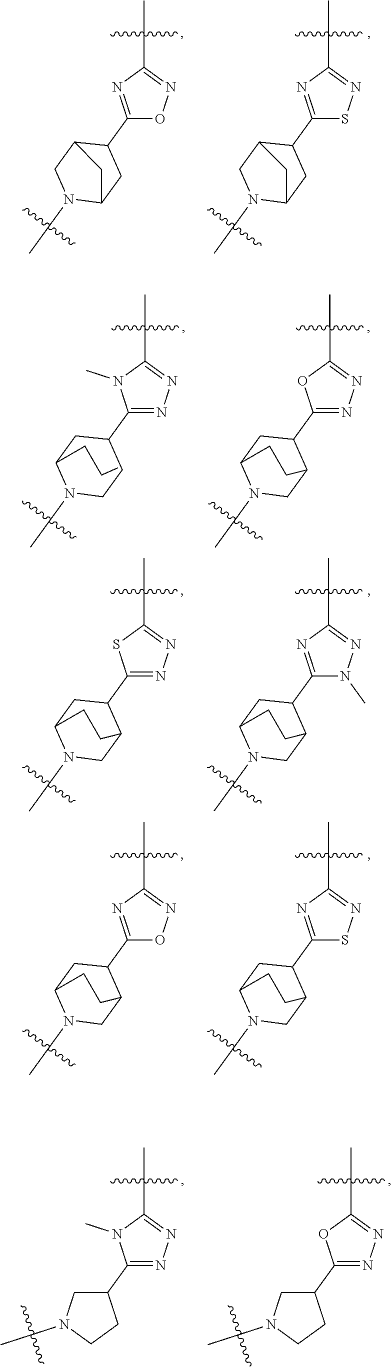

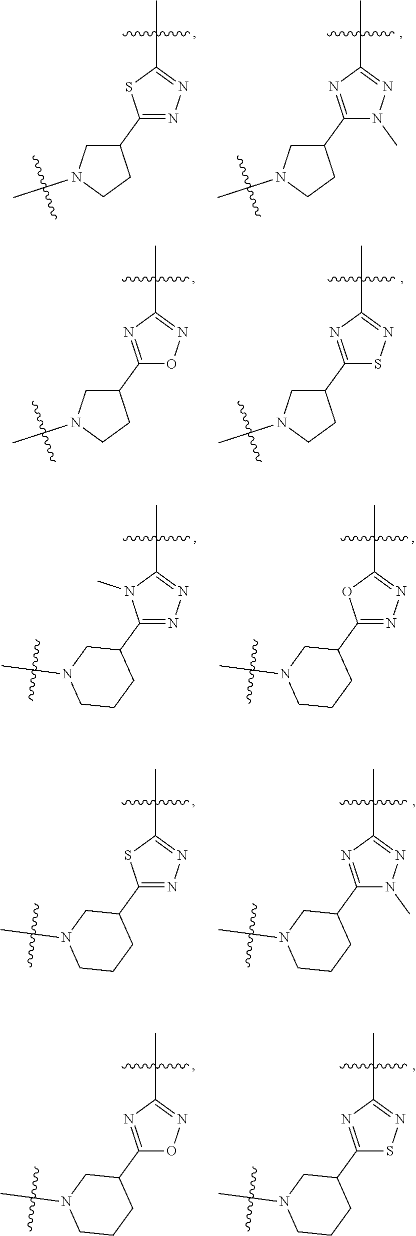

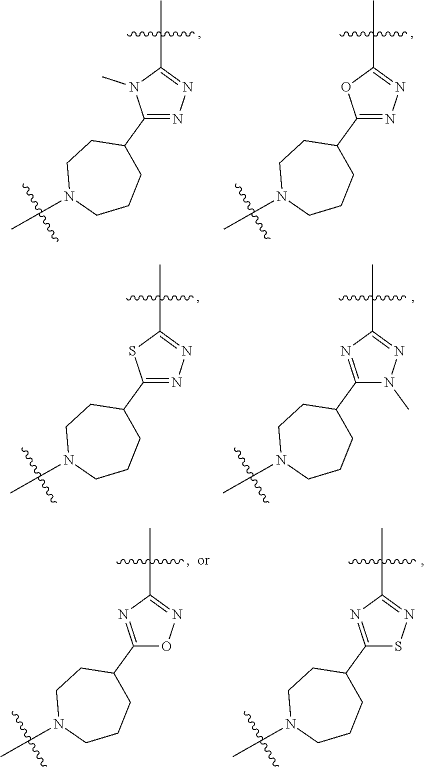 [SMILES: *c1nnc(C2CCCN(*)CC2)n1C ; *c1nnc(C2CCCN(*)CC2)o1 ; *c1nnc(C2CCCN(*)CC2)s1 ; *c1nc(C2CCCN(*)CC2)n(C)n1 ; *c1noc(C2CCCN(*)CC2)n1 ; *c1nsc(C2CCCN(*)CC2)n1]

and Cy₃ may have n substituents selected from R₇.

In some embodiments of the disclosure, Cy₄ is a C₅-C₆ cycloalkyl or a 5-6 membered heterocycloalkyl, o is ≥1, and R₈ is not hydrogen.

In some embodiments of the disclosure, Cy₄ is selected from C₃-C₄ cycloalkyl or 4-membered heterocycloalkyl.

In some embodiments of the disclosure, Cy₄ is selected from

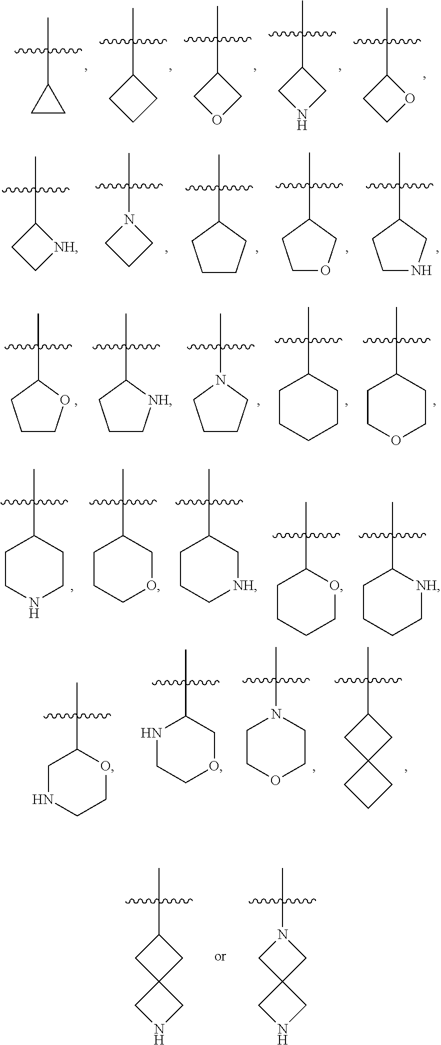

and Cy₄ may have o substituents selected from R₈; preferably, Cy₄ is selected from

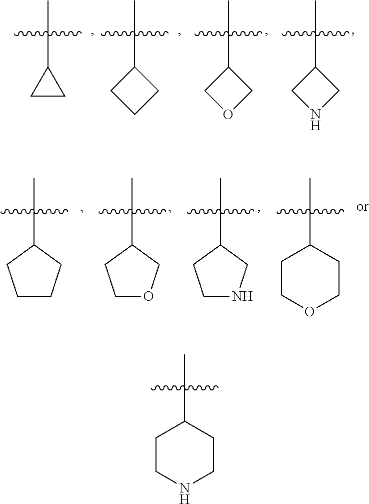

and Cy₄ may have o substituents selected from R₈; more preferably, Cy₄ is selected from

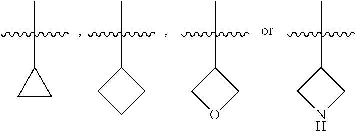

and Cy₄ may have o substituents selected from R₈.

In some embodiments of the disclosure, L₂ represents a single bond, —CH₂—, or —O—; preferably, L₂ represents a single bond.

In some embodiments of the disclosure, R₈ is each independently selected from hydrogen, halogen, or C₁-C₆ alkyl.

In some embodiments of the disclosure,

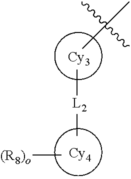

represents

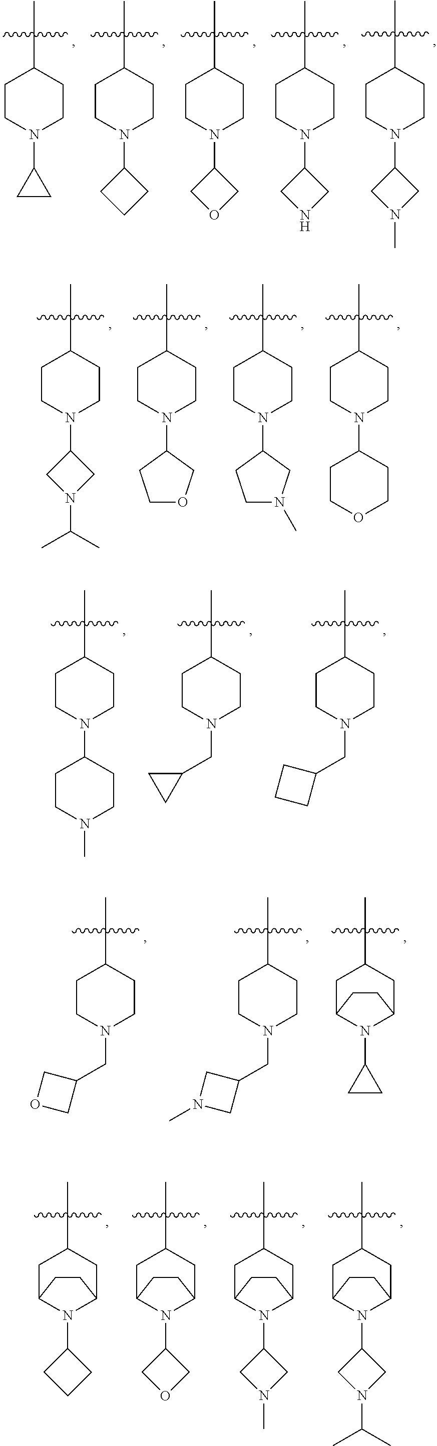

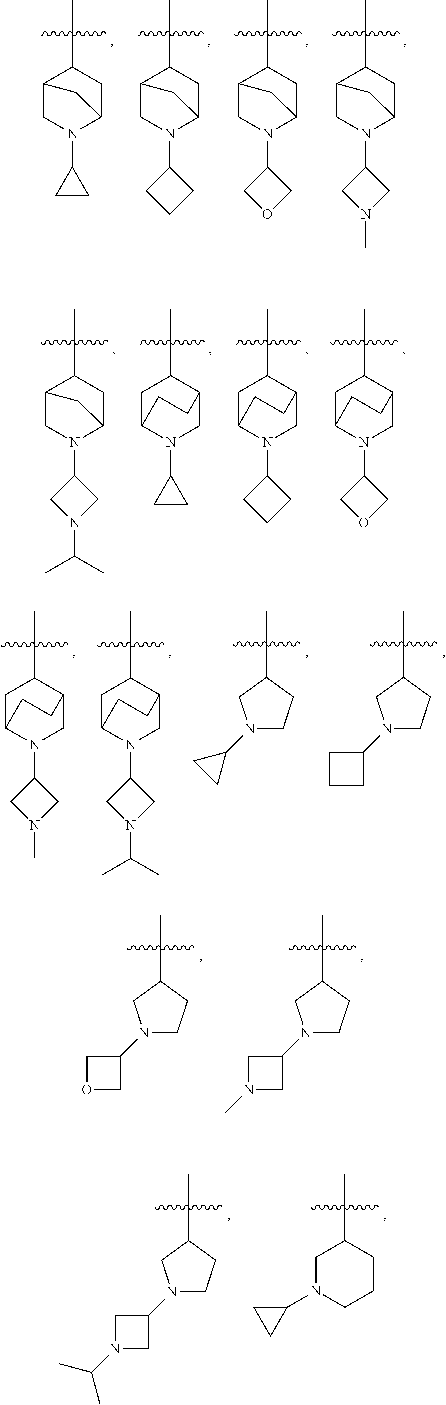

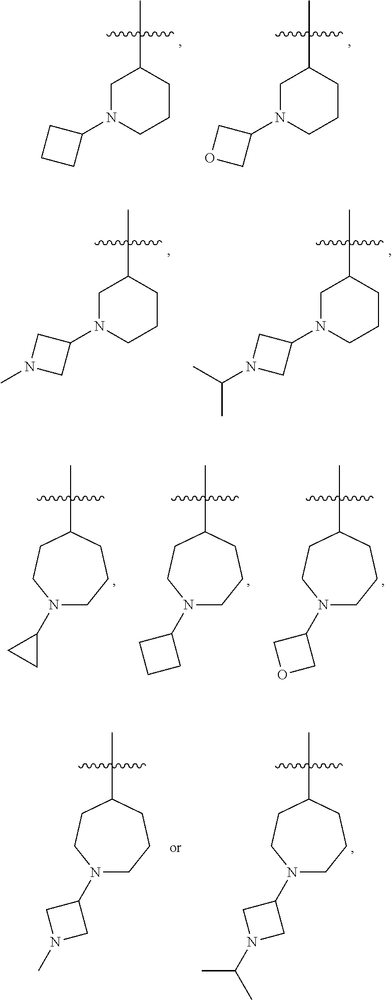

and Cy₃ may have n substituents selected from R₇.

In some embodiments of the disclosure,

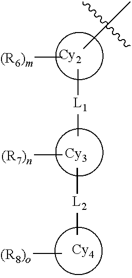

represents

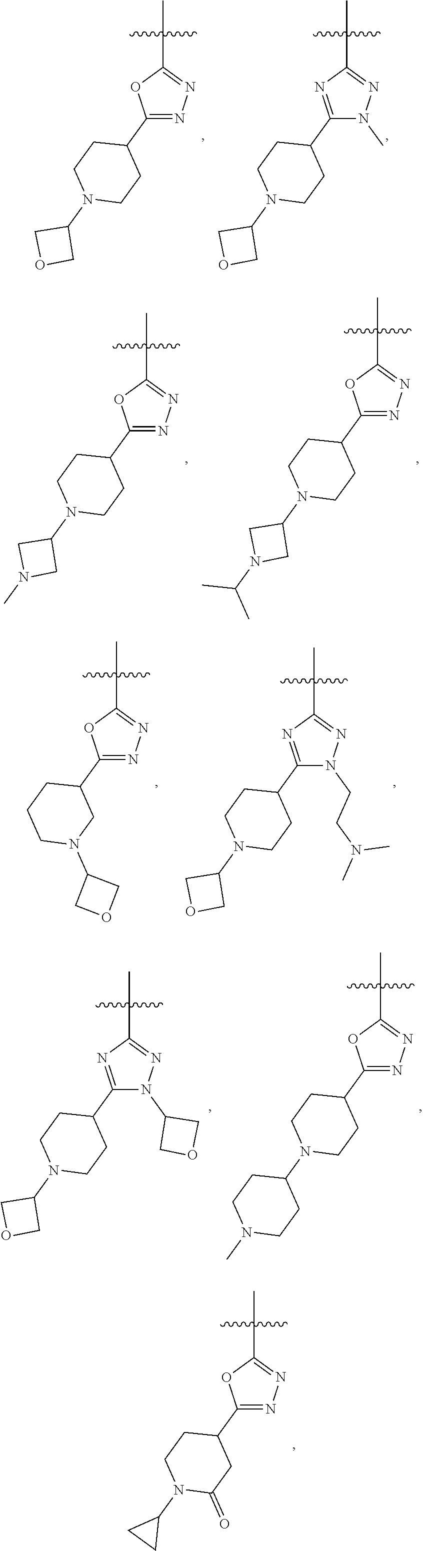

In some embodiments of the disclosure, m, n, o, p, q, t are each independently 0, 1, or 2.

In some embodiments of the disclosure, Ra, Ra′ are each independently selected from hydrogen, C₁-C₆ alkyl, C₃-C₈ cycloalkyl, or 4- to 8-membered heterocycloalkyl; preferably, Ra and Ra′ are each independently selected from hydrogen or C₁-C₆ alkyl; more preferably, Ra and Ra′ are each independently selected from hydrogen or C₁-C₃ alkyl.

In one aspect, the disclosure provides a compound having a structure represented by formula (III), or an isotopic derivative, stereoisomer, or a pharmaceutically acceptable salt thereof:

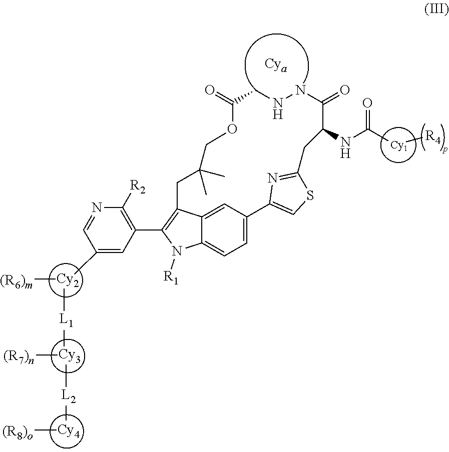

wherein:
R₁ represents ethyl or —CH₂CF₃;
R₂ represents C₁-C₆ alkyl, which may be substituted with 0 or 1 C₁-C₃ alkoxy;
Cy₁ represents a C₃-C₈ cycloalkyl group or a 4-8 membered heterocycloalkyl group; preferably, Cy₁ represents C₃-C₈ cycloalkyl;
R₄ each independently represents hydrogen, halogen, or C₁-C₃ alkyl;
Cy₂ represents a 5-membered heteroaryl group;
Cy₃ represents a 4-12-membered heterocycloalkyl;
R₆ is each independently selected from hydrogen, C₁-C₆ alkyl, C₃-C₈ cycloalkyl or 4-8-membered heterocycloalkyl, wherein each of the C₁-C₆ alkyl, C₃-C₈ cycloalkyl, 4-8-membered heterocycloalkyl is independently optionally substituted with 0, 1, or 2 substituents selected from halogen, oxo, —ORa, —NRaRa′, cyano, C₁-C₆alkyl, C₃-C₈ cycloalkyl or 4-8-membered heterocycloalkyl;
R₇ is each independently selected from: hydrogen, halogen, oxo, —ORa, —NRaRa′, cyano, and C1-C6 alkyl, wherein the C1-C6 alkyl is independently optionally substituted with 0, 1, or 2 substituents selected from halogen, oxo, —ORa, —NRaRa′, and cyano;
L₁ represents a single bond or —(C1-C6)alkylene-, wherein any methylene in the —(C1-C6)alkylene- may be replaced by carbonyl, NRa, O, or S;
Cy₄ is selected from C3-C12 cycloalkyl or 4-12-membered heterocycloalkyl, which may be a mono ring, spiro ring, bridged ring, or fused ring; and when Cy4 is a C5-C6 cycloalkyl or a 5-6-membered heterocycloalkyl, o≥1 and R₈ is not hydrogen;
L₂ represents a single bond or —(C₁-C₆)alkylene-, wherein any methylene group in the —(C₁-C₆)alkylene- may optionally be replaced by a carbonyl, NRa, O or S;
R₈ is each independently selected from: hydrogen, halogen, oxo, —ORa, —NRaRa′, cyano, and C₁-C₆ alkyl, wherein the C₁-C₆ alkyl may be substituted with 0, 1, 2, 3, or 4 substituents selected from halogen, oxo, —ORa, —NRaRa′, and cyano;
m, n, o, p each independently represent 0, 1, or 2;
Ra, Ra′ each independently represent hydrogen, C₁-C₆ alkyl, C₃-C₈ cycloalkyl, or 4-8-membered heterocycloalkyl;
the alkyl, cycloalkyl, heterocycloalkyl, and alkylene groups are each independently optionally substituted with 0, 1, 2, 3, 4, 5, or 6 halogen atoms.

In some embodiments, the compound represented by formula (III), or an isotopic derivative, stereoisomer, or a pharmaceutically acceptable salt thereof, has a structure represented by formula (III′):

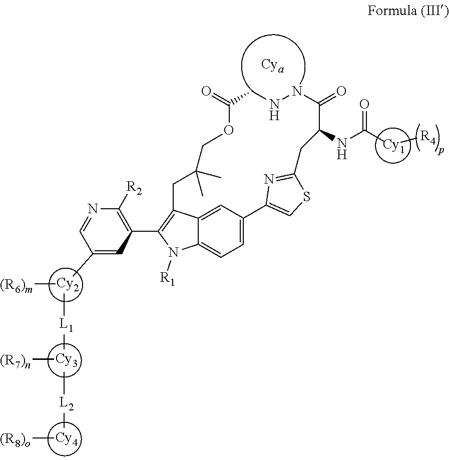

wherein, R₁, R₂, R₄, R₆, R₇, R₈, Cya, Cy₁, Cy₂, Cy₃, Cy₄, L₁, L₂, m, n, o, p are as defined in formula (III).

In some embodiments of the disclosure, Cya represents

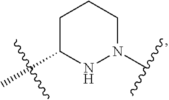 [SMILES: *N1CCC[C@@H](C)N1]

which is optionally substituted with 0, 1, 2, or 3 substituents selected from halogen or C1-C3 alkyl; preferably, Cya represents

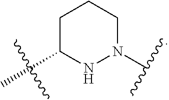 [SMILES: *N1CCC[C@@H](C)N1]

In some embodiments of the disclosure, Cya represent

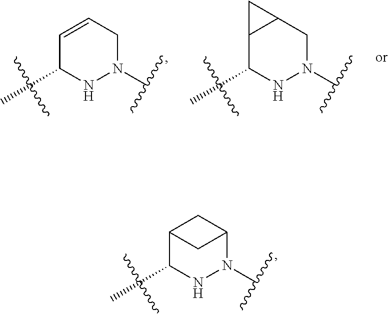

which is optionally substituted with 0, 1, 2, or 3 substituents selected from halogen or C1-C3 alkyl; preferably, Cya represents

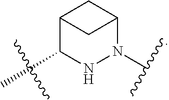 [SMILES: *N1N[C@H](C)C2CC1C2]

In some embodiments of the disclosure, R₂ represents 1-methoxyethyl; preferably, R₂ represents

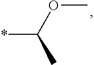

wherein, * represents the site where R₂ is attached to the corresponding position in formula (III).

In some embodiments of the disclosure, Cy₁ represents a C₃-C₈ cycloalkyl group; preferably, Cy₁ represents a cyclopropyl group.

In some embodiments of the disclosure,

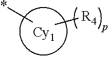

represents

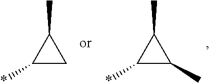

wherein, * represents the site where Cy₁ is attached to the corresponding position in formula (III).

In some embodiments of the disclosure, Cy₂ is not

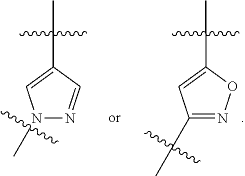

In some embodiments of the disclosure, Cy₂ is not pyrazole or isoxazole.

In some embodiments of the disclosure,

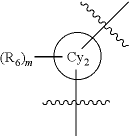

is selected from

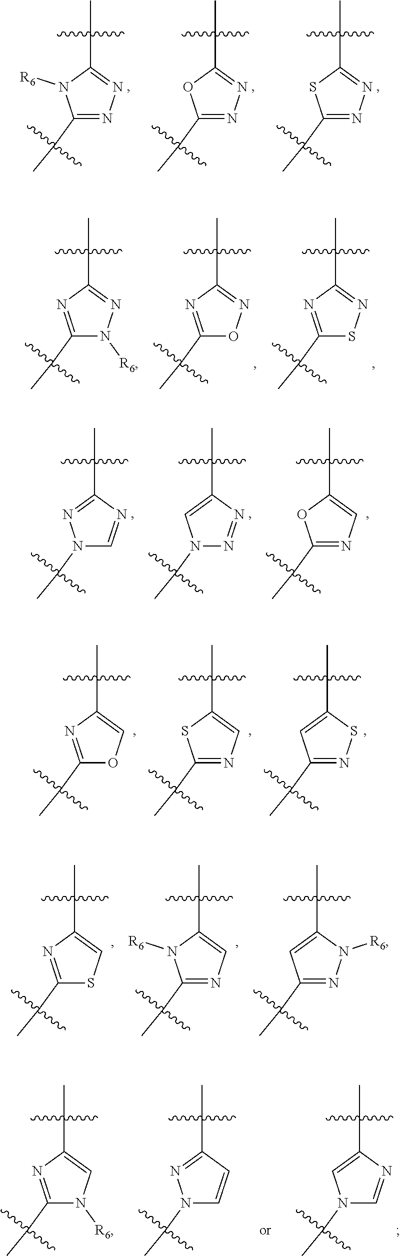

preferably,

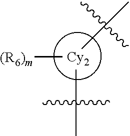

is selected from

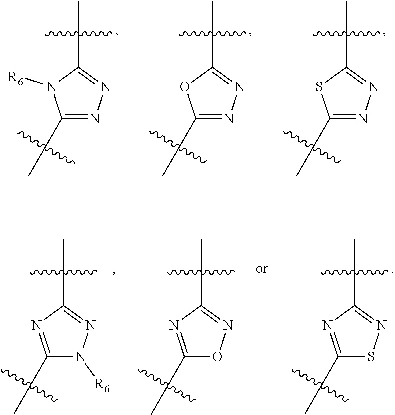

In some embodiments of the disclosure, R₆ is each independently selected from hydrogen, C₁-C₆ alkyl, or C₃-C₈ cycloalkyl, each of the C₁-C₆ alkyl and C₃-C₈ cycloalkyl being independently optionally substituted with 0, 1, or 2 substituents selected from halogen, C₁-C₆ alkyl, or C₃-C₆ cycloalkyl; more preferably, R₆ is each independently selected from hydrogen or methyl.

In some embodiments of the disclosure, R₇ is each independently selected from: hydrogen, halogen, oxo, hydroxy, cyano, or C₁-C₆ alkyl.

In some embodiments of the disclosure, L₁ represents a single bond, —CH₂—, —O—, or —NH—.

In some embodiments of the disclosure, Cy₄ is a C₅-C₆ cycloalkyl group or a 5- to 6-membered heterocycloalkyl group, o≥1, and R₈ is not hydrogen.

In some embodiments of the disclosure, Cy₄ is selected from a C₃-C₄ cycloalkyl group or a 4-membered heterocycloalkyl group.

In some embodiments of the disclosure, L₂ represents a single bond, —CH₂—, or —O—; preferably, L₂ represents a single bond.

In some embodiments of the disclosure, R₈ is each independently selected from hydrogen, halogen, or C₁-C₆ alkyl.

In some embodiments of the disclosure, Ra and Ra′ are each independently hydrogen or C₁-C₆ alkyl; preferably, Ra and Ra′ are each independently hydrogen or C₁-C₃ alkyl.

In another aspect, the disclosure provides a compound having the structure of formula (IV), or an isotopic derivative thereof, a stereoisomer thereof, or a pharmaceutically acceptable salt thereof:

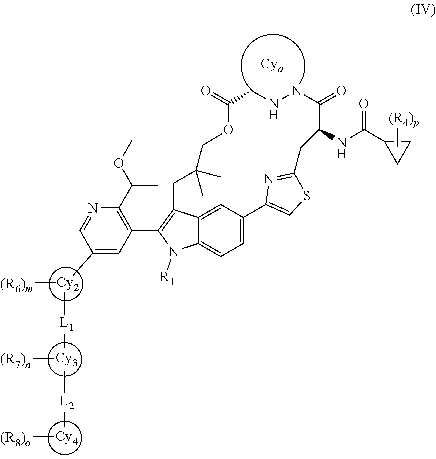

wherein:
R₁ represents ethyl or —CH₂CF₃;
R₄ each independently represents hydrogen, halogen, or C₁-C₃ alkyl;

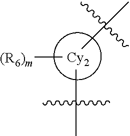

is selected from

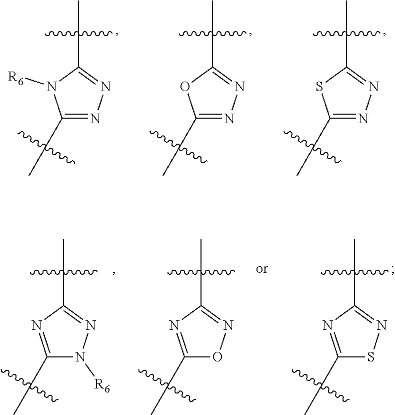

Cy₃ is selected from 4- to 8-membered heterocycloalkyl;
Cy₄ is selected from C₃-C₄ cycloalkyl or 4-membered heterocycloalkyl;
R₆ is each independently selected from hydrogen and methyl;
R₇ is each independently selected from hydrogen, halogen, oxo, or C₁-C₆ alkyl;
R₈ is each independently selected from hydrogen, halogen, or C₁-C₆ alkyl;
L₁ represents a single bond, —CH₂—, or —O—;
L₂ represents a single bond, —CH₂—, or —O—;
wherein n, o, and p are each independently 0, 1, or 2;
Ra, Ra′ are each independently hydrogen or C₁-C₆ alkyl; preferably, Ra, Ra′ are each independently hydrogen or C₁-C₃ alkyl;
the alkyl, cycloalkyl, heterocycloalkyl, and alkylene groups may each independently be substituted with 0, 1, 2, 3, 4, 5, or 6 halogen atoms.

In some embodiments, the compound of formula (IV), or an isotopic derivative thereof, a stereoisomer thereof, or a pharmaceutically acceptable salt thereof, has the structure of formula (IV′):

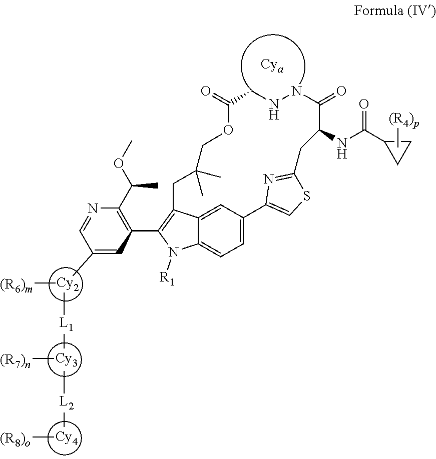

wherein, R₁, R₄, R₆, R₇, R₈, Cya, Cy₂, Cy₃, Cy₄, L₁, L₂, m, n, o, p are as defined in Formula (IV).

In some embodiments of the disclosure,

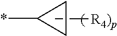

represents

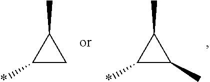

wherein, * represents the site where Cy₁ is attached to the corresponding position in the formula.

In certain preferred embodiments of the disclosure, Cya represents

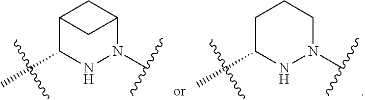

In another aspect, the disclosure provides a compound having the following structure, or an isotopic derivative thereof, a stereoisomer thereof, or a pharmaceutically acceptable salt thereof.

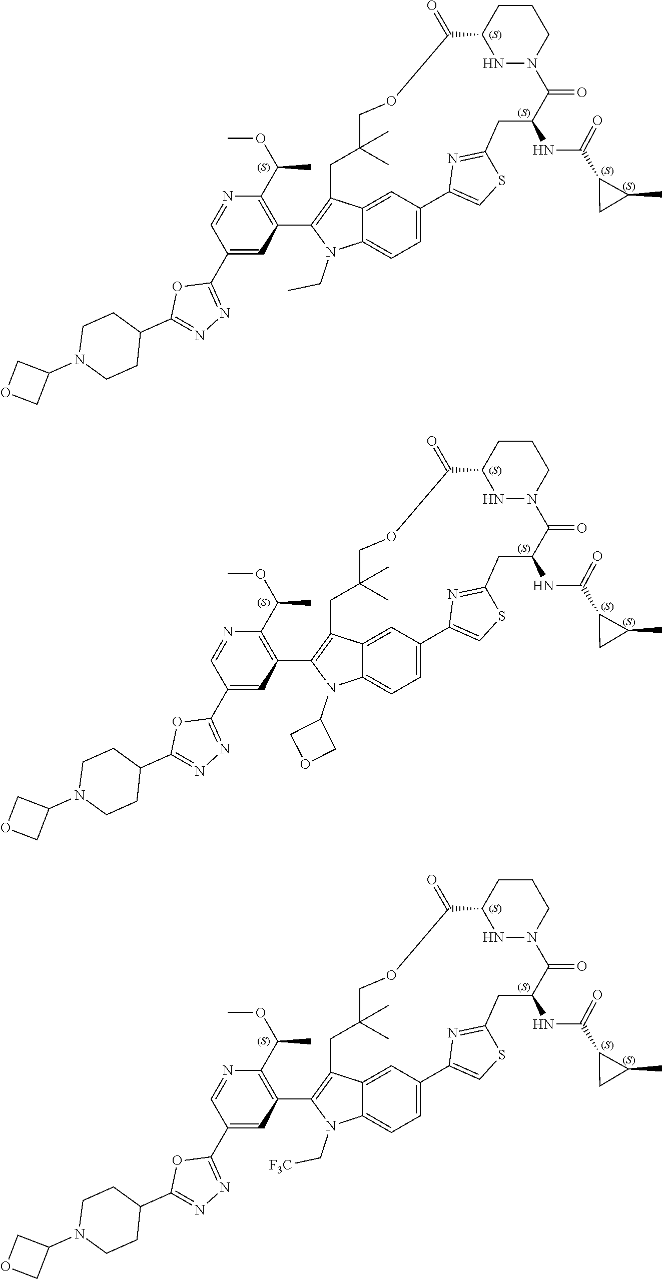

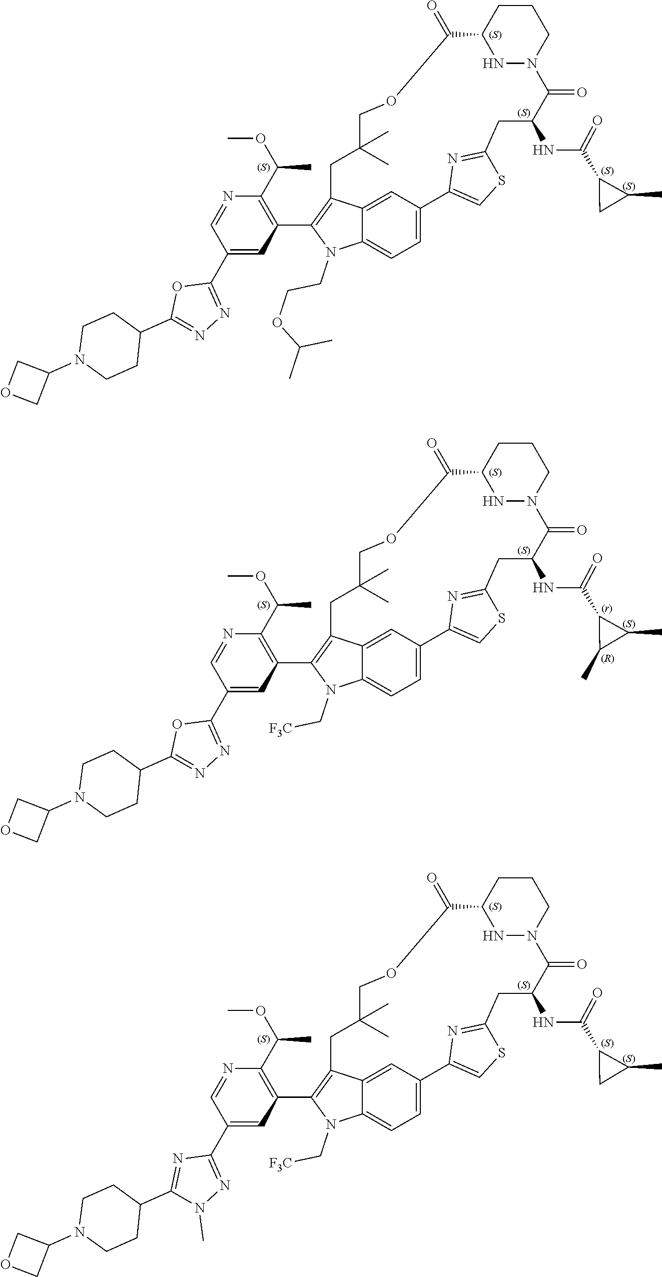

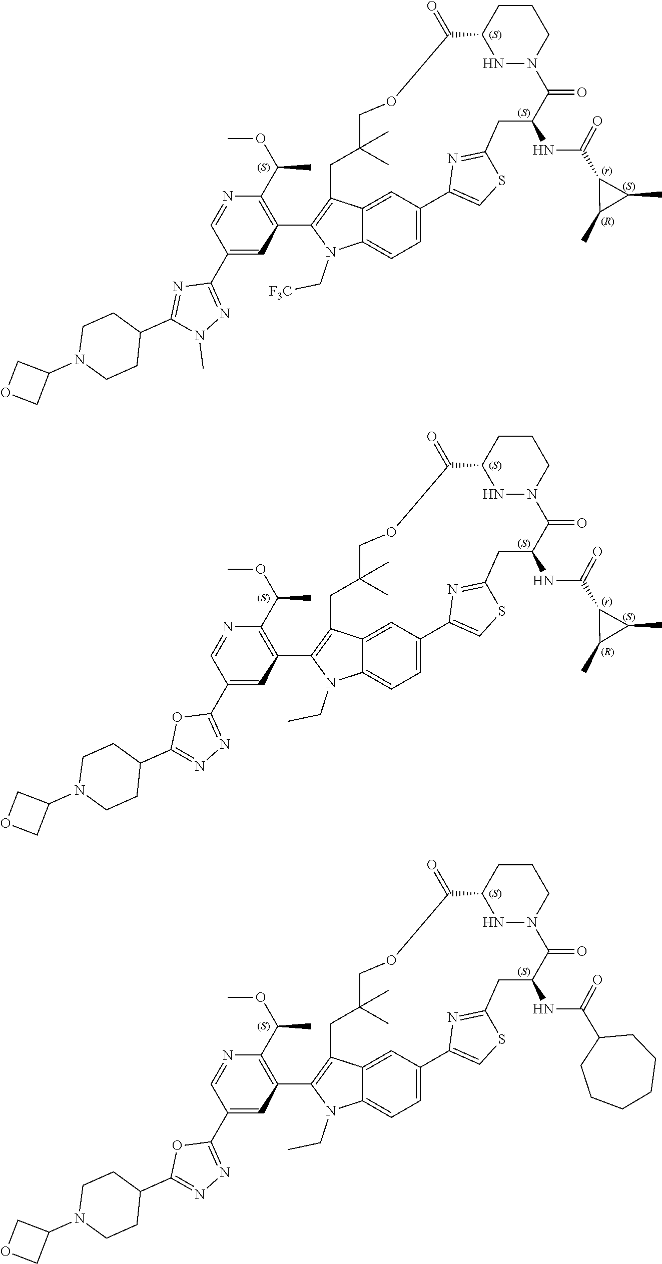

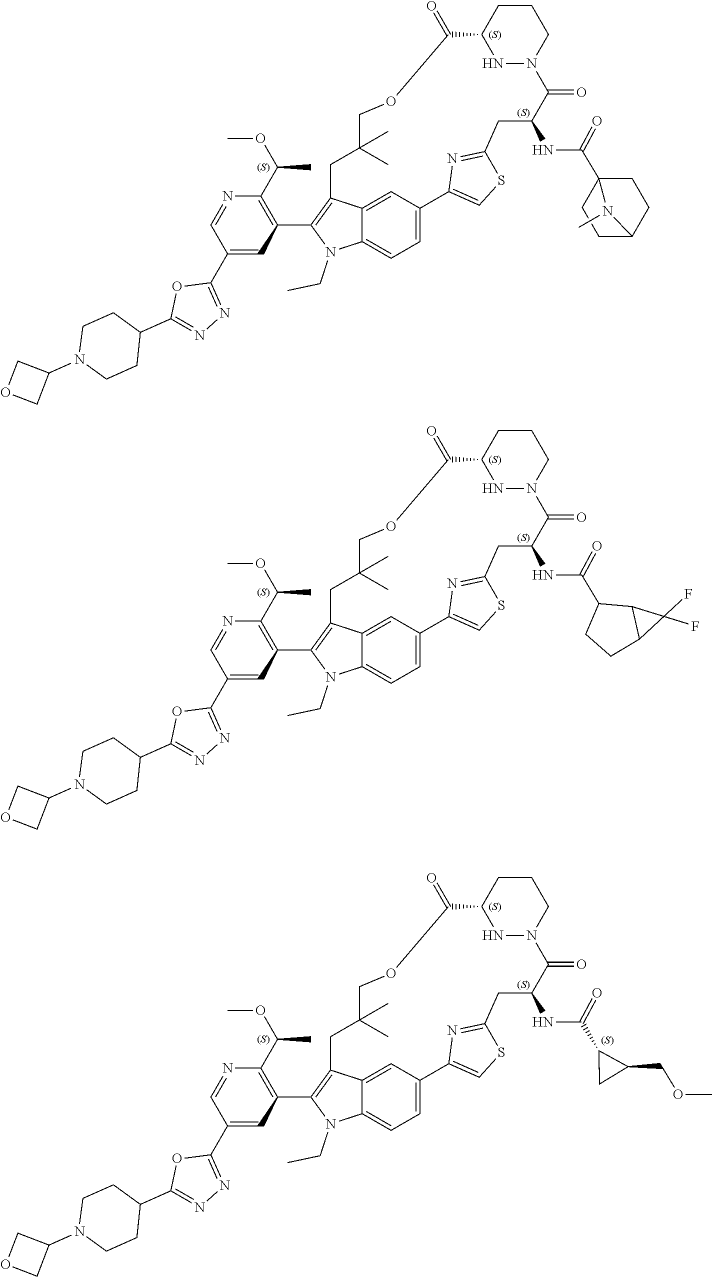

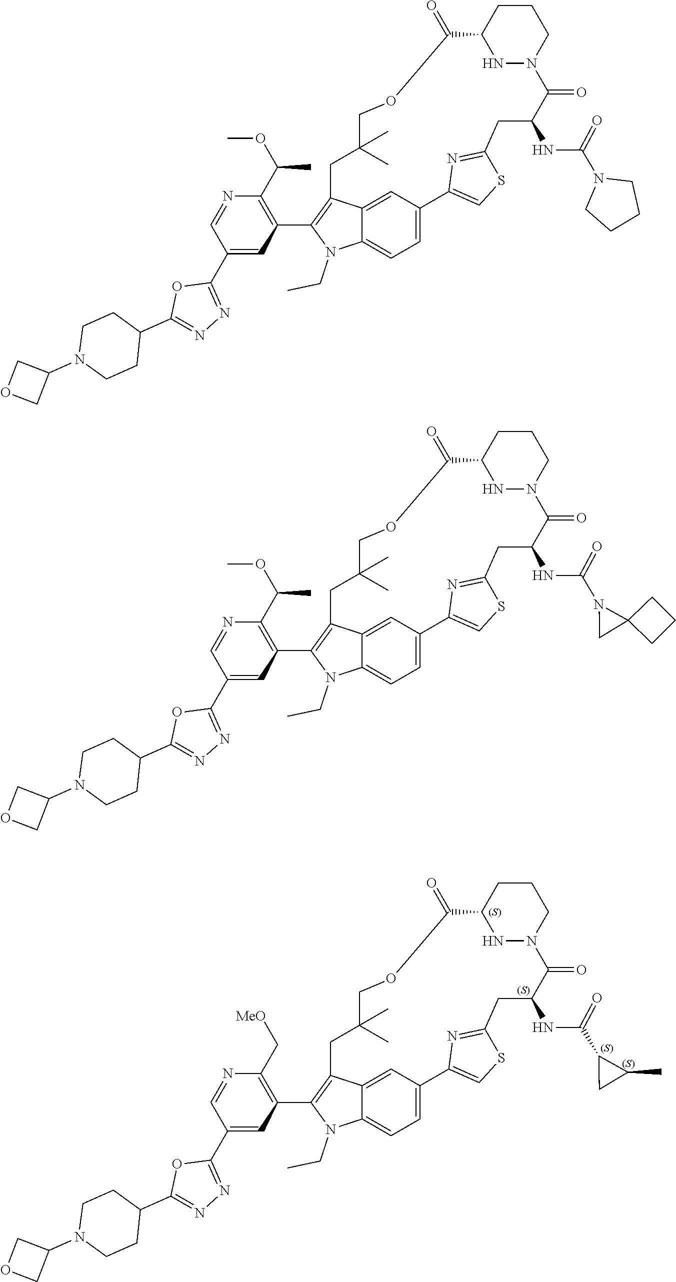

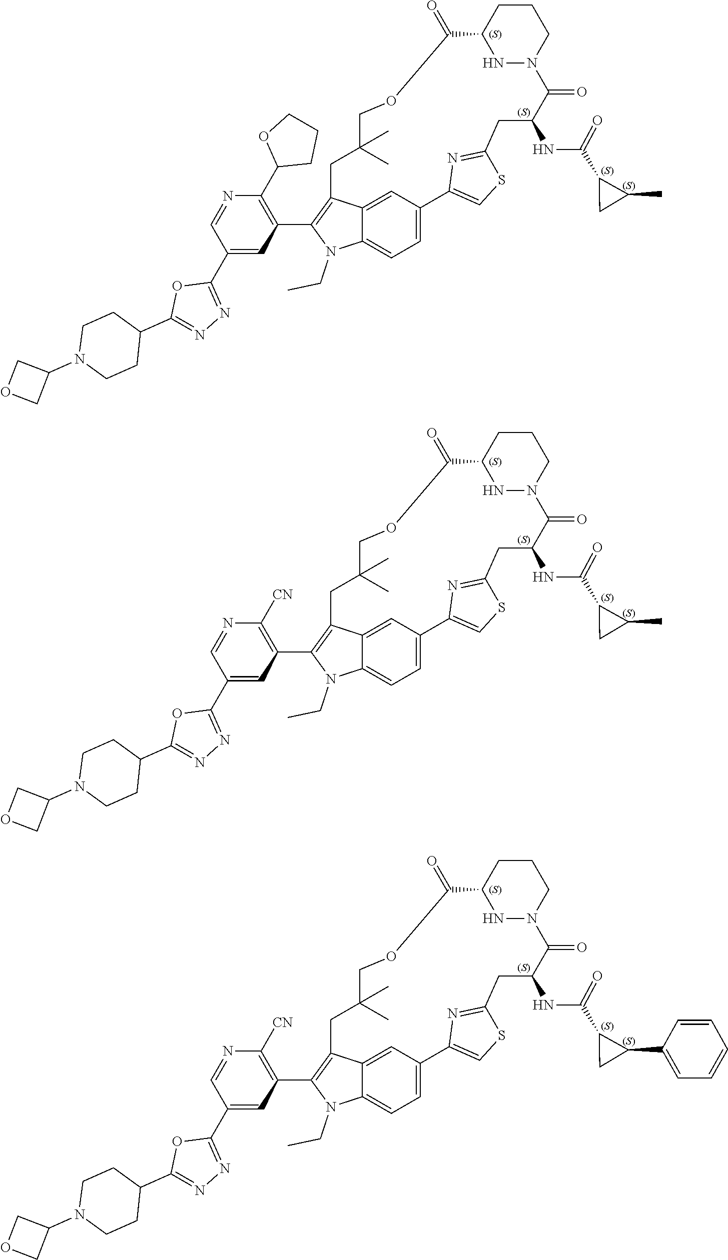

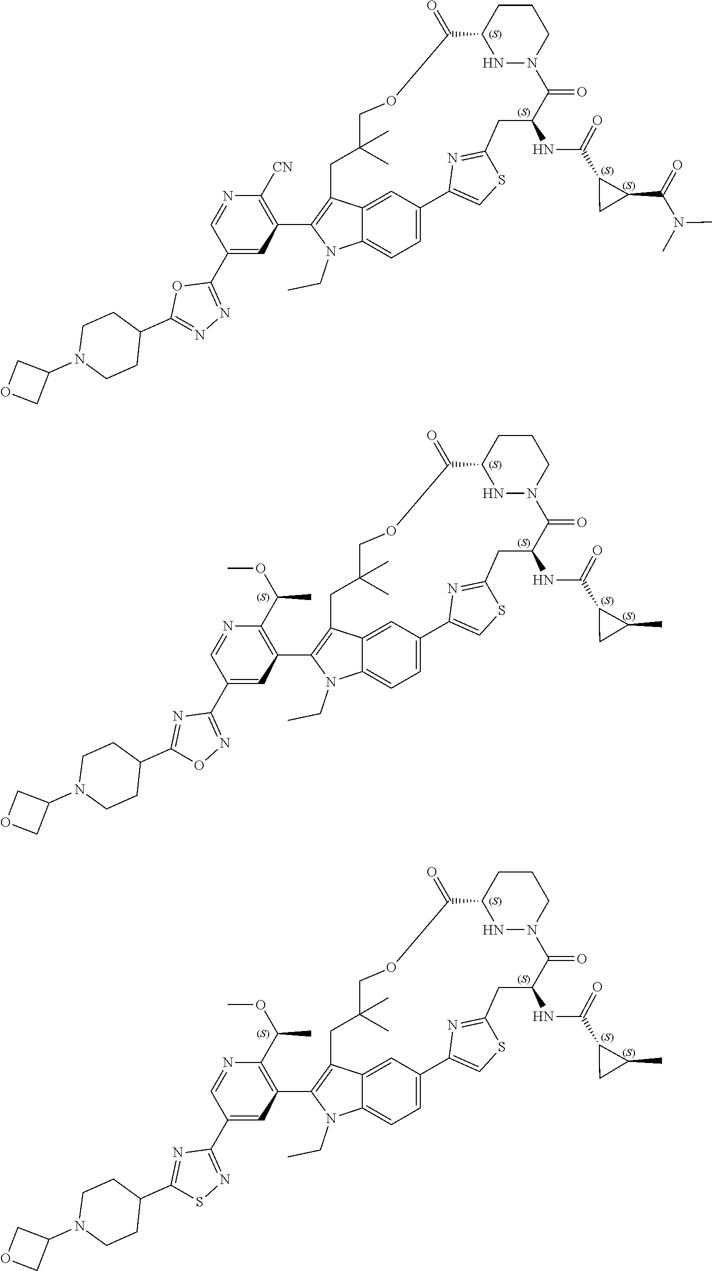

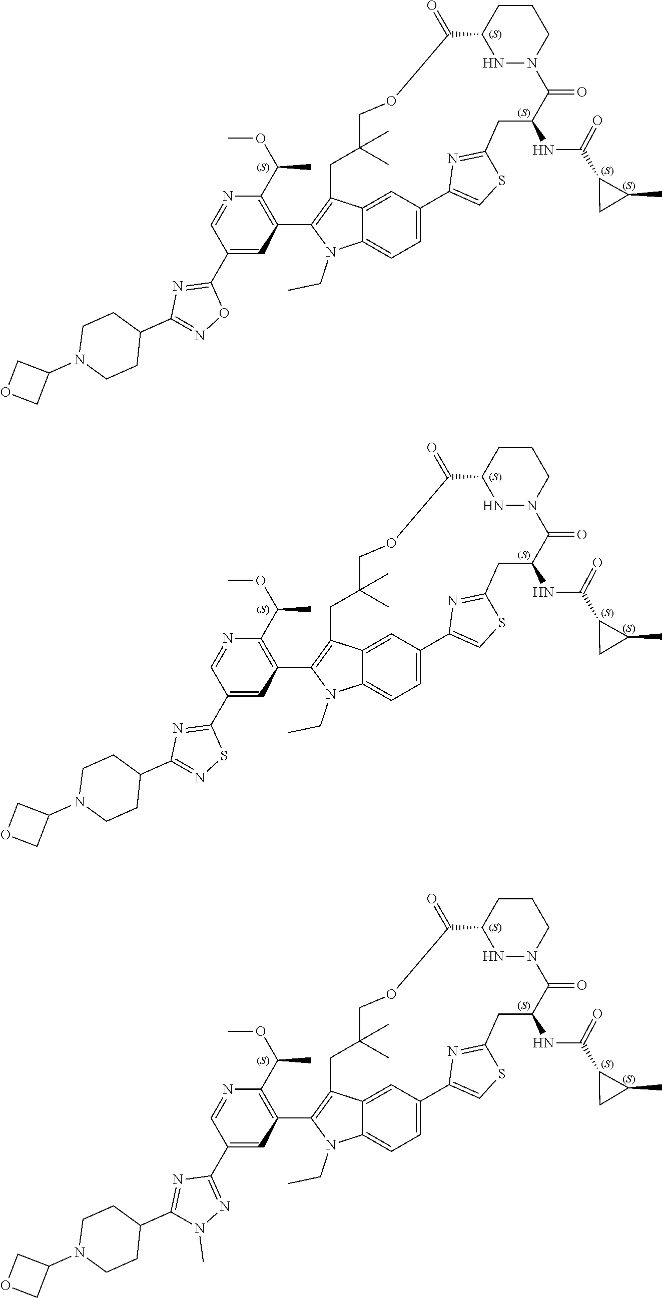

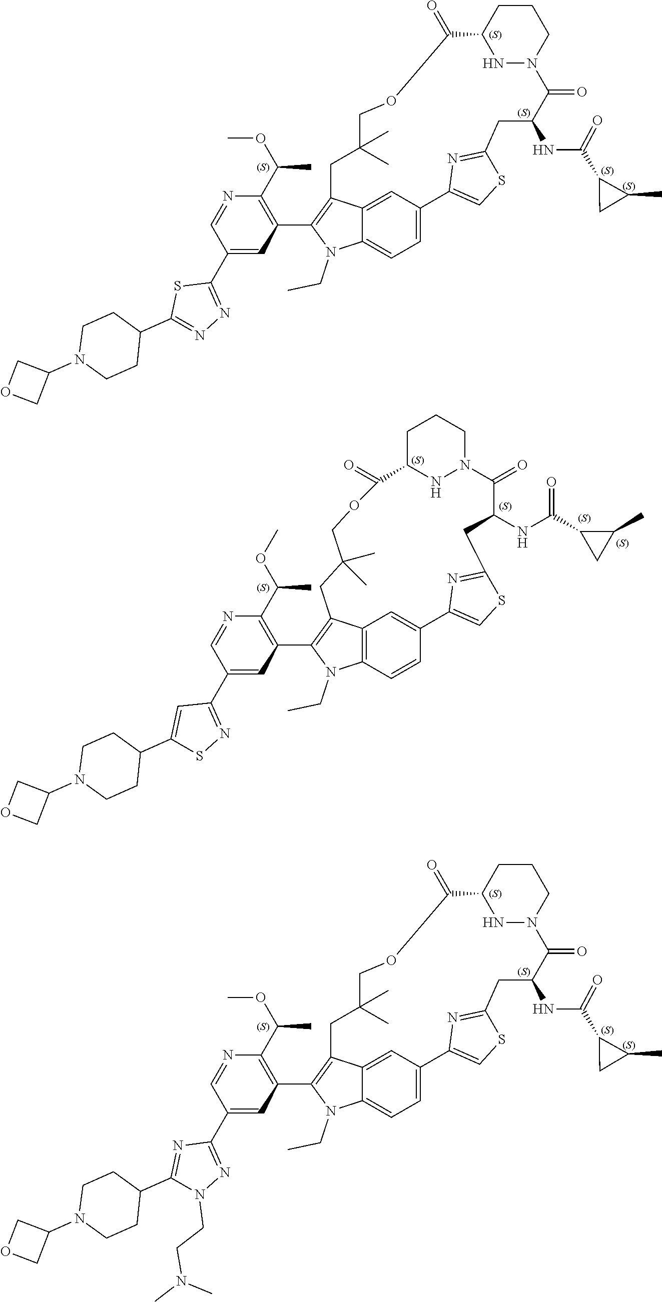

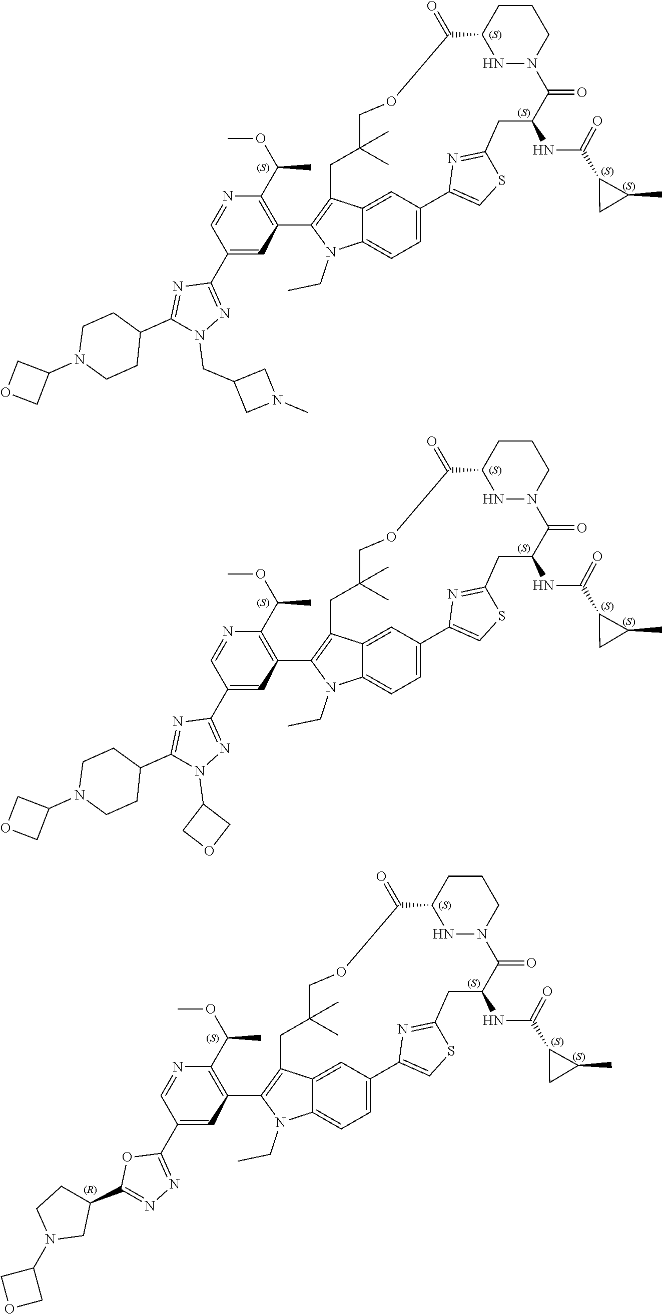

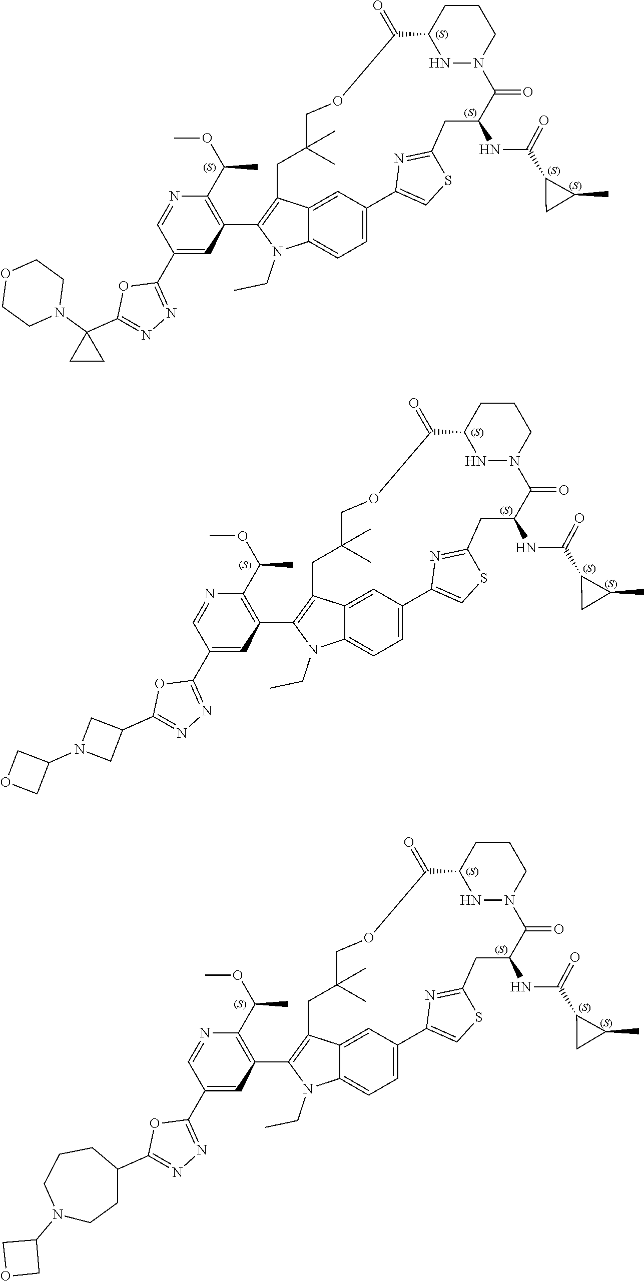

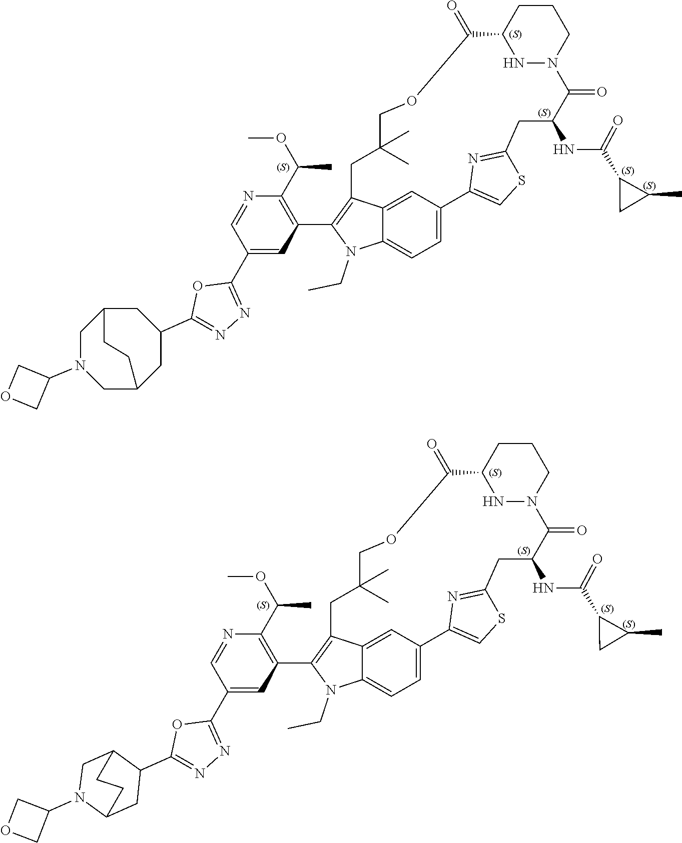

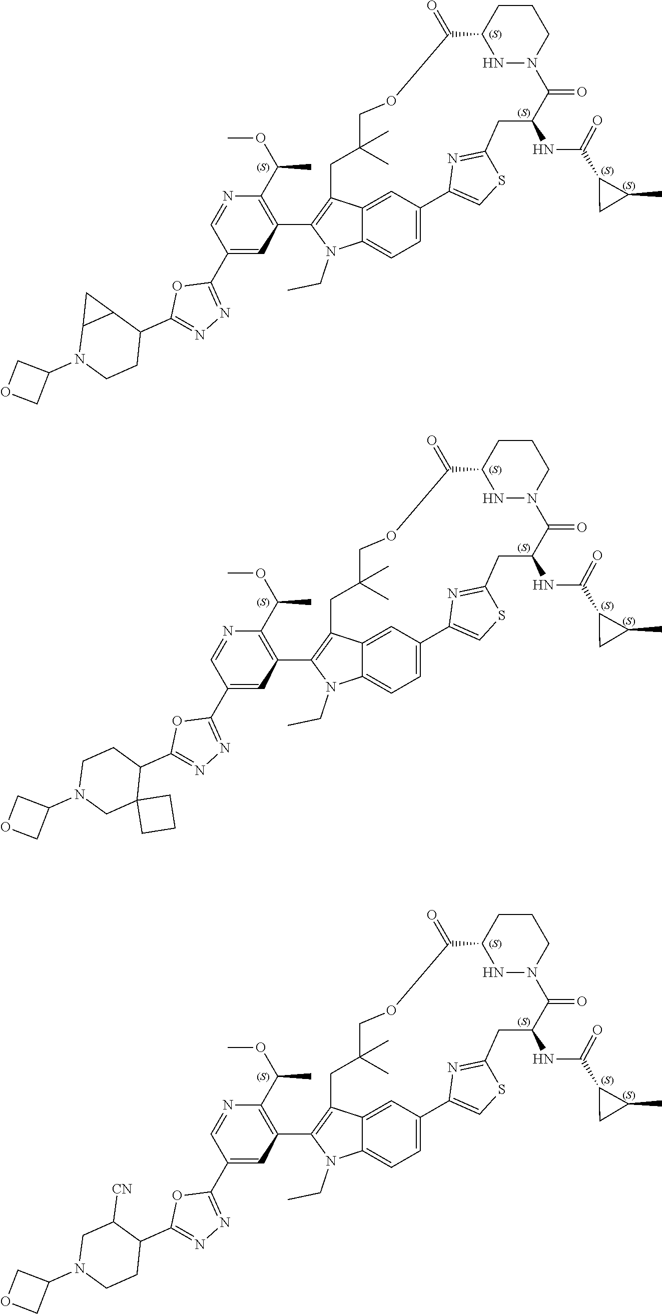

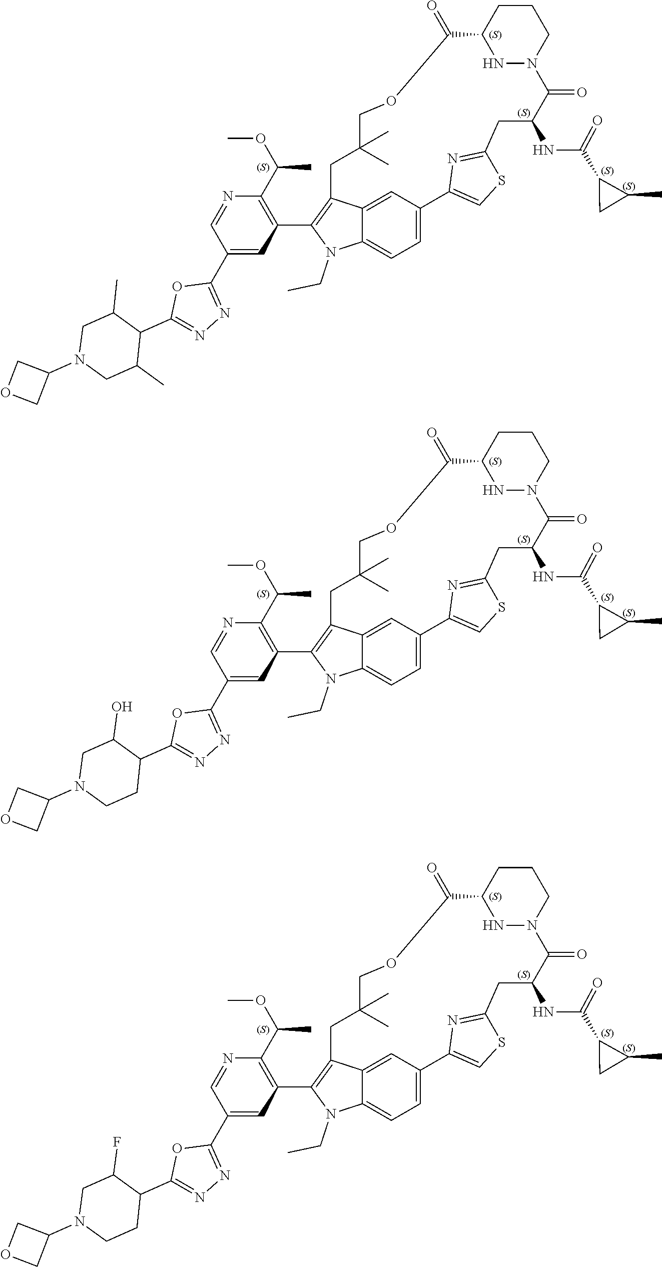

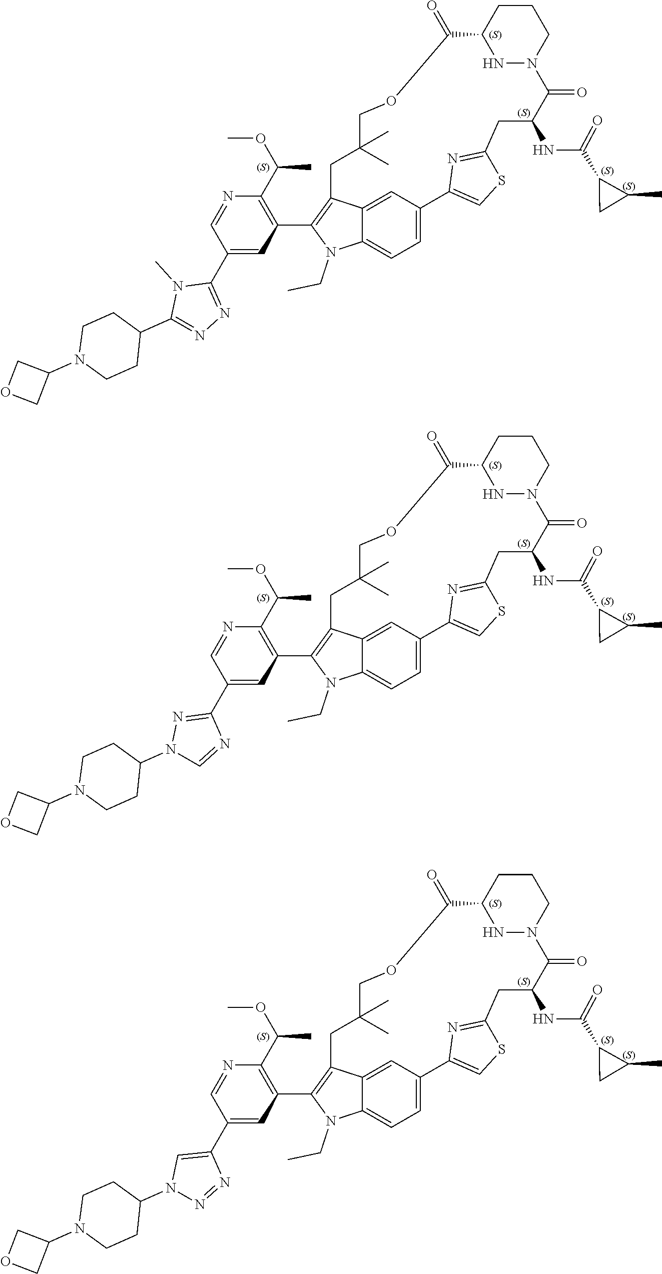

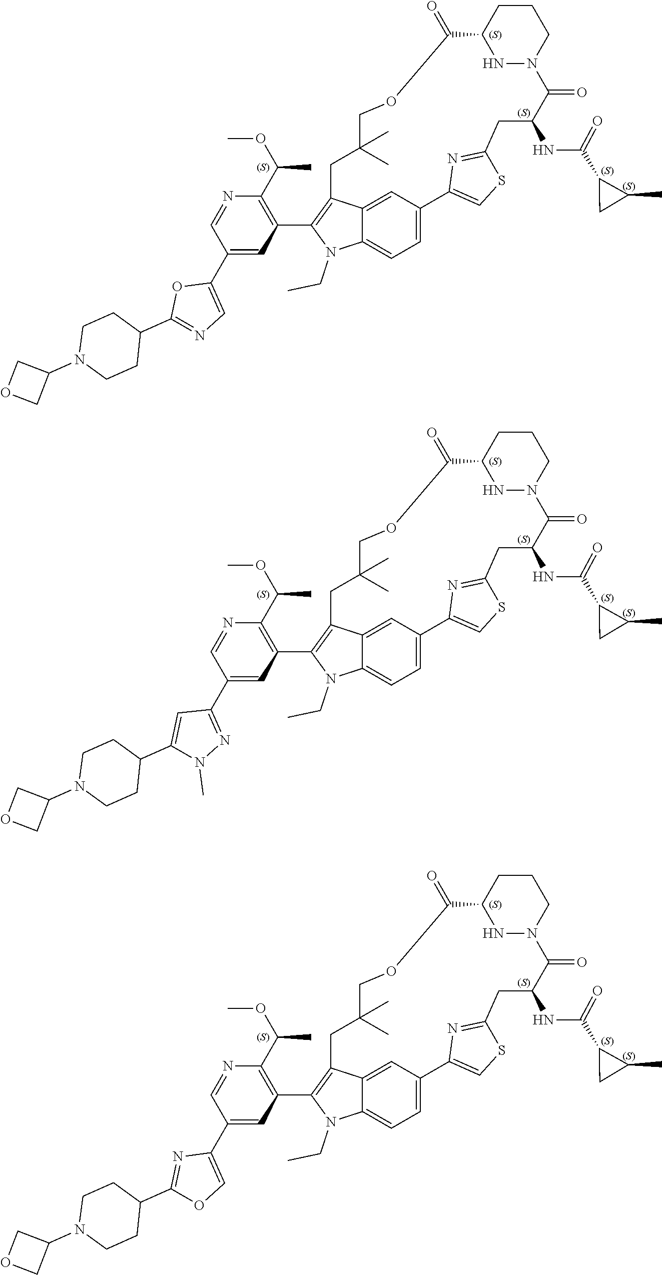

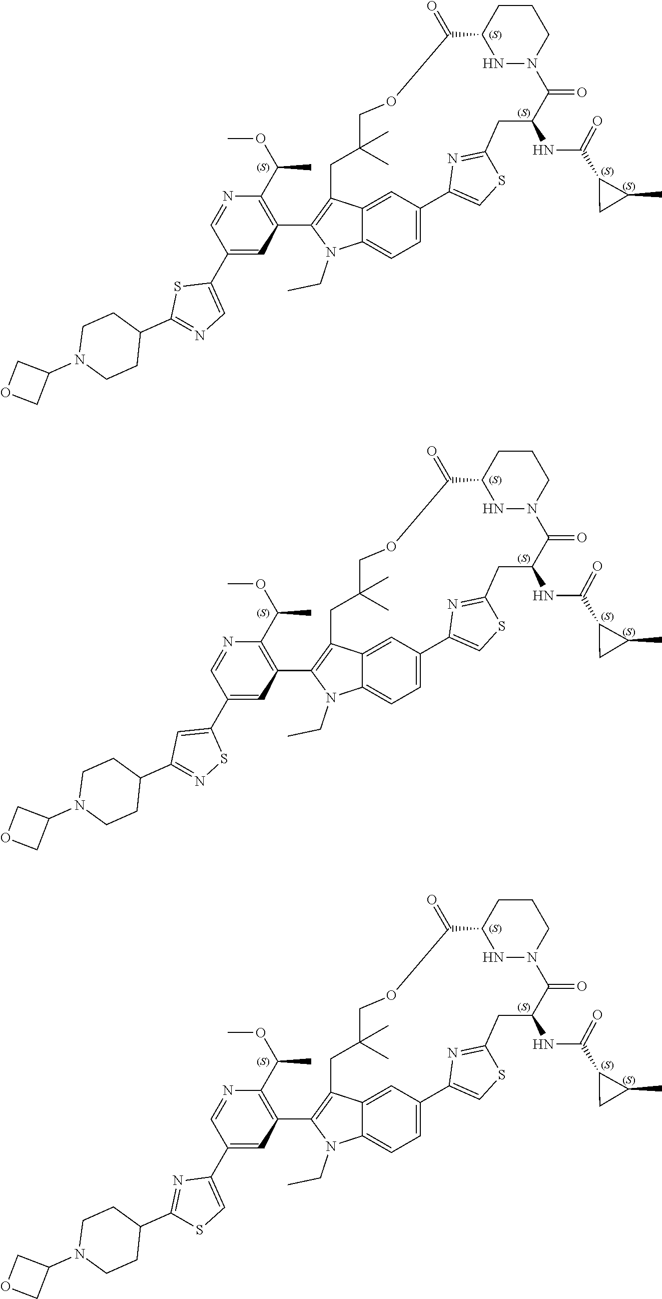

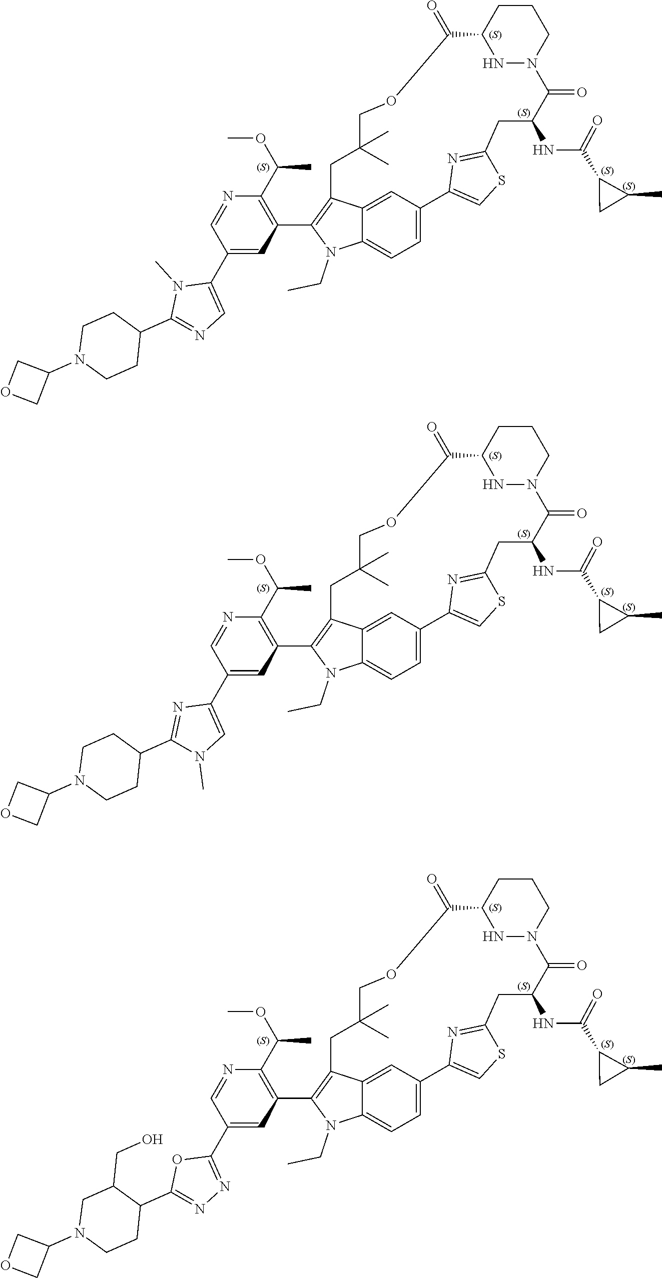

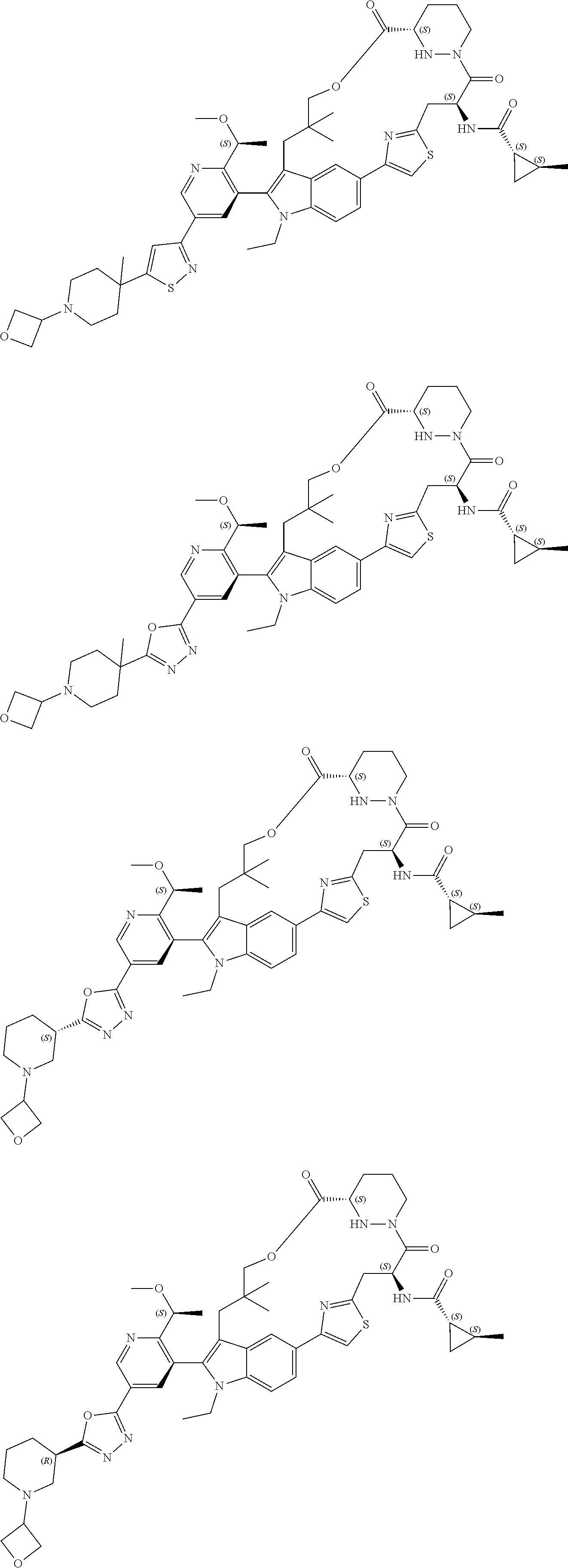

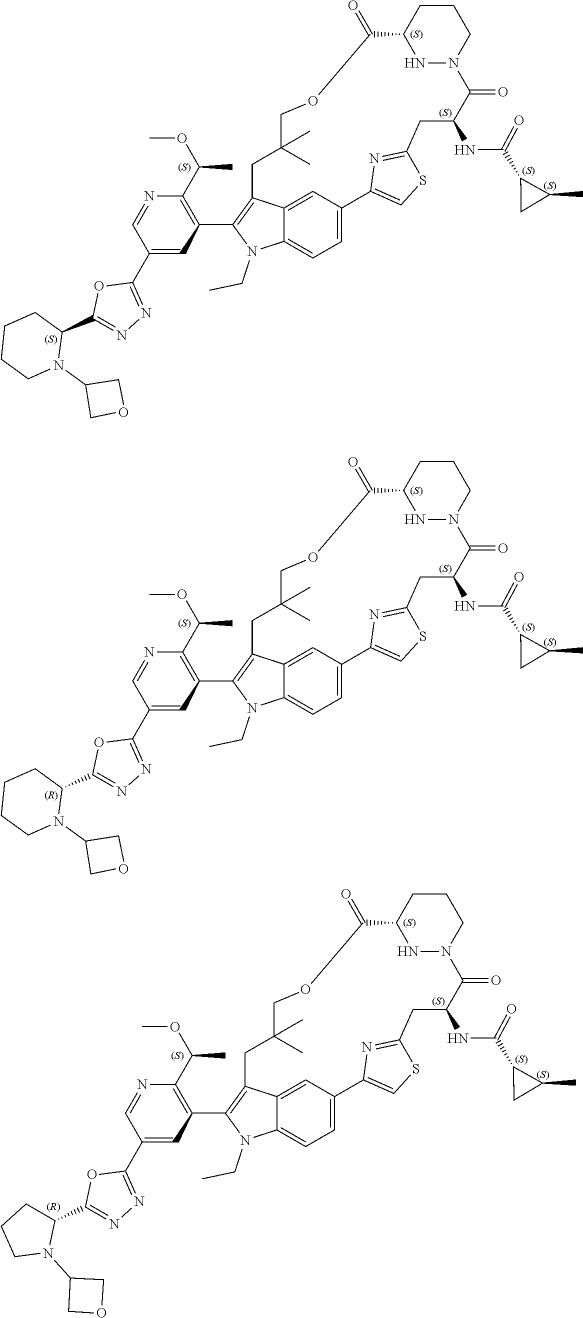

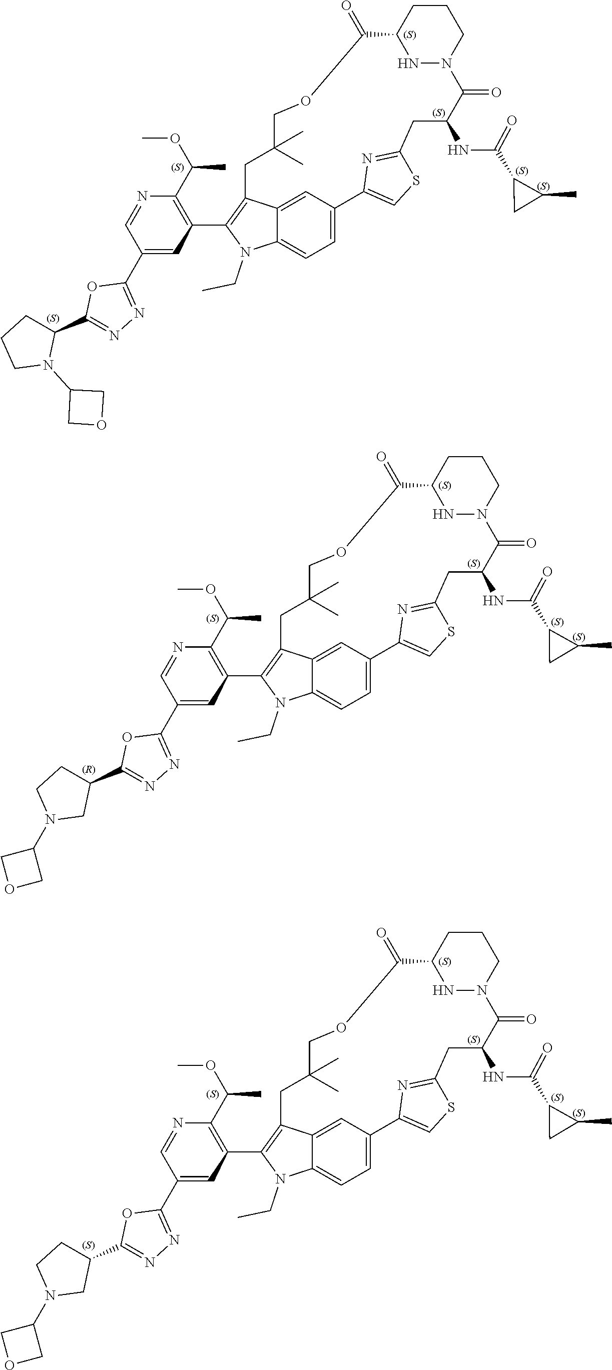

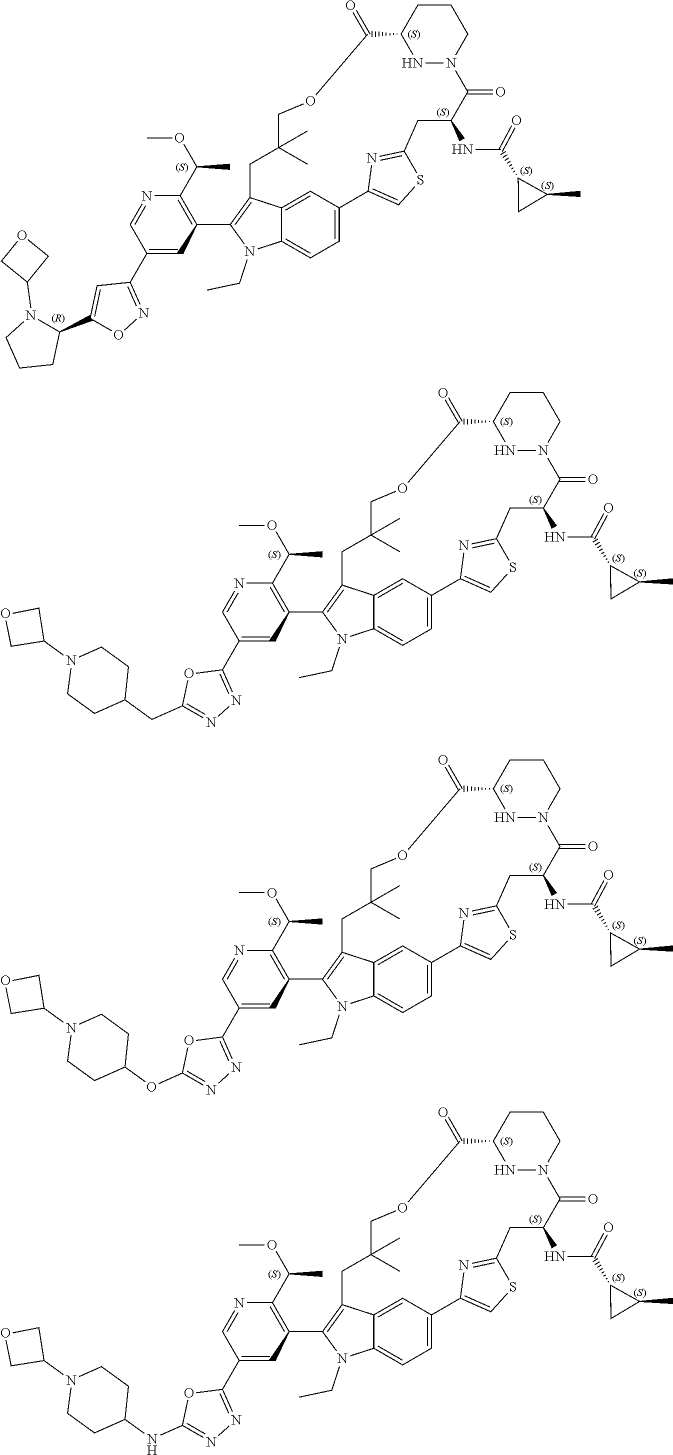

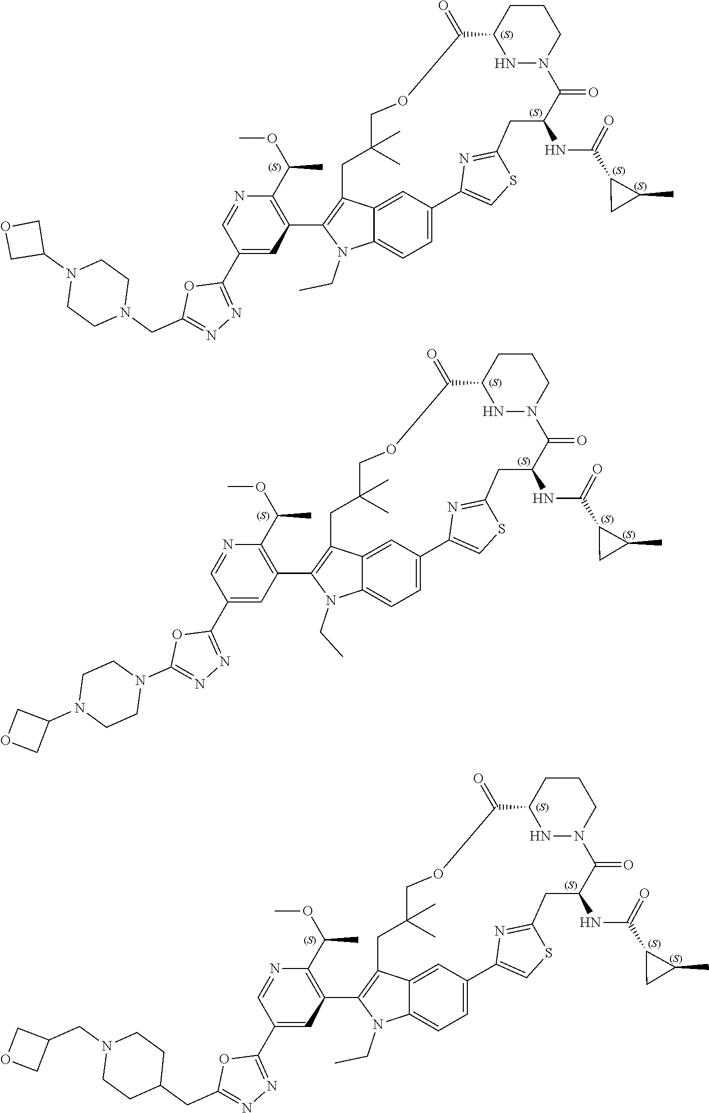

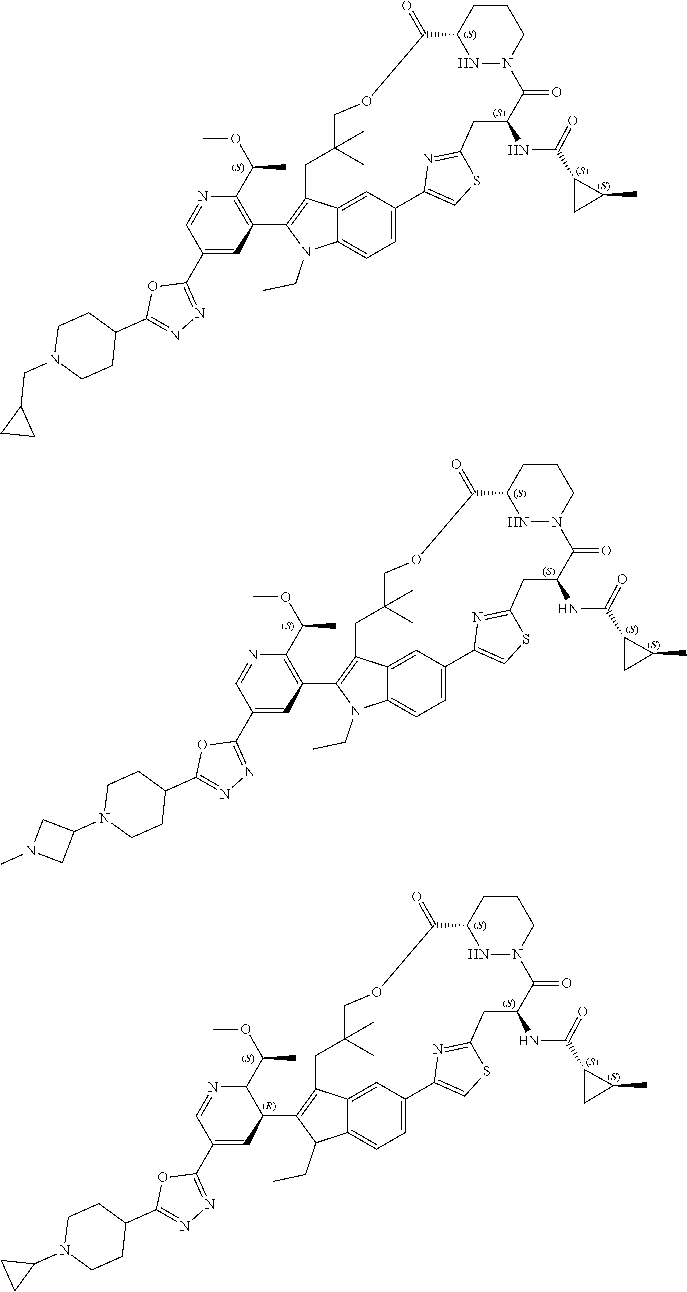

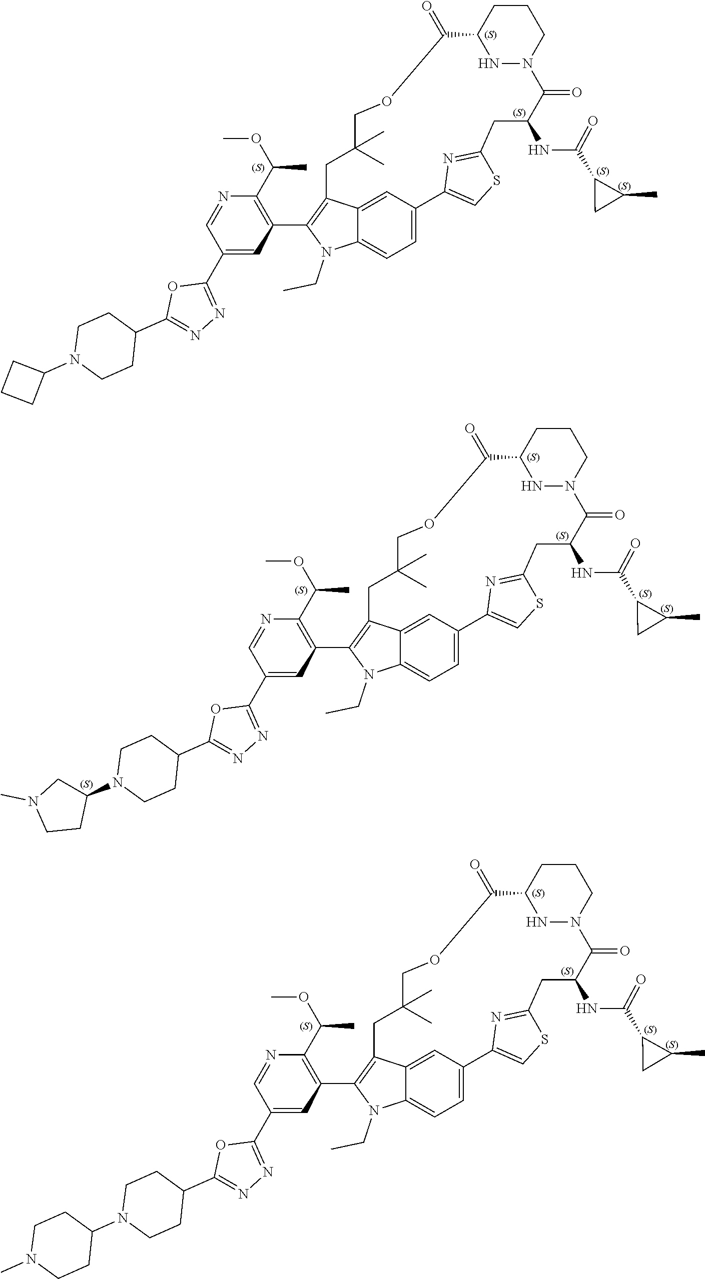

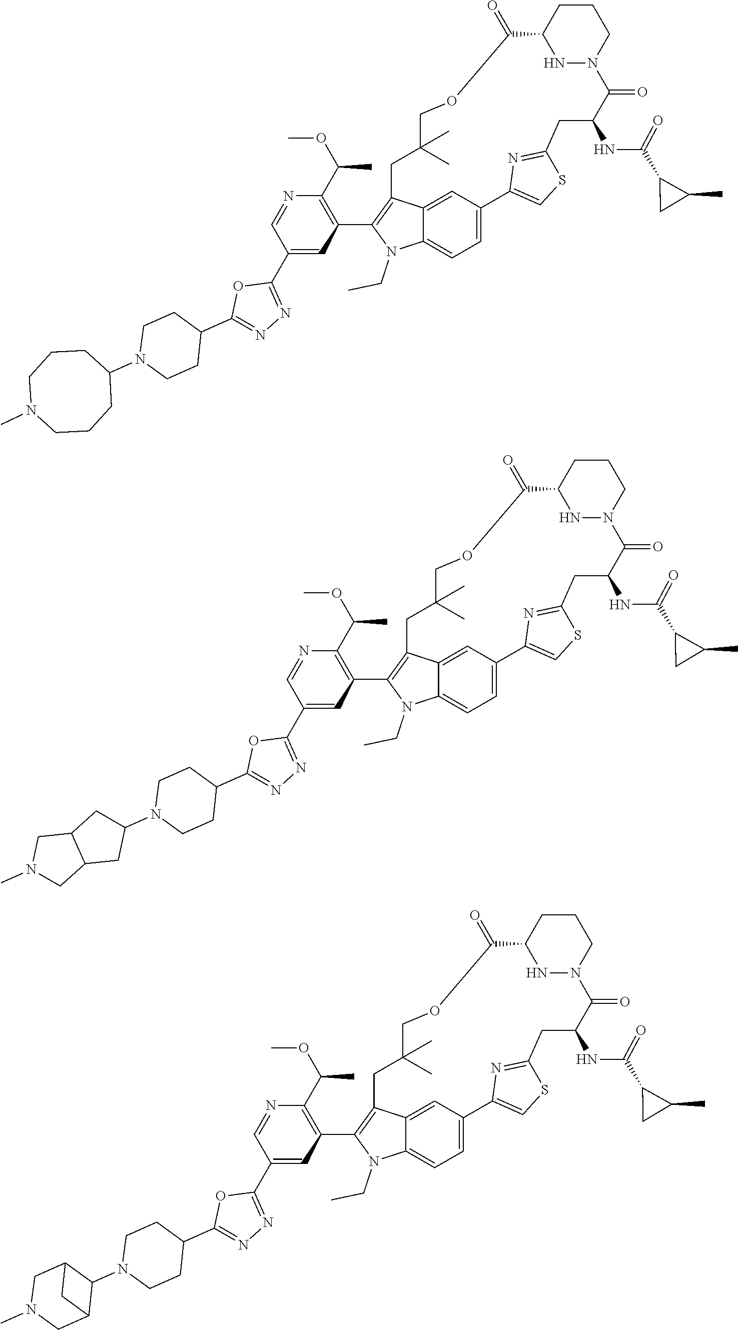

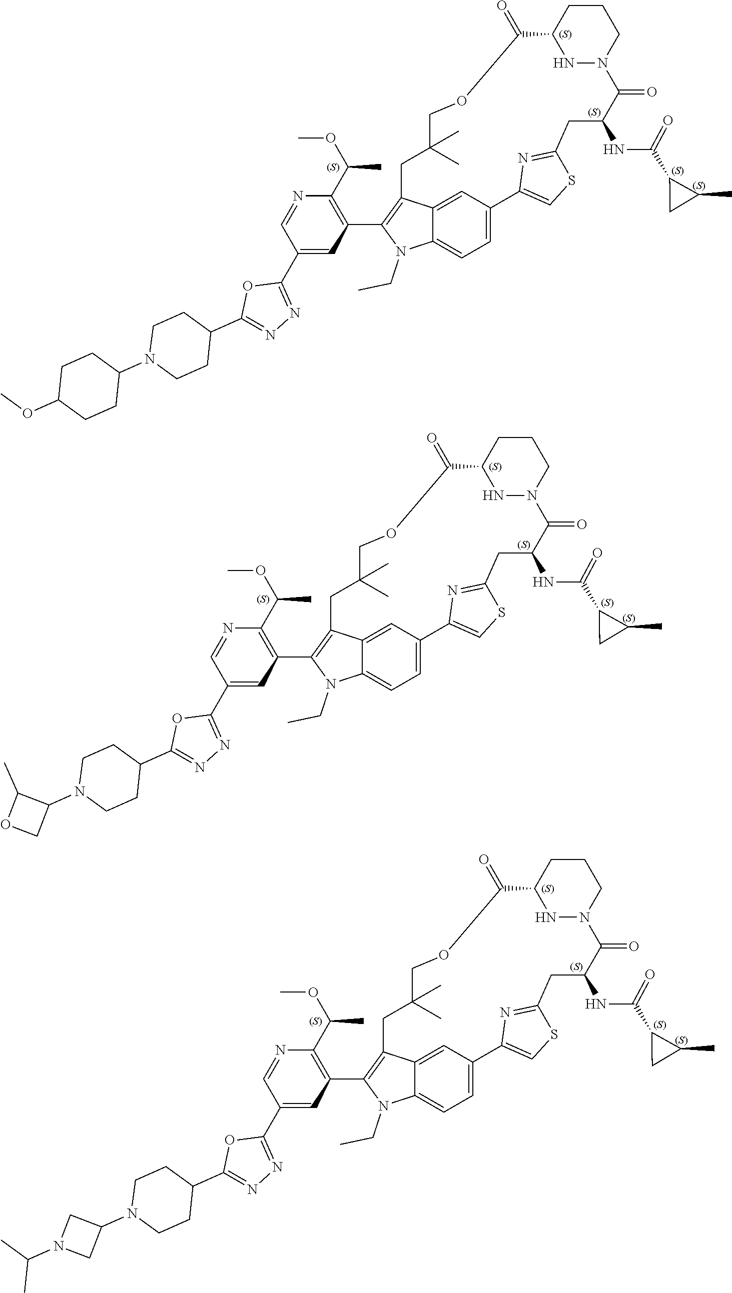

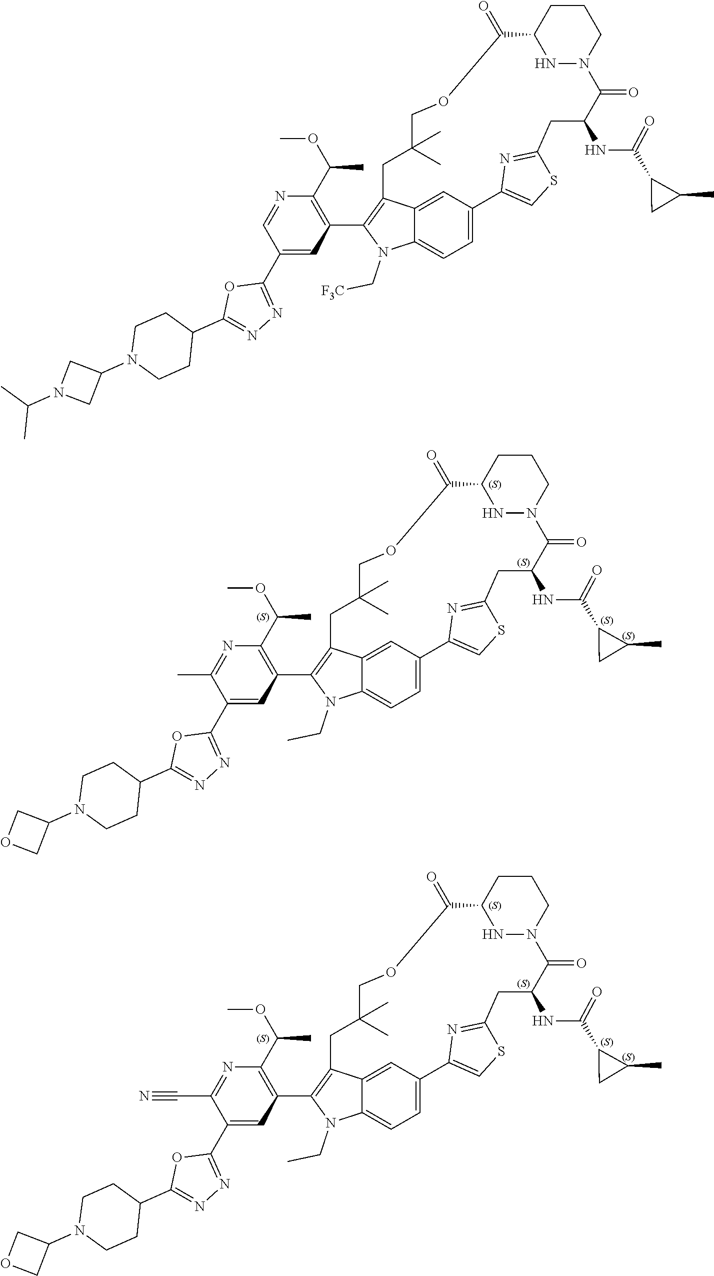

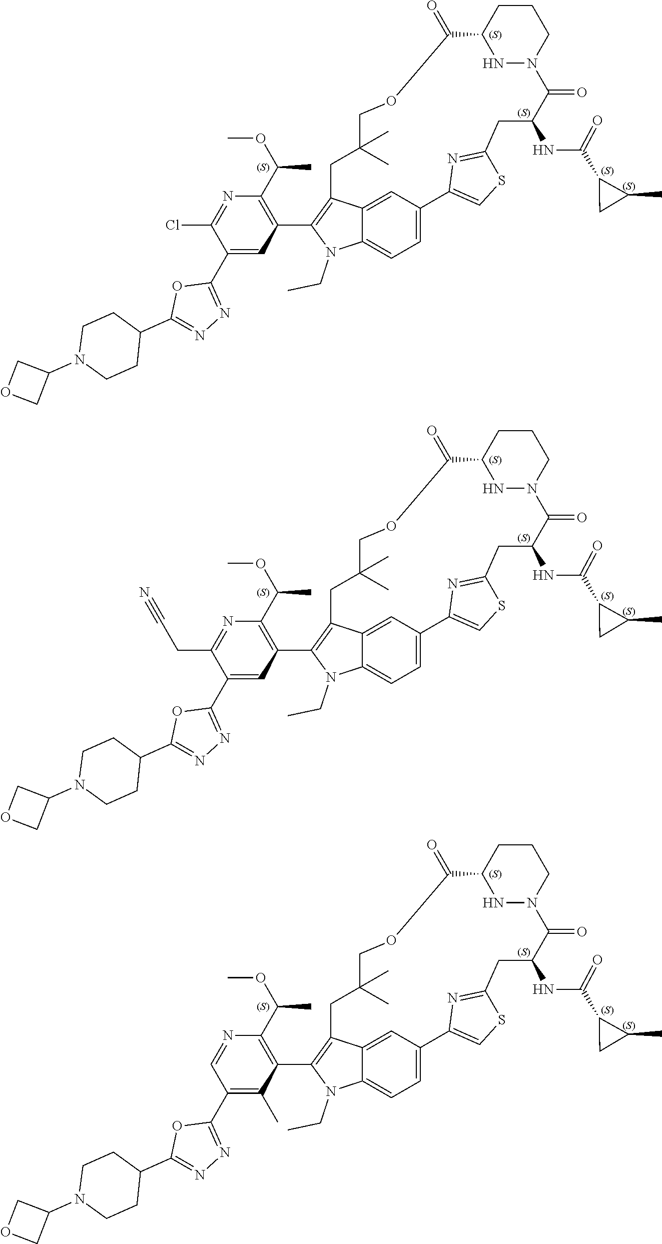

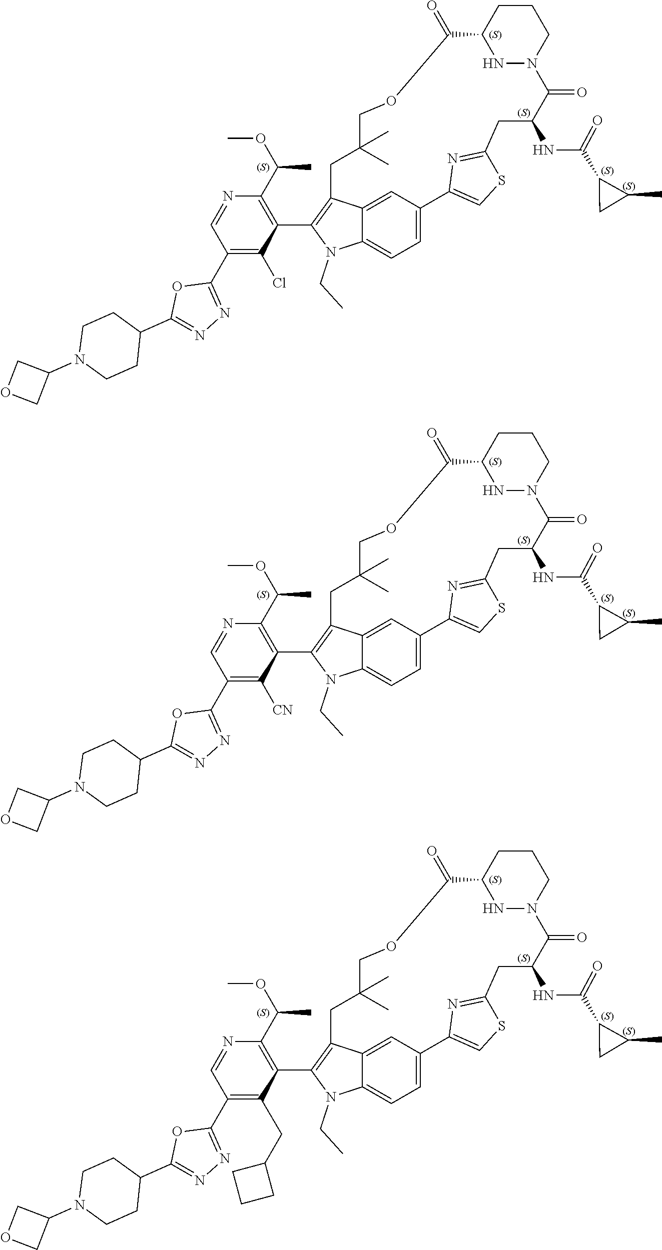

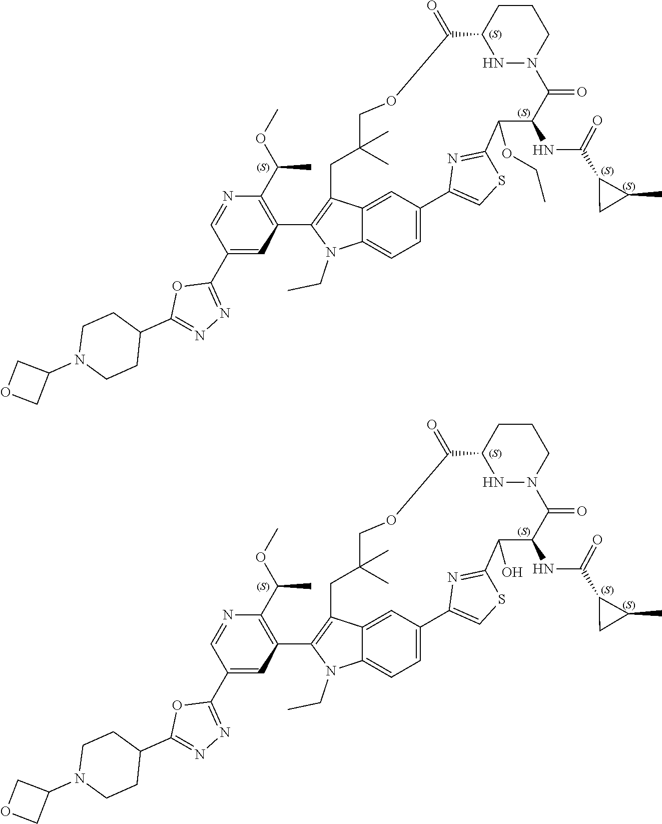

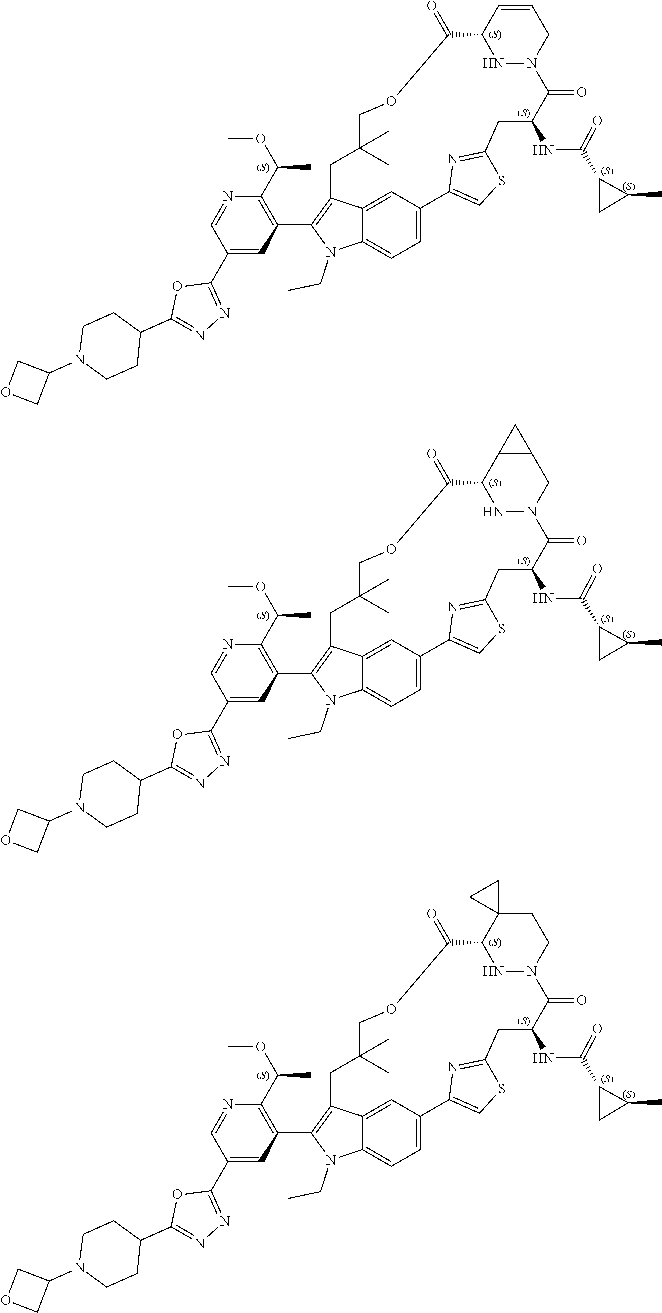

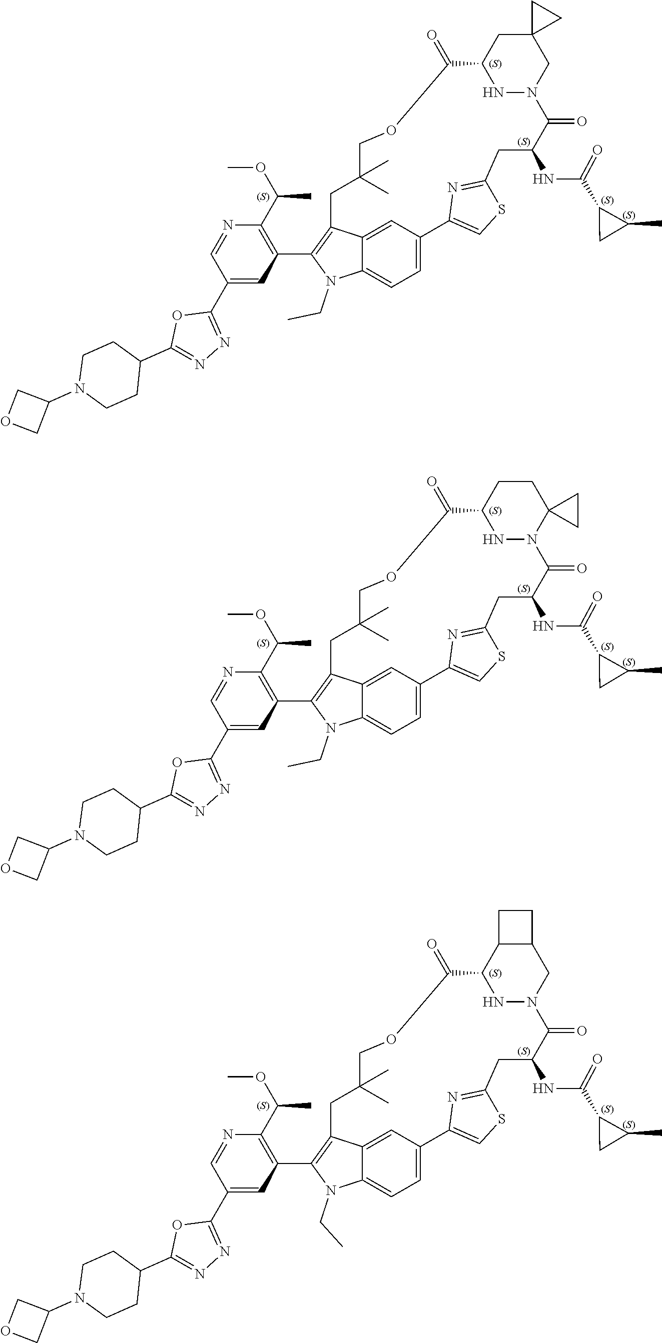

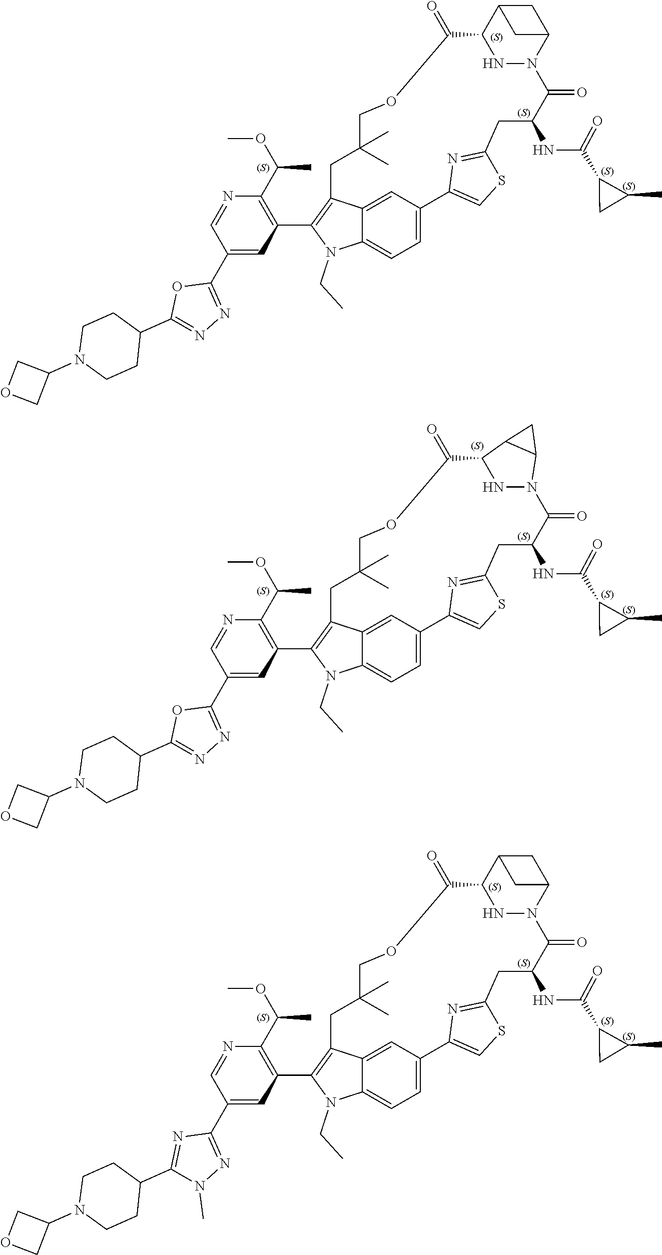

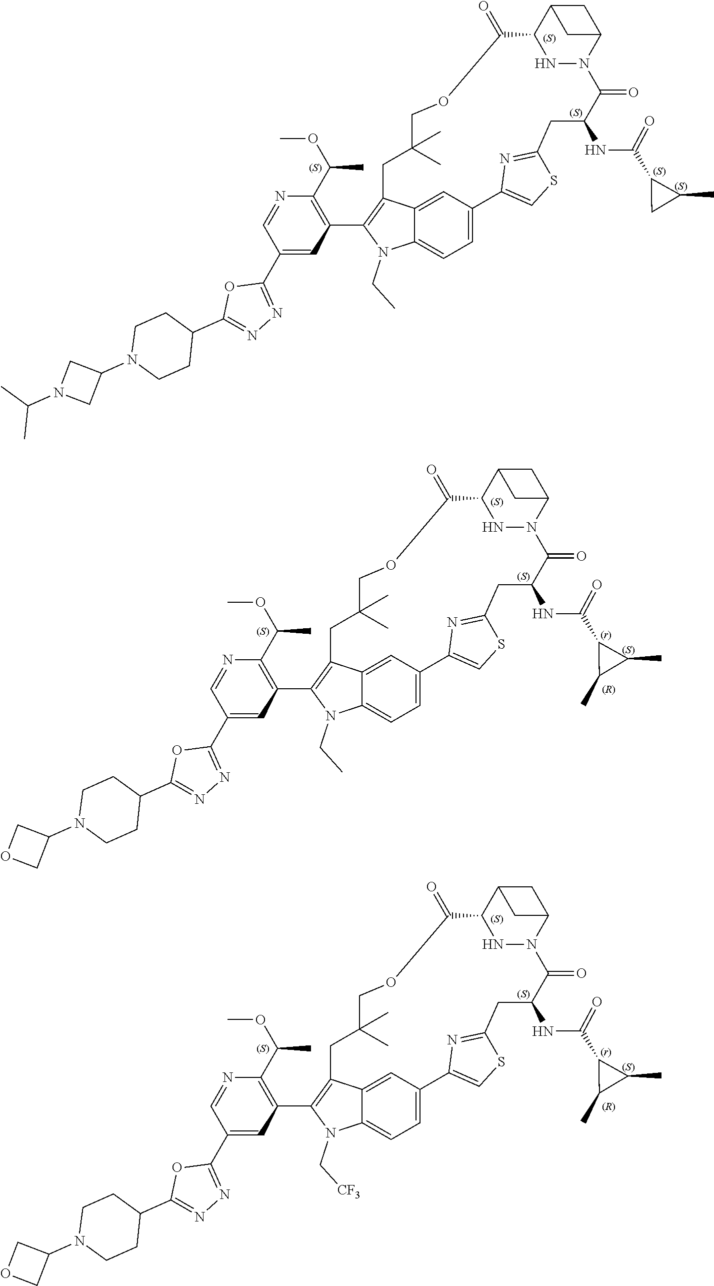

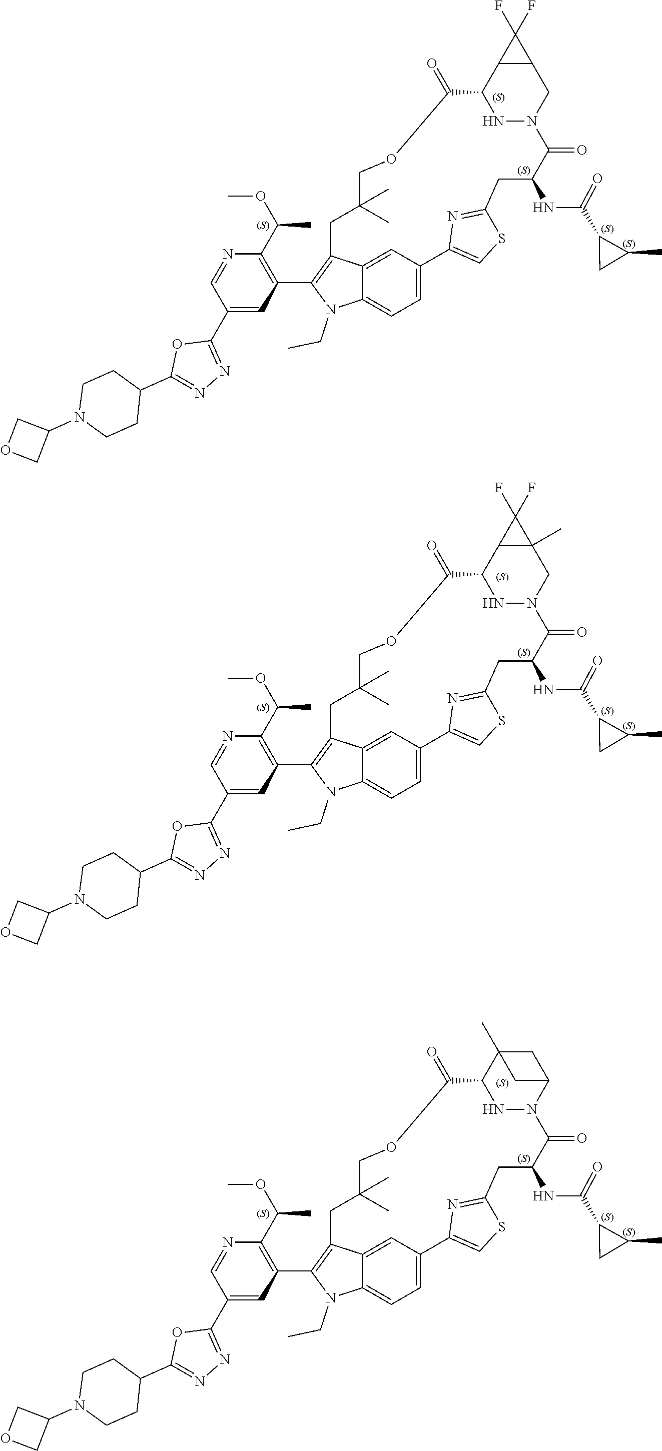

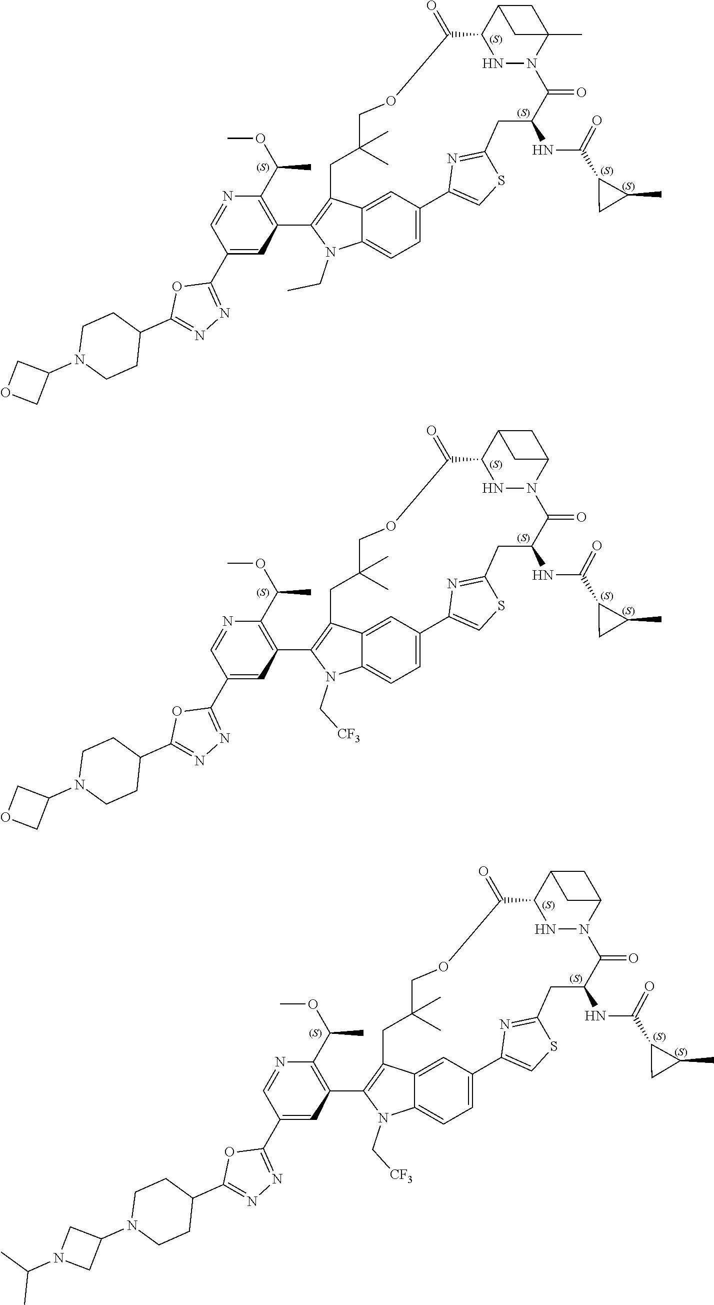

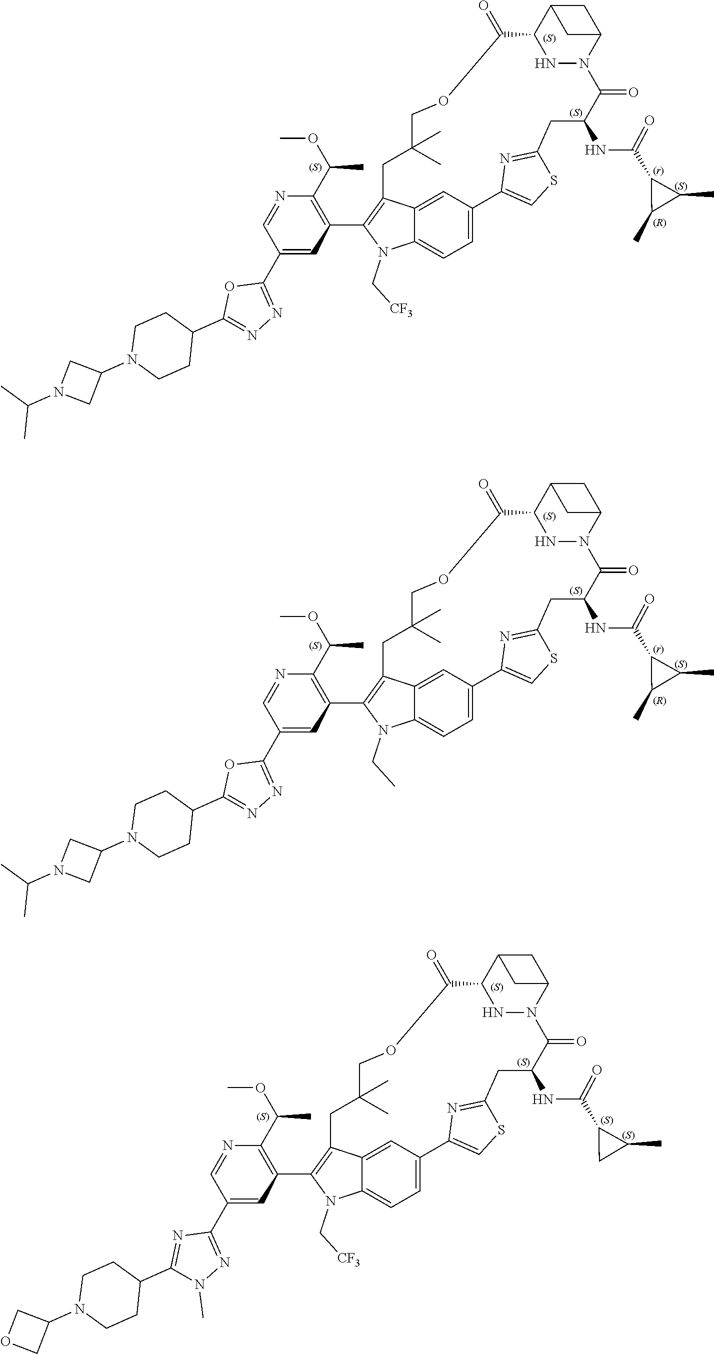

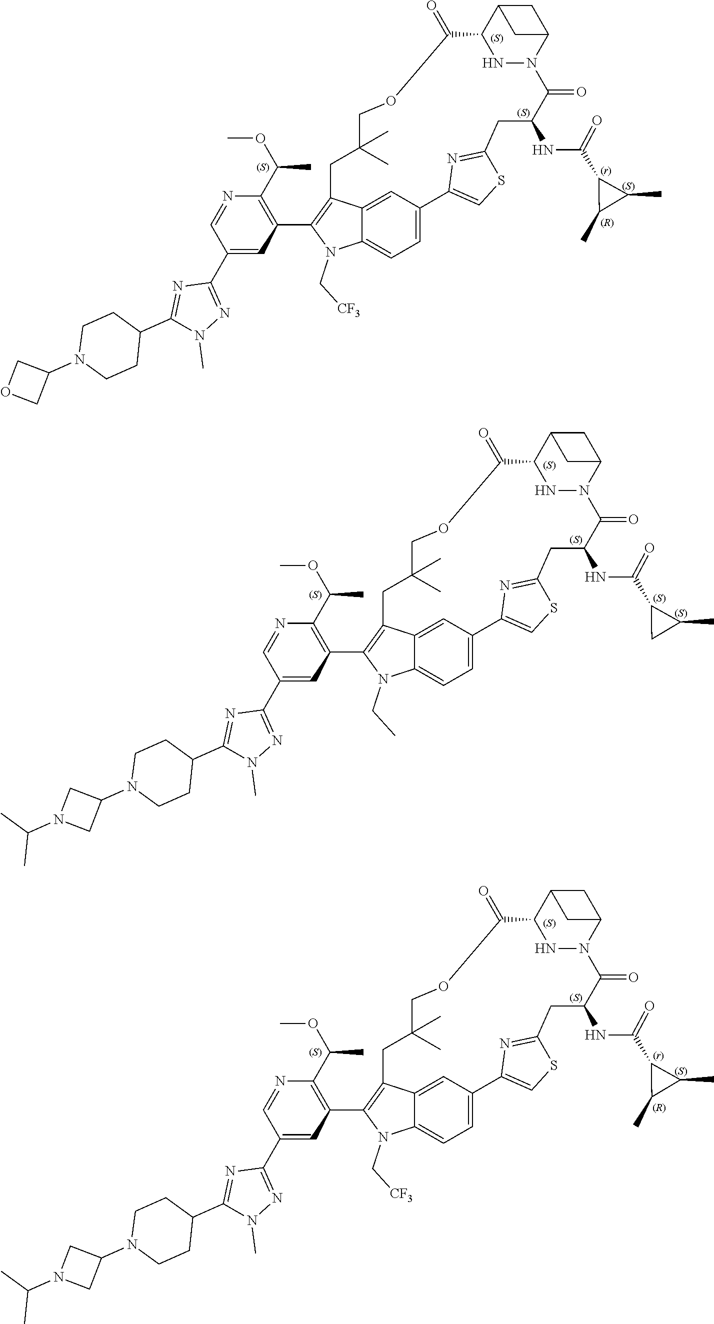

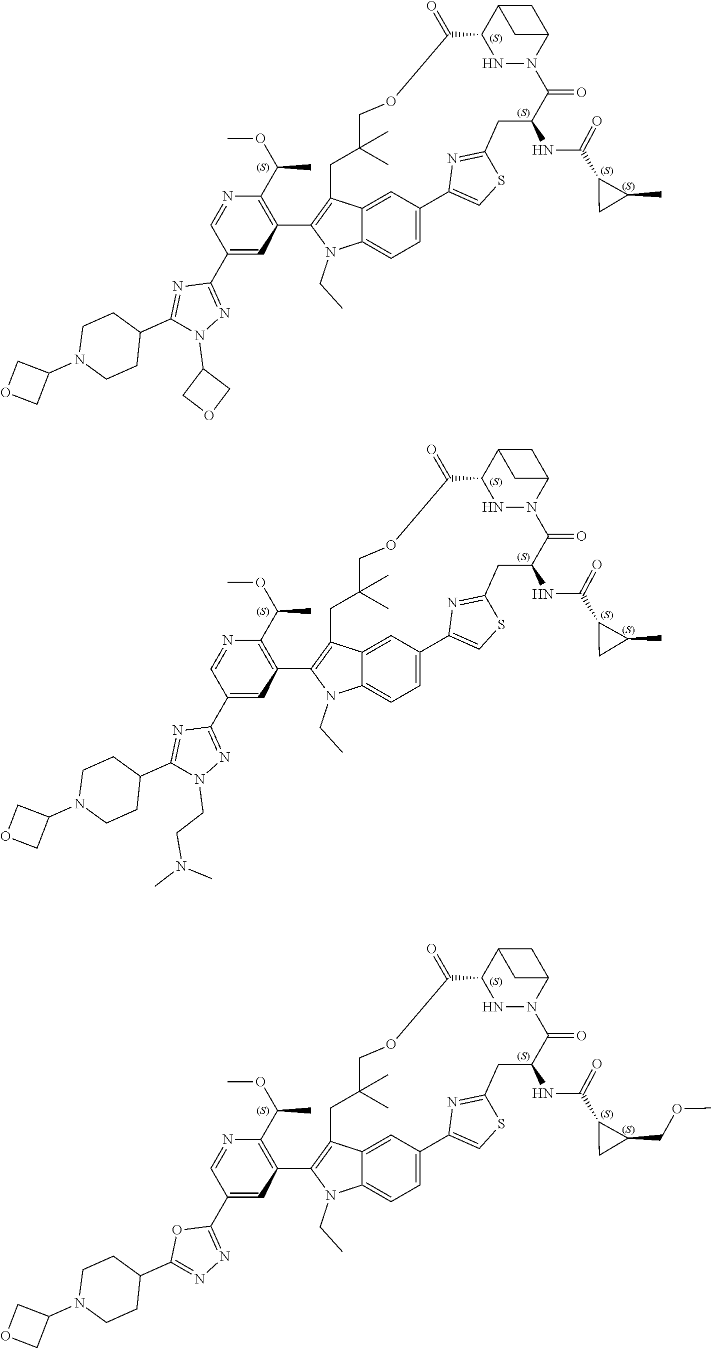

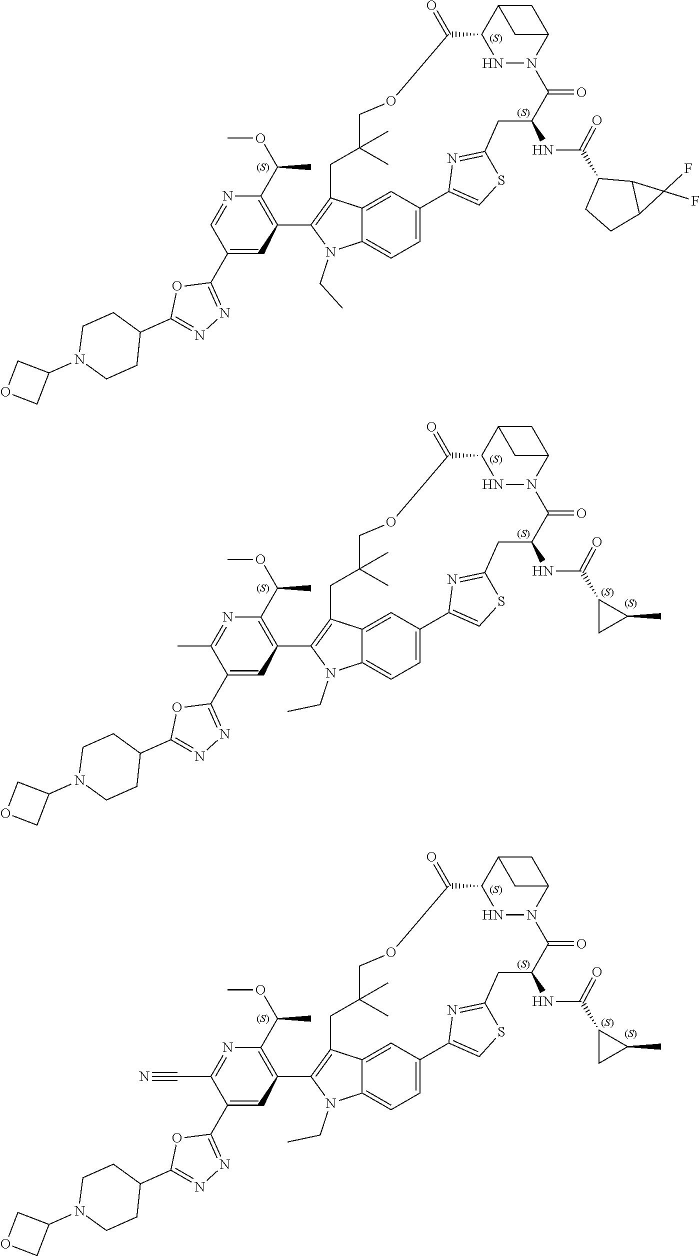

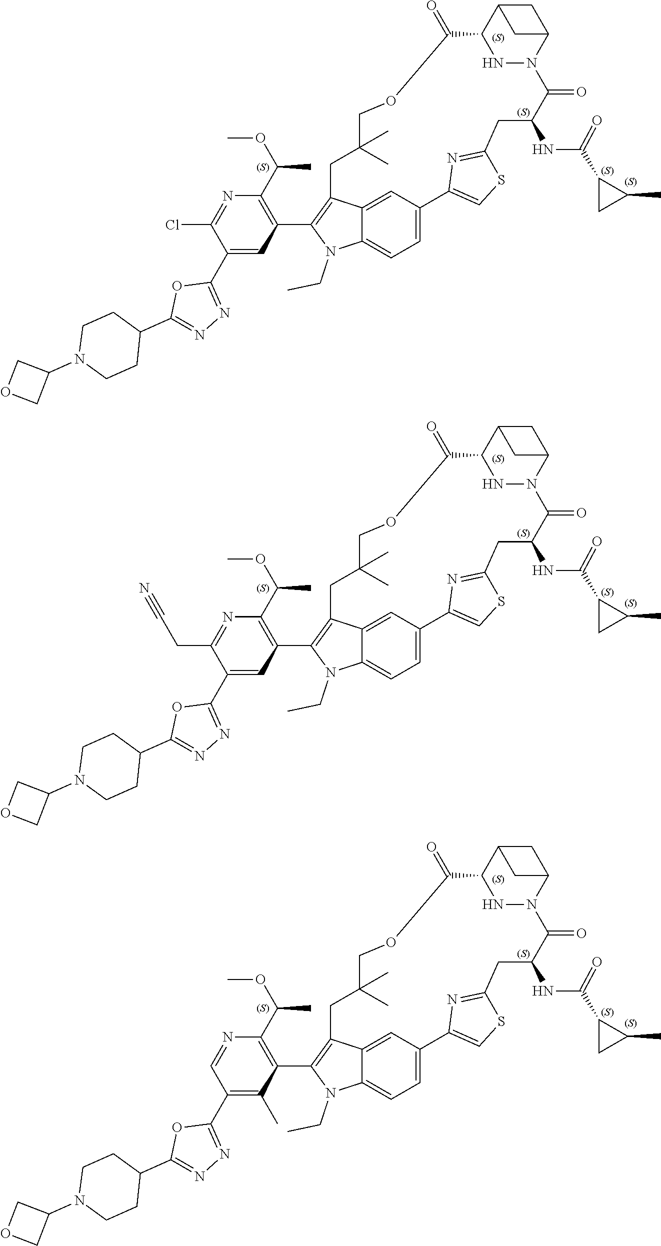

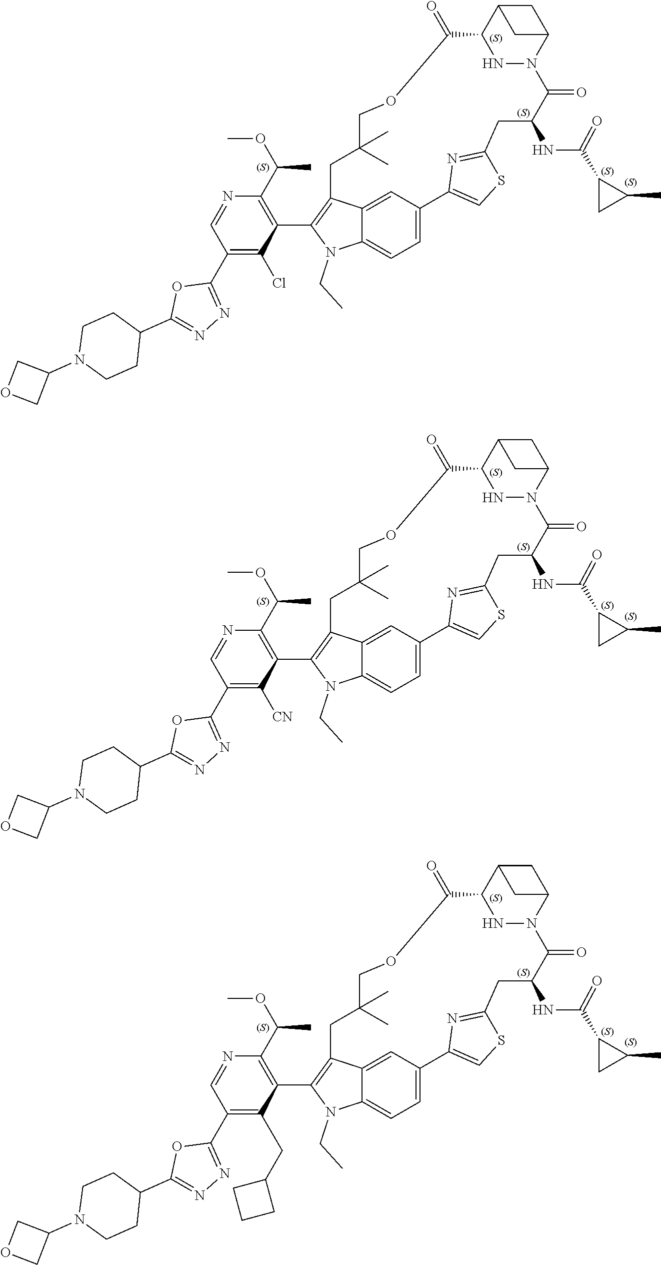

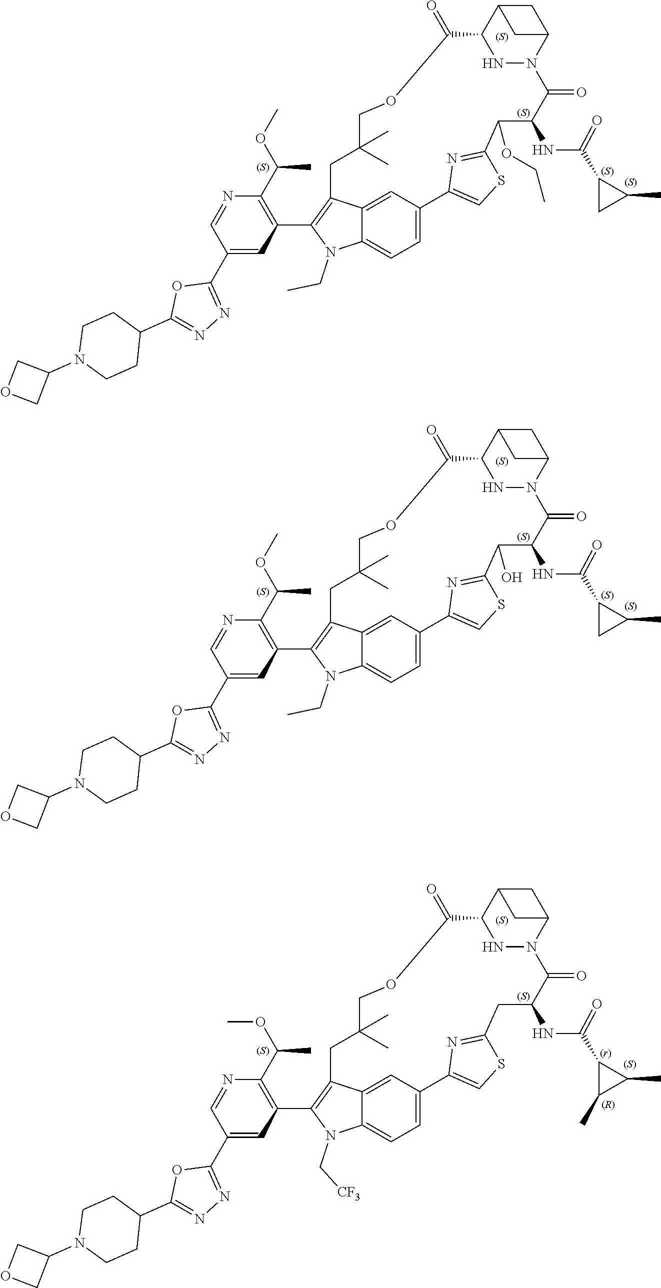

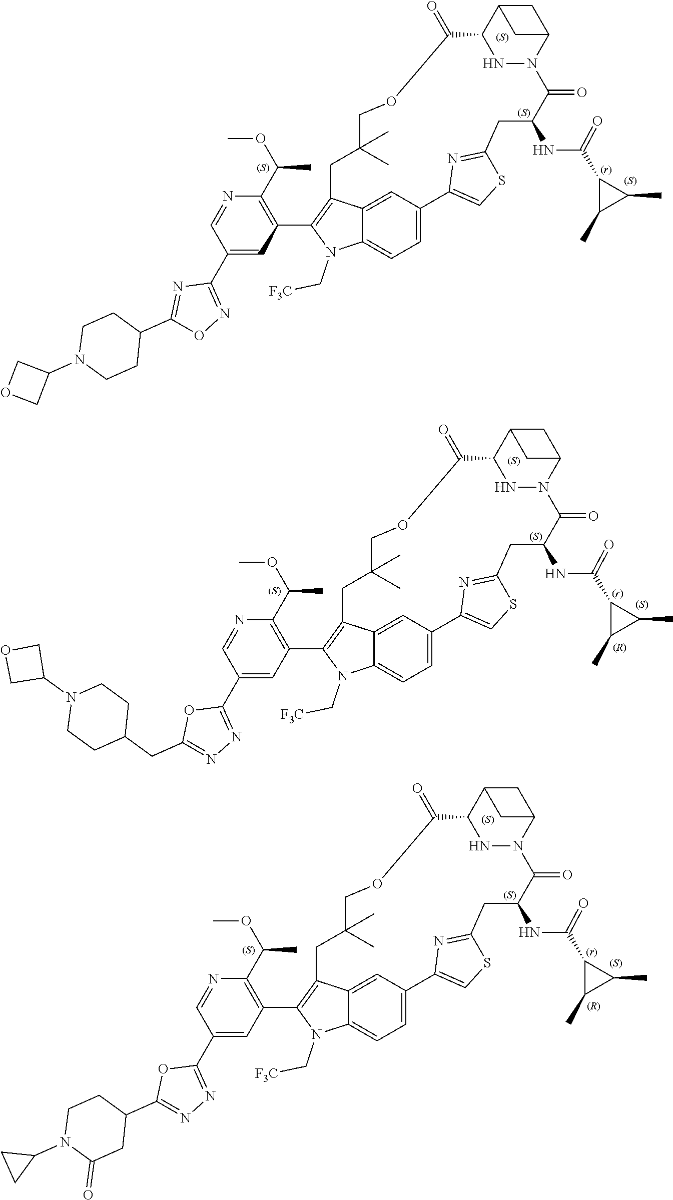

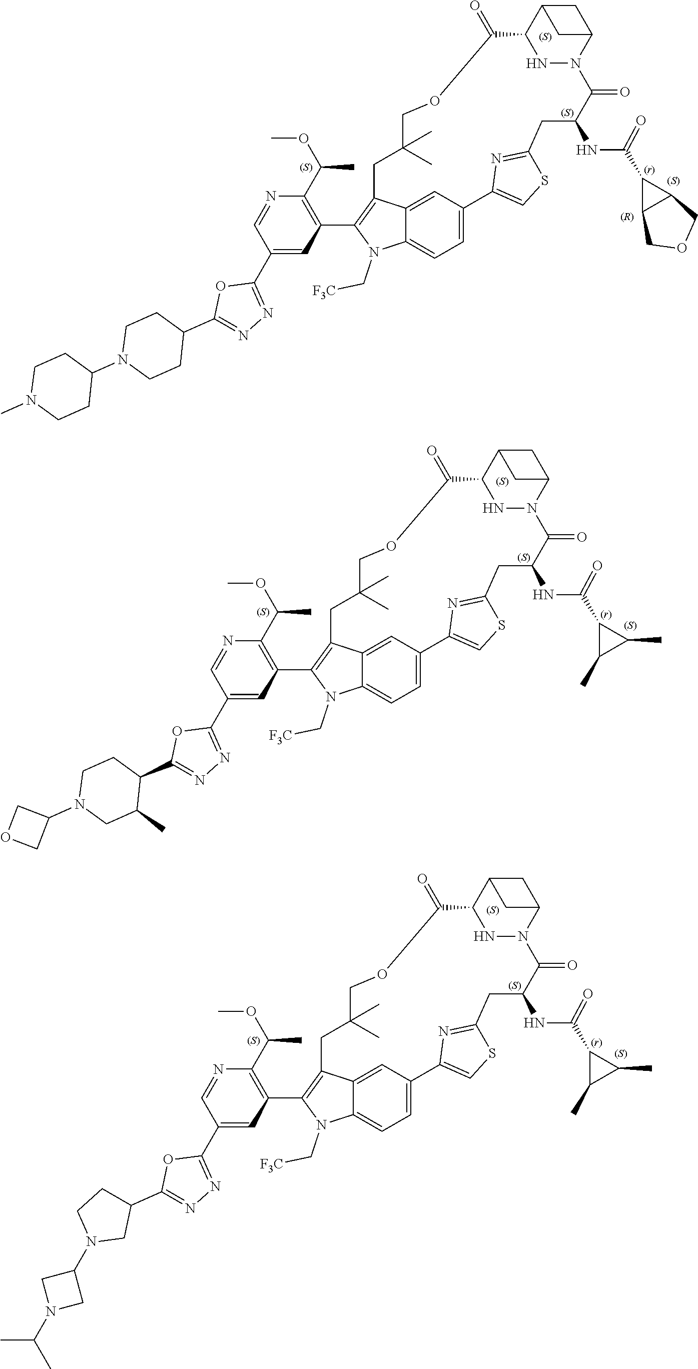

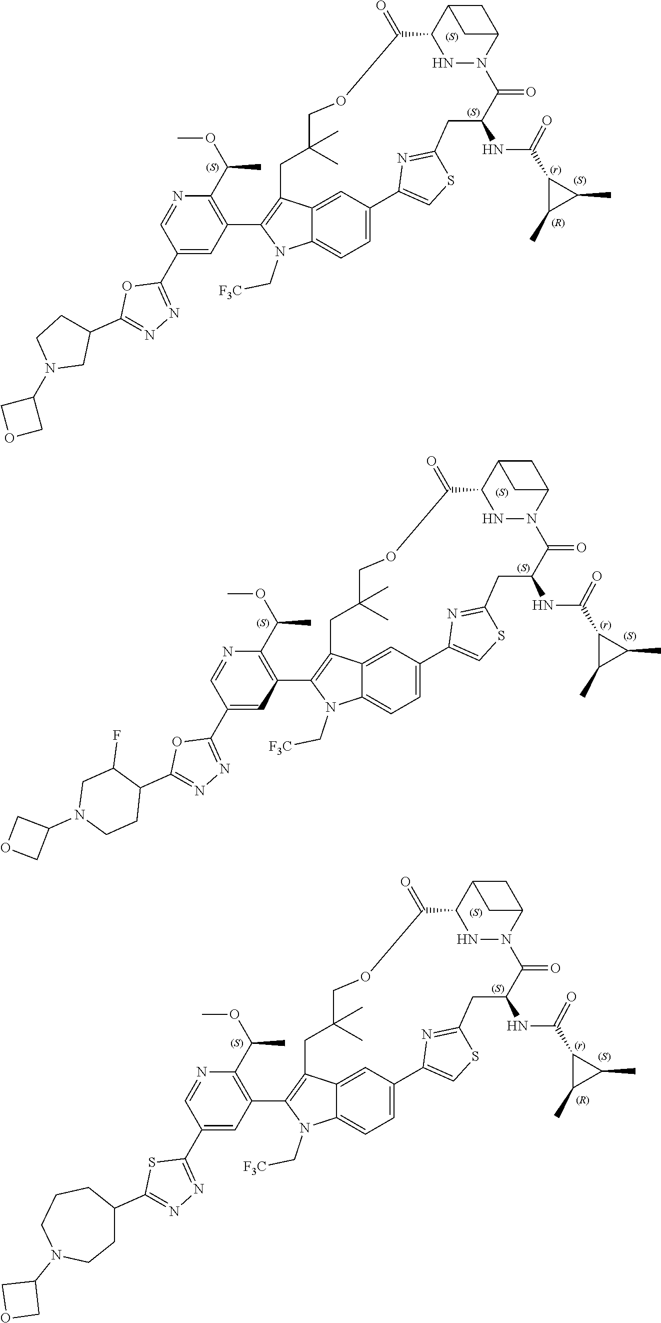

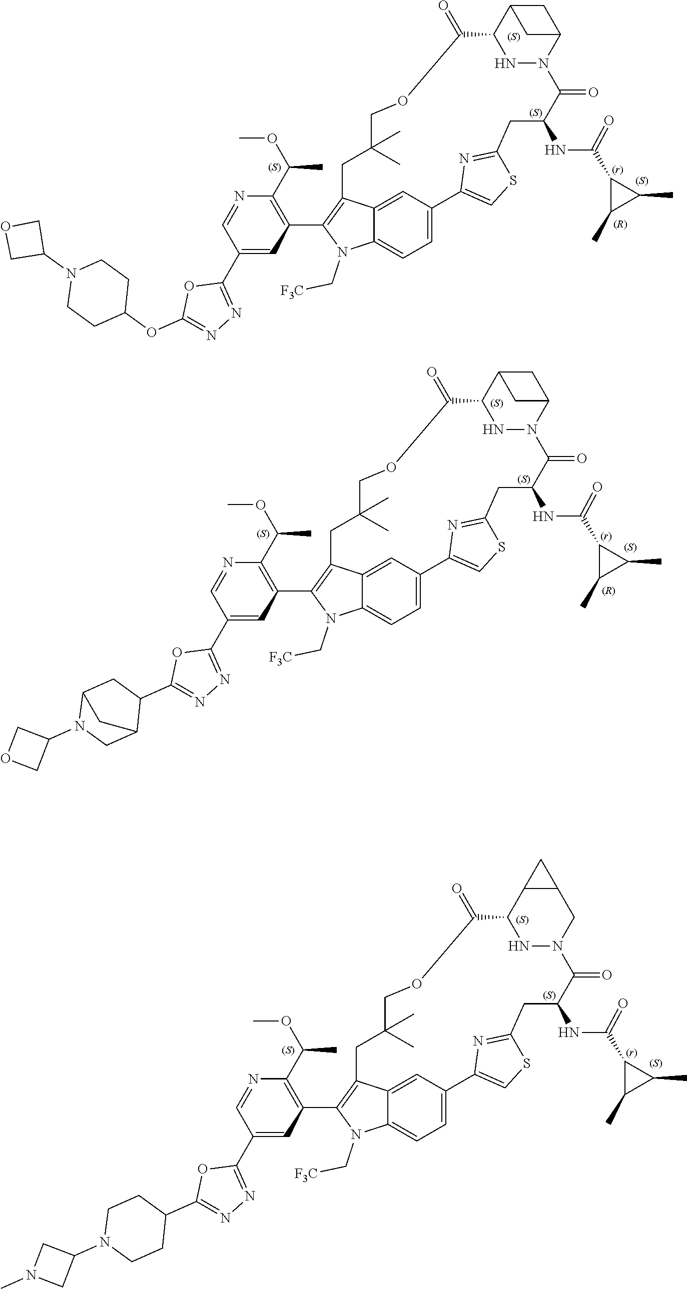

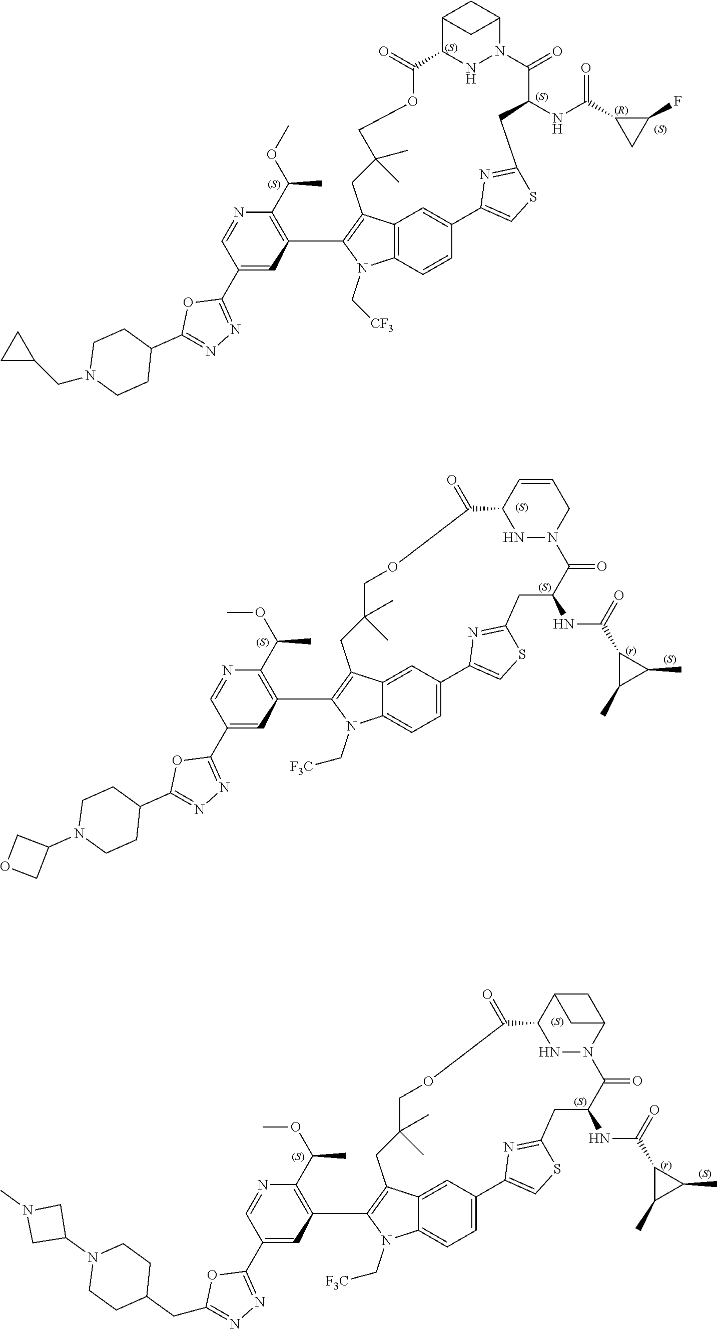

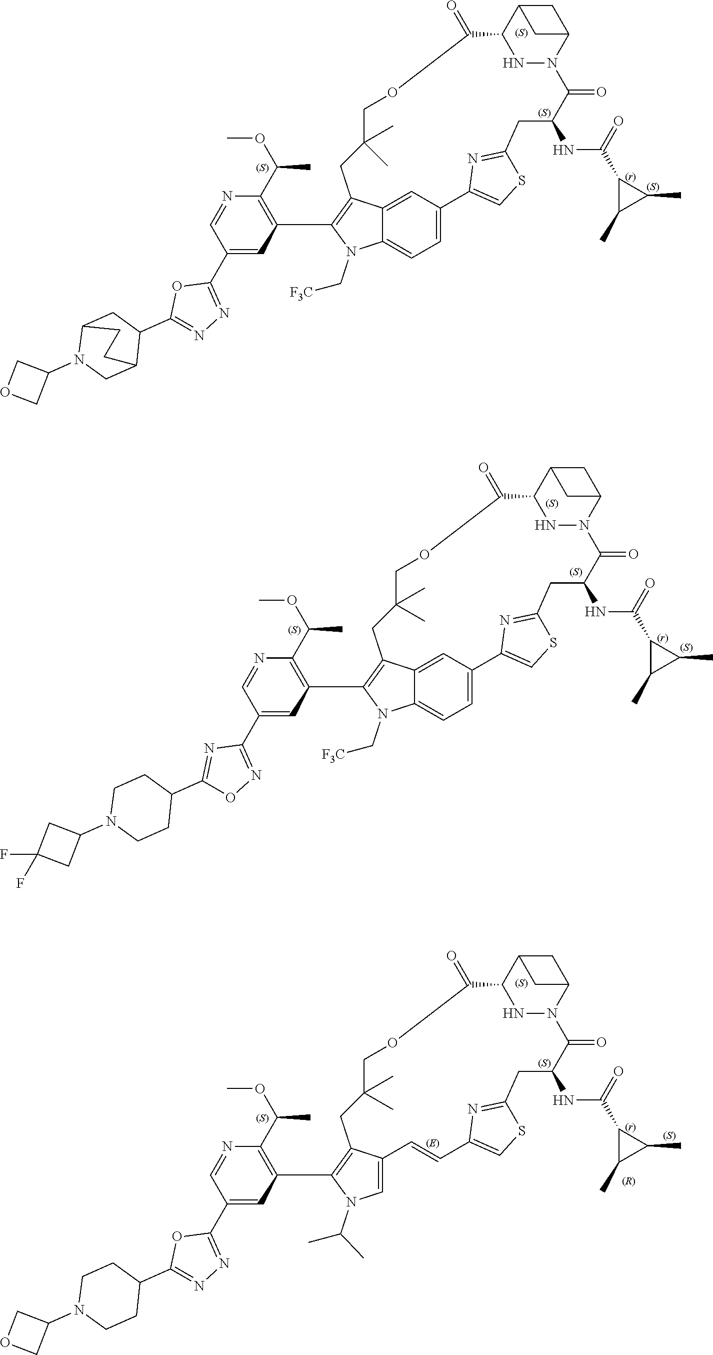

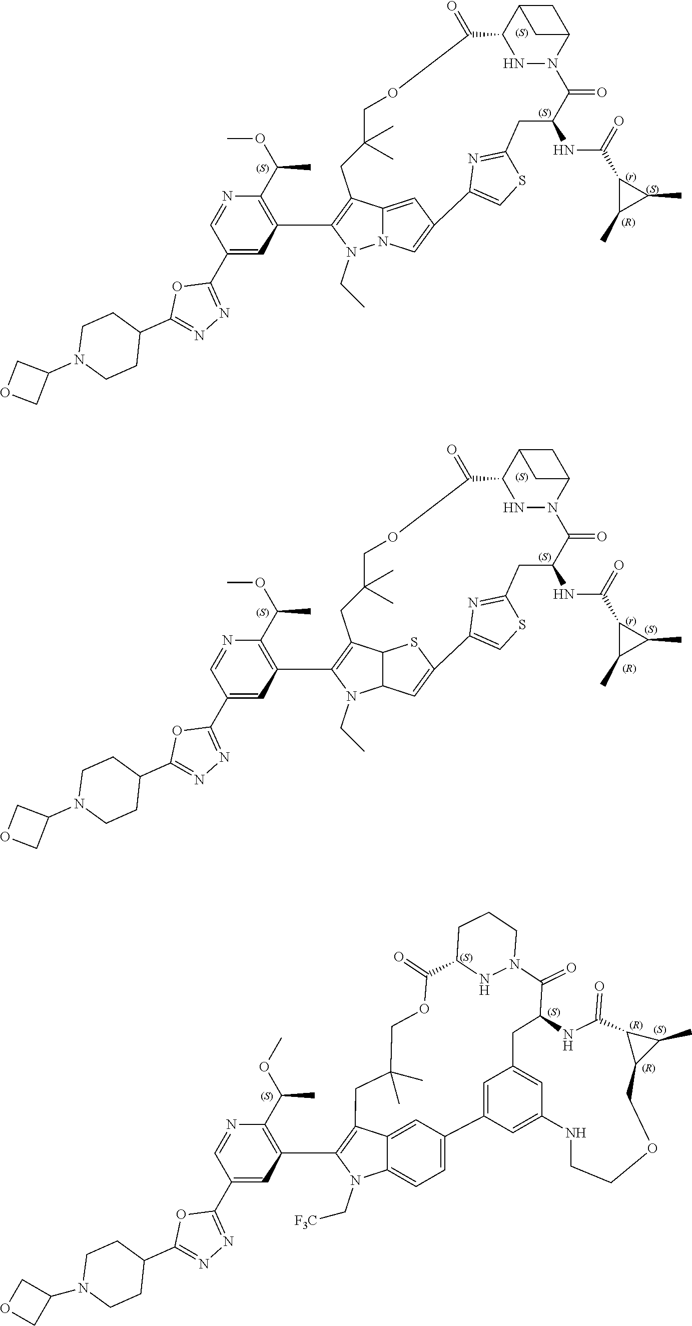

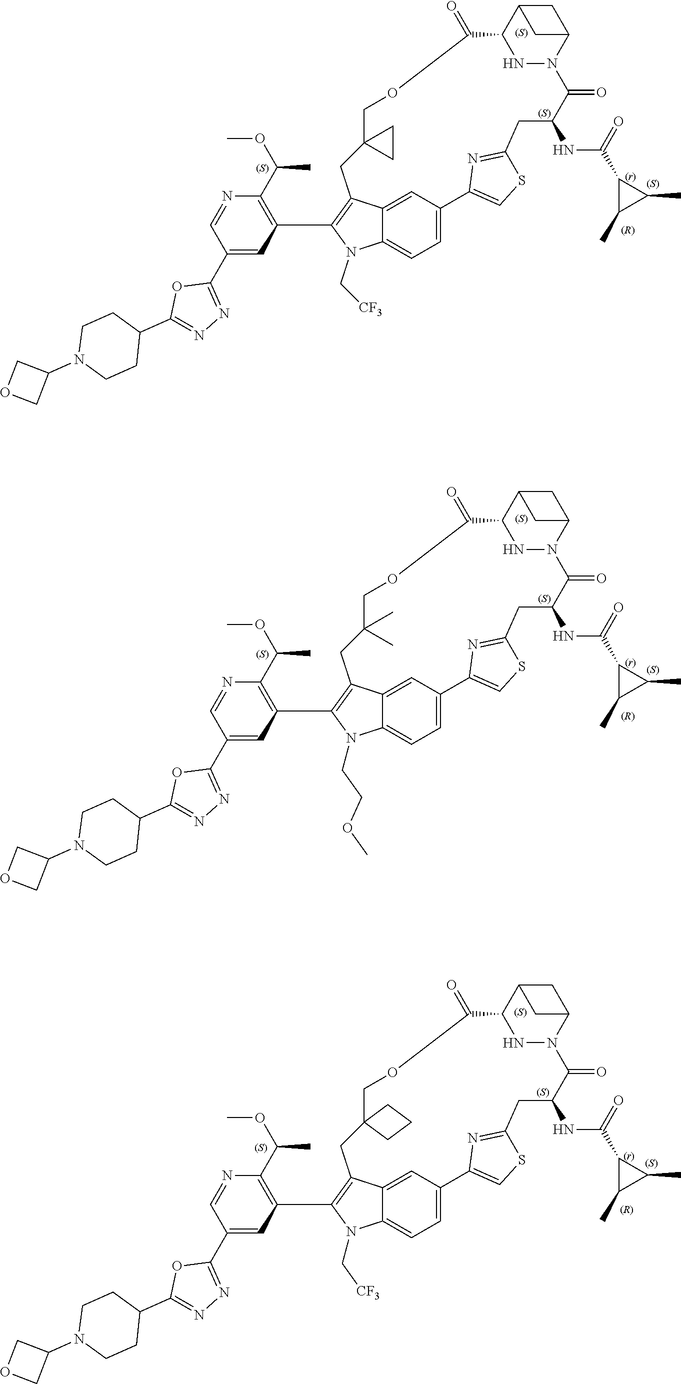

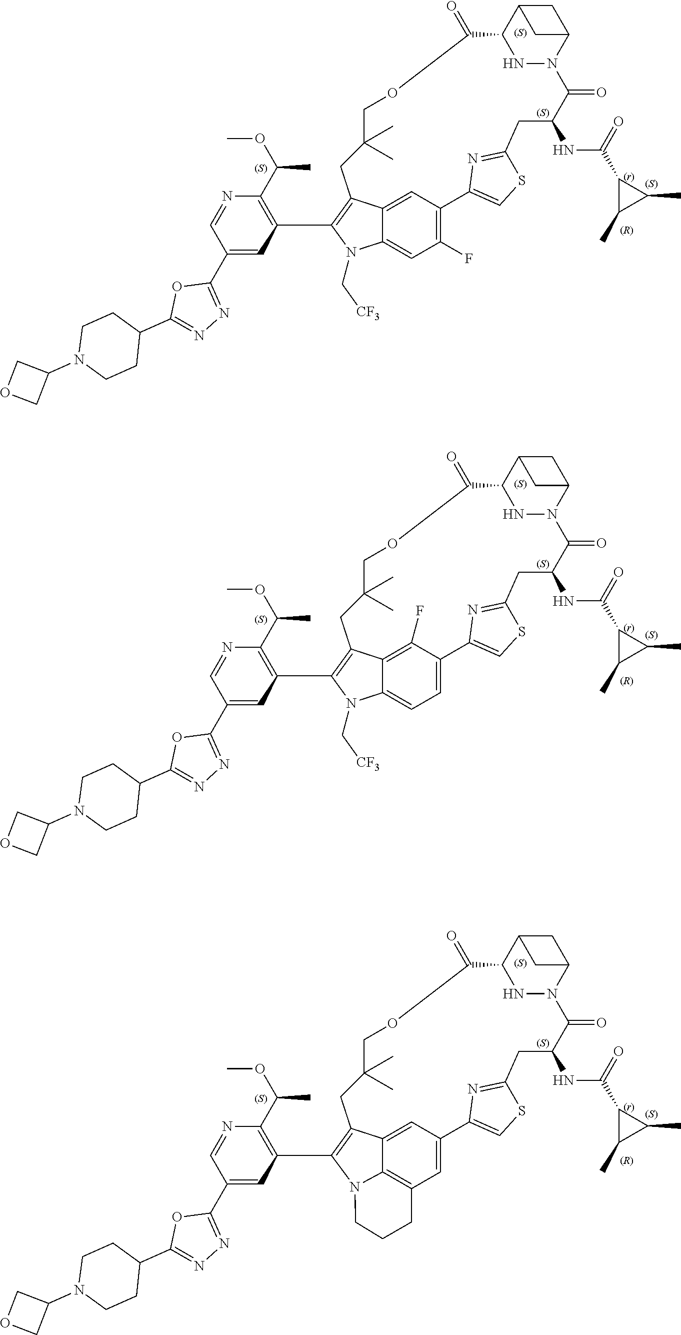

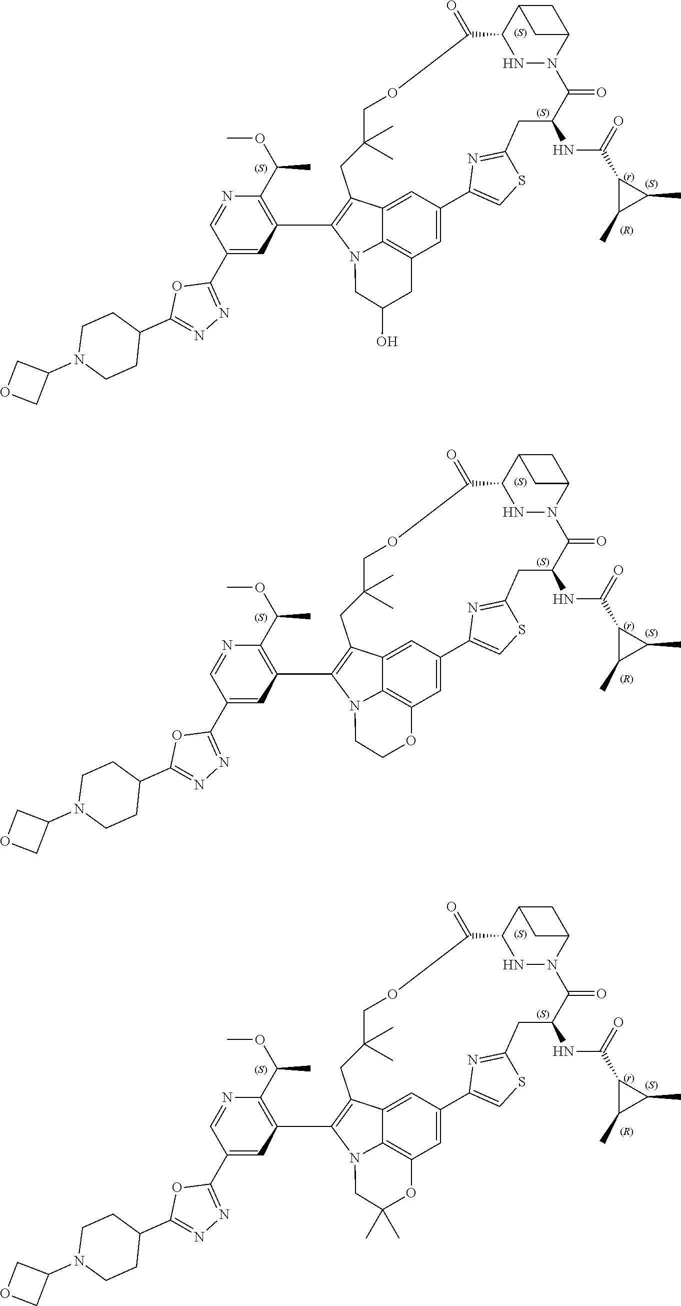

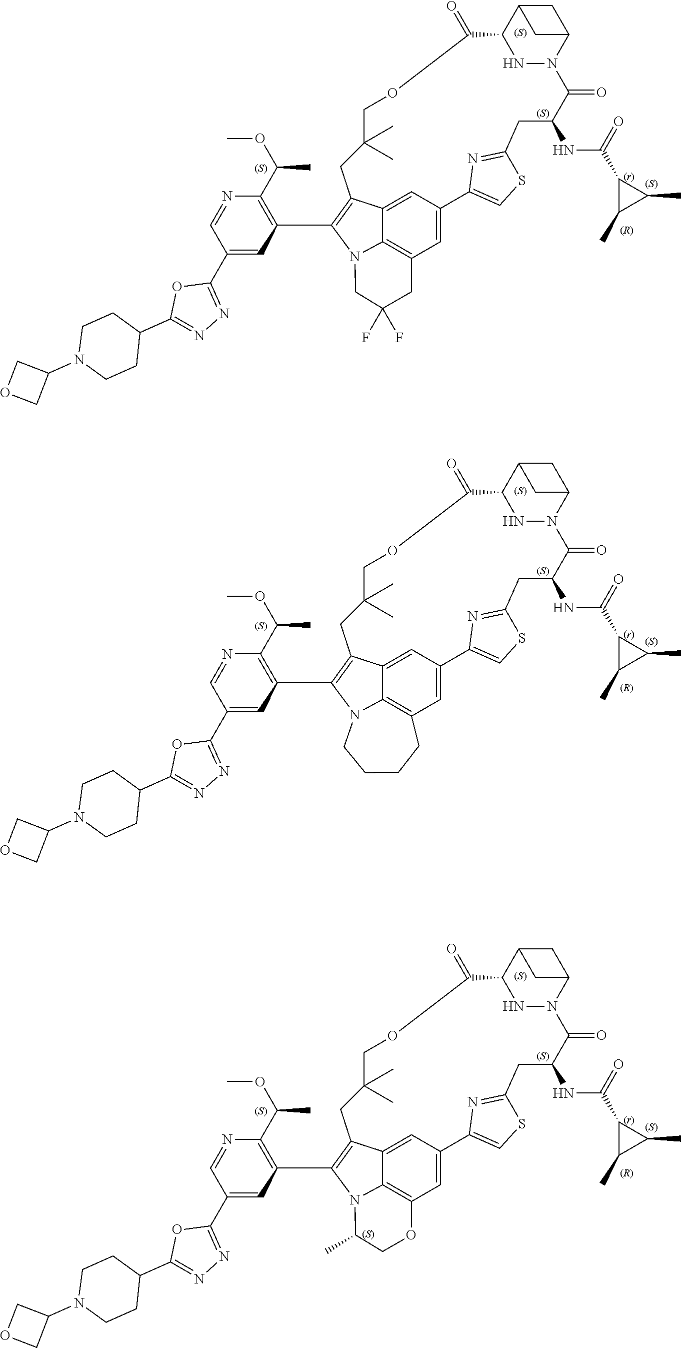

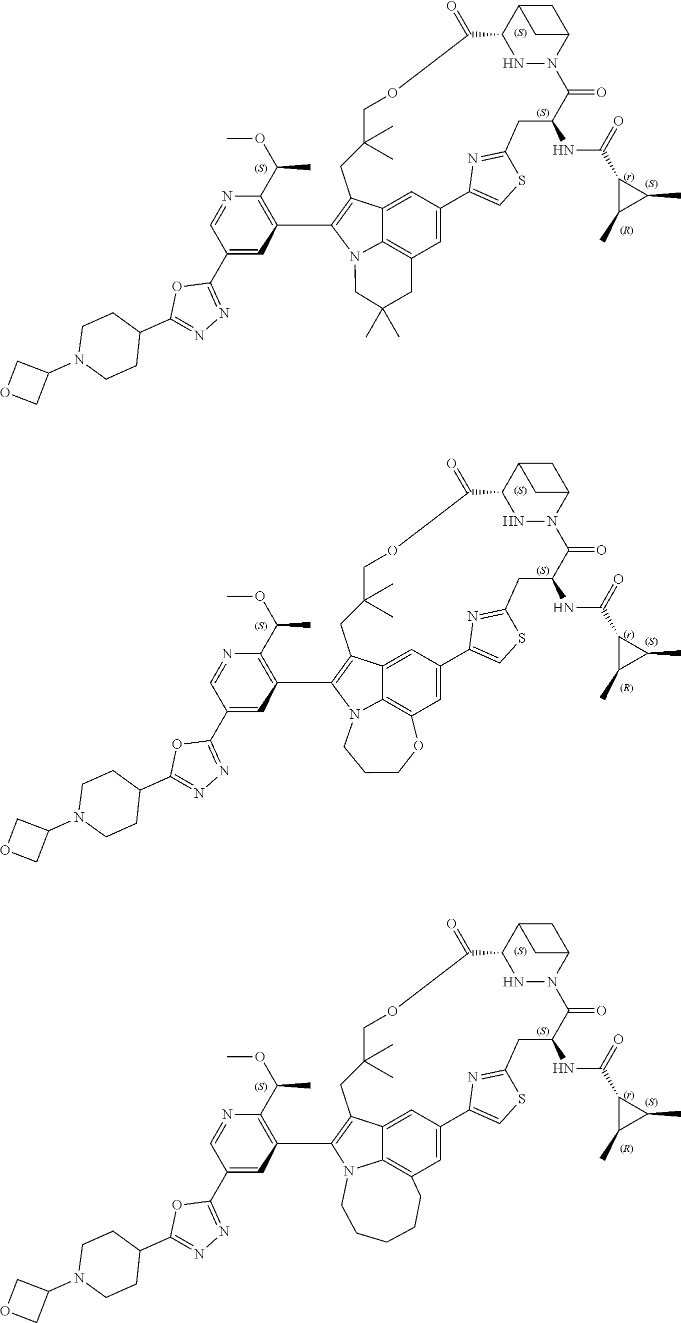

In another aspect, the disclosure provides a pharmaceutical composition comprising the aforementioned compound, or an isotopic derivative, stereoisomer, or a pharmaceutically acceptable salt thereof, and at least one pharmaceutically acceptable carrier.

In another aspect, the disclosure provides the use of the aforementioned compound, or an isotopic derivative, stereoisomer, or a pharmaceutically acceptable salt thereof, or a pharmaceutical composition in the manufacture of a medicament for the prevention and/or treatment of cancer, tumors, inflammatory diseases, autoimmune diseases, or immune-mediated diseases.

In yet another aspect, the disclosure provides the use of the aforementioned compound, or an isotopic derivative, stereoisomer, or a pharmaceutically acceptable salt thereof, or a pharmaceutical composition in the prevention and/or treatment of cancer, tumors, inflammatory diseases, autoimmune diseases, or immune-mediated diseases.

In a further aspect, the disclosure provides a method for the prevention and/or treatment of cancer, tumors, inflammatory diseases, autoimmune diseases, or immune-mediated diseases, comprising administering to a patient in need thereof a therapeutically effective amount of the aforementioned compound, or an isotopic derivative, stereoisomer, or a pharmaceutically acceptable salt thereof, and/or a pharmaceutical composition.

In some embodiments, the disease, tumor, inflammatory disease, autoimmune disease, or immune-mediated disease in the above-mentioned use or method is a RAS protein-associated disease; in some preferred embodiments, the RAS protein is one or more selected from KRAS protein, NRAS protein, or HRAS protein.

In certain embodiments, the symptom, tumor, inflammatory disease, autoimmune disease, or immune-mediated disease in the above-mentioned use or method comprises a RAS mutation. In certain preferred embodiments, the RAS mutation comprises one or more of a KRAS mutation, an NRAS mutation, or an HRAS mutation. Specifically, the RAS mutation is located at position 12, 13, and/or 61. More specifically, the RAS mutation comprises one or more of KRAS G12C, KRAS G12D, KRAS G12V, KRAS G12R, KRAS G13D, or NRAS Q61L.

It should be noted in particular that, in the present specification, when reference is made to a “compound” having the structure of Formula (A), Formula (A′), Formula (B), Formula (B′), Formula (I), Formula (I′), Formula (III), Formula (III′), Formula (IV), or Formula (IV′), such reference generally also encompasses its stereoisomers, diastereomers, enantiomers, racemic mixtures, and isotopic derivatives.

As is well known to those skilled in the art, salts, solvates, and hydrates of a compound are alternative forms of the compound, and can be converted into the compound under certain conditions. Accordingly, it should be noted in particular that, in the present specification, when reference is made to a compound having the structure of Formula (A), Formula (A′), Formula (B), Formula (B′), Formula (I), Formula (I′), Formula (III), Formula (III′), Formula (IV), or Formula (IV′), such reference generally also includes pharmaceutically acceptable salts thereof, and further includes solvates and hydrates thereof.

Similarly, in the present specification, reference to a compound generally also includes prodrugs, metabolites, and N-oxides thereof.

The pharmaceutically acceptable salts of the disclosure may be formed using, for example, the following inorganic or organic acids. “Pharmaceutically acceptable salt” means a salt which, within the scope of reasonable medical judgment, is suitable for use in contact with tissues of humans and lower animals, without undue toxicity, irritation, allergic reaction, etc., can be called a reasonable benefit/risk ratio. The salts can be prepared in situ during the final isolation and purification of the compounds of the disclosure, or alone by reacting the free base or acid with a suitable reagent, as outlined below. For example, a free base function can be reacted with a suitable acid. Examples of pharmaceutically acceptable inorganic acid addition salts are amino acids with inorganic acids (e.g., hydrochloric, hydrobromic, phosphoric, sulfuric, and perchloric) or organic acids (e.g., acetic, oxalic, maleic, tartaric, lemon acid, succinic acid or malonic acid), or by using other methods known in the art such as ion exchange. Other pharmaceutically acceptable salts include adipate, sodium alginate, ascorbate, aspartate, benzenesulfonate, benzoate, bisulfate, borate, butyrate, camphorate, camphorsulfonate, citrate, cyclopentanepropionate, digluconate, lauryl sulfate, ethylate, formate, fumarate, glucoheptonate, glycerin phosphate, gluconate, hernisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectate, persulfate, 3-phenylpropionate, phosphate, bitter salt, pivalate, propionate, stearate, succinate, sulfate, tartrate, thiocyanate, p-toluene sulfonate, undecanoate, valerate, etc. Representative alkali or alkaline earth metal salts include those of sodium, lithium, potassium, calcium, magnesium, and the like. Other pharmaceutically acceptable salts include, where appropriate, nontoxic ammonium salts, quaternary ammonium salts, and amine cations formed with counterions, for example, halides, hydroxides, carboxylates, sulfates, phosphates, nitrates, lower alkylsulfonates and arylsulfonates.

The pharmaceutically acceptable salts of the disclosure can be prepared by conventional methods, for example, by dissolving the compound of the present invention in a water-miscible organic solvent (such as acetone, methanol, ethanol and acetonitrile), adding an excess of organic acid or inorganic acid aqueous solution, so that the salt is precipitated from the resulting mixture, the solvent and remaining free acid are removed therefrom, and the precipitated salt is isolated.

The precursors or metabolites described in the disclosure may be precursors or metabolites known in the art, as long as the precursors or metabolites are transformed into compounds through in vivo metabolism. For example, “prodrugs” refer to those prodrugs of the compounds of the disclosure which, within the scope of sound medical judgment, are suitable for use in contact with human and lower animal tissues without undue toxicity, irritation, allergic response, etc., qualified as having a reasonable benefit/risk ratio and valid for its intended use. The term “prodrug” refers to a compound that is rapidly transformed in vivo to yield the parent compound of the above formula, for example by in vivo metabolism, or N-demethylation of a compound of the disclosure.

“Solvate” as used herein means a physical association of a compound of the present disclosure with one or more solvent molecules, whether organic or inorganic. This physical association includes hydrogen bonding. In some cases, for example, when one or more solvent molecules are incorporated into the crystal lattice of a crystalline solid, solvates will be able to be isolated. Solvent molecules in solvates may exist in regular and/or disordered arrangements. Solvates may contain stoichiometric or non-stoichiometric amounts of solvent molecules. “Solvate” encompasses both solution-phase and isolatable solvates. Exemplary solvates include, but are not limited to, hydrates, ethanolates, methanolates, and isopropanolates. Solvation methods are well known in the art.

The “stereoisomerism” described in the disclosure is divided into conformational isomerism and configurational isomerism, and configurational isomerism can also be divided into cis-trans isomerism and optical isomerism (that is, optical isomerism). Due to the rotation or twisting of carbon and carbon single bonds in organic molecules of a certain configuration, a stereoisomerism phenomenon in which each atom or atomic group of the molecule has a different arrangement in space, the common structures are alkanes and cycloalkanes. Such as the chair conformation and boat conformation that appear in the structure of cyclohexane. “Stereoisomer” means when a compound of the disclosure contains one or more asymmetric centers and is thus available as racemates and racemic mixtures, single enantiomers, diastereomeric mixtures and single diastereomers. The compound of the disclosure has an asymmetric center, and each asymmetric center can produce two optical isomers, and the scope of the disclosure includes all possible optical isomers and diastereoisomer mixtures and pure or partially pure compounds. The compounds described herein may exist in tautomeric forms having different points of attachment of hydrogens by displacement of one or more double bonds. For example, a ketone and its enol form are keto-enol tautomers. Each tautomer and mixtures thereof are included in the compounds of the present invention. All enantiomers, diastereoisomers, racemates, mesoforms, cis-trans isomers, tautomers, geometric isomers, epimers of compounds of (A), (A′), (B), (B′), (I), (I′), (III), (III′), (IV), and (IV′) and mixtures thereof are included in the scope of the disclosure.

An “isotopic derivative” of the disclosure refers to a molecule in which a compound is isotopically labeled. Isotopes commonly used for isotopic labeling are: hydrogen isotopes, 2H and 3H; carbon isotopes: 11C, 13C and 14C; chlorine isotopes: 35Cl and 37Cl; fluorine isotopes: 18F; iodine isotopes: 123I and 125I; nitrogen isotopes: 13N and 15N; Oxygen isotopes: 15O, 17O and 18O and sulfur isotope 35S. These isotope-labeled compounds can be used to study the distribution of pharmaceutical molecules in tissues. Especially deuterium 3H and carbon 13C are more widely used due to their easy labeling and convenient detection. Substitution of certain heavy isotopes, such as deuterium (2H), can enhance metabolic stability, prolong half-life and thus achieve the purpose of reducing doses and provide therapeutic advantages. Isotopically labeled compounds are generally synthesized starting from labeled starting materials and carried out in the same way as non-isotopically labeled compounds using known synthetic techniques.

The disclosure also provides the use of the compound of the present invention in the preparation of medicaments for preventing and/or treating cancer, tumor, inflammatory disease, autoimmune disease or immune-mediated disease.

In addition, the disclosure provides a pharmaceutical composition for preventing and/or treating cancer, tumor, inflammatory disease, autoimmune disease, neurodegenerative disease, attention-related disease or immune-mediated disease, which comprises the disclosure compounds as active ingredients. The pharmaceutical composition may optionally comprise a pharmaceutically acceptable carrier.

In addition, the disclosure provides a method of preventing and/or treating cancer, tumor, inflammatory disease, autoimmune disease, neurodegenerative disease, attention-related disease or immune-mediated disease, which comprises administering the compound of the disclosure to the mammals in need thereof.

Representative examples of inflammatory, autoimmune, and immune-mediated diseases may include, but are not limited to, arthritis, rheumatoid arthritis, spondyloarthritis, gouty arthritis, osteoarthritis, juvenile arthritis, Other Arthritis Conditions, Lupus, Systemic Lupus Erythematosus (SLE), Skin Related Disorders, Psoriasis, Eczema, Dermatitis, Atopic Dermatitis, Pain, Pulmonary Disease, Lung Inflammation, Adult Respiratory Distress Syndrome (ARDS), pulmonary sarcoidosis, chronic pulmonary inflammatory disease, chronic obstructive pulmonary disease (COPD), cardiovascular disease, atherosclerosis, myocardial infarction, congestive heart failure, myocardial ischemia-reperfusion injury, inflammatory bowel disease, Crohn's disease, ulcerative colitis, irritable bowel syndrome, asthma, Sjogren's syndrome, autoimmune thyroid disease, urticaria (rubella), multiple sclerosis, scleroderma, organ transplant rejection, xenograft, idiopathic thrombocytopenic purpura (ITP), Parkinson's disease, Alzheimer's disease, diabetes-related diseases, inflammation, pelvic inflammatory disease, allergic rhinitis, allergic bronchitis, allergic sinusitis, leukemia, lymphoma, B Cell Lymphoma, T Cell Lymphoma, Myeloma, Acute Lymphocytic Leukemia (ALL), Chronic Lymphocytic Leukemia (CLL), Acute Myeloid Leukemia (AML), Chronic Myelogenous Leukemia (CML), Hairy Cell Leukemia, He Jie King's disease, non-Hodgkin's lymphoma, multiple myeloma, myelodysplastic syndrome (MDS), myeloproliferative neoplasm (MPN), diffuse large B-cell lymphoma, and follicular lymphoma.

Representative examples of cancer or tumor may include, but are not limited to, skin cancer, bladder cancer, ovarian cancer, breast cancer, stomach cancer, pancreatic cancer, prostate cancer, colon cancer, lung cancer, bone cancer, brain cancer, neuroblastoma, rectal cancer, colon cancer, familial adenomatous polyposis carcinoma, hereditary nonpolyposis colorectal cancer, esophagus cancer, lip cancer, larynx cancer, hypopharyngeal cancer, tongue cancer, salivary gland cancer, stomach cancer, adenocarcinoma, medullary thyroid cancer, Papillary thyroid cancer, renal cancer, renal parenchymal cancer, ovarian cancer, cervical cancer, uterine body cancer, endometrial cancer, choriocarcinoma, pancreatic cancer, prostate cancer, testicular cancer, urinary cancer, melanoma, brain tumors such as Glioblastoma, astrocytoma, meningioma, medulloblastoma and peripheral neuroectodermal tumor, Hodgkin lymphoma, non-Hodgkin lymphoma, Burkitt lymphoma, acute lymphoblastic leukemia (ALL), chronic lymphocytic leukemia (CLL), acute myeloid leukemia (AML), chronic myelogenous leukemia (CML), adult T-cell leukemia lymphoma, diffuse large B-cell lymphoma (DLBCL), hepatocellular carcinoma, gallbladder Carcinoma, bronchial carcinoma, small cell lung cancer, non-small cell lung cancer, multiple myeloma, basal cell tumor, teratoma, retinoblastoma, choroidal melanoma, seminoma, rhabdomyosarcoma, craniopharyngioma, Osteosarcoma, chondrosarcoma, sarcoma, liposarcoma, fibrosarcoma, Ewing sarcoma, or plasmacytoma.

When the compound of the disclosure or a pharmaceutically acceptable salt thereof is administered in combination with another anticancer agent or immune checkpoint inhibitor for the treatment of cancer or tumors, the compound of the disclosure or a pharmaceutically acceptable salt thereof can provide enhanced anticancer effects.

Representative examples of anticancer agents useful in the treatment of cancer or tumors may include, but are not limited to, cell signal transduction inhibitor, chlorambucil, melphalan, cyclophosphamide, ifosfamide, busulfan, carmustine, lomustine, streptozotocin, cisplatin, carboplatin, oxaliplatin, dacarbazine, temozolomide, procarbazine, methotrexate, fluorouracil, cytarabine, gemcitabine, Mercaptopurine, fludarabine, vin-blastine, vincristine, vinorelbine, paclitaxel, docetaxel, topotecan, irinotecan, etoposide, trabectedin, dactinomycin, doxorubicin, epirubicin, daunorubicin, mitoxantrone, bleomycin, mitomycin C, ixabepilone, tamoxifen, flutamide, gonadorelin analogs, methadone progesterone, prednisone, dexamethasone, methylprednisolone, thalidomide, interferon alpha, leucovorin, sirolimus, sirolimus ester, everolimus, afatinib, alisertib, amuvatinib, apatinib, axitinib, bortezomib, bosutinib, britinib, cabozantinib, cediranib, crenolanib, kezhuotinib, dabrafenib, dabrafenib, Cotinib, danucitinib, dasatinib, dovitinib, erlotinib, foretinib, ganetespib, gefitinib, ibrutinib, icotinib, imatinib, iniparib, la Patinib, lenvatinib, linifanib, linsitinib, masitinib, momelotinib, motisanib, neratinib, nilotinib, niraparib, oprozomib, olaparib, pazopanib, pictilisib, ponatinib, quizartinib, regorafenib, rigosertib, rucaparib, ruxolitinib, saracatinib, saridegib, sorafenib, sunitinib, tiratinib, tivantinib, tivozanib, tofacitinib, trametinib, vandetanib, veliparib, vemurafenib, vimodegib, volasertib, alemtuzumab, bevacizumab, berentuzumab vedotin, catumaxumab Antibodies, cetuximab, denosumab, gemtuzumab, ipilimumab, nimotuzumab, ofatumumab, panitumumab, rituximab, tositumomab, trastuzumab, PI3K inhibitor, CSF1R inhibitor, A2A and/or A2B receptor antagonist, IDO inhibitor, anti-PD-1 antibody, anti-PD-L1 antibody, LAG3 antibody, TIM-3 antibody and an anti-CTLA-4 antibody, or any combination thereof.

When administered in combination with other therapeutic agents for the treatment of inflammatory disease, autoimmune disease and immune-mediated disease, the compounds of the disclosure, or pharmaceutically acceptable salts thereof provide enhanced therapeutic effect.

Representative examples of therapeutic agents useful in the treatment of inflammatory, autoimmune, and immune-mediated diseases can include, but are not limited to, steroidal agents (e.g., prednisone, prednisone, prednisone, methylphenidate, cortisone, hydroxycortisone, betamethasone, dexamethasone, etc.), methotrexate, leflunomide, anti-TNFα agents (eg, etanercept, infliximab, adalib monoclonal antibody, etc.), calcineurin inhibitors (e.g., tacrolimus, pimecrolimus, etc.), and antihistamines (e.g., diphenhydramine, hydroxyzine, loratadine, ebastine, ketotifen, cetirizine, levocetirizine, fexofenadine, etc.), and at least one or more therapeutic agents selected from them can be included in the pharmaceutical composition of the disclosure.

Other features of the disclosure will become apparent in the course of the description of exemplary embodiments of the disclosure which are given to illustrate the disclosure and are not intended to be limiting thereof. In the following examples the methods disclosed in the disclosure were used for preparation, separation and characterization.

The compounds of the disclosure can be prepared in a variety of methods known to those skilled in the art of organic synthesis, the methods described below as well as synthetic methods known in the art of synthetic organic chemistry or by variations thereof known to those skilled in the art may be used for the synthesis of the compounds of the disclosure. Preferred methods include, but are not limited to, those described below. Reactions are performed in solvents or solvent mixtures appropriate to the kit materials used and to the transformations effected. Those skilled in the art of organic synthesis will appreciate that the functionality present on the molecule is consistent with the proposed transitions. This sometimes requires judgment to alter the order of synthetic steps or starting materials to obtain the desired compound of the disclosure.

Unless otherwise stated, the terms used in the present application, including the specification and claims, are defined as follows. If not stated otherwise, conventional methods of mass spectrometry, NMR, HPLC, protein chemistry, biochemistry, recombinant DNA techniques and pharmacology are used. In this application, the use of “or” or “and” means “and/or” if not stated otherwise.

In the specification and claims, a given chemical formula or name shall encompass all stereoisomers and optical isomers thereof and racemates in which such isomers exist. Unless otherwise indicated, all chiral (enantiomers and diastereoisomers) and racemic forms are within the scope of the disclosure. Various geometric isomers of C═C double bonds, C═N double bonds, ring systems, etc. may also exist in the compounds, and all such stable isomers are encompassed by the disclosure. The disclosure describes cis- and trans- (or E- and Z-) geometric isomers of the compounds of the disclosure and which may be isolated as a mixture of isomers or as separated isomeric forms. The compounds of the disclosure may be isolated in optically active or racemic forms. All methods used to prepare the compounds of the disclosure and intermediates prepared therein are considered part of the disclosure. When preparing enantiomeric or diastereomeric products, they may be separated by customary methods, for example by chromatography or fractional crystallization. Depending on the process conditions, the end products of the disclosure are obtained in free (neutral) or salt form. The free forms and salts of these end products are within the scope of the disclosure. A compound can be converted from one form to another, if desired. A free base or acid can be converted into a salt; a salt can be converted into the free compound or another salt; a mixture of isomeric compounds of the disclosure can be separated into the individual isomers. The compounds of the disclosure, their free forms and salts, may exist in various tautomeric forms in which the hydrogen atoms are transposed to other parts of the molecule and thus the chemical bonds between the atoms of the molecule are rearranged. It is to be understood that all tautomeric forms which may exist are included within the disclosure.

In the disclosure, when a listed linking group does not specify its direction of connectivity, the direction is considered to be arbitrary. For example, when L in

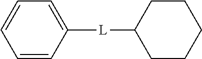

is —C(O)NH—, the group —C(O)NH— may connect a phenyl group and a cyclohexyl group in the left-to-right reading order to form

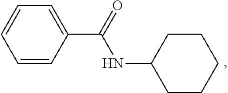 [SMILES: O=C(NC1CCCCC1)c1ccccc1]

or in the opposite reading order to form

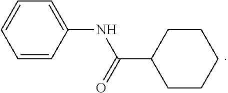 [SMILES: O=C(Nc1ccccc1)C1CCCCC1]

Such combinations of the linking group and the connected groups are permitted only when they result in a stable compound. In some preferred embodiments of the disclosure, the connectivity follows the left-to-right reading order.

Unless otherwise defined, the definitions of the substituents in the disclosure are independent and not interrelated, for example (listed but not exhaustive), in one aspect, for the substituents Ra (or Ra′) in different definitions of the substituents, the definitions are independent of each other. Specifically, when one definition is selected for R^{a} (or R^{a′}) in one substituent, it does not mean that R^{a} (or R^{a′}) has the same definition in other substituents. More specifically, for example (but not exhaustively) for NR^{a}R^{a′}, when R^{a} (or R^{a′}) is defined as hydrogen, it does not mean that in —C(O)—NR^{a}R^{a′}, R^{a} (or R^{a′}) must be hydrogen. In another aspect, when there is more than one R^{a} (or R^{a′}) in a certain substituent, these R^{a} (or R^{a′}) are also independent. For example, in the substituent —(CR^{a}R^{a′})m-O—(CR^{a}R^{a′})n-, when m+n is greater than or equal to 2, the m+n pieces of R^{a} (or R^{a′}) are independent, and they can be have the same or different meanings.

Unless otherwise defined, as used in the disclosure, the expression “substituted with x A substituents or B substituents” has the same meaning as “substituted with x substituents selected from A and B,” and when the number of substituents is greater than one, the x substituents may be the same or different. For example, “R₁ may be substituted with 0, 1, or 2 Rₓ substituents” means that R₁ may optionally be substituted with 0, 1, or 2 substituents selected from Rₓ, and when the number of substituents is greater than one, such substituents may be the same or different substituents represented by Rₓ. For example, “R₁ may be substituted with 0, 1, or 2 hydrogen, C₁-C₃ alkyl, or C₃-C₆ cycloalkyl substituents” means that R₁ may optionally be substituted with 0, 1, or 2 substituents selected from hydrogen, C₁-C₃ alkyl, or C₃-C₆ cycloalkyl, and when the number of substituents is greater than 1, the substituents may be the same or different. For instance, when the number of substituents is 2, the two substituents may be, for example, two H, two C₁-C₃ alkyl, one H and one C₁-C₃ alkyl, or one C₁-C₃ alkyl and one C₃-C₆ cycloalkyl.

Unless otherwise defined, when a substituent is designated as “optionally substituted” herein, the substituent refers to, for example, a substituent selected from the following: alkyl, hydroxyalkyl, aminoalkyl, cycloalkyl, heterocycloalkyl, aryl, heterocyclic group, halogen, hydroxy, alkoxy, oxo, alkanoyl, aryloxy, alkanoyloxy, amino, alkylamino, arylamino, arylalkylamino, disubstituted amino (wherein the two amino substituents are independently selected from alkyl, aryl, or arylalkyl), alkanoylamino, arylcarbonylamino, arylalkanoylamino, substituted alkanoylamino, substituted arylamino, substituted arylalkanoylamino, thio, alkylthio, arylthio, arylalkylthio, arylthiocarbonyl, arylalkylthiocarbonyl, alkylsulfonyl, arylsulfonyl, arylalkylsulfonyl, aminosulfonyl (e.g., —SO₂NH₂), substituted sulfonamido, nitro, cyano, carboxy, carbamoyl (e.g., —CONH), substituted carbamoyl (e.g., —CONHalkyl, —CONHaryl, —CONHarylalkyl or those having two substituents on nitrogen independently selected from alkyl, aryl, or arylalkyl), alkoxycarbonyl, aryl, substituted aryl, guanidino, and heterocyclic groups such as indolyl, imidazolyl, furyl, thienyl, thiazolyl, pyrrolidinyl, pyridyl, pyrimidinyl, pyrrolidinyl, piperidinyl, morpholinyl, piperazinyl, homopiperazinyl, and substituted heterocyclic groups.

Unless otherwise defined, as used herein, the term “single bond,” “bond,” or “direct bond” refers to two atoms being connected through a saturated covalent bond. For example, when L represents a single bond, “A-L-B” indicates that A and B are connected through a saturated covalent bond, i.e., “A-B.” As another example, when L represents a single bond, “—CH₂-L-NH—” indicates that —CH₂— and —NH— are connected through a saturated covalent bond, i.e., “—CH₂—NH—.”

As used herein, the term “alkyl” is intended to include saturated aliphatic hydrocarbon groups having a specified number of carbon atoms, which may be either branched or straight-chained. For example, “C1-C6 alkyl” refers to an alkyl group containing from 1 to 6 carbon atoms. Examples of alkyl groups include, but are not limited to, methyl (Me), ethyl (Et), propyl (e.g., n-propyl and isopropyl), butyl (e.g., n-butyl, isobutyl, and tert-butyl), and pentyl (e.g., n-pentyl, isopentyl, and neopentyl). The alkyl group may be unsubstituted or substituted, and when substituted, may be substituted at any available position. The substituents are preferably selected from one or more of deuterium, halogen, hydroxyl, amino, cyano, alkyl, alkoxy, haloalkyl, cycloalkyl, heterocycloalkyl, aryl, and heteroaryl groups. In the present disclosure, the alkyl group is preferably a C1-C6 alkyl, more preferably a C1-C4 alkyl.

As used herein, the term “alkylene” is intended to refer to saturated aliphatic hydrocarbon bridges, either straight- or branched-chain, optionally containing or not containing a cycloalkyl group, derived by removal of two hydrogen atoms from the same or two different carbon atoms of a parent alkane. For example, “C0-C6 alkylene” refers to alkylene bridges having 0 (i.e., a bond), 1, 2, 3, 4, 5, or 6 carbon atoms. Examples of alkylene include, but are not limited to, methylene (—CH₂—), ethylene (—CH₂CH₂—), propylene (e.g., —(CH₂)₃—, —(CHCH₃)CH₂—, —(CHCH₂CH)—), butylene (e.g., —(CH₂)—, —CH₂CH(CH₂CH₃)—, —CH₂(CHCH₂CH)—), pentylene (e.g., —(CH₂)—, —CH₂CH(CH(CH₃)₂)—, —CH₂(CHCH₂CH)CH₂—), and hexylene (e.g., —(CH₂)—, —CH₂CH₂CH(CH(CH₃))—, —CH₂(CHCH(CH₃)CH)CH₂—). The alkylene is preferably selected from C0-C6, C0-C4, C0-C3, C1-C6, C1-C4, or C1-C3 alkylene groups, more preferably those not comprising cycloalkyl moieties.

The term “cycloalkyl” refers to a saturated hydrocarbon ring, which may be monocyclic, polycyclic, or branched. Exemplary C3-C12 cycloalkyl groups include, but are not limited to, cyclopropyl, cyclobutyl, cyclopentyl, cyclohexyl, and norbornyl. Branched cycloalkyls, such as 1-methylcyclopropyl and 2-methylcyclopropyl, are included. Polycyclic structures include bicyclic and tricyclic cycloalkyls, which may be bridged, spiro, or fused. Cycloalkyl groups may be unsubstituted or substituted at any available position, and the substituents are preferably selected from one or more of halogen, hydroxyl, amino, cyano, oxo, alkyl, alkoxy, haloalkyl, cycloalkyl, heterocycloalkyl, aryl, and heteroaryl. In the disclosure, C₃-C₁₂ cycloalkyl groups are preferred, more preferably C₃-C₈.

Similarly, the term “heterocycloalkyl” refers to a saturated cyclic structure in which at least one carbon atom in a cycloalkyl ring is replaced by a heteroatom selected from N, O, S, or P. The nitrogen may optionally be quaternized, and N or S may be oxidized (e.g., to NO, SO, or SO₂). The term includes monocyclic, bicyclic, and tricyclic heterocyclic structures, including spiro, fused, and bridged heterocycles. The heterocycloalkyl may be unsubstituted or substituted, and when substituted, may be substituted at any available position. The substituents are preferably selected from one or more of halogen, hydroxyl, amino, cyano, oxo, alkyl, alkoxy, haloalkyl, cycloalkyl, heterocycloalkyl, aryl, and heteroaryl groups. In the disclosure, 4- to 12-membered heterocycloalkyl groups are preferred, more preferably 4- to 8-membered ones.

In the disclosure, the term “fused ring” refers to a polycyclic group formed by two or more cyclic structures sharing two adjacent atoms.

In the disclosure, the term “bridged ring” refers to a polycyclic group in which two rings share more than two ring atoms.

In the disclosure, the term “spiro ring” refers to a polycyclic group formed by two monocyclic structures sharing a single carbon atom, referred to as a spiro atom.

The term “alkenyl” denotes a straight or branched chain hydrocarbon group containing one or more double bonds and generally having a length of 2 to 20 carbon atoms. For example, “C₂-C₆ alkenyl” contains two to six carbon atoms. Alkenyl groups include, but are not limited to, e.g. vinyl, propenyl, butenyl, 1-methyl-2-buten-1-yl, and the like. Herein, alkenyl is preferably C₂-C₆ alkenyl.

The term “cycloalkenyl” refers to a monocyclic or bicyclic cyclic alkenyl group. Monocyclic cycloalkenyl refers to C₃-C₈ cyclic alkenyl, including but not limited to cyclopropenyl, cyclobutenyl, cyclopentenyl, cyclohexenyl and norbornenyl. Branched cycloalkenyl groups such as 1-methylcyclopropenyl and 2-methylcyclopropenyl are included within the definition of “cycloalkenyl”. Bicyclic cycloalkenyl groups include bridged, spiro, or fused cycloalkenyl groups.

The term “alkynyl” denotes a straight or branched chain hydrocarbon group containing one or more triple bonds and generally having a length of 2 to 20 carbon atoms. For example, “C₂-C₆ alkynyl” contains two to six carbon atoms. Representative alkynyl groups include, but are not limited to, for example, ethynyl, 1-propynyl, 1-butynyl, and the like. Herein, the alkynyl group is preferably a C₂-C₆ alkynyl group.

The term “alkoxy” or “alkyloxy” refers to —O-alkyl. “C₁-C₆ alkoxy” (or alkyloxy) is intended to include C₁, C₂, C₃, C₄, C₅, C₆ alkoxy. Examples of alkoxy include, but are not limited to, methoxy, ethoxy, propoxy (e.g., n-propoxy and isopropoxy), and tert-butoxy. Herein, the alkoxy group is preferably an alkoxy group having 1 to 6, more preferably 1 to 4 carbon atoms. Similarly, “alkylthio” or “thiothio” denotes an alkyl group as defined above having the indicated number of carbon atoms attached through a sulfur bridge; e.g. methyl-S— and ethyl-S—. The alkoxy group may be unsubstituted or substituted, and when substituted, it may be substituted at any available position. Preferred substituents are selected from one or more of deuterium, halogen, hydroxyl, amino, cyano, alkyl, alkoxy, haloalkyl, cycloalkyl, heterocycloalkyl, aryl, and heteroaryl.

The term “carbonyl” refers to an organic functional group (C═O) consisting of two atoms, carbon and oxygen, linked by a double bond.

The term “aryl”, alone or as part of a larger moiety such as “aralkyl”, “arylalkoxy” or “aryloxyalkyl”, refers to a monocyclic, bicyclic or tricyclic ring system having a total of 5 to 12 ring members, wherein at least one ring in the system is aromatic and wherein each ring in the system contains 3 to 7 ring members. In certain embodiments of the disclosure, “aryl” refers to an aromatic ring system which includes, but is not limited to, phenyl, biphenyl, indanyl, 1-naphthyl, 2-naphthyl, and tetrahydronaphthyl. The term “aralkyl” or “arylalkyl” refers to an alkyl residue attached to an aryl ring, non-limiting examples of which include benzyl, phenethyl, and the like. A fused aryl group can be attached to another group at a suitable position on the cycloalkyl ring or aromatic ring. Dashed lines drawn from ring systems indicate that bonds may be attached to any suitable ring atom. The aryl group may be unsubstituted or substituted, and when substituted, it may be substituted at any available position. The substituents are preferably selected from one or more of deuterium, halogen, hydroxyl, amino, cyano, alkyl, alkoxy, haloalkyl, cycloalkyl, heterocycloalkyl, aryl, and heteroaryl.

As used herein, the term “heterocycle” refers to a cyclic structure containing one or more heteroatoms, which may be fully saturated, partially saturated, or fully unsaturated, and includes heteroaryl rings.

The term “heteroaryl” means a stable 5-membered, 6-membered, or 7-membered aromatic monocyclic ring or aromatic bicyclic ring or a 7-membered, 8-membered, 9-membered, 10-membered, 11-membered, 12-membered aromatic polycyclic heterocycle, which is fully unsaturated or partially unsaturated, and contains carbon atoms and 1, 2, 3 or 4 heteroatoms independently selected from N, O and S; and includes cycloalkane or heterocycloalkane with a structure in which aromatic rings such as benzene rings or heteroaromatic rings such as pyridine are condensed. The position of the structure as a substituent may be located on a cycloalkane, a heterocycloalkane, an aromatic ring or a heteroaryl ring. Nitrogen and sulfur heteroatoms can be optionally oxidized. The nitrogen atom is substituted or unsubstituted (i.e. N or NR, where R is H or another substituent if defined). A heterocycle can be attached to its pendant group at any heteroatom or carbon atom that results in a stable structure. The heterocyclyl groups described herein may be substituted on carbon or nitrogen atoms if the resulting compound is stable. The nitrogen in the heterocycle can optionally be quaternized. Preferably, when the total number of S and O atoms in the heterocycle exceeds 1, then these heteroatoms are not adjacent to each other. Preferably, the total number of S and O atoms in the heterocycle is not greater than one. When the term “heterocycle” is used, it is intended to include heteroaryl. Examples of heteroaryl groups include, but are not limited to, acridinyl, azetidinyl, aziocinyl, benzimidazolyl, benzofuryl, benzothiofuranyl, benzothienyl, benzooxa azolyl, benzoxazolyl, benzothiazolyl, benzotriazolyl, benzotetrazolyl, benzisoxazolyl, benzisothiazolyl, benzimidazolyl, carbazolyl, 4aH-carbazolyl, carbolinyl, chromanyl, chromenyl, cinnolinyl, decahydroquinolyl, 2H,6H-1,5,2-dithiazinyl, dihydrofuro[2,3-b]tetrahydrofuryl, furyl, furanyl, imidazolidinyl, imidazolinyl, imidazolyl, 1H-indazolyl, imidazopyridyl, indolenyl, indolinyl, Indolazinyl, indolyl, 3H-indolyl, isatinoyl, isobenzofuryl, isochromanyl, isoindazolyl, isoindolinyl, isoindolyl, isoindolyl quinolinyl, isothiazolyl, isothiazolopyridyl, isoxazolyl, isoxazolopyridyl, methylenedioxyphenyl, morpholinyl, diazanaphthyl, octahydroisoquinoline oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolopyridyl, oxazolidinyl, diazaphenyl, oxindolyl, pyrimidinyl, phenanthridinyl, phenanthrolinyl, phenazinyl, phenothiazinyl, phenoxathiyl, phenoxazinyl, phthalazinyl, piperazinyl, piperidinyl, piperidinyl, 4-piperidinyl, piperonyl, pteridinyl, purinyl, pyranyl, pyrazinyl, pyrazolidinyl, pyrazolinyl, pyrazolopyridyl, pyrazolyl, pyridazinyl, pyridoxazolyl, pyridimidazolyl, pyridothiazolyl, pyridyl, pyrimidinyl, pyrrolidinyl, pyrrolinyl, 2-pyrrolidonyl, 2H-pyrrolyl, pyrrolyl, quinazolinyl, quinolinyl, 4H-quinazinyl, quinoxalinyl, quinuclidinyl, tetrazolyl, tetrahydrofuryl, tetrahydroisoquinolyl, tetrahydroquinolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthryl, thiazolyl, thienyl, thiazolopyridyl, thienothiazolyl, thienooxa Azolyl, thienoimidazolyl, thienyl, triazinyl, 1,2,3-triazolyl, 1,2,4-triazolyl, 1,2,5-triazolyl, 1,3,4-triazolyl and xanthenyl, quinolinyl, isoquinolyl, phthalazinyl, quinazolinyl, indolyl, isoindolyl, indolinyl, 1H-indazolyl, benzo Imidazolyl, 1,2,3,4-tetrahydroquinolinyl, 1,2,3,4-tetrahydroisoquinolinyl, 5,6,7,8-tetrahydro-quinolinyl, 2,3-Dihydro-benzofuryl, chromanyl, 1,2,3,4-tetrahydro-quinoxalinyl and 1,2,3,4-tetrahydro-quinazolinyl. The term “heteroaryl” may also include biaryl structures formed by the above-defined “aryl” and a monocyclic “heteroaryl”, such as but not limited to “-phenyl bipyridyl-”, “- “phenyl bipyrimidyl”, “-pyridyl biphenyl”, “-pyridyl bipyrimidyl-”, “-pyrimidyl biphenyl-”; wherein the disclosure also includes condensed ring and spiro compound containing, for example, the above-mentioned heterocycles.

The term “substituted” as used herein means that at least one hydrogen atom is replaced by a non-hydrogen group, provided that normal valences are maintained and that the substitution results in a stable compound. A cyclic double bond, as used herein, is a double bond formed between two adjacent ring atoms (e.g., C═C, C═N or N═N).

In the present disclosure, the one or more halogens may each be independently selected from fluorine, chlorine, bromine and iodine.

“Halo” or “halogen” includes fluoro, chloro, bromo and iodo. “Haloalkyl”/“haloalkylene” is intended to include branched and straight chain saturated alkyl/alkylene groups having the indicated number of carbon atoms substituted with one or more halogens. Examples of haloalkyl include, but are not limited to, fluoromethyl, difluoromethyl, trifluoromethyl, trichloromethyl, pentafluoroethyl, pentachloroethyl, 2,2,2-trifluoroethyl, heptafluoro propyl and heptachloropropyl. Examples of haloalkyl also include “fluoroalkyl” intended to include branched and straight-chain saturated aliphatic hydrocarbon groups having the specified number of carbon atoms and substituted with 1 or more fluorine atoms. “Halocycloalkyl”/“haloheterocycloalkyl” is intended to include cycloalkyl/heterocycloalkyl having the indicated number of carbon atoms substituted with one or more halogens. In the disclosure, the halogen atom is preferably fluorine or chlorine, more preferably fluorine.

“Haloalkoxy” or “haloalkyloxy” means a haloalkyl group as defined above having the indicated number of carbon atoms attached through an oxygen bridge. For example, “haloC₁-C₆ alkoxy” is intended to include C₁, C₂, C₃, C₄, C₅, C₆ haloalkoxy. Examples of haloalkoxy include, but are not limited to, trifluoromethoxy, 2,2,2-trifluoroethoxy, and pentafluoroethoxy. Similarly, “haloalkylthio” or “thiohaloalkoxy” denotes a haloalkyl group as defined above having the indicated number of carbon atoms attached through a sulfur bridge; for example trifluoromethyl-S— and pentafluoroethyl —S—.

In the present disclosure, the expression Cₓ₁-Cₓ₂ is used when referring to some substituent groups, which means that the number of carbon atoms in the substituent groups may be x1 to x2. For example, C₀-C₈ means that the group contains 0, 1, 2, 3, 4, 5, 6, 7 or 8 carbon atoms, C₁-C₈ means that the group contains 1, 2, 3, 4, 5, 6, 7 or 8 carbon atoms, C₂-C₈ means that the group contains 2, 3, 4, 5, 6, 7 or 8 carbon atoms, C₃-C₈ means that the group contains 3, 4, 5, 6, 7 or 8 carbon atoms, C₄-C₈ means that the group contains 4, 5, 6, 7 or 8 carbon atoms, C₀-C₆ means that the group contains 0, 1, 2, 3, 4, 5 or 6 carbon atoms, C₁-C₆ means that the group contains 1, 2, 3, 4, 5 or 6 carbon atoms, C₂-C₆ means that the group contains 2, 3, 4, 5 or 6 carbon atoms, C₃-C₆ means that the group contains 3, 4, 5 or 6 carbon atoms.

In this disclosure, the expression “x1-x2 membered ring” is used when referring to cyclic groups such as aryl, heteroaryl, cycloalkyl, and heterocycloalkyl, which means that the number of the ring atoms of the group can be x1 to x2. For example, the 3-12 membered cyclic group may be a 3, 4, 5, 6, 7, 8, 9, 10, 11 or 12 membered ring, and the number of ring atoms may be 3, 4, 5, 6, 7, 8, 9, 10, 11 or 12; 3-6-membered ring means that the cyclic group can be 3, 4, 5 or 6-membered ring, and the number of ring atoms can be 3, 4, 5 or 6; 3-8 membered ring means that the cyclic group can be 3, 4, 5, 6, 7 or 8-membered ring, and the number of ring atoms can be 3, 4, 5, 6, 7 or 8; 3-9 membered ring means that the cyclic group can be a 3, 4, 5, 6, 7, 8 or 9-membered ring, and the number of ring atoms can be 3, 4, 5, 6, 7, 8 or 9; 4-7 membered ring means that the cyclic group can be a 4, 5, 6 or 7-membered ring, and the number of ring atoms can be 4, 5, 6 or 7; 5-8-membered ring means that the cyclic group can be 5, 6, 7 or 8-membered ring, the number of ring atoms can be 5, 6, 7 or 8; 5-12 membered ring means that the ring group can be 5, 6, 7, 8, 9, 10, 11 or 12-membered ring, the number of ring atoms can be 5, 6, 7, 8, 9, 10, 11 or 12; 6-12 membered ring means that the ring group can be 6, 7, 8, 9, 10, 11- or 12-membered rings, the number of ring atoms may be 6, 7, 8, 9, 10, 11 or 12. The ring atoms may be carbon atoms or heteroatoms, for example selected from N, O and S. When the ring is heterocyclic, the heterocyclic ring may contain 1, 2, 3, 4, 5, 6, 7, 8, 9, 10 or more ring heteroatoms, for example selected from N, O and S of heteroatoms.

Where nitrogen atoms (e.g. amines) are present on compounds of the disclosure, these nitrogen atoms can be converted to N-oxides by treatment with oxidizing agents (e.g. mCPBA and/or hydrogen peroxide) to obtain other compounds of the disclosure. Accordingly, both shown and claimed nitrogen atoms are considered to cover both the shown nitrogen and its N-oxides to obtain the derivatives of the disclosure.

When any variable occurs more than one time in any composition or formula of a compound, its definition on each occurrence is independent of its definition at every other occurrence. Thus, for example, if a group is shown to be substituted with 0-3 R, then said group may be optionally substituted with up to three R groups, and R at each occurrence is independently selected from the definition of R. Also, combinations of substituents and/or variables are permissible only if such combinations result in stable compounds.

The term “patient” as used herein refers to an organism being treated by the methods of the disclosure. Such organisms preferably include, but are not limited to, mammals (e.g., murine, ape, monkey, equine, bovine, porcine, canine, feline, etc.) and most preferably refer to humans.

The term “effective amount” as used herein means an amount of a drug or agent (i.e., a compound of the disclosure) that will elicit a biological or medical response in a tissue, system, animal or human being sought, e.g., by a researcher or clinician. Furthermore, the term “therapeutically effective amount” means an amount which results in improved treatment, cure, prevention or alleviation of a disease, disorder or side effect, or a reduction in the rate of disease or disorder progression. An effective amount may be given in one or more administrations, applications or doses and is not intended to be limited by a particular formulation or route of administration. The term also includes within its scope amounts effective to enhance normal physiological function.

As used herein, the term “treating” includes any effect that results in amelioration of a condition, disease, disorder, etc., such as alleviation, reduction, regulation, amelioration or elimination, or amelioration of the symptoms thereof.

The term “pharmaceutically acceptable” is used herein to refer to those compounds, substances, compositions and/or dosage forms: within the scope of sound medical judgment, they are suitable for use in contact with human and animal tissues without excessive toxicity, irritation, allergic reactions, and/or other problems or complications, commensurate with a reasonable benefit/risk ratio.

The phrase “pharmaceutically acceptable carrier” as used herein means a pharmaceutical substance, composition or vehicle, such as a liquid or solid filler, diluent, excipient, manufacturing aid (e.g. lubricant, talc, magnesium stearate, calcium stearate, or zinc stearate, or stearic acid) or solvent-encapsulated substances involved in the carrying or transport of a subject compound from one organ or body part to another. Each carrier must be “acceptable” in the sense of being compatible with the other ingredients of the formulation and not injurious to the patient.

The term “pharmaceutical composition” means a composition comprising a compound of the disclosure together with at least one other pharmaceutically acceptable carrier. “Pharmaceutically acceptable carrier” means a medium generally accepted in the art for delivering a biologically active agent to an animal, particularly a mammal, including (i.e.) an adjuvant, excipient or vehicle, such as a diluent, preservatives, fillers, flow regulators, disintegrants, wetting agents, emulsifiers, suspending agents, sweeteners, flavoring agents, fragrances, antibacterial agents, antifungal agents, lubricants and dispersants, which depends on the mode of administration and the nature of the dosage form.

The term “acceptable”, as used herein, means that a formulation ingredient or active ingredient does not have an undue adverse effect on health for the general purpose of treatment.

The term “cancer”, as used herein, refers to an abnormal growth of cells that cannot be controlled and, under certain conditions, is capable of metastasizing (spreading). Cancers of this type include, but are not limited to, solid tumors (e.g., bladder, bowel, brain, chest, uterus, heart, kidney, lung, lymphoid tissue (lymphoma), ovary, pancreas or other endocrine organs (e.g., thyroid), prostate, skin (melanoma), or blood cancer (such as non-leukemic leukemia).

The term “administration in combination” or similar terms, as used herein, refers to the administration of several selected therapeutic agents to a patient, in the same or different modes of administration at the same or different times.

The term “enhancing” or “capable of enhancing”, as used herein, means that a desired result is capable of being increased or prolonged, either in potency or duration. Thus, in relation to enhancing the therapeutic effect of a drug, the term “capable of potentiating” refers to the ability of the drug to increase or prolong its potency or duration in the system. As used herein, “potency value” refers to the ability to maximize the enhancement of another therapeutic drug in an ideal system.

The term “immune disease” refers to a disease or condition of an adverse or deleterious reaction to an endogenous or exogenous antigen. The result is usually dysfunction of the cells, or destruction thereof and dysfunction, or destruction of organs or tissues that may produce immune symptoms.

The term “subject” or “patient” includes mammals and non-mammals. Mammals include, but are not limited to, mammals: humans, non-human primates such as orangutans, apes, and monkeys; agricultural animals such as cattle, horses, goats, sheep, and pigs; domestic animals such as rabbits and dogs; experimental animals include rodents, such as rats, mice and guinea pigs. Non-mammalian animals include, but are not limited to, birds, fish, and the like. In a preferred aspect, the selected mammal is a human.

The term “treatment”, “course of treatment” or “therapy” as used herein includes alleviating, suppressing or ameliorating the symptoms or conditions of a disease; inhibiting the development of complications; ameliorating or preventing the underlying metabolic syndrome; inhibiting the development of diseases or symptoms, such as controlling the development of a disease or condition; alleviating a disease or a symptom; causing a disease or a symptom to regress; alleviating a complication caused by a disease or a symptom, or preventing and/or treating a sign caused by a disease or a symptom.

The term “inhibit” or “reduce,” or any variations of these terms, encompasses any measurable decrease or complete inhibition to achieve the intended result. For example, the activity may be decreased relative to normal by about, at most about, or at least about 5%, 10%, 15%, 20%, 25%, 30%, 35%, 40%, 45%, 50%, 55%, 60%, 65%, 70%, 75%, 80%, 85%, 90%, 95%, 99% or more, or by any range derivable therefrom.

As used herein, the term “wild-type” refers to an entity having the structure or activity observed in nature in a “normal” state or condition, as opposed to a mutated, diseased, or altered state. It will be understood by those skilled in the art that wild-type genes and polypeptides typically exist in a variety of different forms, such as allelic variants.

As used herein, the terms “antagonist” and “inhibitor” are used interchangeably and refer to a compound that inhibits the biological function of a target protein by inhibiting the activity or expression of a protein such as K-Ras, H-Ras, or N-Ras G12C. Accordingly, the terms “antagonist” and “inhibitor” are defined in the context of the biological action of the target protein. While preferred antagonists herein specifically interact (e.g., bind) with the target, the definition expressly encompasses compounds that inhibit the biological activity of the target protein by interacting with other members of a signal transduction pathway of which the target protein is a member. Preferred biological activities inhibited by the antagonist are those associated with the initiation, growth, or spread of tumors.

As used herein, a compound or pharmaceutical composition, upon administration, can improve a disease, symptom, or condition, particularly by improving its severity, delaying onset, slowing disease progression, or reducing the duration of the condition. The term “improve” as used herein encompasses effects attributable to or associated with administration, whether the administration is fixed or temporary, continuous or intermittent.

The composition may be prepared separately according to conventional mixing, granulation, or coating methods, and the pharmaceutical composition of the disclosure may contain, by weight or volume, about 0.1% to about 99%, about 5% to about 90%, or about 1% to about 20% of the compound of the disclosure or a pharmaceutically acceptable salt thereof. In some embodiments, the amount of the compound or pharmaceutically acceptable salt thereof described herein may be from 1% to 95% by weight of the total pharmaceutical composition.

The composition may be provided in a dosage form suitable for intra-articular, oral, parenteral (e.g., intravenous, intramuscular), rectal, dermal, subcutaneous, topical, transdermal, sublingual, intranasal, vaginal, intravesical, intraurethral, intrathecal, epidural, otic, or ophthalmic administration, or for administration by injection, inhalation, or direct contact with nasal, urogenital, genital, or oral mucosa. Accordingly, the pharmaceutical composition may be presented, for example, as tablets, capsules, pills, powders, granules, suspensions, emulsions, solutions, gels (including hydrogels), pastes, ointments, creams, plasters, syrups, osmotic delivery devices, suppositories, enemas, injections, implants, sprays, formulations suitable for iontophoretic delivery, or aerosols. The composition may be formulated in accordance with conventional pharmaceutical practice.

As used herein, the term “administration” refers to the act of delivering a composition (e.g., a compound or a formulation comprising the compound described herein) to a subject or system. Administration to an animal subject (e.g., a human) may be carried out via any appropriate route. For example, in some embodiments, administration may be bronchial (including bronchial instillation), buccal, enteral, intradermal, intra-arterial, intragastric, intraosseous, intramuscular, intranasal, intraperitoneal, intrathecal, intravenous, intravesical, transmucosal, intranasal, oral, rectal, subcutaneous, sublingual, topical, tracheal (including intratracheal instillation), transdermal, vaginal, or intravitreal.

The formulation may be prepared in a form suitable for systemic administration or for topical or local administration. Systemic formulations include those designed for injection (e.g., intramuscular, intravenous, or subcutaneous injection) or for transdermal, transmucosal, or oral administration. Formulations will generally include a diluent, and in some cases, may include adjuvants, buffers, preservatives, and the like. The compound or a pharmaceutically acceptable salt thereof may also be administered in the form of a liposomal composition or as a microemulsion.

For injection, the formulation may be prepared in conventional forms such as a liquid solution or suspension, a solid form suitable for preparing a solution or suspension in a liquid immediately prior to injection, or an emulsion. Suitable excipients include, for example, water, saline, dextrose, and glycerol. These compositions may also contain an amount of non-toxic auxiliary substances such as wetting or emulsifying agents, pH buffering agents, and the like, for example, sodium acetate, sorbitan monolaurate, and the like.

Systemic administration may also include relatively non-invasive methods such as suppositories, transdermal patches, transmucosal delivery, and intranasal administration. Oral administration is also suitable for the compounds of the disclosure or pharmaceutically acceptable salts thereof. It will be understood by those skilled in the art that suitable forms include syrups, capsules, and tablets.

Each compound described herein, or a pharmaceutically acceptable salt thereof, may be formulated in various ways known in the art. For example, in a combination therapy, the first agent and the second agent may be formulated together or separately. Other modes of combination therapy are also described herein.

A drug formulated individually or separately may be packaged together in the form of a kit. Non-limiting examples include, for instance, a kit containing two pills, one pill and a powder, a suppository or a liquid in a vial, or two types of topical creams. The kit may optionally include components useful for administering a unit dose to a subject, such as a vial for reconstituting a powder form, a syringe for injection, a customized intravenous (IV) delivery system, an inhaler, and the like. In addition, a unit-dose kit may contain instructions relating to the preparation and administration of the composition. The kit may be manufactured for a single-use unit dose for one subject, for multiple uses for a specific subject (with a fixed dose, or wherein the potency of individual compounds or their pharmaceutically acceptable salts may vary over the course of treatment), or may contain multiple doses suitable for administration to multiple subjects (“bulk packaging”). The components of the kit may be assembled in cartons, blister packs, bottles, tubes, and the like.

Oral formulations include tablets comprising a mixture of the active ingredient with a non-toxic, pharmaceutically acceptable excipient. The excipient may be, for example, an inert diluent or filler (e.g., sucrose, sorbitol, sugar, mannitol, microcrystalline cellulose, starch including potato starch, calcium carbonate, sodium chloride, lactose, calcium phosphate, calcium sulfate, or sodium phosphate); a granulating agent and disintegrant (e.g., cellulose derivatives, including microcrystalline cellulose, starch including potato starch, cross-linked carboxymethyl cellulose sodium, alginates or alginic acid); a binder (e.g., sucrose, glucose, sorbitol, gum arabic, alginic acid, sodium alginate, gelatin, starch, pregelatinized starch, microcrystalline cellulose, magnesium aluminum silicate, sodium carboxymethyl cellulose, methyl cellulose, optionally substituted hydroxypropyl methyl cellulose, ethyl cellulose, polyvinylpyrrolidone, or polyethylene glycol); and a lubricant, glidant, and anti-adherent (e.g., magnesium stearate, zinc stearate, stearic acid, silicon dioxide, hydrogenated vegetable oil, or talc). Other pharmaceutically acceptable excipients may include colorants, flavoring agents, plasticizers, humectants, buffering agents, and the like.

Two or more compounds may be mixed together in a tablet, capsule, or other carrier, or may be kept separate. In one example, the first compound is contained on the inside of the tablet, and the second compound is positioned on the outside, whereby the majority of the second compound is released before the first compound is released.

Oral formulations may also be provided in the form of chewable tablets, or in the form of hard gelatin capsules in which the active ingredient is mixed with an inert solid diluent, such as potato starch, lactose, microcrystalline cellulose, calcium carbonate, calcium phosphate, or kaolin; or in the form of soft gelatin capsules in which the active ingredient is mixed with water or an oil medium, such as peanut oil, liquid paraffin, or olive oil. Powders, granules, and pellets may be prepared using the ingredients mentioned above for tablets and capsules, in a conventional manner, for example, using a mixer, fluid-bed apparatus, or spray-drying equipment.

Controlled release through dissolution or diffusion may be achieved by appropriately coating tablets, capsules, pills, or granular formulations of the compound, or by incorporating the compound or its pharmaceutically acceptable salt into an appropriate matrix. The controlled-release coating may include one or more of the coating substances mentioned above, such as shellac, beeswax, glycowax, castor wax, carnauba wax, stearyl alcohol, monostearin, distearin, palmitostearin, ethylcellulose, acrylic resins, dl-polylactic acid, cellulose acetate butyrate, polyvinyl chloride, polyvinyl acetate, vinylpyrrolidone, polyethylene, polymethacrylate, methyl methacrylate, 2-optionally substituted hydroxy methacrylate, methacrylate hydrogel, 1,3-butanediol, ethylene glycol methacrylate, or polyethylene glycol. In controlled-release matrix formulations, the matrix material may also include, for example, hydroxy methylcellulose, carnauba wax and stearyl alcohol, carbopol 934, silicone, tristearin, methyl acrylate-methyl methacrylate, polyvinyl chloride, polyethylene, or halofluorocarbon.

Liquid forms suitable for incorporation into the compounds of the disclosure or pharmaceutically acceptable salts and compositions thereof for oral administration include aqueous solutions, suitably flavored syrups, aqueous or oily suspensions, emulsions flavored with edible oils such as cottonseed oil, sesame oil, coconut oil, or peanut oil, as well as elixirs and similar pharmaceutical carriers.

In general, when administered to humans, the oral dosage of any compound of the disclosure or a pharmaceutically acceptable salt thereof will depend on the nature of the compound and can be readily determined by those skilled in the art. The dosage may be, for example, about 0.001 mg to about 2000 mg per day, about 1 mg to about 1000 mg per day, about 5 mg to about 500 mg per day, about 100 mg to about 1500 mg per day, about 500 mg to about 1500 mg per day, about 500 mg to about 2000 mg per day, or any range derived therefrom. In some embodiments, the daily dose for oral administration may be, for example, in the range of about 0.001 mg to about 2000 mg per kilogram of human body weight, administered as a single dose or in divided doses. On the other hand, in some circumstances, it may be necessary to use a dosage outside the stated limits.

In some embodiments, the pharmaceutical composition may additionally comprise an additional compound having antiproliferative activity. Depending on the mode of administration, the compound or a pharmaceutically acceptable salt thereof will be formulated into a composition suitable for facilitating delivery. Each compound or pharmaceutically acceptable salt thereof in the combination therapy may be formulated in various ways known to those skilled in the art. For example, the first agent and the second agent in the combination therapy may be formulated together or separately. Preferably, the first agent and the second agent are formulated together so that the agents can be administered simultaneously or nearly simultaneously.

It should be understood that the compounds and pharmaceutical compositions of the disclosure may be formulated and used in combination therapy, that is, they may be formulated with or administered simultaneously with, prior to, or after one or more other desired therapeutic agents or medical procedures. The particular combination of each therapy (therapeutic agent or procedure) used in a combination regimen should take into account the compatibility of the desired therapeutic agent or procedure with the desired therapeutic effect to be achieved. It should also be understood that the therapies employed may achieve the desired effect against the same condition, or they may achieve different effects (e.g., controlling any adverse effects).

As described herein, administration of each drug in the combination therapy may independently be from once to four times daily, for a duration ranging from one day to one year, and may even continue for the lifetime of the subject. Chronic/long-term administration is also applicable.

Unless otherwise defined, all technical and scientific terms used herein have the same meaning as commonly understood by one of ordinary skill in the art. Furthermore, any methods and materials similar or equivalent to those described herein can be applied to the methods of the disclosure. The preferred methods and materials described herein are merely exemplary.

### Experiments

### EXAMPLE

### General Process

When the preparation route is not included, the raw materials and reagents used in the disclosure are known products, which can be synthesized according to methods known in the art, or can be obtained by purchasing commercially available products. All commercially available reagents were used without further purification.

Room temperature means 20-30 °C.

There is no special description in the reaction examples, and the reactions are all carried out under a nitrogen atmosphere. The nitrogen atmosphere refers to a nitrogen balloon of about 1 L connected to the reaction flask.

The hydrogenation reaction is usually vacuumized and filled with hydrogen, and the operation is repeated 3 times. The hydrogen atmosphere means that the reaction bottle is connected with a hydrogen balloon of about 1 L.

Microwave reactions use the Biotage® Initiator+ Microwave Reactor.

The structures of the compounds of the disclosure were determined by nuclear magnetic resonance (NMR) and mass spectroscopy (MS). NMR shifts (δ) are given in units of 10⁻⁶ (ppm). The determination of NMR is to use (Bruker Ascend™ 500 type) nuclear magnetic analyzer, the measurement solvent is deuterated dimethyl sulfoxide (DMSO-d6), deuterated chloroform (CDCl3), deuterated methanol (CD3OD), and the internal standard is tetramethylsilane (TMS). The following abbreviations are used for the multiplicity of NMR signals: s=singlet, brs=broad singlet, d=doublet, t=triplet, m=multiplet. Coupling constants are listed as J values, measured in Hz.

As to reverse-phase preparative chromatography, a Thermo (UltiMate 3000) reverse-phase preparative chromatograph was used. The flash column chromatography uses Agela (FS-9200T) automatic column passing machine, and the silica gel prepacked column uses Santai SEPAFLASH@prepacked column. Yantai Huanghai HSGF254 or Qingdao GF254 silica gel plates are used for thin-layer chromatography silica gel plates, and the specifications used for thin-layer chromatography separation and purification products are 0.4 mm to 0.5 mm.

LC-MS analysis method is as follows:

1) Mass spectrometry method: Thermo Fisher MSQ PLUS mass spectrometer, ESI source, positive ion mode. Ion source parameter settings: drying gas temperature is 350 °C; drying gas flow rate is 10 L/min; MS Range: 120-1000.

2) Liquid phase conditions: Chromatographic column: Waters XBridge (3.5 μm, 50 mm×4.6 mm); mobile phase A is an aqueous solution containing 0.1% ammonium bicarbonate, mobile phase B is an acetonitrile solution, and perform linear gradient elution in the following table 1; Flow rate: 2 mL/min; column temperature: 30 °C; UV detection wavelength: 214 nm, 254 nm, 280 nm; injection volume 2 μL.

TABLE 1
Gradient elution conditions

| Time/min | Mobile phase A/% | Mobile phase B/% |
|---|---|---|
| 0 | 95 | 5 |
| 1.4 | 5 | 95 |
| 2.8 | 5 | 95 |
| 2.820 | 95 | 5 |
| 3 | 95 | 5 |

HPLC analysis method is as follows:

Chromatographic column: Waters XBridge phenyl (3.5 μm, 150 mm×4.6 mm); mobile phase A is aqueous solution containing 0.1% ammonium bicarbonate, mobile phase B is acetonitrile solution, and linear gradient elution is carried out in the following table 2; flow rate: 1 mL/min; column temperature: 30 °C; UV detection wavelength: 214 nm, 254 nm, 280 nm; injection volume 2 μL.

TABLE 2
Gradient elution conditions

| Time/min | Mobile phase A/% | Mobile phase B/% |
|---|---|---|
| 0 | 95 | 5 |
| 15 | 5 | 95 |
| 20 | 5 | 95 |
| 20.2 | 95 | 5 |
| 24 | 95 | 5 |

The synthesis methods of some intermediates in the disclosure are as follows:

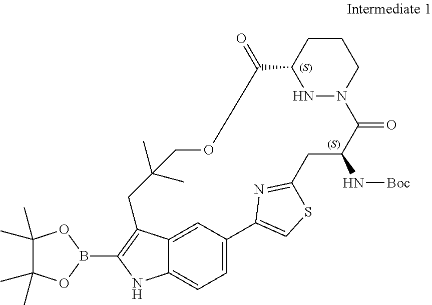

Intermediate 1 was prepared by the following steps:

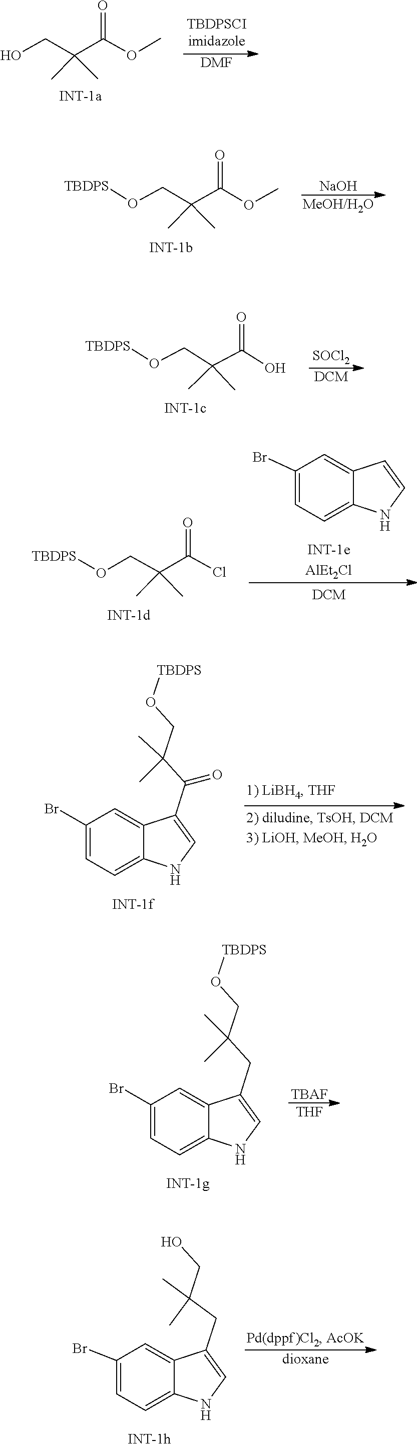

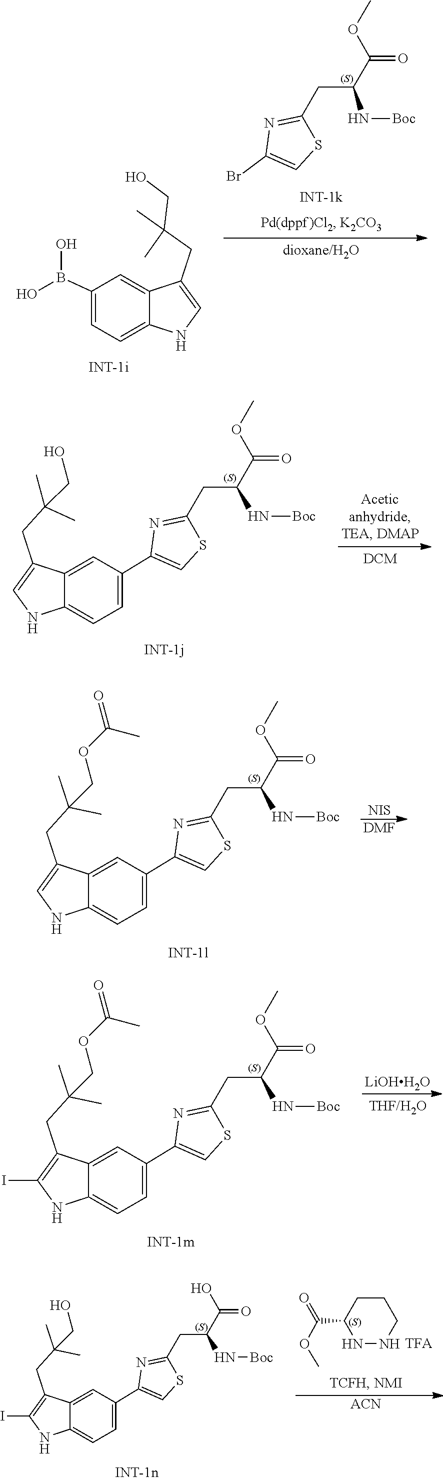

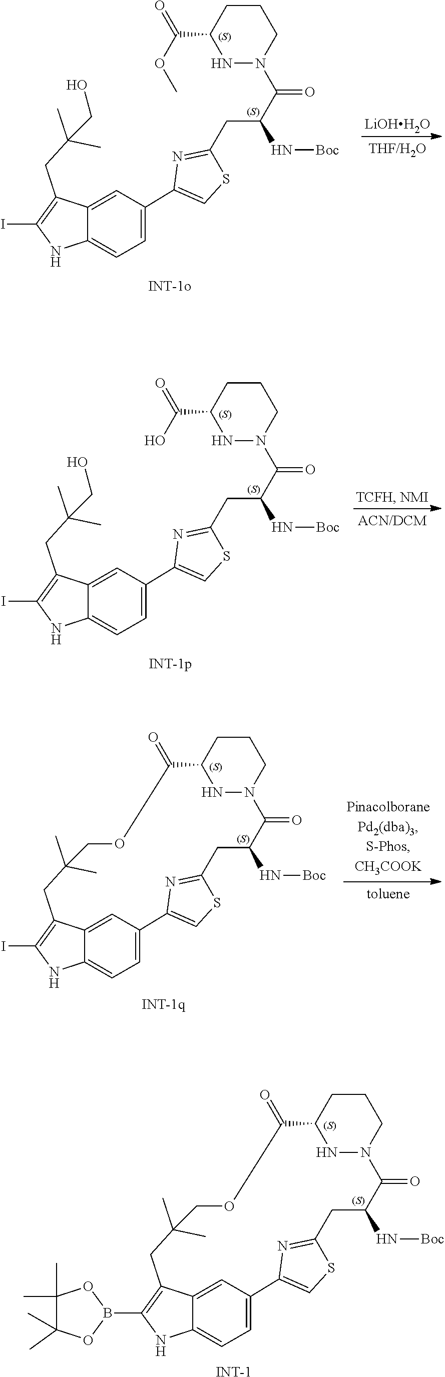

Step 1: Dissolve 2,2-dimethyl-3-hydroxypropionic acid methyl ester INT-1a (100 g, 756.67 mmol) in N,N-dimethylformamide (1 L). Add imidazole (128.79 g, 1.89 mol) and stir to dissolve. Add tert-butyldiphenylsilyl chloride (228.78 g, 832.34 mmol) dropwise at 20 °C. After the addition was complete, continue stirring for 4 hours. After the reaction was complete, the reaction solution was poured into 3 L of ice water, and the suspension was extracted with ethyl acetate (1 L*2). The organic phase was washed with water 3 times and concentrated under reduced pressure to obtain a colorless oil INT-1b, which was directly used in the next step without purification. ESI-MS (m/z): 371.2 [M+H]⁺;

Step 2: Add the residual liquid INT-1b obtained in the previous step into methanol (2 L), add 360 g of the prepared 33% sodium hydroxide aqueous solution, and stir at 20 °C for 17 hours. After the reaction was completed, 1 L of water was added, and the methanol was removed under reduced pressure. The residual liquid was extracted with petroleum ether (1 L*5). After extraction, the pH value of the aqueous phase was adjusted to 4-5 with hydrochloric acid. A large amount of white solid precipitated. Stirring was continued for 30 minutes, and the mixture was filtered and dried to obtain a white solid INT-1c (269 g, yield 90%). ESI-MS (m/z): 357.8 [M+H]⁺;

Step 3: Dissolve INT-1c (130 g, 364.63 mmol) in dichloromethane (500 mL), add thionyl chloride (130.14 g, 1.09 mol, 79.35 mL) at room temperature, add N,N-dimethylformamide (0.05 mL) dropwise, stir at 60 °C for 3 hours, and when the reaction was completed, remove dichloromethane and remaining thionyl chloride under reduced pressure, add petroleum ether (300 mL) to the residual liquid and continue to distill until no fraction was evaporated to obtain a light yellow oil INT-1d, which was directly used in the next step without purification.

Step 4: Dissolve 5-bromoindole INT-1e (64.8 g, 331 mmol) in dichloromethane (400 mL), add diethylaluminum chloride solution (198 mL, 2M in hexanes) at 0 °C, stir for 30 minutes after the addition was complete, and add the dichloromethane solution of INT-1d obtained in the previous step dropwise into the reaction flask. After the addition was complete, stirring was continued for 2 hours. After the reaction was completed, the reaction solution was slowly poured into an iced sodium potassium tartrate aqueous solution (1 L) and stirred for 16 hours. After the system was stable, concentrate under reduced pressure to remove dichloromethane. The residual liquid was extracted with ethyl acetate (1 L*2), washed with water, and the organic phase was rotary evaporated to obtain a brown oil. The oil was added to a mixed solution of petroleum ether/ethyl acetate=10/1 (2 L), stirred at 20 °C to precipitate a solid, and filtered to obtain a yellow solid INT-1f (139 g, yield 78%). ESI-MS (m/z): 534.8 [M+H]⁺;

Step 5: INT-1f (100 g, 187.07 mmol) was dissolved in tetrahydrofuran (500 mL), and lithium borohydride (12.23 g, 561.21 mmol) was added under ice bath conditions. After the addition was completed, the mixture was stirred for 20 minutes. After the system was stable, the temperature was raised to 60 °C and stirred overnight. After the starting material disappeared, the reaction solution was slowly added to ice water (200 mL) for quenching, and extracted with ethyl acetate (500 mL*3). The organic phase was washed with water, dried, and concentrated under reduced pressure. The residual liquid was dissolved in dichloromethane (500 mL), and 2,6-dimethyl-1,4-dihydro-3,5-pyridinedicarboxylic acid diethyl ester (28.43 g, 112.24 mmol) and p-toluenesulfonic acid (21.35 g, 112.24 mmol) were added. The mixture was stirred at room temperature for 3 hours. After the reaction was completed, the mixture was concentrated under reduced pressure to remove dichloromethane. The residual liquid was dissolved in methanol (500 mL). To the precipitate (100 mL), add the pre-prepared 14% aqueous lithium hydroxide solution (14% LiOH) and stir at room temperature for 3 hours. Filter with suction and dry at room temperature to obtain a yellow solid INT-1g (84 g, yield 86.26%). ESI-MS (m/z): 520.2 [M+H]⁺;

Step 6: Dissolve INT-1g (50 g, 96 mmol) in tetrahydrofuran (250 mL), add tetrabutylammonium fluoride (197 mL, 1 M in THF), and stir at 60 °C overnight. After the reaction was complete, add the reaction solution to water (300 mL), extract with ethyl acetate (200 mL*3), wash with water, and concentrate under reduced pressure to obtain a brown oil. The obtained residual liquid was dissolved in methanol (40 mL), and water (20 mL) was added. The mixed solution was washed with petroleum ether (40 mL*5) and concentrated under reduced pressure to remove methanol. The residual liquid was extracted with ethyl acetate (50 mL*2), and the organic phase was washed with water and dried to obtain INT-1h (25 g, yield 90.40%) as a light yellow oil. ESI-MS (m/z): 282.8 [M+H]⁺;

Step 7: Compound INT-1h (25 g, 88.7 mmol) was dissolved in dioxane (250 mL), and potassium acetate (21.7 g, 221.8 mmol), [1,1′-bis(diphenylphosphino)ferrocene]palladium dichloride (3.24 g, 4.4 mmol), and bis(neopentyl glycolato)diboron (24.1 g, 106.4 mmol) were added. The mixture was reacted at 90 °C for 4 hours under nitrogen protection. The reaction of the raw materials was completed by LCMS monitoring. The mixture was filtered through celite and the filtrate was concentrated. Dichloromethane (200 mL) was added to the concentrate, and 15% aqueous sodium hydroxide solution (17.7 g, 3548 mmol) was added and the mixture was concentrated under reduced pressure until the aqueous phase was clear. The mixture was filtered and the aqueous phase was extracted with dichloromethane once. The aqueous phase was adjusted to pH 3-4 with hydrochloric acid under ice bath conditions. A large amount of yellow solid precipitated. The mixture was stirred for 30 minutes and filtered to obtain a yellow solid compound INT-1i (17.96 g, yield 82.1%). ESI-MS (m/z): 248.4 [M+H]⁺;

Step 8: Compound INT-1i (35 g, 142 mmol) and compound INT-1k (51.8 g, 142 mmol) were dissolved in dioxane (350 mL) and water (17.5 mL). Potassium carbonate (39.2 g, 284 mmol) and [1,1′-bis(diphenylphosphino)ferrocene]palladium dichloride (5.2 g, 7.1 mmol) were added. The mixture was reacted at 90 °C under nitrogen for 17 hours. LCMS monitored the complete reaction of the raw materials. The reaction solution was filtered through celite and concentrated under reduced pressure. The residue was dissolved in ethyl acetate (300 mL), washed with water, and dried to obtain compound INT-1j as a brown oil, which was used directly in the next reaction without further treatment. ESI-MS (m/z): 488.4 [M+H]⁺;

Step 9: The crude product of compound INT-1j was dissolved in dichloromethane (700 mL). Add 1,4-dimethylbenzyl alcohol (866 mg, 7.1 mmol) and triethylamine (43.0 g, 426 mmol), and then add acetic anhydride (14.5 g, 142 mmol) dropwise at 0 °C. After the addition is complete, remove the ice bath and allow the temperature to rise naturally. Stir for 1-2 hours. After the reaction was complete, wash the reaction solution with water, dry it, and concentrate it to obtain a brown oil. Purify it by silica gel column chromatography (petroleum ether/ethyl acetate=4:1) to obtain INT-1l (62.3 g, 83.2% yield) as a light yellow oil. ESI-MS (m/z): 530.5 [M+H]⁺;

Step 10: Dissolve compound INT-1l (62.3 g, 117.9 mmol) in N,N-dimethylformamide (620 mL), add N-iodosuccinimide (26.5 g, 117.9 mmol), and react overnight at 10 °C. LCMS monitoring showed that the reaction of the raw materials was complete. The reaction solution was slowly poured into ice water (3000 mL) and stirred to produce solids. Filter, wash with water, and air-dry to obtain yellow solid compound INT-1m (64.2 g, yield 87%). ESI-MS (m/z): 656.3 [M+H]⁺;

Step 11: Dissolve compound INT-1m (64 g, 99.1 mmol) in tetrahydrofuran (640 mL) and water (128 mL). Add lithium hydroxide monohydrate (11.86 g, 282.4 mmol). Stir at 70 °C for 1 hour. Complete reaction of the starting material was monitored by LCMS. Add water (300 mL) to the reaction solution and concentrate under reduced pressure. Then, methyltetrahydrofuran (200 mL) was added, and the pH was adjusted to 4-5 with 4M hydrochloric acid. The mixture was extracted with methyltetrahydrofuran (200 mL*3). The organic phases were combined, washed three times with brine, and thoroughly dried to obtain a yellow solid compound INT-1n (56.5 g, yield 95%). ESI-MS (m/z): 600.5 [M+H]⁺;

Step 12: Compound INT-1n (57 g, 95.0 mmol), 1-methylimidazole (38.9 g, 475 mmol) and (S)-hexahydropyridazine-3-carboxylic acid methyl ester trifluoroacetate (52.7 g, 142.5 mmol) were dissolved in acetonitrile (800 mL). A solution of N,N,N′,N′-tetramethylchloroformamidine hexafluorophosphate (40.0 g, 142.5 mmol) in acetonitrile (400 mL) was added dropwise at 0 °C and stirred for 1 hour. LCMS monitored the complete reaction of the raw materials. Water (1000 mL) was added to the reaction solution, and the mixture was extracted with dichloromethane (1000 mL*3). The mixture was dried to obtain a yellow solid compound INT-1o (56.5 g, 95% yield). ESI-MS (m/z): 726.3 [M+H]⁺;

Step 13: Dissolve compound INT-1o (56.5 g, 77.8 mmol) in tetrahydrofuran (560 mL) and water (112 mL), add lithium hydroxide (4.66 g, 194.7 mmol), and react at 10 °C for 2 hours. LCMS monitoring showed that the reaction of the raw material was complete. Add water (300 mL) and adjust the pH to 5-6 with 4M hydrochloric acid. Concentrate, extract with methyltetrahydrofuran, wash with brine, and after sufficient separation, completely remove the solvent by rotary evaporation to obtain yellow solid compound INT-1p (55.4 g, yield 88.16%). ESI-MS (m/z): 712.6 [M+H]⁺;

Step 14: Add N,N,N′,N′-tetramethylchloroformamidine hexafluorophosphate (59.1 g, 210.8 mmol) and 1-methylimidazole (26.5 g, 323.2 mmol) to acetonitrile (2000 mL), stir to dissolve, add a THF solution of compound INT-1p (100 g/1000 mL, 140.5 mmol) dropwise at 10-20 °C, stir and react for 1-2 hours. LCMS monitored the complete reaction of the raw materials, evaporate the solvent, add water (1000 mL) to the residue, extract with dichloromethane (1000 mL*3), adjust the pH to 3-4 with hydrochloric acid, and spin-dry the organic phase to obtain a yellow solid. Recrystallization from isopropanol gave compound INT-1q (59 g, yield 60%). ESI-MS (m/z): 694.6 [M+H]⁺;

Step 15: Compound INT-1q (37 g, 53.35 mmol), 2-dicyclohexylphosphine-2′,6′-dimethyl-biphenyl (6.6 g, 16.0 mmol), tris(dibenzylideneacetone)dipalladium (5.86 g, 6.40 mmol), and potassium acetate (18.3 g, 186.7 mmol) were dissolved in toluene (370 mL). Pinacolborane (34.1 g, 266.7 mmol, 38.7 mL) was added dropwise under nitrogen protection. The mixture was reacted at 50 °C under nitrogen protection for 3 hours. The reaction of the raw materials was completed as monitored by LCMS. The reaction solution was filtered and purified by silica gel column chromatography to obtain a yellow solid compound INT-1 (31 g, yield 82%). ESI-MS (m/z): 694.8 [M+H]⁺ ∘

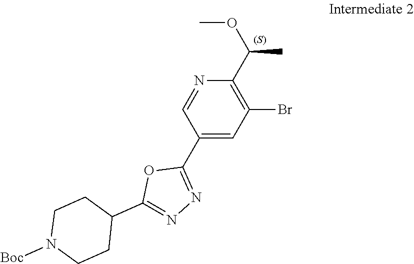

Intermediate 2 was prepared by the following steps:

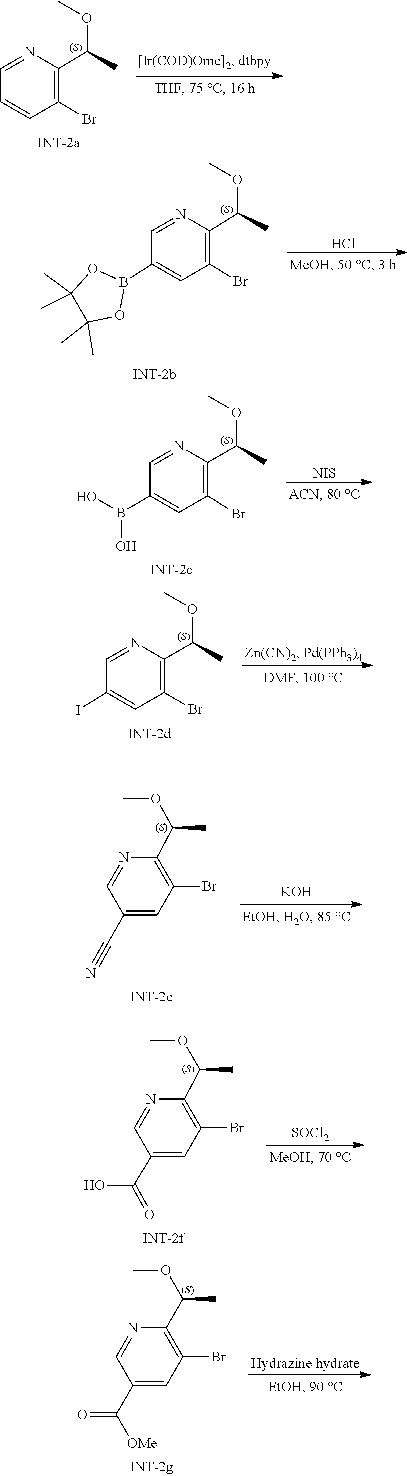

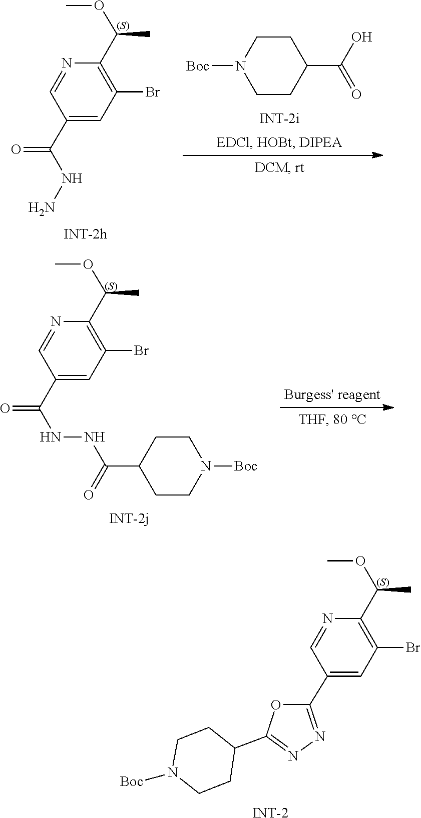

Step 1: Compound INT-2a (43 g, 199 mmol), bis(pinacolato)diboron (55.6 g 219 mmol), methoxy(cyclooctadiene)iridium dimer (1.30 g, 1.99 mmol) and 4,4-di-tert-butylbipyridine (2.67 g, 9.95 mmol) were added to tetrahydrofuran (500 mL). The temperature was raised to 75 °C under nitrogen and the reaction was stirred for 16 hours. LCMS monitoring showed that the raw materials were completely converted. Excess tetrahydrofuran was removed by rotary evaporation to obtain a brown residue INT-2b, which was used directly in the next reaction without purification. ESI-MS (m/z): 358.3 [M+H]⁺ ∘

Step 2: The residual liquid INT-2b obtained in the previous reaction was added to methanol (200 mL), and concentrated hydrochloric acid (100 mL) was added thereto. The reaction solution was refluxed for 3 hours. The starting material disappeared after monitoring by LCMS. The methanol was removed by rotary evaporation. Water (200 mL) was added to the residual liquid, and the pH was adjusted to 13 with 30% sodium hydroxide solution. The product was extracted with dichloromethane to remove impurities. The aqueous phase was cooled to 0-5 °C and the pH was adjusted to 6-7 with hydrochloric acid. The solid was washed out by continued stirring, filtered, and dried to obtain the white solid compound INT-2c (41.3 g, yield 80%). ESI-MS (m/z): 276.3 [M+H]⁺ ∘

Step 3: Compound INT-2c (41.3 g, 159 mmol) was added to acetonitrile (400 mL) and dissolved. N-iodosuccinimide (35.8 g, 239 mmol) was added, and the temperature was raised to 80 °C. The mixture was stirred overnight. LCMS showed that the starting material disappeared. The reaction solution was rotary evaporated to remove acetonitrile, and ethyl acetate (300 mL) was added to the residue. The mixture was washed with water, dried, and purified by silica gel column chromatography (petroleum ether/ethyl acetate=10:1) to obtain a white solid compound INT-2d (45.6 g, yield 85%). ESI-MS (m/z): 358.1 [M+H]⁺ ∘

Step 4: Dissolve compound INT-2d (5 g, 14.6 mmol) in N,N-dimethylformamide (50 mL), and add zinc cyanide (1.03 g, 8.8 mmol) and tetrakis(triphenylphosphine)palladium (1.69 g, 1.46 mmol) in sequence. Stir at 100 °C overnight under nitrogen protection. LCMS monitoring showed that the reaction of the raw material was complete. Add ammonia water (5 mL) to quench the reaction. Extract with ethyl acetate (200 mL*2). Combine the organic phases, wash with saturated brine, dry over anhydrous sodium sulfate, filter, and concentrate. The residue was purified by column chromatography (petroleum ether/ethyl acetate=10:1) to give INT-2e (2.1 g, yield 59.6%) as a colorless oil. ESI-MS (m/z): 241.2 [M+H]⁺ ∘

Step 5: Compound INT-2e (2.1 g, 8.7 mmol) was dissolved in a mixed solution of ethanol (20 mL) and water (4 mL). Potassium hydroxide (0.54 g, 9.6 mmol) was added. The reaction solution was refluxed for 16 hours. LCMS monitoring showed the disappearance of the starting material. The reaction solution was concentrated to obtain compound INT-2f (2.26 g, 100% yield) as a white solid. ESI-MS (m/z): 259.3 [M+H]⁺ ∘

Step 6: Dissolve compound INT-2f (770 mg, 2.96 mmol) in methanol (10 mL) and add thionyl chloride (1.06 g, 8.9 mmol). The reaction solution was stirred at 70 °C for 3 hours. LCMS monitoring showed that the reaction of the starting material was complete. The reaction solution was concentrated to obtain a light yellow solid compound INT-2g (800 mg, yield 98.6%). ESI-MS (m/z): 274.1 [M+H]⁺ ∘

Step 7: Dissolve compound INT-2g (600 mg, 2.19 mmol) in ethanol (6 mL) and add hydrazine hydrate (329 mg, 6.57 mmol). The reaction solution was stirred at 90 °C for 16 hours. LCMS monitoring showed that the reaction of the starting material was complete, and the reaction solution was concentrated. The residue was purified by column chromatography (dichloromethane/methanol=20:1) to give INT-2h (550 mg, 91.7% yield) as a colorless oil. ESI-MS (m/z): 274.2 [M+H]⁺ ∘

Step 8: Compound INT-2h (300 mg, 1.09 mmol) was dissolved in N,N-dimethylformamide (3 mL), and INT-2i (376 mg, 1.64 mmol), 1-hydroxybenzotriazole (222 mg, 1.64 mmol), N,N-diisopropylethylamine (424 mg, 3.28 mmol) and 1-(3-dimethylaminopropyl)-3-ethylcarbodiimide hydrochloride (315 mg, 1.64 mmol) were added in sequence. The reaction solution was stirred at room temperature for 16 hours. LCMS monitored the complete reaction of the raw materials. Water was added to the system, and the mixture was extracted with dichloromethane. The organic phase was dried, dried over anhydrous sodium sulfate, filtered, and concentrated. The residue was purified by column chromatography (dichloromethane/methanol=10:1) to give compound INT-2j (480 mg, yield 90.4%) as a pale yellow oil. ESI-MS (m/z): 485.3 [M+H]⁺ ∘

Step 9: Dissolve compound INT-2j (480 mg, 0.99 mmol) in tetrahydrofuran (5 mL) and add Burgess reagent (354 mg, 1.48 mmol). The reaction solution was stirred at 80 °C for 16 hours. LCMS monitoring showed that the reaction of the starting material was complete, and the reaction solution was concentrated. The residue was purified by column chromatography (dichloromethane/methanol=20:1) to give compound INT-2 (320 mg, yield 69.2%) as a colorless oil. ESI-MS (m/z): 467.2 [M+H]⁺ ∘

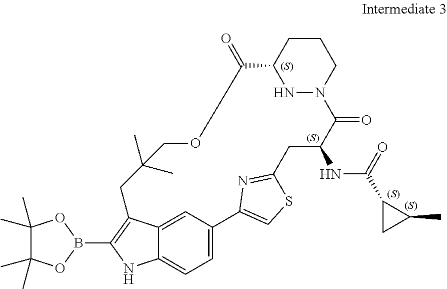

Intermediate 3 was prepared by the following steps:

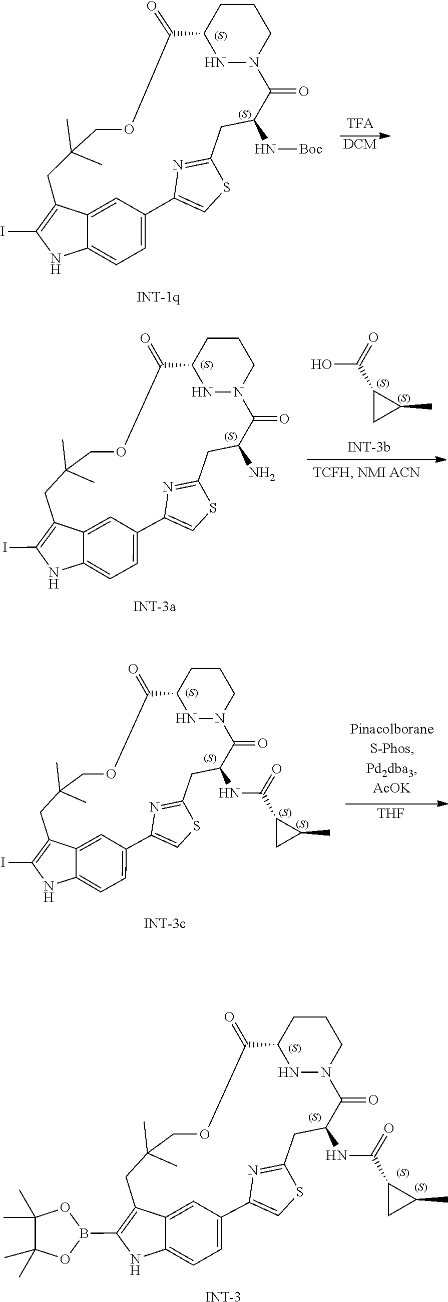

Step 1: Dissolve compound INT-1q (1.7 g, 2.45 mmol) in dichloromethane (20 mL), add trifluoroacetic acid (5 mL), and react at room temperature for 2 hours. LCMS monitoring showed that the reaction of the raw material was complete. The reaction solution was directly concentrated under reduced pressure, and the residue was dissolved in DCM (50 mL) and washed twice with saturated NaHCO₃ aqueous solution. The organic phase was washed with water, dried over sodium sulfate, filtered, and concentrated to obtain a yellow solid compound INT-3a (1.3 g, yield 89.4%). ESI-MS (m/z): 594.7 [M+H]⁺ ∘

Step 2: Compound INT-3a (1.3 g, 2.19 mmol) and compound INT-3b (0.24 g, 2.41 mmol) were dissolved in acetonitrile (30 mL). N,N,N′,N′-tetramethylchloroformamidine hexafluorophosphate (922 mg, 3.29 mmol) and 1-methylimidazole (414 mg, 5.04 mmol) were added at 0 °C. The reaction was incubated at 0 °C for 1 hour. LCMS monitored the complete reaction of the raw materials. The reaction solution was poured into water (50 mL) and extracted with dichloromethane (50 mL*3). The organic phase was washed with water and purified by column to obtain a white solid compound INT-3c (1.3 g, yield 87.9%/). ESI-MS (m/z): 675.7 [M+H]⁺ ∘

Step 3: Compound INT-3c (1.1 g, 1.63 mmol), 2-dicyclohexylphosphine-2′,6′-dimethyl-biphenyl (200 mg, 0.188 mmol), tris(dibenzylideneacetone)dipalladium (179 mg, 0.195 mmol), and potassium acetate (559 mg, 5.7 mmol) were dissolved in toluene (30 mL). Pinacolborane (1.04 g, 8.14 mmol) was added dropwise under nitrogen protection. The mixture was reacted at 50 °C for 3 hours under nitrogen protection. The reaction of the raw materials was complete as monitored by LCMS. The reaction solution was filtered and purified by silica gel column chromatography to obtain a yellow solid compound INT-3 (990 mg, yield 90%). ESI-MS (m/z): 676.9 [M+H]⁺ ∘

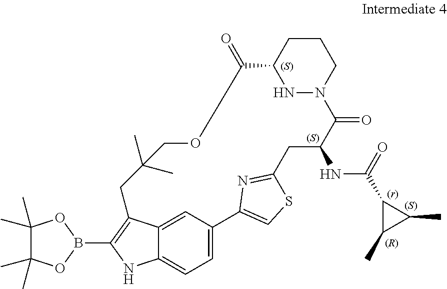

Intermediate 4 was prepared by the following steps:

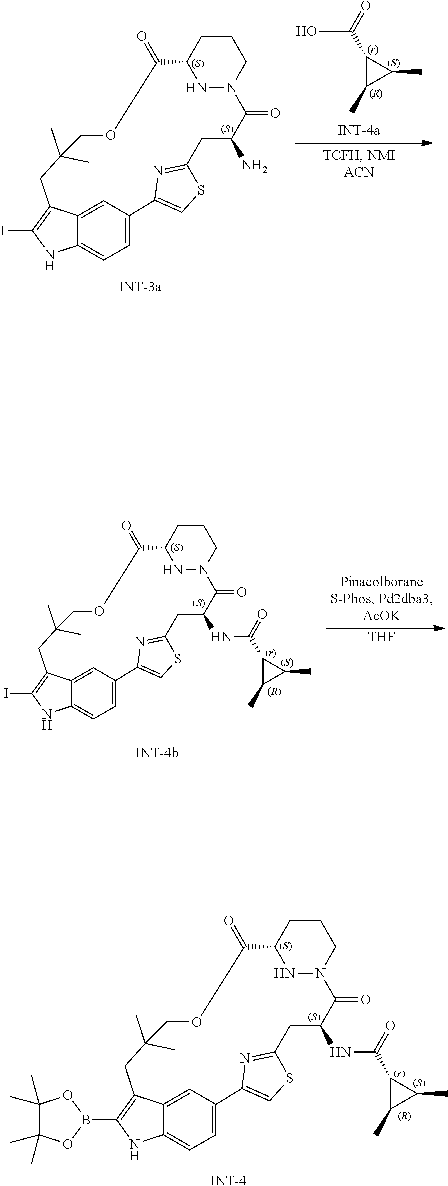

Step 1: Compound INT-3a (2.2 g, 3.71 mmol) and compound INT-4a (0.47 g, 4.08 mmol) were dissolved in dichloromethane (50 mL). N,N,N′,N′-tetramethylchloroformamidine hexafluorophosphate (1.56 g, 5.56 mmol) and 1-methylimidazole (0.70 g, 8.53 mmol) were added at 0 °C. The reaction was incubated at 0 °C for 1 hour. LCMS monitored the complete reaction of the raw materials. The reaction solution was poured into water (50 mL) and extracted with dichloromethane (50 mL*3). The organic phase was washed with water and purified by column to obtain white solid compound INT-4b (2.3 g, yield 90.0%). ESI-MS (m/z): 690.2 [M+H]⁺ ∘

Step 2: Compound INT-4b (2.1 g, 3.05 mmol), 2-dicyclohexylphosphine-2′,6′-dimethyl-biphenyl (375 mg, 0.91 mmol), tris(dibenzylideneacetone)dipalladium (335 mg, 0.365 mmol), and potassium acetate (1.05 g, 10.7 mmol) were dissolved in toluene (30 mL). Pinacolborane (1.95 g, 15.2 mmol) was added dropwise under nitrogen protection. The mixture was reacted at 50 °C for 3 hours under nitrogen protection. The reaction of the raw materials was complete as monitored by LCMS. The reaction solution was filtered and purified by silica gel column chromatography to obtain a yellow solid compound INT-4 (1.8 g, yield 85.7%). ESI-MS (m/z): 690.3 [M+H]⁺ ∘

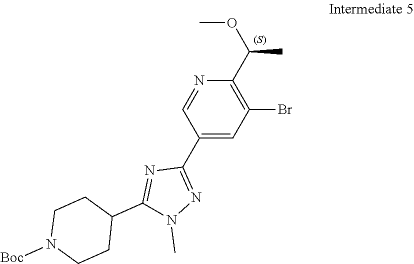

Intermediate 5 was prepared by the following steps:

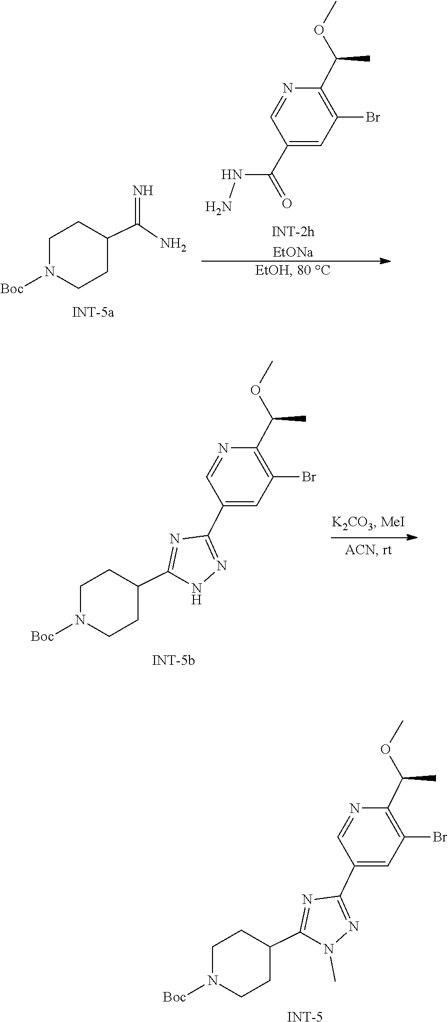

Step 1: Dissolve INT-5a (663 mg, 2.92 mmol) in ethanol (8 mL), add sodium ethoxide (199 mg, 2.92 mmol) at room temperature, and stir at room temperature for 30 minutes. The solid was removed by filtration, and then a solution of INT-2h (400 mg, 1.46 mmol) in ethanol was added to the filtrate, and the reaction solution was stirred at 85 °C for 16 hours. After the reaction was completed, the reaction mixture was concentrated under reduced pressure. The residue was purified by silica gel column chromatography (dichloromethane/methanol=20:1) to obtain a light yellow solid compound INT-5b (516 mg, yield 75.8%). ESI-MS (m/z): 466.3 [M+H]⁺ ∘

Step 2: Dissolve INT-5b (516 mg, 1.11 mmol) in acetonitrile (8 mL). Add potassium carbonate (459 mg, 3.33 mmol) and iodomethane (236 mg, 1.66 mmol) at room temperature. Stir the reaction mixture at room temperature for 16 hours. After the reaction, add water and extract with dichloromethane. Dry the organic phase with anhydrous sodium sulfate, filter, and concentrate. The residue was purified by silica gel column chromatography (dichloromethane/methanol=20:1) to give INT-5 (382 mg, yield 71.9%) as a colorless oil. ESI-MS (m/z): 480.3 [M+H]⁺ ∘

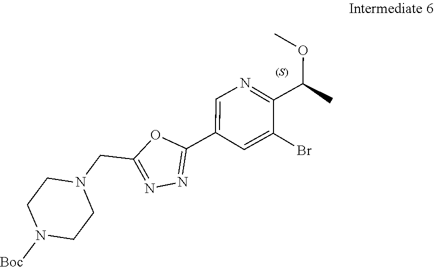

Intermediate 6 was prepared by the following steps:

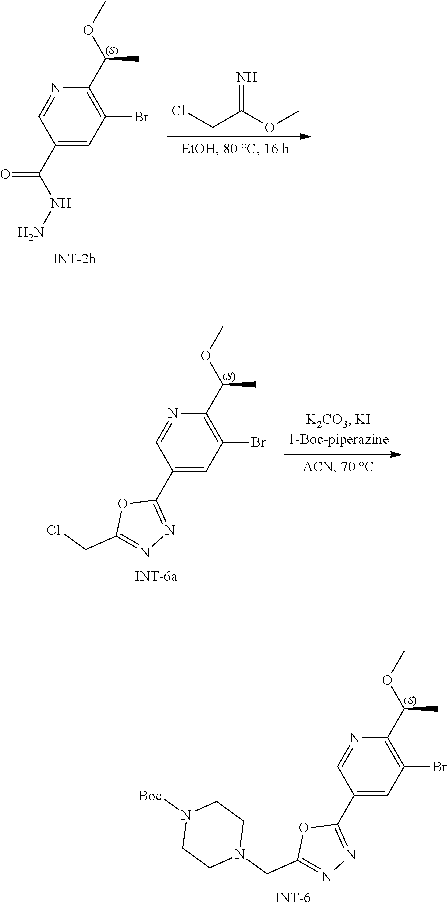

Step 1: Dissolve INT-2h (2.1 g, 7.66 mmol) and methyl 2-chloroacetimidate hydrochloride (2.0 g, 13.79 mmol) in anhydrous ethanol (40 mL). Stir the reaction mixture at 80 °C for 16 h. After the reaction was completed, the reaction solution was concentrated to obtain a crude product, which was then purified by silica gel column chromatography (petroleum ether/ethyl acetate=1:1) to obtain a colorless oily compound INT-6a (2.1 g, yield 82.4%). ESI-MS (m/z): 332.4 [M+H]⁺ ∘

Step 2: INT-6a (188 mg, 0.57 mmol), 1-tert-butyloxycarbonylpiperazine (211 mg, 1.13 mmol), potassium carbonate (156 mg, 1.13 mmol), and potassium iodide (47 mg, 0.28 mmol) were dissolved in acetonitrile (5 mL), and the reaction solution was stirred at 70 °C for 3 h. After the reaction was completed, the reaction solution was extracted with ethyl acetate (40 mL), and the organic phase was dried and concentrated to obtain a crude product. The crude product was purified by silica gel column chromatography (dichloromethane/methanol=20:1) to obtain a colorless oily compound NT-6 (218 mg, yield 80.0%). ESI-MS (m/z): 482.2 [M+H]⁺ ∘

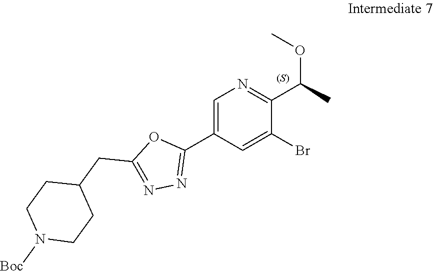

By replacing INT-2i in the synthesis step of intermediate INT-2 with 1-tert-butyloxycarbonyl-4-piperidinylacetic acid, compound INT-7 can be obtained using similar methods and reaction steps. ESI-MS (m/z): 481.2 [M+H]⁺ ∘

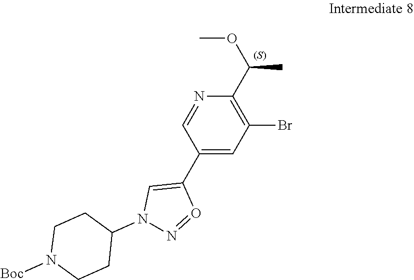

Intermediate 8 was prepared by the following steps:

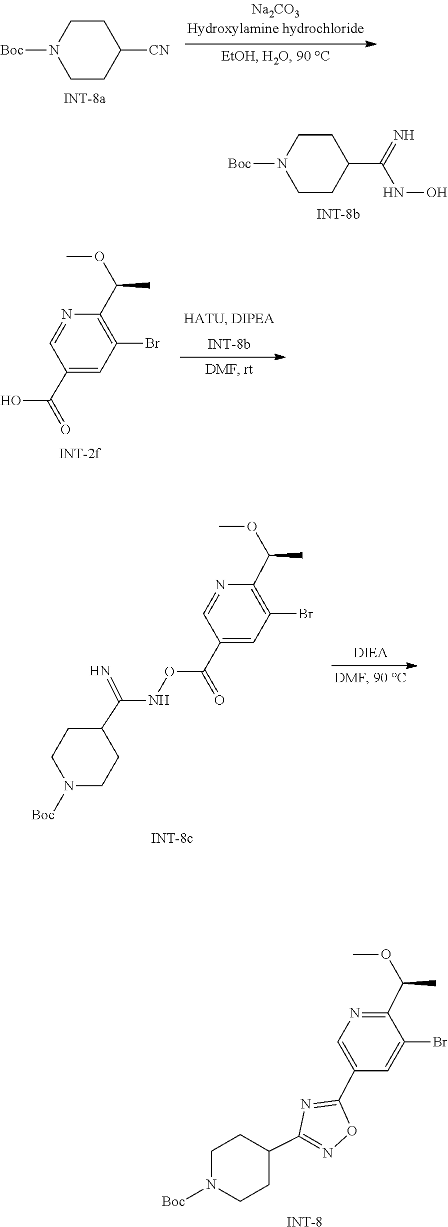

Step 1: Dissolve compound INT-8a (1 g, 7.93 mmol) in ethanol (10 mL) and water (5 mL). Add hydroxylamine hydrochloride (2.2 g, 31.7 mmol) and sodium carbonate (2.52 g, 23.8 mmol) in sequence. The reaction solution was stirred at 90 °C overnight. LCMS monitored the completion of the reaction. The reaction solution was concentrated, and the residue was purified by column chromatography (dichloromethane/methanol=10:1) to obtain a white solid compound INT-8 b (901 mg, yield 71.4%). ESI-MS (m/z): 160.2 [M+H]⁺ ∘

Step 2: Compound INT-2f (409 mg, 1.40 mmol) was dissolved in N,N-dimethylformamide (6 mL), and INT-8b (202 mg, 1.68 mmol), 2-(7-azobenzotriazole)-N,N,N′,N′-tetramethyluronium hexafluorophosphate (639 mg, 1.68 mmol) and N,N-diisopropylethylamine (543 mg, 4.20 mmol) were added in sequence. The reaction solution was stirred at room temperature for 4 hours. LCMS monitored the complete reaction of the raw materials. Water was added to the system, and the mixture was extracted with dichloromethane. The organic phase was dried, dried over anhydrous sodium sulfate, filtered, and concentrated. The residue was purified by column chromatography (petroleum ether/ethyl acetate=1:2) to give INT-8c (407 mg, yield 59.9%) as a colorless oil. ESI-MS (m/z): 485.4 [M+H]⁺ ∘

Step 3: Dissolve compound INT-8c (407 mg, 0.84 mmol) in N,N-dimethylformamide (4 mL), add N,N-diisopropylethylamine (325 mg, 2.52 mmol), and stir the reaction solution at 90 °C for 16 hours. LCMS monitoring showed that the reaction of the raw material was complete. Water was added to the system, and the mixture was extracted with dichloromethane. The organic phase was dried, dried over anhydrous sodium sulfate, filtered, and concentrated. The residue was purified by column chromatography (petroleum ether/ethyl acetate=2:3) to give INT-8 (149 mg, yield 38.0%) as a colorless oil. ESI-MS (m/z): 467.4 [M+H]⁺ ∘

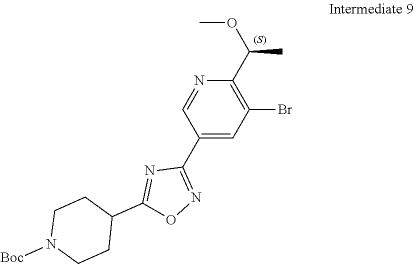

Intermediate 9 was prepared by the following steps:

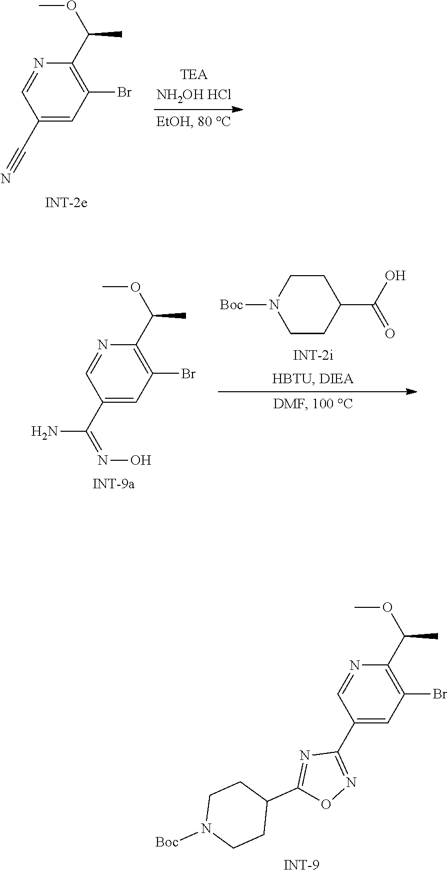

Step 1: Dissolve compound INT-2e (150 mg, 0.62 mmol) in ethanol (6 mL), and add hydroxylamine hydrochloride (86 mg, 1.24 mmol) and triethylamine (126 mg, 1.24 mmol) in sequence. The reaction solution was stirred at 80 °C for 3 hours. LCMS monitoring showed that the reaction of the raw material was complete. The reaction solution was concentrated to obtain a crude colorless oily compound INT-9a, which was used directly in the next reaction without purification. ESI-MS (m/z): 274.2 [M+H]⁺ ∘

Step 2: Dissolve compound INT-9a (350 mg, 1.28 mmol) in N,N-dimethylformamide (5 mL), and add INT-2i (293 mg, 1.28 mmol), benzotriazole-N,N,N′,N′-tetramethyluronium hexafluorophosphate (533 mg, 1.40 mmol) and N,N-diisopropylethylamine (495 mg, 3.83 mmol) in sequence. The reaction solution was stirred at 100 °C for 16 hours. LCMS monitored the complete reaction of the raw materials. Water was added to the system, extracted with dichloromethane, and the organic phase was dried, dried over anhydrous sodium sulfate, filtered, and concentrated. The residue was purified by column chromatography (petroleum ether/ethyl acetate=1:2) to give INT-16 (315 mg, yield 52.8%) as a colorless oil. ESI-MS (m/z): 467.4 [M+H]⁺ ∘

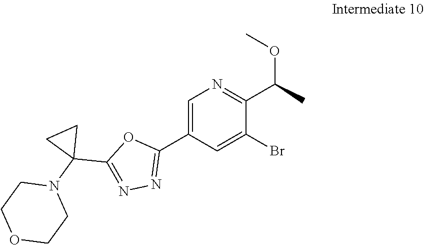 [SMILES: CO[C@@H](C)c1ncc(-c2nnc(C3(N4CCOCC4)CC3)o2)cc1Br]

Intermediate 10 was prepared by the following steps:

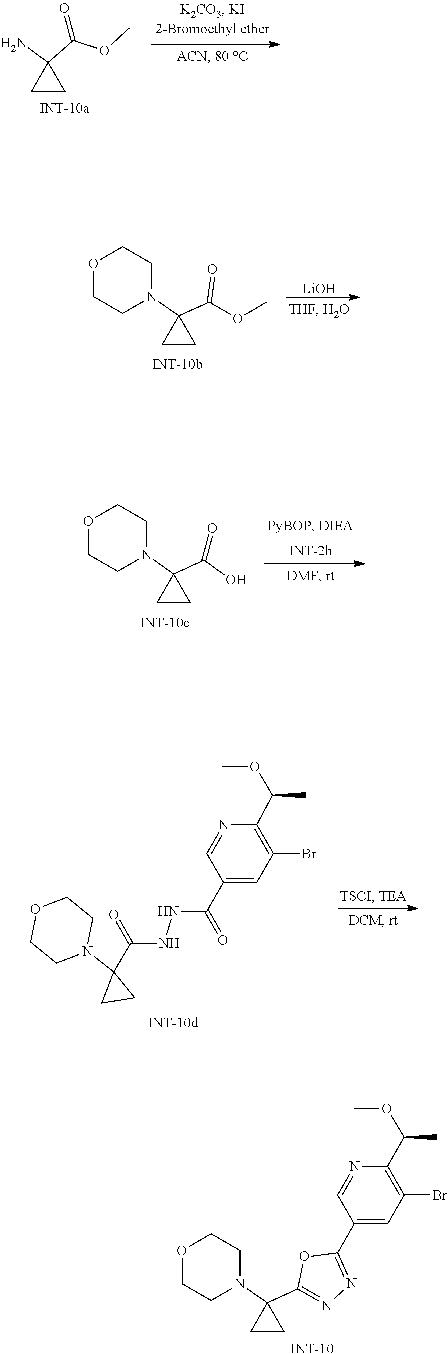

Step 1: Dissolve compound INT-10a (3.0 g, 26.1 mmol) in acetonitrile (30 mL), add potassium carbonate (10.8 g, 78.2 mmol), potassium iodide (43 mg, 0.26 mmol) and 2-bromoethyl ether (6.0 g, 26.1 mmol), and stir the reaction solution at 80 °C for 16 hours. After the reaction was completed, water (50 mL) was added to quench the reaction, and the mixture was extracted with dichloromethane (50 mL*2). The organic phases were combined and washed with saturated brine. The organic phase was concentrated under reduced pressure, and the crude product was purified by silica gel column chromatography (petroleum ether/ethyl acetate=3:1) to obtain INT-10b (3.1 g, yield 64.2%) as a pale yellow oil. ESI-MS (m/z): 186.2 [M+H]⁺ ∘

Step 2: Dissolve compound INT-10b (2.0 g, 10.8 mmol) in tetrahydrofuran (5 mL) and water (5 mL), and add lithium hydroxide monohydrate (544 mg, 13.0 mmol) under ice bath. The reaction mixture was stirred at room temperature for 2 hours. After the reaction was complete, the reaction solution was lyophilized to obtain a white solid compound INT-10c (1.85 g, yield 100%). ESI-MS (m/z): 172.2 [M+H]⁺ ∘

Step 3: Dissolve compound INT-2h (150 mg, 0.55 mmol) in N,N-dimethylformamide (8 mL), and add INT-10c (187 mg, 1.09 mmol), benzotriazol-1-yl-oxytripyrrolidinophosphine hexafluorophosphate (427 mg, 0.82 mmol) and N,N-diisopropylethylamine (212 mg, 1.64 mmol) in sequence. The reaction solution was stirred at room temperature for 4 hours. LCMS monitored the complete reaction of the raw materials, and water was added to the system. The mixture was extracted with dichloromethane, and the organic phase was dried over anhydrous sodium sulfate, filtered, and concentrated. The residue was purified by column chromatography (dichloromethane/methanol=10:1) to give INT-10d (200 mg, yield 85.5%) as a pale yellow oil. ESI-MS (m/z): 427.2 [M+H]⁺ ∘

Step 4: Dissolve compound INT-10d (200 mg, 0.47 mmol) in dichloromethane (5 mL), add p-toluenesulfonyl chloride (448 mg, 2.35 mmol) and triethylamine (476 mg, 4.70 mmol) in sequence, and stir the reaction solution at room temperature for 4 hours.

LCMS monitored the complete reaction of the raw materials, and water was added to the system. The mixture was extracted with dichloromethane, and the organic phase was dried over anhydrous sodium sulfate, filtered, and concentrated. The residue was purified by column chromatography (petroleum ether/ethyl acetate=1:2) to give a light yellow oily compound INT-10 (140 mg, yield 73.0%). ESI-MS (m/z): 409.2 [M+H]⁺ ∘

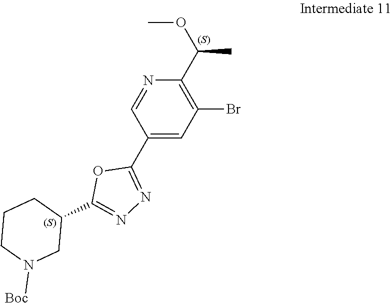

By replacing INT-2i in the synthesis step of intermediate INT-2 with (S)-1-(tert-butyloxycarbonyl)piperidine-3-carboxylic acid, compound INT-11 can be obtained using similar methods and reaction steps. ESI-MS (m/z): 467.2 [M+H]⁺ ∘

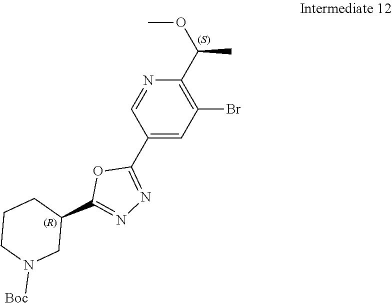

By replacing INT-2i in the synthesis step of intermediate INT-2 with (R)-1-(tert-butyloxycarbonyl)piperidine-3-carboxylic acid, compound INT-12 can be obtained using similar methods and reaction steps. ESI-MS (m/z): 467.2 [M+H]⁺ ∘

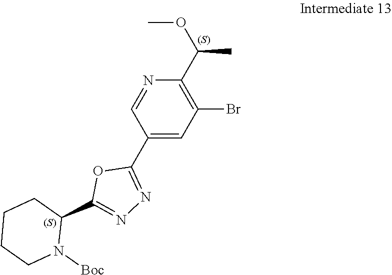

By replacing INT-2i in the synthesis step of intermediate INT-2 with (S)-1-(tert-butyloxycarbonyl)-2-piperidinic acid, compound INT-13 can be obtained using similar methods and reaction steps. ESI-MS (m/z): 467.2 [M+H]⁺ ∘

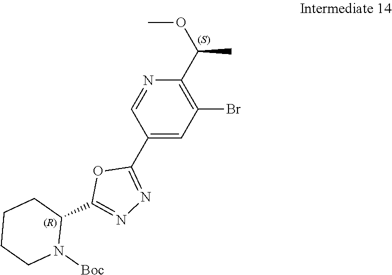

By replacing INT-2i in the synthesis step of intermediate INT-2 with (R)-1-(tert-butyloxycarbonyl)piperidine-2-carboxylic acid, compound INT-14 can be obtained using similar methods and reaction steps. ESI-MS (m/z): 467.2 [M+H]⁺ ∘

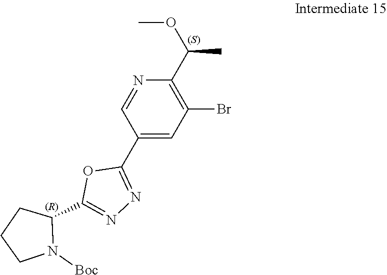

By replacing INT-2i in the synthesis step of intermediate INT-2 with (2R)-1-[(tert-butoxy)carbonyl]pyrrolidine-2-carboxylic acid, compound INT-15 can be obtained using similar methods and reaction steps. ESI-MS (m/z): 453.2 [M+H]⁺ ∘

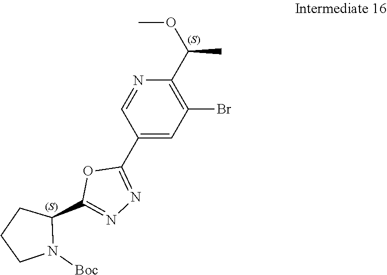

By replacing INT-2i in the synthesis step of intermediate INT-2 with (S)-1-(tert-butyloxycarbonyl)pyrrolidine-2-carboxylic acid, compound INT-16 can be obtained using similar methods and reaction steps. ESI-MS (m/z): 453.2 [M+H]⁺ ∘

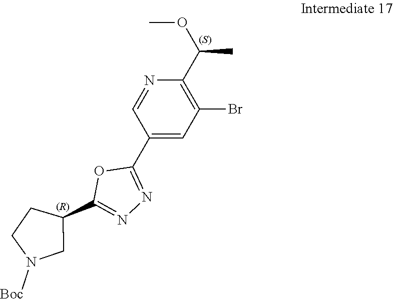

By replacing INT-2i in the synthesis step of intermediate INT-2 with (R)-1-(tert-butyloxycarbonyl)pyrrolidine-3-carboxylic acid, compound INT-17 can be obtained using similar methods and reaction steps. ESI-MS (m/z): 453.2 [M+H]⁺ ∘

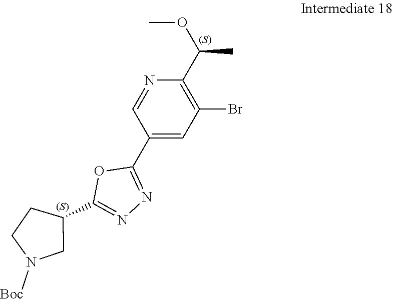

By replacing INT-2i in the synthesis step of intermediate INT-2 with (S)-1-(tert-butyloxycarbonyl)pyrrolidine-3-carboxylic acid, compound INT-18 can be obtained using similar methods and reaction steps. ESI-MS (m/z): 453.2 [M+H]⁺ ∘

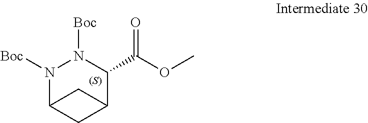

Intermediate 30 was prepared by the following steps:

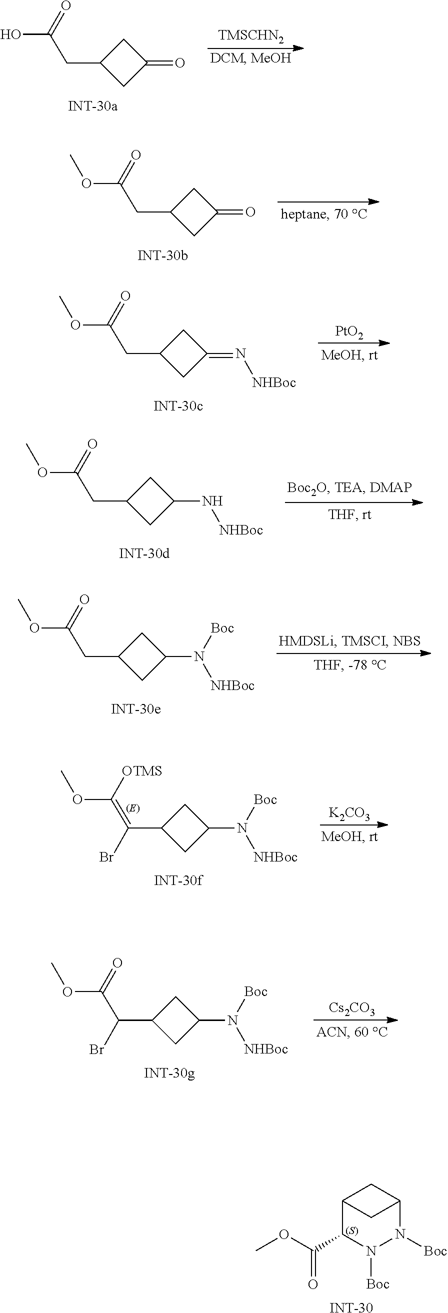

Step 1: Dissolve compound INT-30a (5.0 g, 39.0 mmol) in dichloromethane mL) and methanol (10 mL). Add trimethylsilylated diazomethane (2 M, 29.3 mL) dropwise at 0 °C. After the addition was complete, continue stirring for 1 hour. Monitor the reaction of the starting material by TLC. The residue was concentrated to give compound INT-30b (5.6 g, 100% yield) as a colorless oil. ESI-MS (m/z): 143.4 [M+H]⁺ ∘¹H NMR (500 MHz, Chloroform-d) δ 3.70 (s, 3H), 3.32-3.20 (in, 2H), 2.87-2.77 (m, 3H), 2.68-2.60 (m, 2H).

Step 2: Compound INT-30b (5.6 g, 39.4 mmol) was dissolved in n-heptane (60 mL), and tert-butyl carbazate (5.5 g, 41.4 mmol) was added thereto. The temperature was raised to 70 °C and stirred for 16 hours. LCMS monitoring showed that the reaction of the raw material was complete. The reaction solution was concentrated, and the resulting residue was recrystallized from (n-heptane/isopropanol=30/1) to give compound INT-30c as a white solid (9.0 g, yield 89.1%). ESI-MS (m/z): 257.3 [M+H]⁺ ∘

Step 3: Compound INT-30c (3.0 g, 11.7 mmol) was dissolved in methanol (30 mL), and platinum dioxide (300 mg, 10% wt) was added thereto. The reaction solution was stirred under a hydrogen atmosphere for 16 hours. LC-MS monitoring confirmed the complete reaction of the raw material. The reaction solution was filtered through celite, and the filtrate was concentrated to obtain a colorless oily compound INT-30d (3.0 g, yield 99.2%). ESI-MS (m/z): 259.3 [M+H]⁺ ∘

Step 4: Dissolve compound INT-30d (3.0 g, 11.6 mmol) in tetrahydrofuran (30 mL). Add di-tert-butyl dicarbonate (3.0 g, 14.0 mmol), triethylamine (3.5 g, 34.8 mmol), and 4-dimethylaminopyridine (0.14 g, 1.2 mmol) in this order at 0 °C. The reaction mixture was heated to room temperature and stirred for 2 hours. LCMS monitoring indicated that the reaction of the raw materials was complete. Water was added to the system to quench the reaction, and the mixture was extracted with ethyl acetate. The organic phases were combined, washed with saturated brine, dried over anhydrous sodium sulfate, filtered, and concentrated. The resulting residue was purified by silica gel column chromatography (petroleum ether/ethyl acetate=4/1) to give INT-30e (3.2 g, 75.7% yield) as a colorless oil. ESI-MS (m/z): 359.2 [M+H]⁺ ∘

Step 5: Dissolve compound INT-30e (2.0 g, 5.6 mmol) in anhydrous tetrahydrofuran (20 mL). Add lithium bis(trimethylsilyl)amide (1 M, 16.7 mL) dropwise at −70 °C under a nitrogen atmosphere. After the addition was complete, stir at this temperature for 30 minutes. Then, trimethylsilyl chloride (1.8 g 16.7 mmol) was added, and stirring was continued for 1 hour before adding N-bromosuccinimide (3.0 g, 16.7 mmol). The reaction solution was warmed to room temperature and stirred for 16 hours. LC-MS monitoring showed that the reaction of the raw material was complete. Water was added to the system to quench the reaction, and the mixture was extracted with ethyl acetate. The organic phases were combined, washed with saturated brine, dried over anhydrous sodium sulfate, filtered, and concentrated. The obtained crude INT-30f was directly used in the next reaction. ESI-MS (m/z): 509.3 [M+H]⁺ ∘

Step 6: The crude compound INT-30f was dissolved in methanol (20 mL), and potassium carbonate (1.5 g, 11.2 mmol) was added thereto at 0 °C. The reaction mixture was heated to room temperature and stirred for 2 hours. LCMS monitoring confirmed the complete reaction of the raw material. Water was added to the system to quench the reaction, and the mixture was extracted with ethyl acetate. The organic phases were combined, washed with saturated brine, dried over anhydrous sodium sulfate, filtered, and concentrated. The resulting residue was purified by silica gel column chromatography (petroleum ether/ethyl acetate=4/1) to give INT-30g (1.4 g, two-step yield 57.4%) as a colorless oil. ESI-MS (m/z): 437.2 [M+H]⁺ ∘

Step 7: Dissolve compound INT-30g (1.4 g, 3.2 mmol) in acetonitrile (140 mL) and add cesium carbonate (3.1 g, 9.6 mmol). Heat the temperature to 60 °C and stir for 16 hours. LCMS monitoring indicated that the reaction was complete. Water was added to the system to quench the reaction, and the mixture was extracted with ethyl acetate. The organic phases were combined, washed with saturated brine, dried over anhydrous sodium sulfate, filtered, and concentrated. The resulting residue was purified by preparative liquid chromatography and SFC to obtain a white solid compound INT-30 (0.21 g, yield 18.4%). ESI-MS (m/z): 357.2 [M+H]⁺ ∘ ¹H NMR (500 MHz, Chloroform-d) δ 5.24-4.93 (m, 1H), 4.52-4.42 (m, 1H), 3.78-3.71 (m, 3H), 2.93-2.84 (m, 1H), 2.42-2.35 (m, 1H), 2.17-2.09 (m, 1H), 1.61-1.55 (m, 2H), 1.55-1.46 (m, 18H).

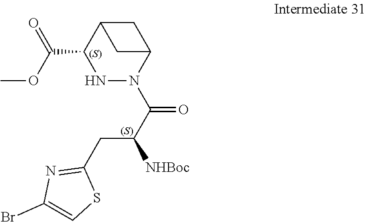

Intermediate 31 was prepared by the following steps:

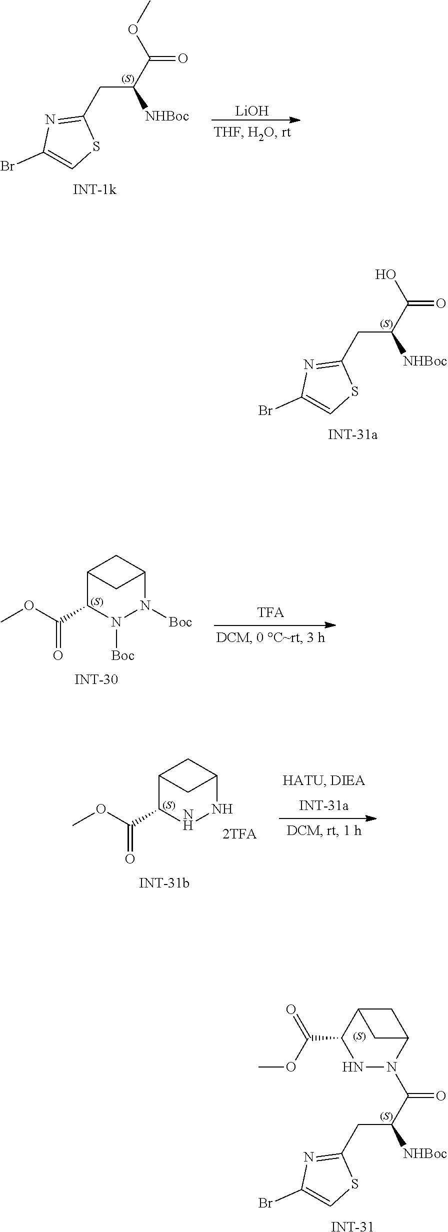

Step 1: Dissolve compound INT-1k (1.0 g, 2.7 mmol) in tetrahydrofuran (5 mL) and water (5 mL). Add lithium hydroxide monohydrate (230 mg, 5.5 mmol) at 0 °C and continue stirring for 2 hours. LCMS monitoring indicated that the reaction of the raw material was complete. The mixture was diluted with water, adjusted to pH 5 with dilute hydrochloric acid, extracted with ethyl acetate, and the organic phase was washed with saturated brine, dried over anhydrous sodium sulfate, and concentrated to obtain a yellow solid compound INT-31a (900 mg, yield 93.6%). ESI-MS (m/z): 350.8 [M+H]⁺ ∘

Step 2: Compound INT-30 (100 mg, 0.24 mmol) was dissolved in dichloromethane (1 mL), and trifluoroacetic acid (1 mL) was added thereto under ice bath. The reaction mixture was stirred at room temperature for 2 hours. After the reaction was complete, the reaction solution was concentrated to obtain trifluoroacetate salt of INT-31b as a colorless oil (91 mg, yield 100%). ESI-MS (m/z): 157.3 [M+H]⁺ ∘

Step 3: Dissolve compound INT-31b (91 mg, 0.24 mmol) in dichloromethane (5 mL), and add diisopropylethylamine (118 mg, 0.91 mmol), compound INT-31a (80 mg, 0.23 mmol) and 2-(7-azobenzotriazole)-N,N,N′,N′-tetramethyluronium hexafluorophosphate (104 mg, 0.27 mmol) in sequence at room temperature. The reaction solution is stirred at room temperature for 2 hours. LCMS monitoring shows that the reaction of the raw materials is complete. Water was added to the system to quench the reaction, and the mixture was extracted with ethyl acetate. The organic phases were combined, washed with saturated brine, dried over anhydrous sodium sulfate, filtered, and concentrated. The resulting residue was purified by silica gel column chromatography (petroleum ether/ethyl acetate=1/1) to give INT-31 (100 mg, 89.7% yield) as a colorless oil. ESI-MS (m/z): 489.2 [M+H]⁺ ∘

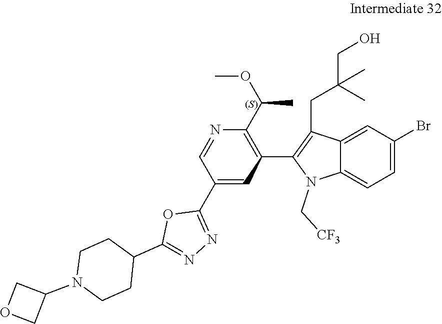

Intermediate 32 was prepared by the following steps:

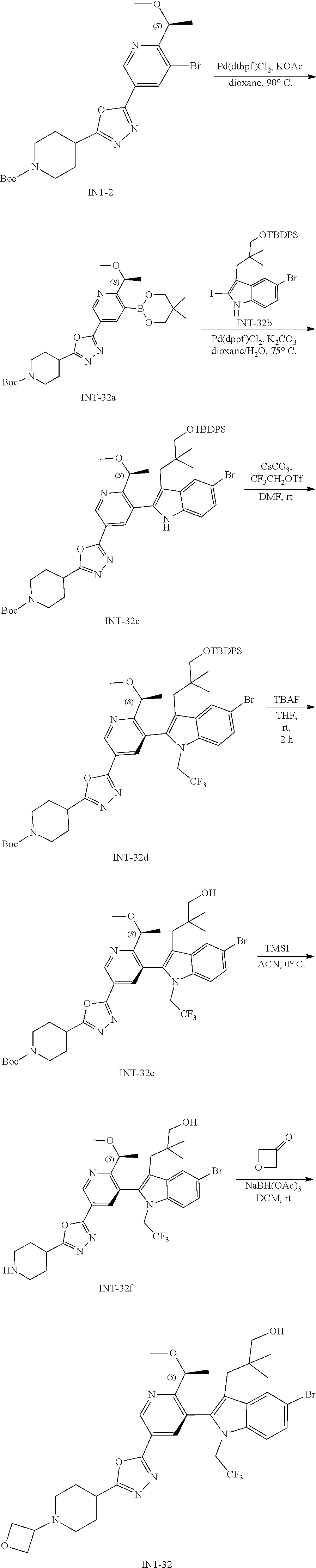

Step 1: Dissolve compound ENT-2 (10 g, 21.4 mmol) in 1,4-dioxane (300 mL), and add potassium acetate (4.2 g, 42.8 mmol), bis(neopentyl glycolato)diboron (7.3 g, 32.1 mmol) and [1,1′-bis(di-tert-butylphosphino)ferrocene]palladium dichloride (1.4 g, 2.14 mmol) in sequence. Stir the mixture at 85 °C under a nitrogen atmosphere for 16 hours. The reaction was complete when monitored by LCMS. Water was added to the system, extracted with ethyl acetate, and the organic phases were combined, washed with saturated brine, dried over anhydrous sodium sulfate, and concentrated to obtain a crude brown oily product of INT-32a, which was used directly in the next reaction without purification. ESI-MS (m/z): 501.2 [M+H]⁺ ∘

Step 2: Dissolve the crude compound INT-32a obtained above in 1,4-dioxane (150 mL) and water (15 mL), and add INT-32b (8.5 g, 13.2 mmol), potassium carbonate (5.5 g, 39.4 mmol), and [1,1′-bis(diphenylphosphino)ferrocene]palladium dichloride (0.96 g, 1.3 mmol) in sequence. The reaction solution was stirred at 85 °C under nitrogen atmosphere for 16 hours. The reaction was completed by LCMS monitoring. Water was added to the system, extracted with ethyl acetate, and the organic phases were combined, washed with saturated brine, dried over anhydrous sodium sulfate, and concentrated. The resulting residue was purified by silica gel column chromatography (petroleum ether/ethyl acetate=1/1) to give compound INT-32c (4.1 g, two-step yield 20.5%) as a colorless oil. ESI-MS (m/z): 906.7 [M+H]⁺ ∘

Step 3: Compound INT-32c (1.4 g, 1.5 mmol) was dissolved in N,N-dimethylformamide (15 mL), and cesium carbonate (1.5 g, 4.5 mmol) and 2,2,2-trifluoroethyl trifluoromethanesulfonate (1.0 g, 4.5 mmol) were added thereto. The reaction solution was stirred at room temperature for 16 hours. After the reaction was complete, water (150 mL) was added to the reaction system, and the mixture was extracted with ethyl acetate (100 mL*2). The organic phases were combined, washed with saturated brine, dried over anhydrous sodium sulfate, filtered, and concentrated. The residue was purified by silica gel column chromatography (petroleum ether/ethyl acetate=2/1) to obtain a light yellow solid compound INT-32d (770 mg, yield 51.3%). ESI-MS (m/z): 988.7 [M+H]⁺ ∘

Step 4: Compound INT-32d (770 mg, 0.78 mmol) was dissolved in tetrahydrofuran (3 mL), and tetrabutylammonium fluoride (1 M, 3.9 mL) was added thereto. The reaction mixture was stirred at room temperature for 16 hours. After the reaction was complete, water (20 mL) was added to the reaction system, and the mixture was extracted with ethyl acetate (20 mL*2). The organic phases were combined, washed with saturated brine, dried over anhydrous sodium sulfate, filtered, and concentrated. The residue was purified by silica gel column chromatography (petroleum ether/ethyl acetate=1/3) to obtain a light yellow solid compound INT-32e (526 mg, yield 90.0%). ESI-MS (m/z): 750.6 [M+H]⁺ ∘

Step 5: Compound INT-32e (400 mg, 0.54 mmol) was dissolved in acetonitrile (4 mL), and trimethylsilyl iodide (190 mg, 0.8 mmol) was added thereto under ice-cooling. The reaction mixture was stirred in an ice bath for 1 hour. After the reaction was complete, aqueous sodium bicarbonate solution (20 mL) was added to the reaction system, and the mixture was extracted with ethyl acetate (20 mL*2). The organic phases were combined, washed with saturated brine, dried over anhydrous sodium sulfate, filtered, and concentrated. The residue was purified by silica gel column chromatography (dichloromethane/methanol=10/1) to obtain a light yellow solid compound INT-32f (325 mg, yield 93.8%). ESI-MS (m/z): 650.6 [M+H]⁺ ∘

Step 6: Compound NT-32f (325 mg, 0.5 mmol) was dissolved in dichloromethane (5 mL), and 3-oxetanone (72 mg, 1.0 mmol) was added thereto at room temperature. The reaction mixture was stirred at room temperature for 10 minutes, and then sodium triacetoxyborohydride (318 mg, 1.5 mmol) was added and stirring was continued for 2 hours. After the reaction was complete, sodium bicarbonate aqueous solution (20 mL) was added to the reaction system, and the mixture was extracted with dichloromethane (20 mL*2). The organic phases were combined, washed with saturated brine, dried over anhydrous sodium sulfate, filtered, and concentrated. The residue was purified by silica gel column chromatography (dichloromethane/methanol=20/1) to obtain a light yellow solid compound INT-32 (225 mg, yield 77.9%). ESI-MS (m/z): 706.6 [M+H]⁺ ∘

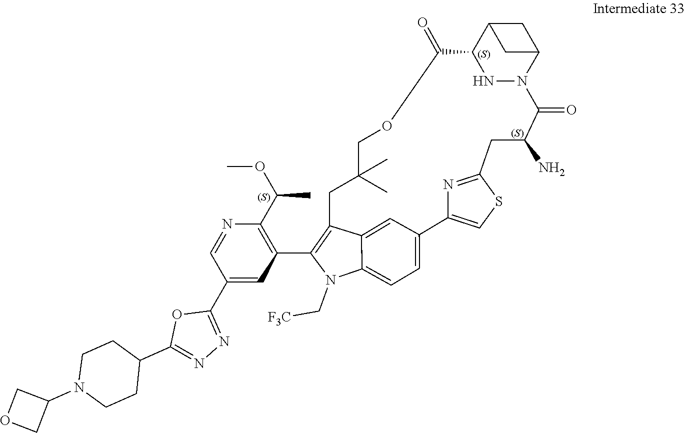

Intermediate 33 was prepared by the following steps:

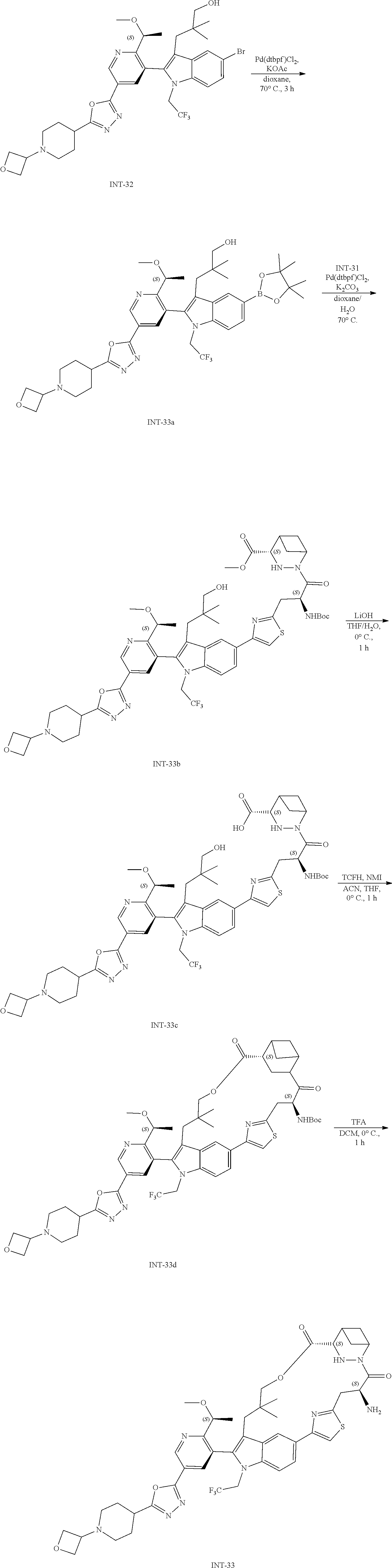

Step 1: Dissolve compound INT-32 (220 mg 0.31 mmol) in 1,4-dioxane (5 mL), and add potassium acetate (92 mg, 0.94 mmol), bis(pinacolato)diboron (118 mg, 0.47 mmol) and [1,1′-bis(di-tert-butylphosphino)ferrocene]palladium dichloride (20 mg, 0.031 mmol) in sequence. Stir the mixture at 70 °C under a nitrogen atmosphere for 16 hours. The reaction was complete when monitored by LCMS. Water was added to the system, and the mixture was extracted with ethyl acetate. The organic phases were combined, washed with saturated brine, dried over anhydrous sodium sulfate, filtered, and concentrated. The residue was purified by silica gel column chromatography (dichloromethane/methanol=20/1) to give a light yellow solid compound INT-33a (173 mg, yield 73.9%/). ESI-MS (m/z): 754.6 [M+H]⁺ ∘

Step 2: Dissolve compound INT-33a (101 mg, 0.13 mmol) in 1,4-dioxane (3 mL) and water (0.3 mL), and add NT-31 (66 mg 0.13 mmol), potassium carbonate (46 mg, 0.34 mmol), and [1,1′-bis(di-tert-butylphosphino)ferrocene]palladium dichloride (8.7 mg, 0.013 mmol) in sequence. The reaction solution was stirred at 70 °C under nitrogen atmosphere for 16 hours. The reaction was complete when monitored by LCMS. Water was added to the system, extracted with ethyl acetate, and the organic phases were combined, washed with saturated brine, dried over anhydrous sodium sulfate, and concentrated. The resulting residue was purified by silica gel column chromatography (dichloromethane/methanol=20/1) to give compound INT-33b (118 mg, yield 85.0a) as a pale yellow solid. ESI-MS (m/z): 1036.7 [M+H]⁺ ∘

Step 3: Dissolve compound INT-33b (118 mg, 0.11 mmol) in tetrahydrofuran (2 mL) and water (1 mL). Add lithium hydroxide monohydrate (14 mg, 0.33 mmol) at 0 °C and continue stirring for 1 hour. LCMS monitoring indicated that the reaction of the raw material was complete. The mixture was diluted with water, adjusted to pH 5 with dilute hydrochloric acid, extracted with ethyl acetate, and the organic phase was washed with saturated brine, dried over anhydrous sodium sulfate, and concentrated to obtain a light yellow solid compound INT-33c (101 mg, yield 90.0%). ESI-MS (m/z): 1022.7 [M+H]⁺ ∘

Step 4: Add N,N,N′,N′-tetramethylchloroformamidine hexafluorophosphate (56 mg, 0.2 mmol) and 1-methylimidazole (41 mg, 0.5 mmol) to acetonitrile (3 mL), stir to dissolve, and add a solution of compound INT-1p (101 mg, 0.1 mmol) in THF (1 mL) dropwise at room temperature. After the addition was complete, stir and react for 1 hour. LCMS monitored the complete reaction of the raw materials. Water was added to the system, extracted with ethyl acetate, and the organic phases were combined, washed with saturated brine, dried over anhydrous sodium sulfate, and concentrated. The resulting residue was purified by silica gel column chromatography (dichloromethane/methanol=20/1) to give compound INT-33d (72 mg, yield 65.0%) as a pale yellow solid. ESI-MS (m/z): 1004.7 [M+H]⁺ ∘

Step 5: Compound INT-33d (30 mg, 0.03 mmol) was dissolved in dichloromethane (2 mL), and trifluoroacetic acid (1 mL) was added thereto under ice bath. The reaction mixture was stirred at room temperature for 2 hours. After the reaction was complete, saturated sodium bicarbonate solution was added to the reaction system under ice bath to adjust the pH to 8. The mixture was extracted with dichloromethane (20 mL*2). The organic phases were combined and washed with saturated brine, dried over anhydrous sodium sulfate, filtered and concentrated to obtain a light yellow solid compound INT-33 (25 mg, yield 92.6%). ESI-MS (m/z): 904.7 [M+H]⁺ ∘

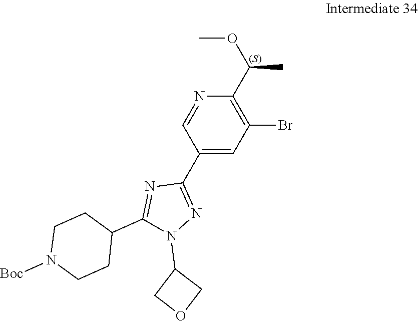

Intermediate 34 was prepared by the following steps:

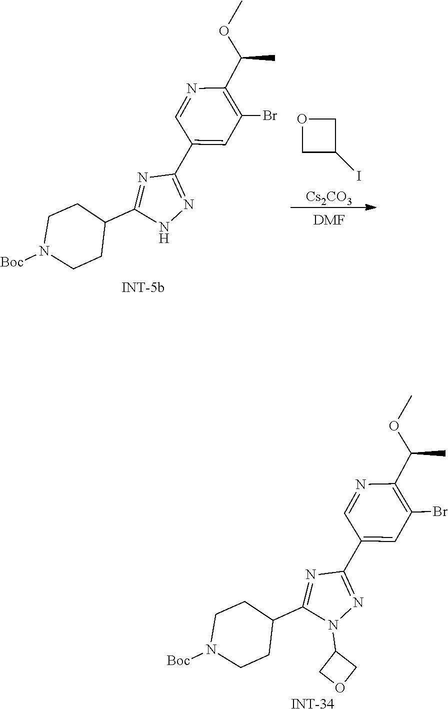

Step 1: Dissolve INT-5b (300 mg, 0.64 mmol) in N,N-dimethylformamide (6 mL) Add cesium carbonate (629 mg, 1.93 mmol) and 3-iodooxetane (355 mg, 1.93 mmol) at room temperature. Stir the reaction solution at 90 °C for 48 hours. After the reaction was completed, add water and extract with ethyl acetate. Dry the organic phase with anhydrous sodium sulfate, filter, and concentrate. The residue was purified by silica gel column chromatography (petroleum ether/ethyl acetate=2:3) to give compound INT-34 (287 mg, yield 85.4%) as a colorless oil. ESI-MS (m/z): 522.5 [M+H]⁺ ∘

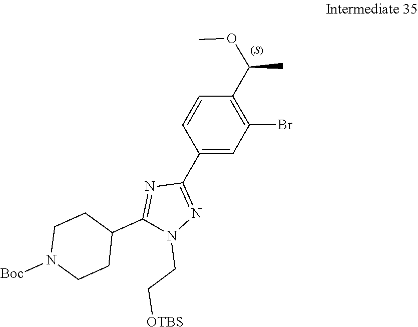

By replacing 3-iodooxetane in the synthesis step of intermediate INT-34 with tert-butyl-(2-iodoethoxy)dimethylsilane, compound INT-35 can be obtained using similar methods and reaction steps. ESI-MS (m/z): 624.7 [M+H]⁺ ∘

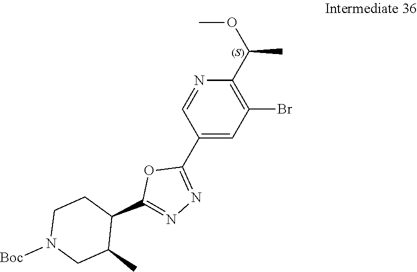

By replacing INT-2i in the synthesis step of intermediate INT-2 with cis-1-[(tert-butoxy)carbonyl]-3-methylpiperidine-4-carboxylic acid, compound INT-36 can be obtained using similar methods and reaction steps. ESI-MS (m/z): 481.3 [M+H]⁺ ∘

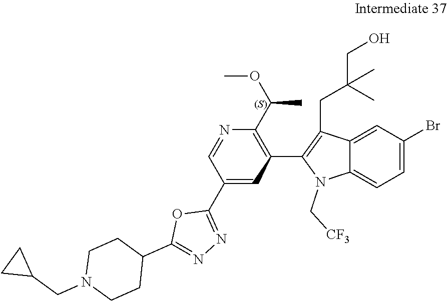

By replacing 3-oxetanone in the synthesis step of intermediate INT-32 with cyclopropylcarboxaldehyde, compound INT-37 can be obtained using similar methods and reaction steps. ESI-MS (m/z): 704.3 [M+H]⁺ ∘

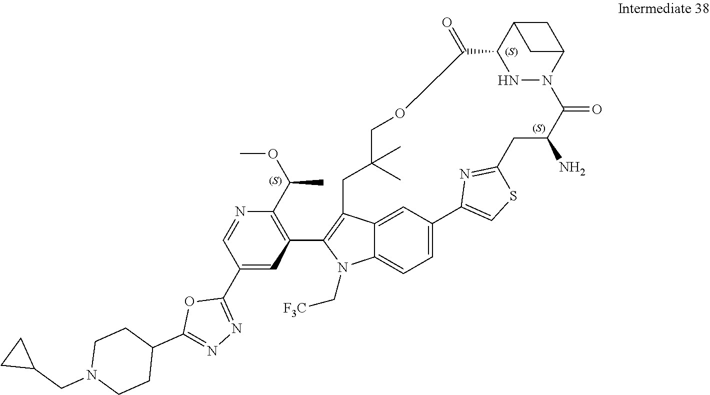

By replacing INT-32 in the synthesis step of intermediate INT-33 with INT-37, similar methods and reaction steps can be used to obtain compound INT-38. ESI-MS (m/z): 902.6 [M+H]⁺ ∘

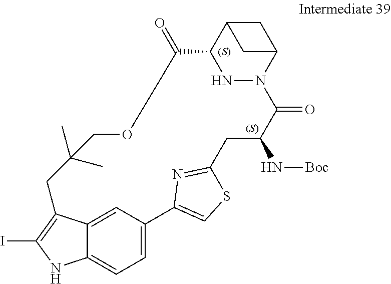

By replacing the (S)-hexahydropyridazine-3-carboxylic acid methyl ester trifluoroacetate in the synthesis step of the intermediate INT-1q with INT-31b, the compound INT-39 can be obtained using similar methods and reaction steps. ESI-MS (m/z): 706.4 [M+H]⁺ ∘

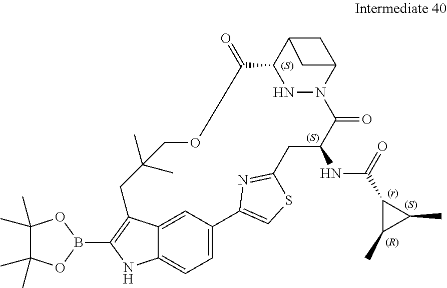

By replacing INT-1q in the synthesis step of intermediate INT-3 with INT-39 and replacing INT-3b with INT-4a, compound INT-40 can be obtained using similar methods and reaction steps. ESI-MS (m/z): 702.6 [M+H]⁺ ∘

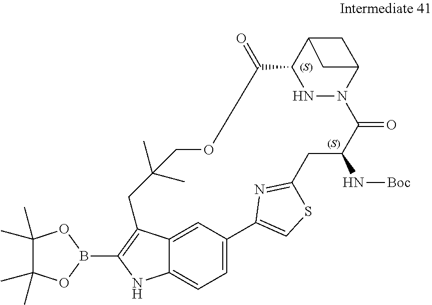

By replacing (S)-hexahydropyridazine-3-carboxylic acid methyl ester trifluoroacetate in the synthesis step of intermediate INT-1 with INT-31b, compound INT-41 can be obtained using similar methods and reaction steps. ESI-MS (m/z): 706.6 [M+H]⁺ ∘

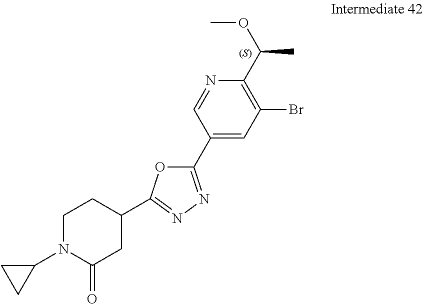

Intermediate 42 was prepared by the following steps:

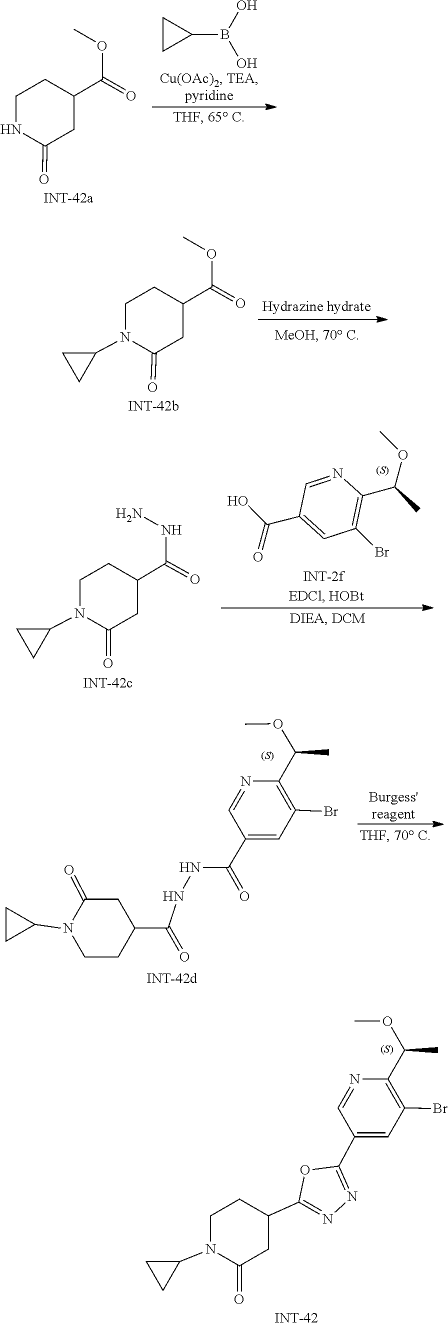

Step 1: Dissolve compound INT-42a (400 mg, 2.55 mmol) in tetrahydrofuran (8 mL), and add cyclopropylboronic acid (1.31 g, 15.27 mmol), copper acetate (1.39 g, 7.64 mmol), pyridine (805 mg, 10.18 mmol), and triethylamine (2.06 g, 20.36 mmol) in sequence. The reaction mixture was stirred at 65 °C under an oxygen atmosphere for 24 hours. Water and ethyl acetate were added thereto, and the reaction liquid was filtered through Celite. The organic phase was separated, washed with saturated brine, dried over anhydrous sodium sulfate, filtered, and concentrated. The residue was purified by silica gel column chromatography (petroleum ether/ethyl acetate=1/4) to give compound INT-42b (366 mg, yield 72.9%) as a brown oil. ESI-MS (m/z): 198.4 [M+H]⁺ ∘

Step 2: Dissolve compound INT-42b (550 mg, 2.19 mmol) in methanol (2 mL) and add hydrazine hydrate (0.5 mL). The reaction solution was stirred at 70 °C for 16 hours. LCMS monitoring showed that the reaction of the raw material was complete. The reaction solution was concentrated to obtain crude compound INT-42c (438 mg). ESI-MS (m/z): 198.4 [M+H]⁺ ∘

Step 3: Compound INT-42c (370 mg, 1.88 mmol) was dissolved in dichloromethane (8 mL), and INT-2f (488 mg, 1.88 mmol), 1-hydroxybenzotriazole (431 mg, 2.81 mmol), N,N-diisopropylethylamine (727 mg, 5.63 mmol) and 1-(3-dimethylaminopropyl)-3-ethylcarbodiimide hydrochloride (539 mg, 2.81 mmol) were added in sequence. The reaction solution was stirred at room temperature for 16 hours. LCMS monitored the complete reaction of the raw materials. Water was added to the system, and the mixture was extracted with dichloromethane. The organic phase was dried, dried over anhydrous sodium sulfate, filtered, and concentrated. The residue was purified by reverse phase column chromatography (acetonitrile/water=1:1) to give INT-42d as a white solid (517 mg, yield 62.7%). ESI-MS (m/z): 439.7 [M+H]⁺ ∘

Step 4: Dissolve compound INT-42d (517 mg, 1.18 mmol) in tetrahydrofuran (8 mL) and add Burgess reagent (421 mg, 1.77 mmol). The reaction solution was stirred at 70 °C for 16 hours. LCMS monitoring showed that the reaction of the starting material was complete, and the reaction solution was concentrated. The residue was purified by reverse phase column chromatography (acetonitrile/water=7:3) to give compound INT-42 (265 mg, yield 53.5%) as a colorless oil. ESI-MS (m/z): 421.5 [M+H]⁺ ∘

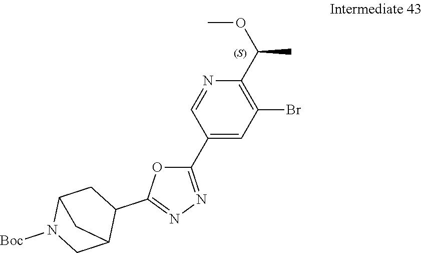

By replacing INT-2i in the synthesis step of intermediate INT-2 with 2-[(tert-butoxy)carbonyl]-2-azabicyclo[2.2.1]heptane-5-carboxylic acid, compound INT-43 can be obtained using similar methods and reaction steps. ESI-MS (m/z): 479.3 [M+H]⁺ ∘

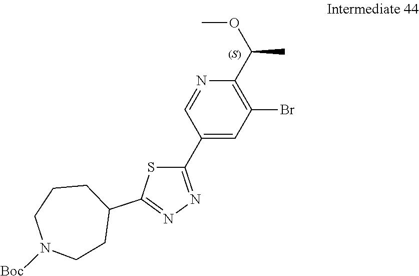

Intermediate 44 was prepared by the following steps:

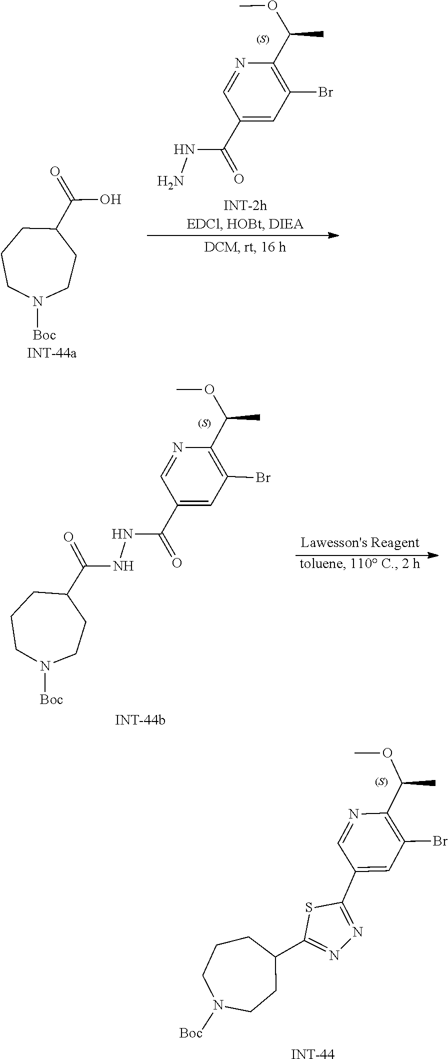

Step 1: Dissolve compound INT-2h (280 mg, 1.02 mmol) in dichloromethane (5 mL), and add INT-44a (249 mg, 1.02 mmol), 1-hydroxybenzotriazole (235 mg, 1.53 mmol), N,N-diisopropylethylamine (396 mg, 3.06 mmol) and 1-(3-dimethylaminopropyl)-3-ethylcarbodiimide hydrochloride (294 mg, 1.53 mmol) in sequence. The reaction solution was stirred at room temperature for 16 hours. LCMS monitored the complete reaction of the raw materials. Water was added to the system, and the mixture was extracted with dichloromethane. The organic phase was dried, dried over anhydrous sodium sulfate, filtered, and concentrated. The residue was purified by column chromatography (dichloromethane/methanol=20:1) to give compound INT-44b (420 mg, yield 82.3%) as a white solid. ESI-MS (m/z): 499.3 [M+H]⁺ ∘

Step 2: Compound INT-44b (50 mg, 0.10 mmol) was dissolved in toluene (5 mL) and Lawesson's reagent (49 mg, 0.12 mmol) was added. The reaction solution was stirred at 110 °C for 16 hours. LCMS monitored the complete reaction of the starting material. Saturated sodium bicarbonate solution (30 mL) was added and stirred for 30 minutes. The mixture was extracted with ethyl acetate, and the organic phase was dried over anhydrous sodium sulfate, filtered, and concentrated. The residue was purified by preparative thin-layer chromatography (petroleum ether/ethyl acetate=1:1) to give compound INT-44 (40 mg, yield 80.3%) as a colorless oil. ESI-MS (m/z): 497.3 [M+H]⁺ ∘

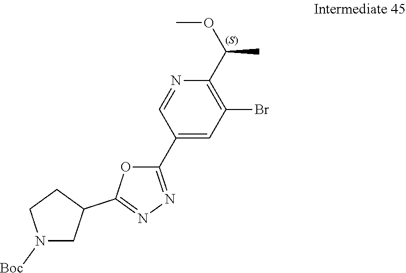

By replacing INT-2i in the synthesis step of intermediate INT-2 with 1-tert-butyloxycarbonylpyrrolidine-3-carboxylic acid, compound INT-45 can be obtained using similar methods and reaction steps. ESI-MS (m/z): 453.2 [M+H]⁺ ∘

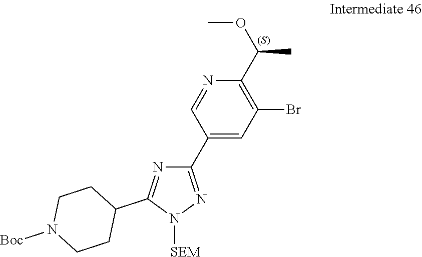

Intermediate 46 was prepared by the following steps:

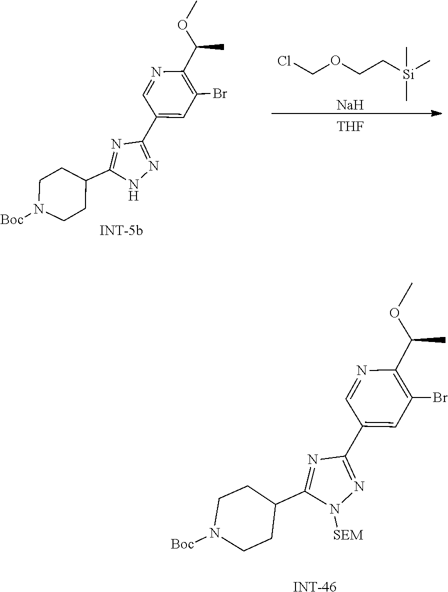

Step 1: Under nitrogen atmosphere and ice bath conditions, sodium hydride (48 mg, 1.2 mmol, 60% dispersion in oil) was added to a tetrahydrofuran solution of compound INT-5b (160 mg, 0.343 mmol). The reaction solution was stirred at room temperature for 2 hours, cooled to 0 °C, and 2-(trimethylsilyl)ethoxymethyl chloride (114 mg, 0.686 mmol) was added dropwise. After the addition was complete, the temperature was raised to room temperature and stirring was continued for 16 hours. LCMS monitored the complete reaction of the raw material. The reaction system was added with water (40 mL), extracted with ethyl acetate (40 mL), and the organic phase was dried over anhydrous sodium sulfate, filtered, and concentrated. The residue was purified by preparative liquid chromatography to give compound INT-46 (134 mg, yield 65.6%). ESI-MS (m/z): 596.4 [M+H]⁺ ∘

### Example 1

### (1S,2S)—N-((6³S,4S,Z)-1¹-ethyl-1²-(2-((S)-1-methoxyethyl)-5-(5-(1-(oxetan-3-yl)piperidin-4-yl)-1,3,4-oxadiazol-2-yl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-2-methylcyclopropane-1-carboxamide

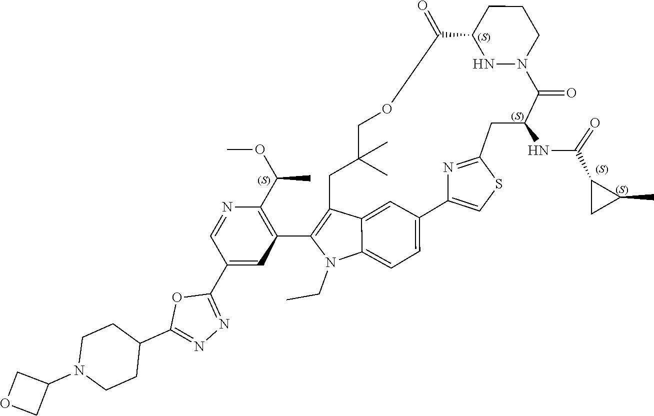

Example 1 was prepared by the following steps:

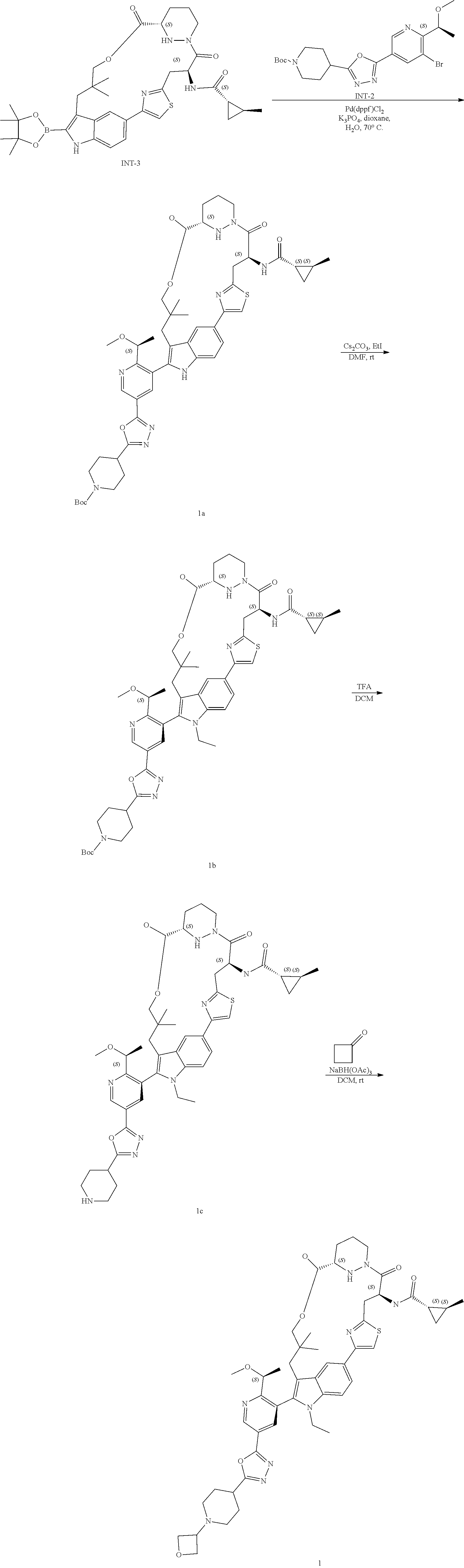

Step 1: Dissolve compound INT-3 (111 mg, 0.16 mmol) in a mixed solution of 1,4-dioxane (3 mL) and water (0.3 mL), and add INT-2 (70 mg, 0.15 mmol), [1,1′-bis(diphenylphosphino)ferrocene]palladium dichloride (11 mg, 0.015 mmol) and potassium phosphate (95 mg, 0.45 mmol) in sequence. The reaction mixture was stirred at 70 °C under nitrogen protection for 16 hours. After the reaction was complete, water (20 mL) was added to the reaction system, and the mixture was extracted with ethyl acetate (20 mL*2). The organic phases were combined, washed with saturated brine, dried over anhydrous sodium sulfate, filtered, and concentrated. The residue was purified by preparative thin-layer chromatography (dichloromethane/methanol=20:1) to obtain compound 1a as a light yellow solid (67 mg, yield 47.8%). ESI-MS (m/z): 936.7 [M+H]⁺ ∘

Step 2: Compound 1a (67 mg, 0.072 mmol) was dissolved in N,N-dimethylformamide (2 mL), and cesium carbonate (47 mg, 0.144 mmol) and iodoethane (22 mg, 0.144 mmol) were added thereto. The reaction mixture was stirred at room temperature for 16 hours. After the reaction was complete, water (20 mL) was added to the reaction system, and the mixture was extracted with ethyl acetate (20 mL*2). The organic phases were combined, washed with saturated brine, dried over anhydrous sodium sulfate, filtered, and concentrated. The residue was purified by preparative thin-layer chromatography (ethyl acetate) to obtain a light yellow solid compound 1b (20 mg, yield 29.0%). ESI-MS (m/z): 964.7 [M+H]⁺ ∘

Step 3: Compound 1b (20 mg, 0.021 mmol) was dissolved in dichloromethane (2 mL), and trifluoroacetic acid (1 mL) was added under ice-cooling. The reaction mixture was stirred at room temperature for 2 hours. After the reaction was complete, saturated sodium bicarbonate solution was added to the reaction system under ice bath to adjust the pH to 8. The mixture was extracted with dichloromethane (10 mL*2). The organic phases were combined and washed with saturated brine, dried over anhydrous sodium sulfate, filtered and concentrated to obtain a light yellow solid compound 21c (17 mg, yield 95.0%). ESI-MS (m/z): 864.7 [M+H]⁺ ∘

Step 4: Dissolve the crude compound 1c (17 mg, 0.02 mmol) in dichloromethane (2 mL), add 3-oxetanone (4 mg, 0.06 mmol) at room temperature, and stir the reaction solution at room temperature for 10 min. Then, sodium acetate borohydride (21 mg, 0.10 mmol) was slowly added to the reaction solution, and the reaction solution was stirred at room temperature for 3 hours. The reaction was complete as determined by LCMS. Saturated aqueous ammonium chloride was added to the reaction system to quench the reaction, and the mixture was extracted with dichloromethane. The organic phases were combined and concentrated, and the residue was purified by preparative liquid chromatography to obtain compound 1 as a white solid (8 mg, yield 43.5%). ESI-MS (m/z): 920.7 [M+H]⁺; LC-MS retention time RT=1.63 min. HPLC retention time RT=13.45 min.

¹H NMR (500 MHz, DMSO-d₆) δ 9.33 (d, J=2.0 Hz, 1H), 8.57-8.52 (m, 2H), 8.30 (d, J=2.0 Hz, 1H), 7.84 (s, 1H), 7.78 (dd, J=8.5, 1.5 Hz, 1H), 7.62 (d, J=8.5 Hz, 1H), 5.56 (t, J=9.0 Hz, 1H), 5.12-5.08 (m, 1H), 4.53 (t, J=6.0 Hz, 2H), 4.43 (t, J=6.0 Hz, 2H), 4.41-4.35 (m, 2H), 4.25-4.17 (m, 2H), 4.15-4.08 (m, 1H), 3.58 (s, 2H), 3.42-3.39 (m, 1H), 3.34-3.32 (m, 1H), 3.30 (s, 3H), 3.15-3.06 (m, 2H), 3.03-2.99 (m, 1H), 2.79-2.69 (m, 3H), 2.46-2.41 (m, 1H), 2.11-2.06 (m, 3H), 1.99-1.92 (m, 2H), 1.86-1.76 (m, 5H), 1.53-1.48 (m, 2H), 1.40 (d, J=6.0 Hz, 3H), 1.10-1.05 (m, 4H), 0.93-0.82 (m, 6H), 0.58-0.54 (m, 1H), 0.36 (s, 3H).

### Example 2

(1r,2R,3S)—N-((6³S,4S,Z)-1¹-ethyl-1²-(2-((S)-1-methoxyethyl)-5-(5-(1-(oxetan-3-yl)piperidin-4-yl)-1,3,4-oxadiazol-2-yl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-2,3-dimethylcyclopropane-1-carboxamide

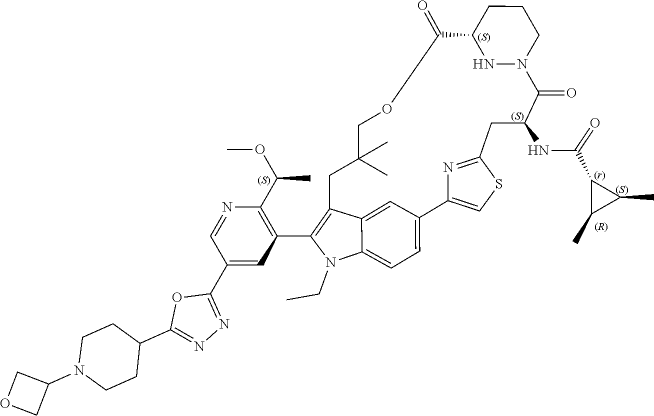

By replacing INT-3 in the synthesis steps of compound 1 with INT-4, compound 2 can be obtained using similar methods and reaction steps. ESI-MS (m/z): 934.8 [M+H]⁺; LC-MS retention time RT=1.68 min.

¹H NMR (500 MHz, DMSO-d₆) δ 9.33 (d, J=2.0 Hz, 1H), 8.53 (d, J=1.5 Hz, 1H), 8.42 (d, J=8.5 Hz, 1H), 8.31 (d, J=2.0 Hz, 1H), 7.84 (s, 1H), 7.80-7.77 (m, 1H), 7.62 (d, J=8.5 Hz, 1H), 5.55 (t, J=9.0 Hz, 1H), 5.11-5.06 (m, 1H), 4.53 (t, J=6.0 Hz, 2H), 4.43 (t, J=6.0 Hz, 2H), 4.40-4.35 (m, 2H), 4.25-4.17 (m, 2H), 4.14-4.07 (m, 1H), 3.58 (s, 2H), 3.43-3.40 (m, 2H), 3.30 (s, 3H), 3.18-3.14 (m, 1H), 3.11-3.06 (m, 1H), 3.02-2.98 (m, 1H), 2.77-2.70 (m, 3H), 2.47-2.41 (m, 1H), 2.12-2.06 (m, 3H), 2.00-1.92 (m, 3H), 1.86-1.77 (m, 4H), 1.55-1.49 (m, 1H), 1.40 (d, J=6.0 Hz, 3H), 1.20-1.14 (m, 2H), 1.10-1.05 (m, 6H), 0.93-0.86 (m, 6H), 0.36 (s, 3H).

### Example 3

### (1S,2S)—N-((6³S,4S,Z)-1¹-ethyl-1²-(2-((S)-1-methoxyethyl)-5-(1-methyl-5-(1-(oxetan-3-yl)piperidin-4-yl)-1H-1,2,4-triazol-3-yl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-2-methylcyclopropane-1-carboxamide

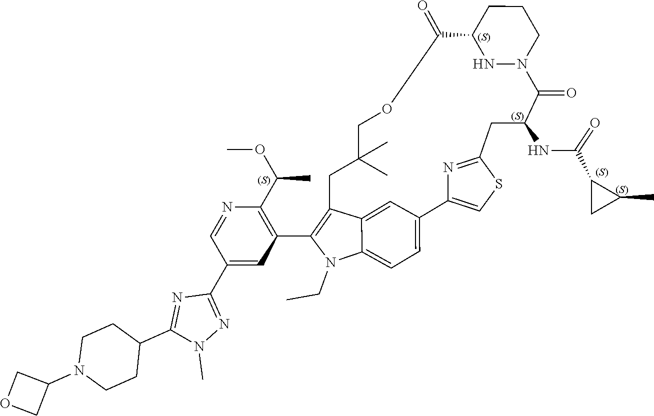

By replacing INT-2 in the synthesis steps of compound 1 with INT-5, compound 3 can be obtained using similar methods and reaction steps. ESI-MS (m/z): 933.8 [M+H]⁺; LC-MS retention time RT=1.64 min.

¹H NMR (500 MHz, DMSO-d₆) δ 9.28 (d, J=2.0 Hz, 1H), 8.57-8.50 (m, 2H), 8.14 (d, J=2.0 Hz, 1H), 7.83 (s, 1H), 7.77 (dd, J=8.5, 2.0 Hz, 1H), 7.60 (d, J=8.5 Hz, 1H), 5.56 (t, J=9.0 Hz, 1H), 5.12-5.06 (m, 1H), 4.54 (t, J=6.5 Hz, 2H), 4.43 (t, J=6.5 Hz, 2H), 4.39-4.31 (m, 2H), 4.26-4.18 (m, 2H), 4.16-4.08 (m, 1H), 3.90 (s, 3H), 3.58 (s, 2H), 3.44-3.39 (m, 1H), 3.32-3.30 (m, 1H), 3.28 (s, 3H), 3.18-3.10 (m, 1H), 3.03-2.94 (m, 2H), 2.81-2.75 (m, 3H), 2.42-2.35 (m, 1H), 2.12-2.05 (m, 1H), 1.94-1.74 (m, 9H), 1.55-1.47 (m, 2H), 1.40 (d, J=6.0 Hz, 3H), 1.09-1.04 (m, 4H), 0.91-0.84 (m, 6H), 0.59-0.53 (m, 1H), 0.37 (s, 3H).

### Example 4

(1r,2R,3S)—N-((6³S,4S,Z)-1¹-ethyl-1²-(2-((S)-1-methoxyethyl)-5-(1-methyl-5-(1-(oxetan-3-yl)piperidin-4-yl)-1H-1,2,4-triazol-3-yl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-2,3-dimethylcyclopropane-1-carboxamide

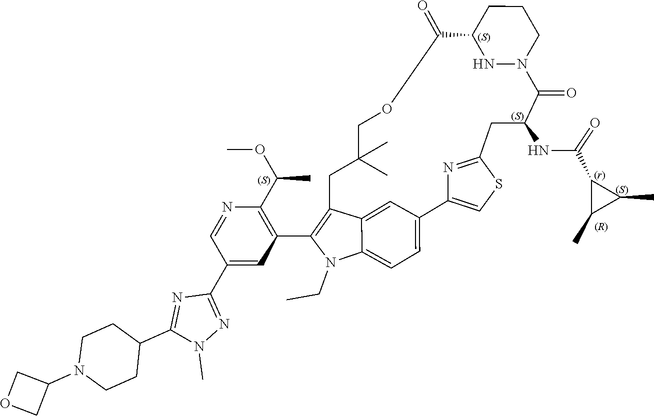

Compound 4 can be obtained by replacing INT-2 in the synthesis step of compound 1 with INT-5 and replacing INT-3 with INT-4 using similar methods and reaction steps. ESI-MS (m/z): 947.8 [M+H]+; LC-MS retention time RT=1.69 min. HPLC retention time RT=12.09 min.

¹H NMR (500 MHz, DMSO-d₆) δ 9.27 (d, J=2.0 Hz, 1H), 8.53-8.50 (m, 1H), 8.42 (d, J=9.0 Hz, 1H), 8.14 (d, J=2.0 Hz, 1H), 7.84 (s, 1H), 7.77 (dd, J=8.5, 1.5 Hz, 1H), 7.60 (d, J=8.5 Hz, 1H), 5.55 (t, J=9.0 Hz, 1H), 5.11-5.05 (m, 1H), 4.54 (t, J=6.0 Hz, 2H), 4.43 (t, J=6.0 Hz, 2H), 4.37-4.31 (m, 2H), 4.25-4.17 (m, 2H), 4.16-4.09 (m, 1H), 3.90 (s, 3H), 3.58 (s, 2H), 3.42-3.41 (m, 1H), 3.28 (s, 3H), 3.17-3.12 (m, 1H), 3.03-2.96 (m, 2H), 2.80-2.74 (m, 3H), 2.43-2.37 (m, 1H), 2.10-2.05 (m, 1H), 1.92-1.78 (m, 9H), 1.55-1.48 (m, 1H), 1.40 (d, J=6.0 Hz, 3H), 1.19-1.14 (m, 3H), 1.10-1.05 (m, 6H), 0.90-0.85 (m, 6H), 0.37 (s, 3H).

### Example 5

### (1S,2S)—N-((6³S,4S,Z)-1²-(2-((S)-1-methoxyethyl)-5-(5-(1-(oxetan-3-yl)piperidin-4-yl)-1,3,4-oxadiazol-2-yl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-2-methylcyclopropane-1-carboxamide

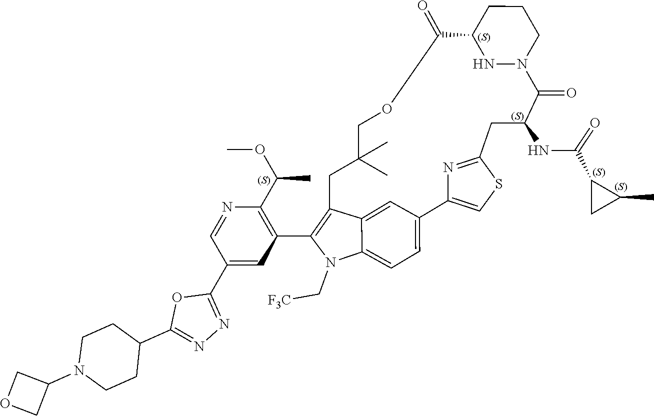

By replacing the iodoethane in the synthesis step of compound 1 with 2,2,2-trifluoroethyl trifluoromethanesulfonate, compound 5 can be obtained using a similar method and reaction steps. ESI-MS (m/z): 974.8 [M+H]⁺; LC-MS retention time RT=1.70 min.

¹H NMR (500 MHz, DMSO-d₆) δ 9.34 (d, J=2.0 Hz, 1H), 8.60-8.52 (m, 2H), 8.27 (d, J=2.0 Hz, 1H), 7.91-7.85 (m, 2H), 7.80 (d, J=8.5 Hz, 1H), 5.74-5.61 (m, 1H), 5.54 (t, J=9.0 Hz, 1H), 5.11-5.04 (m, 1H), 4.92-4.81 (m, 1H), 4.56-4.51 (m, 2H), 4.46-4.41 (m, 2H), 4.38-4.32 (m, 1H), 4.27-4.20 (m, 2H), 3.58 (s, 2H), 3.43-3.40 (m, 2H), 3.33 (s, 3H), 3.18-3.04 (m, 3H), 2.78-2.70 (m, 3H), 2.47-2.42 (m, 1H), 2.13-2.05 (m, 3H), 2.00-1.93 (m, 2H), 1.86-1.76 (m, 4H), 1.54-1.48 (m, 2H), 1.42 (d, J=6.0 Hz, 3H), 1.10-1.03 (m, 4H), 0.93 (s, 3H), 0.90-0.84 (m, 1H), 0.59-0.53 (m, 1H), 0.32 (s, 3H).

### Example 6

(1r,2R,3S)—N-((6³S,4S,Z)-1²-(2-((S)-1-methoxyethyl)-5-(5-(1-(oxetan-3-yl)piperidin-4-yl)-1,3,4-oxadiazol-2-yl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-2,3-dimethylcyclopropane-1-carboxamide

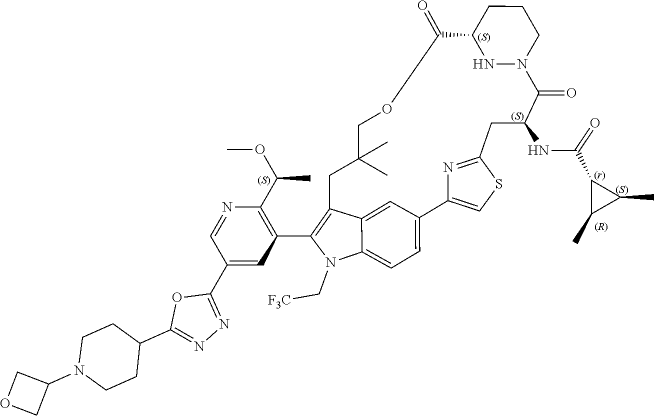

Compound 6 can be obtained by replacing INT-3 in the synthesis step of compound 1 with INT-4 and iodoethane with 2,2,2-trifluoroethyl trifluoromethanesulfonate, using similar methods and reaction steps. ESI-MS (m/z): 988.8 [M+H]⁺; LC-MS retention time RT=1.75 min.

¹H NMR (500 MHz, DMSO-d₄) δ 9.34 (d, J=2.0 Hz, 1H), 8.54 (d, J=1.5 Hz, 1H), 8.43 (d, J=9.0 Hz, 1H), 8.27 (d, J=2.0 Hz, 1H), 7.91 (s, 1H), 7.87 (dd, J=8.5, 1.5 Hz, 1H), 7.81 (d, J=8.5 Hz, 1H), 5.71-5.61 (m, 1H), 5.53 (t, J=9.0 Hz, 1H), 5.10-5.04 (m, 1H), 4.91-4.82 (m, 1H), 4.55-4.52 (m, 2H), 4.45-4.41 (m, 2H), 4.37-4.32 (m, 1H), 4.26-4.19 (m, 2H), 3.58 (s, 2H), 3.44-3.39 (m, 2H), 3.33 (s, 3H), 3.18-3.04 (m, 3H), 2.77-2.70 (m, 3H), 2.47-2.42 (m, 1H), 2.12-2.06 (m, 3H), 2.00-1.94 (m, 2H), 1.87-1.77 (m, 4H), 1.56-1.48 (m, 1H), 1.42 (d, J=6.0 Hz, 3H), 1.20-1.15 (m, 3H), 1.09 (d, J=6.0 Hz, 3H), 1.08-1.05 (m, 3H), 0.93 (s, 3H), 0.32 (s, 3H).

### Example 7

### (1S,2S)—N-((6³S,4S,Z)-1¹-ethyl-1²-(5-(5-(1-(1-isopropylazetidin-3-yl)piperidin-4-yl)-1,3,4-oxadiazol-2-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-2-methylcyclopropane-1-carboxamide

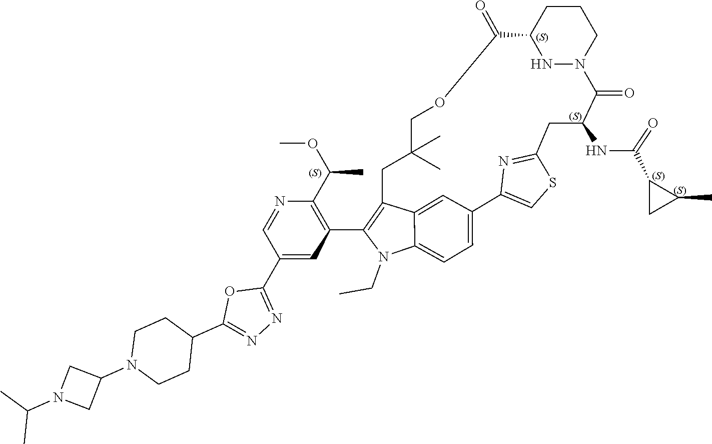

Example 7 was prepared by the following steps:

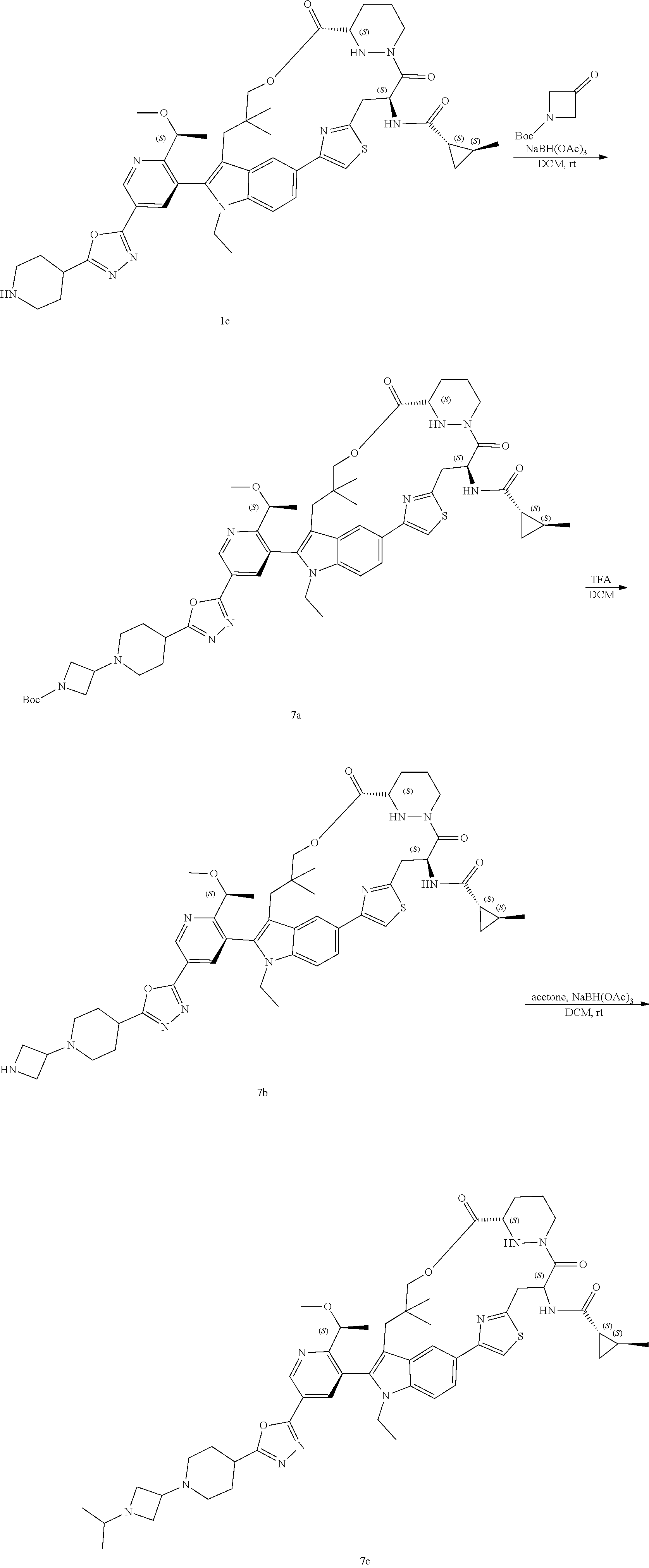

Step 1: Compound 1c (50 mg, 0.06 mmol) was dissolved in dichloromethane (2 mL). 1-tert-Butyloxycarbonyl-3-azetidinone (31 mg, 0.18 mmol) was added at room temperature. The reaction mixture was stirred at room temperature for 10 min. Then, sodium acetate borohydride (38 mg, 0.18 mmol) was slowly added to the reaction solution, and the reaction solution was stirred at room temperature for 16 hours. The reaction was complete as determined by LCMS. Saturated aqueous ammonium chloride was added to the reaction system to quench the reaction, and the mixture was extracted with dichloromethane. The organic phases were combined and concentrated, and the residue was purified by preparative thin-layer chromatography (dichloromethane/methanol=30:1) to give compound 7a as a white solid (49 mg, yield 80.0%/). ESI-MS (m/z): 1019.7 [M+H]⁺;

Step 2: Compound 7a (49 mg, 0.048 mmol) was dissolved in dichloromethane (2 mL), and trifluoroacetic acid (1 mL) was added thereto under ice-cooling. The reaction mixture was stirred at room temperature for 2 hours. After the reaction was complete, saturated sodium bicarbonate solution was added to the reaction system under ice bath to adjust the pH to 8. The mixture was extracted with dichloromethane (10 mL*2). The organic phases were combined and washed with saturated brine, dried over anhydrous sodium sulfate, filtered and concentrated to obtain a light yellow solid compound 7b (42 mg, yield 95.0%). ESI-MS (m/z): 919.7 [M+H]⁺ ∘

Step 3: Dissolve the crude compound 7b (42 mg, 0.046 mmol) in dichloromethane (2 mL), add acetone (13 mg, 0.23 mmol) at room temperature, and stir the reaction mixture at room temperature for 10 min. Then, sodium acetate borohydride (29 mg, 0.138 mmol) was slowly added to the reaction solution, and the reaction solution was stirred at room temperature for 3 hours. The reaction was complete as determined by LCMS. Saturated aqueous ammonium chloride was added to the reaction system to quench the reaction, and the mixture was extracted with dichloromethane. The organic phases were combined and concentrated, and the residue was purified by preparative liquid chromatography to obtain compound 7 as a white solid (8 mg, yield 18.1%). ESI-MS (m/z): 961.7 [M+H]⁺; LC-MS retention time RT=1.70 min.

¹H NMR (500 MHz, DMSO-d₆) δ 9.32 (d, J=2.0 Hz, 1H), 8.57-8.50 (m, 2H), 8.30 (d, J=2.0 Hz, 1H), 7.83 (s, 1H), 7.78 (dd, J=8.5, 1.5 Hz, 1H), 7.61 (d, J=8.5 Hz, 1H), 5.56 (t, J=9.0 Hz, 1H), 5.12-5.06 (m, 1H), 4.42-4.34 (m, 2H), 4.25-4.19 (m, 2H), 4.13-4.07 (m, 1H), 3.58 (s, 2H), 3.30 (s, 3H), 3.17-3.13 (m, 1H), 3.07-2.99 (m, 2H), 2.75-2.70 (m, 5H), 2.45-2.41 (m, 1H), 2.27-2.22 (m, 1H), 2.11-1.90 (m, 7H), 1.83-1.75 (m, 4H), 1.53-1.47 (m, 2H), 1.40 (d, J=6.0 Hz, 3H), 1.24-1.22 (m, 3H), 1.08-1.05 (m, 3H), 0.92-0.83 (m, 12H), 0.58-0.53 (m, 1H), 0.36 (s, 3H).

### Example 8

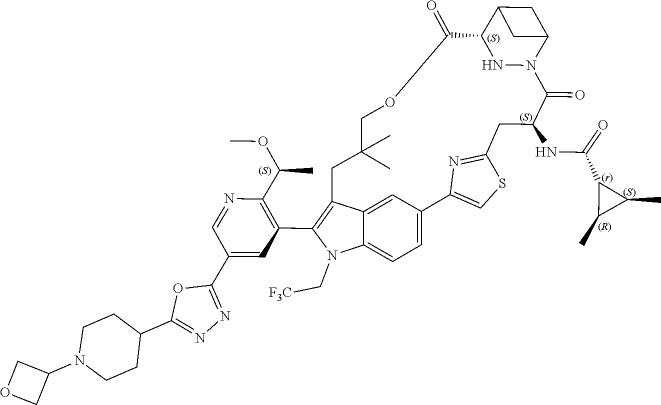

Example 8 was prepared by the following steps:

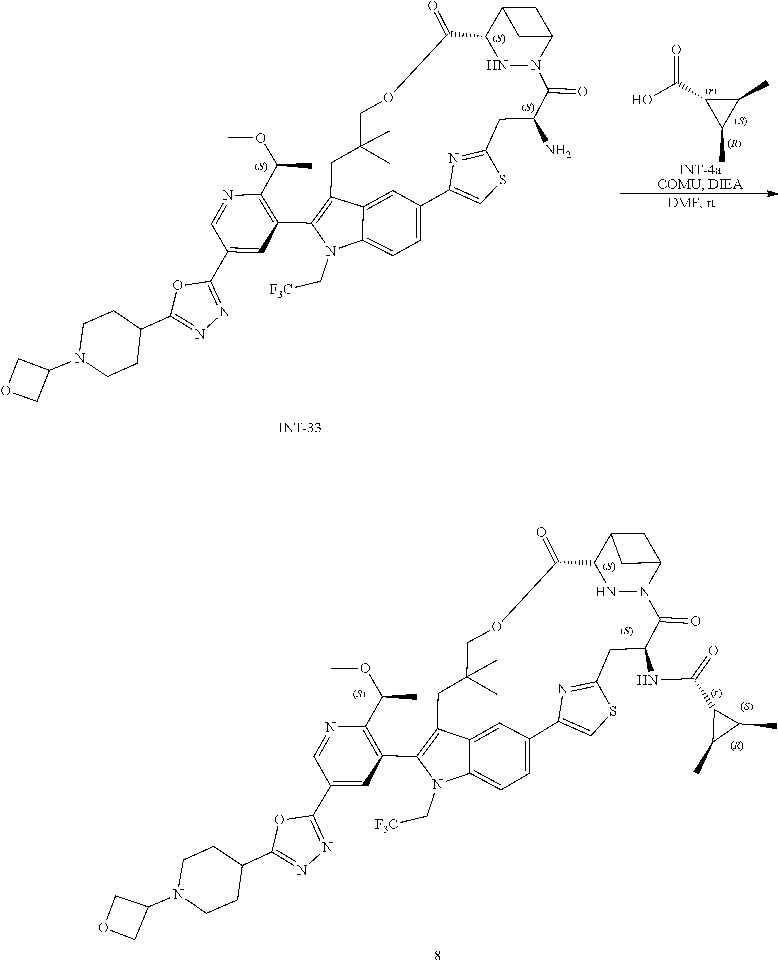

Step 1: Dissolve compound INT-33 (25 mg, 0.028 mmol) and compound INT-4a (6.3 mg, 0.056 mmol) in N,N-dimethylformamide (2 mL). Add N,N-diisopropylethylamine (11 mg, 0.084 mmol) and (1-Cyano-2-ethoxy-2-oxoethylidenaminooxy)dimethylaminomorpholinocarbenium Hexafluorophosphate (18 mg, 0.041 mmol) at room temperature. React at room temperature for 1 hour. LCMS monitoring shows that the reaction of the raw materials was complete. Water was added to the system, extracted with ethyl acetate, and the organic phases were combined, washed with saturated brine, dried over anhydrous sodium sulfate, filtered, and concentrated. The residue was purified by preparative liquid chromatography to obtain compound 8 (8 mg, yield 28.9%) as a white solid. ESI-MS (m/z): 1000.7 [M+H]⁺ ∘LC-MS retention time RT=1.80 min.

¹H NMR (500 MHz, DMSO-d₆) δ 9.34 (d, J=2.0 Hz, 1H), 8.49-8.42 (m, 2H), 8.30-8.24 (m, 1H), 7.91 (s, 1H), 7.89-7.85 (m, 1H), 7.83-7.78 (m, 1H), 5.96-5.90 (m, 1H), 5.70-5.60 (m, 1H), 5.37-5.29 (m, 1H), 4.91-4.82 (m, 1H), 4.75-4.69 (m, 1H), 4.54 (t, J=6.5 Hz, 2H), 4.51-4.46 (m, 1H), 4.43 (t, J=6.5 Hz, 2H), 4.37-4.31 (m, 1H), 3.59-3.53 (m, 2H), 3.44-3.41 (m, 1H), 3.32 (s, 3H), 3.29-3.25 (m, 1H), 3.19-3.04 (m, 3H), 2.76-2.70 (m, 2H), 2.67-2.61 (m, 1H), 2.49-2.43 (m, 1H), 2.38-2.30 (m, 1H), 2.18-2.12 (m, 1H), 2.11-2.05 (m, 2H), 2.00-1.93 (m, 2H), 1.88-1.79 (m, 2H), 1.66-1.58 (m, 1H), 1.42 (d, J=6.0 Hz, 3H), 1.31-1.25 (m, 2H), 1.20-1.16 (m, 2H), 1.12-1.05 (m, 6H), 0.90 (s, 3H), 0.31 (s, 3H).

### Example 9

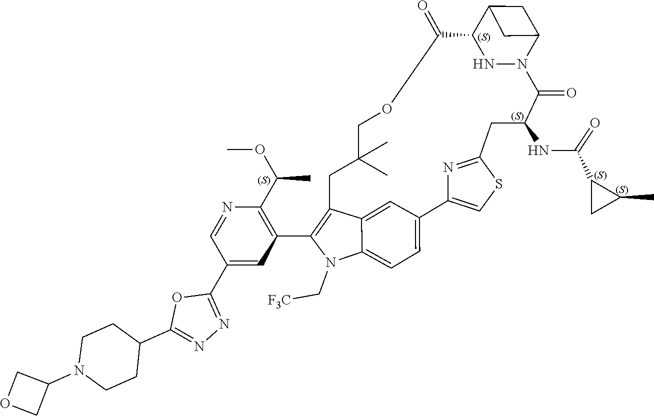

By replacing INT-4a in the synthesis steps of compound 8 with INT-3b, compound 9 can be obtained using similar methods and reaction steps. ESI-MS (m/z): 986.8 [M+H]+; LC-MS retention time RT=1.75 min.

¹H NMR (500 MHz, DMSO-d₆) δ 9.34 (d, J=2.0 Hz, 1H), 8.56 (d, J=9.0 Hz, 1H), 8.48-8.45 (m, 1H), 8.30-8.25 (m, 1H), 7.90 (s, 1H), 7.89-7.85 (m, 1H), 7.82-7.78 (m, 1H), 5.98-5.91 (m, 1H), 5.69-5.60 (m, 1H), 5.38-5.32 (m, 1H), 4.91-4.82 (m, 1H), 4.75-4.70 (m, 1H), 4.53 (t, J=6.5 Hz, 2H), 4.51-4.47 (m, 1H), 4.43 (t, J=6.5 Hz, 2H), 4.38-4.31 (m, 1H), 3.58-3.52 (m, 2H), 3.44-3.38 (m, 1H), 3.32 (s, 3H), 3.27-3.24 (m, 1H), 3.17-3.02 (m, 3H), 2.76-2.70 (m, 2H), 2.66-2.62 (m, 1H), 2.38-2.31 (m, 2H), 2.18-2.12 (m, 1H), 2.12-2.05 (m, 2H), 2.01-1.93 (m, 2H), 1.88-1.79 (m, 3H), 1.66-1.60 (m, 1H), 1.52-1.48 (m, 1H), 1.42 (d, J=6.0 Hz, 3H), 1.29-1.24 (m, 2H), 1.08 (s, 3H), 0.92 (s, 3H), 0.60-0.55 (m, 1H), 0.30 (s, 3H).

### Example 10

(1r,2R,3S)—N-((6³S,4S,Z)-1²-(5-(5-(1-(1-isopropylazetidin-3-yl)piperidin-4-yl)-1,3,4-oxadiazol-2-yl)-2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-2,3-dimethylcyclopropane-1-carboxamide

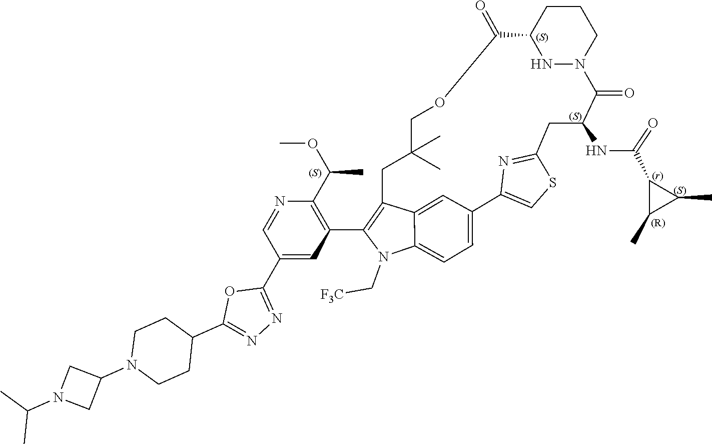

Example 10 was prepared by the following steps:

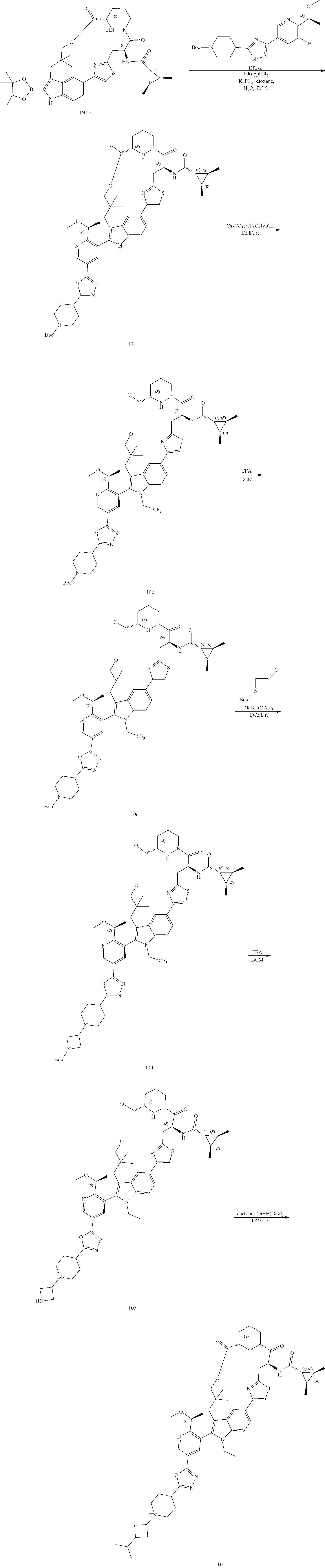

Compound 10d can be obtained Using similar methods and synthesis steps by replacing INT-3 in the synthesis steps of compound 1 with INT-4, replacing iodoethane with 2,2,2-trifluoroethyl trifluoromethanesulfonate, and replacing 3-oxetanone with 1-tert-butoxycarbonyl-3-azetidinone. By replacing 7a in the synthesis steps of compound 7 with compound 10d, compound 10 can be obtained using similar methods and synthesis steps. ESI-MS (m/z): 1029.8 [M+H]+; LC-MS retention time RT=1.90 min.

¹H NMR (500 MHz, DMSO-d₆) δ 9.37-9.31 (m, 1H), 8.58-8.52 (m, 1H), 8.43 (d, J=9.0 Hz, 1H), 8.29-8.24 (m, 1H), 7.92-7.89 (m, 1H), 7.89-7.85 (m, 1H), 7.83-7.78 (m, 1H), 5.71-5.60 (m, 1H), 5.53 (t, J=9.0 Hz, 1H), 5.11-5.03 (m, 1H), 4.91-4.80 (m, 1H), 4.38-4.30 (m, 1H), 4.28-4.17 (m, 2H), 3.62-3.52 (m, 2H), 3.37-3.34 (m, 2H), 3.33 (s, 3H), 3.20-3.12 (m, 1H), 3.09-3.01 (m, 2H), 2.80-2.69 (m, 4H), 2.69-2.62 (m, 2H), 2.47-2.41 (m, 1H), 2.23-2.16 (m, 1H), 2.13-2.02 (m, 3H), 1.98-1.89 (m, 2H), 1.86-1.72 (m, 4H), 1.57-1.47 (m, 1H), 1.44-1.39 (m, 3H), 1.27-1.15 (m, 4H), 1.13-1.04 (m, 6H), 0.97-0.89 (m, 3H), 0.86-0.79 (m, 6H), 0.32 (s, 3H).

### Example 18

### (1S,2S)—N-((6³S,4S,Z)-1¹-ethyl-1²-(2-((S)-1-methoxyethyl)-5-(5-((S)-1-(oxetan-3-yl)piperidin-3-yl)-1,3,4-oxadiazol-2-yl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-2-methylcyclopropane-1-carboxamide

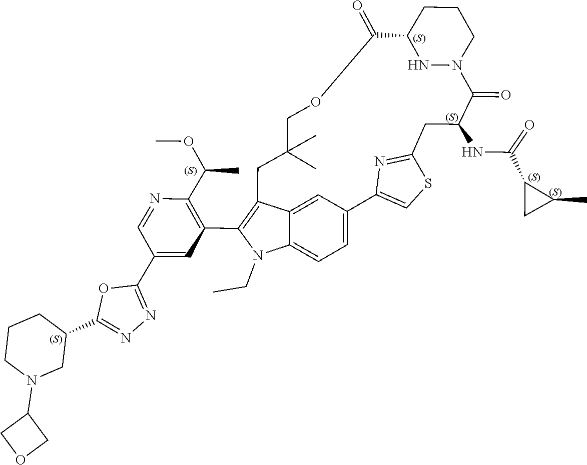

By replacing INT-2 in the synthesis steps of compound 1 with INT-11, compound 18 can be obtained using similar methods and reaction steps. ESI-MS (m/z): 920.8 [M+H]+; LC-MS retention time RT=1.72 min.

¹H NMR (500 MHz, DMSO-d₆) δ 9.34 (d, J=2.0 Hz, 1H), 8.57-8.51 (m, 2H), 8.29 (d, J=2.0 Hz, 1H), 7.83 (s, 1H), 7.80-7.76 (m, 1H), 7.61 (d, J=8.5 Hz, 1H), 5.56 (t, J=9.0 Hz, 1H), 5.12-5.06 (m, 1H), 4.56-4.49 (m, 2H), 4.45-4.35 (m, 4H), 4.26-4.18 (m, 2H), 4.14-4.08 (m, 1H), 3.58 (s, 2H), 3.49-3.43 (m, 1H), 3.33-3.28 (m, 1H), 3.30 (s, 3H), 3.28-3.23 (m, 1H), 3.18-3.13 (m, 1H), 3.03-2.97 (m, 1H), 2.92-2.85 (m, 1H), 2.80-2.72 (m, 1H), 2.46-2.40 (m, 1H), 2.39-2.31 (m, 1H), 2.12-2.04 (m, 2H), 2.03-1.97 (m, 1H), 1.82-1.73 (m, 4H), 1.65-1.58 (m, 1H), 1.53-1.47 (m, 2H), 1.41 (d, J=6.0 Hz, 3H), 1.09-1.04 (m, 4H), 0.94-0.85 (m, 8H), 0.59-0.52 (m, 1H), 0.36 (s, 3H).

### Example 26

### (1S,2S)—N-((6³S,4S,Z)-1¹-ethyl-1²-(2-((S)-1-methoxyethyl)-5-(1-(oxetan-3-yl)-5-(1-(oxetan-3-yl)piperidin-4-yl)-1H-1,2,4-triazol-3-yl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-2-methylcyclopropane-1-carboxamide

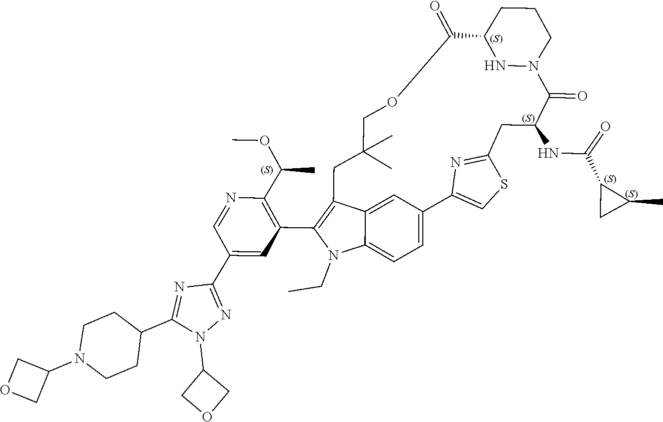

By replacing INT-2 in the synthesis steps of compound 1 with INT-33, compound 26 can be obtained using similar methods and reaction steps. ESI-MS (m/z): 975.1 [M+H]⁺; LC-MS retention time RT=1.64 min.

¹H NMR (500 MHz, DMSO-d₆) δ 9.35 (d, J=2.0 Hz, 1H), 8.54-8.50 (m, 2H), 8.24 (d, J=2.0 Hz, 1H), 7.82 (s, 1H), 7.77 (dd, J=8.5, 1.5 Hz, 1H), 7.61 (d, J=8.5 Hz, 1H), 5.88-5.82 (m, 1H), 5.57 (t, J=9.0 Hz, 1H), 5.10-5.06 (m, 1H), 5.01-4.92 (m, 4H), 4.56-4.52 (m, 2H), 4.45-4.41 (m, 2H), 4.38-4.34 (m, 2H), 4.25-4.14 (m, 3H), 3.60 (s, 2H), 3.43-3.39 (m, 1H), 3.36-3.34 (m, 1H), 3.29 (s, 3H), 3.18-3.12 (m, 1H), 3.06-3.01 (m, 1H), 2.96-2.92 (m, 1H), 2.79-2.73 (m, 3H), 2.43-2.38 (m, 1H), 2.11-2.06 (m, 1H), 2.03-1.96 (m, 1H), 1.92-1.87 (m, 2H), 1.83-1.77 (m, 6H), 1.52-1.48 (m, 2H), 1.41 (d, J=6.0 Hz, 3H), 1.08-1.06 (m, 4H), 0.91 (s, 3H), 0.89-0.86 (m, 3H), 0.57-0.53 (m, 1H), 0.39 (s, 3H).

### Example 27

### (1S,2S)—N-((6³S,4S,Z)-1²-(5-(1-(2-(dimethylamino)ethyl)-5-(1-(oxetan-3-yl)piperidin-4-yl)-1H-1,2,4-triazol-3-yl)-2-((S)-1-methoxyethyl)pyridin-3-yl)-1¹-ethyl-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-2-methylcyclopropane-1-carboxamide

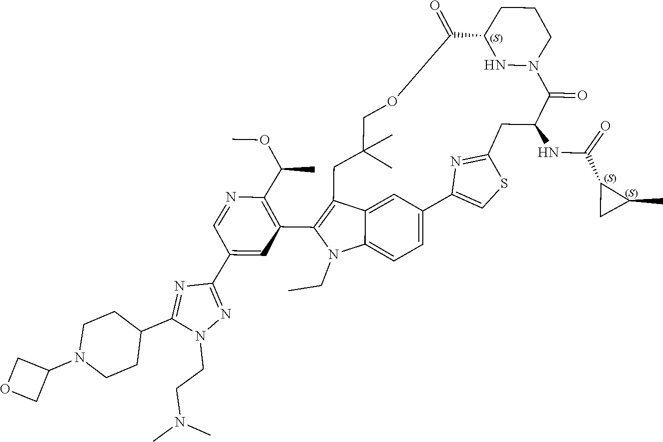

Example 27 was prepared by the following steps:

[chemical structure]

Step 1: Dissolve compound INT-3 (157 mg, 0.23 mmol) in a mixed solution of 1,4-dioxane (5 mL) and water (0.5 mL), and add INT-35 (104 mg, 0.17 mmol), [1,1′-bis(diphenylphosphino)ferrocene]palladium dichloride (24 mg, 0.024 mmol) and potassium phosphate (106 mg, 0.50 mmol) in sequence. The reaction mixture was stirred at 70 °C under nitrogen protection for 16 hours. After the reaction was complete, the reaction solution was concentrated, and the residue was purified by preparative thin-layer chromatography (dichloromethane/methanol=20:1) to obtain compound 27a as a light yellow solid (138 mg, yield 75.8%). ESI-MS (m/z): 1093.8 [M+H]⁺ ∘

Step 3: Compound 27a (138 mg, 0.126 mmol) was dissolved in N,N-dimethylformamide (2 mL), and cesium carbonate (123 mg, 0.378 mmol) and iodoethane (59 mg, 0.378 mmol) were added thereto. The reaction mixture was stirred at room temperature for 16 hours. After the reaction was complete, water (20 mL) was added to the reaction system, and the mixture was extracted with ethyl acetate (40 mL*3). The organic phases were combined, washed with water and saturated brine, dried over anhydrous sodium sulfate, filtered, and concentrated to obtain compound 27b. ESI-MS (m/z): 1121.8 [M+H]⁺ ∘

Step 3: Dissolve the crude compound 27b in tetrahydrofuran (4 mL) and add tetrabutylammonium fluoride (0.25 mL, 1.0 M in THF). The reaction mixture was stirred at room temperature for 16 hours. After the reaction was complete, the product was diluted with ethyl acetate (60 mL), washed with water and saturated brine, dried over anhydrous sodium sulfate, filtered, and concentrated. The residue was purified by preparative thin-layer chromatography (dichloromethane/methanol=15:1) to give compound 27c as a light yellow solid (72 mg, two-step yield 56.6%). ESI-MS (m/z): 1007.8 [M+H]⁺ ∘

Step 4: Compound 27c (72 mg, 0.071 mmol) and N,N-diisopropylethylamine (28 mg, 0.22 mmol) were dissolved in dichloromethane (3 mL), and methanesulfonic anhydride (19 mg, 0.11 mmol) was added thereto under ice-cooling. The reaction mixture was stirred at room temperature for 4 hours. After the reaction was complete, the mixture was diluted with dichloromethane (60 mL), washed with water and saturated brine, dried over anhydrous sodium sulfate, filtered and concentrated to obtain compound 27d. ESI-MS (m/z): 1085.9 [M+H]⁺ ∘

Step 5: Dissolve the crude compound 27d in acetonitrile (3 mL), and add potassium carbonate (30 mg, 0.22 mmol), potassium iodide (12 mg, 0.071 mmol), and dimethylamine (0.11 mL, 2.0 M in MeOH). The reaction mixture was stirred at 70 °C for 24 hours. After the reaction was complete, water (20 mL) was added to the reaction system, and the mixture was extracted with ethyl acetate (40 mL*3). The organic phases were combined, washed with water and saturated brine, dried over anhydrous sodium sulfate, filtered, and concentrated. The residue was purified by preparative thin-layer chromatography (dichloromethane/methanol=15:1) to obtain compound 27e as a light yellow solid (50 mg, two-step yield 67.6%). ESI-MS (m/z): 1034.7 [M+H]⁺ ∘

Step 6: Compound 27e (20 mg, 0.021 mmol) was dissolved in dichloromethane (3 mL), and trifluoroacetic acid (1 mL) was added under ice-cooling. The reaction mixture was stirred at room temperature for 2 hours. After the reaction was complete, saturated sodium bicarbonate solution was added to the reaction system under ice bath to adjust the pH to 8. The mixture was extracted with dichloromethane (30 mL*3). The organic phases were combined, washed with saturated brine, dried over anhydrous sodium sulfate, filtered, and concentrated to obtain compound 27f as a light yellow solid. ESI-MS (m/z): 934.7 [M+H]⁺ ∘

Step 7: The crude compound 27f was dissolved in dichloromethane (3 mL), and 3-oxetanone (11 mg, 0.15 mmol) was added at room temperature. The reaction mixture was stirred at room temperature for 10 minutes. Then, sodium acetate borohydride (51 mg, 0.24 mmol) was slowly added to the reaction solution, and the reaction solution was stirred at room temperature for 3 hours. The reaction was complete as determined by LCMS. Saturated sodium bicarbonate solution was added to the reaction system to quench the reaction, and the mixture was extracted with dichloromethane. The organic phases were combined and concentrated, and the residue was purified by preparative thin-layer chromatography (dichloromethane/methanol=13:1) to give compound 27 as a white solid (10 mg, yield 20.3%). ESI-MS (m/z): 990.9 [M+H]⁺; LC-MS retention time RT=1.68 min. HPLC retention time RT=10.76 min.

¹H NMR (500 MHz, DMSO-d) S 9.32 (s, 1H), 8.55-8.50 (m, 2H), 8.18 (d, J=2.0 Hz, 1H), 7.83 (s, 1H), 7.77 (dd, J=8.5, 1.5 Hz, 1H), 7.61 (d, J=8.5 Hz, 1H), 5.57 (t, J=9.0 Hz, 1H), 5.11-5.06 (m, 1H), 4.62-4.40 (m, 6H), 4.38-4.32 (m, 2H), 4.25-4.11 (m, 3H), 3.59 (s, 2H), 3.46-3.39 (m, 1H), 3.32-3.30 (m, 2H), 3.28 (s, 3H), 3.18-3.13 (m, 1H), 3.06-2.98 (m, 2H), 2.85-2.72 (m, 4H), 2.51-2.51 (m, 6H), 2.42-2.38 (m, 1H), 2.10-2.06 (m, 1H), 1.95-1.73 (m, 9H), 1.53-1.48 (m, 2H), 1.40 (d, J=6.0 Hz, 3H), 1.07 (s, 4H), 0.90 (s, 3H), 0.88-0.85 (m, 3H), 0.57-0.53 (m, 1H), 0.38 (s, 3H).

| Compound | structure | M/Z |
|---|---|---|
| 11 | 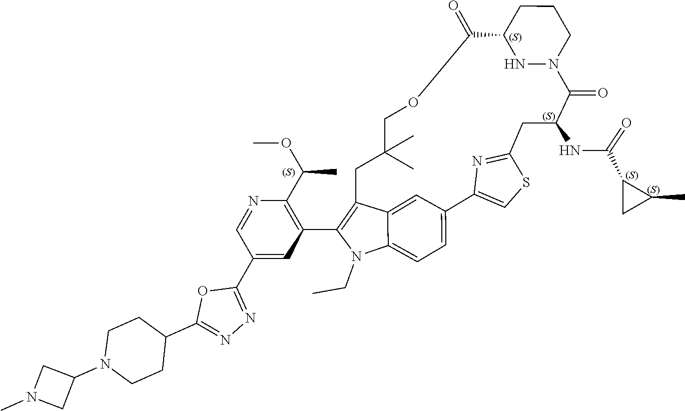 | 933 |
| 12 | 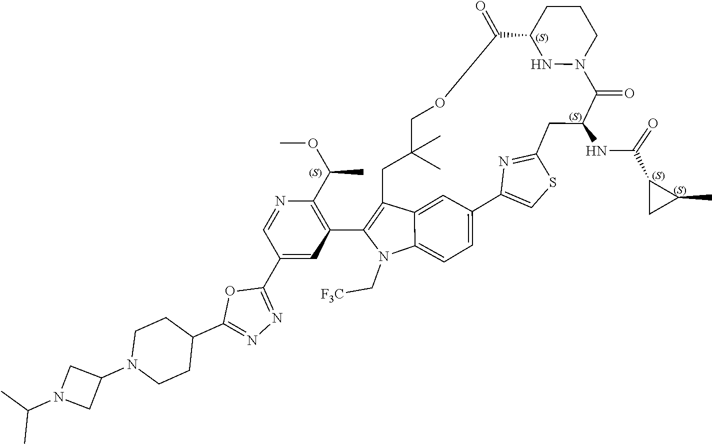 | 1015 |
| 13 | 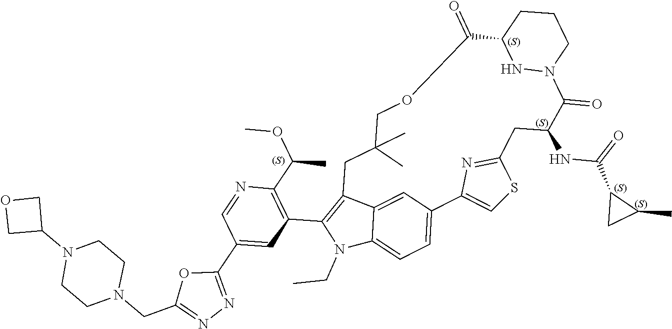 | 935 |
| 14 | 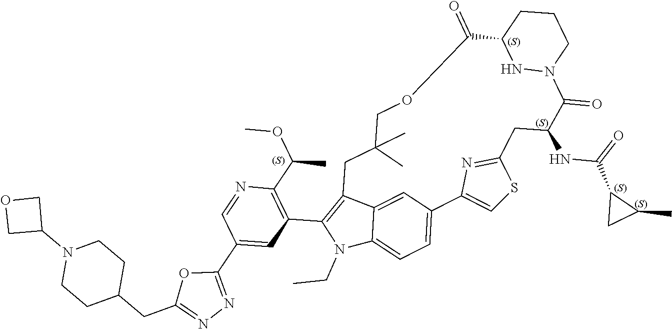 | 934 |
| 15 | 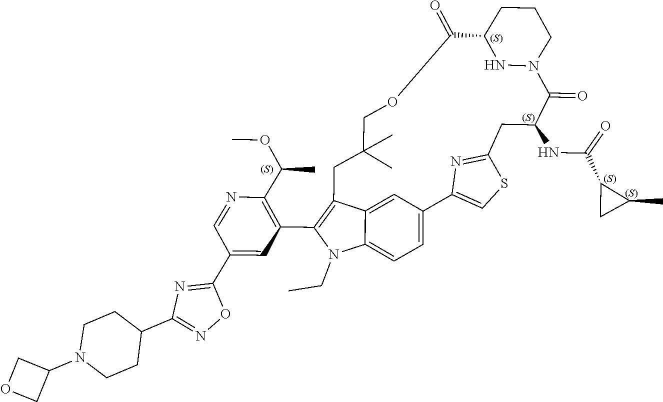 | 920 |
| 16 | 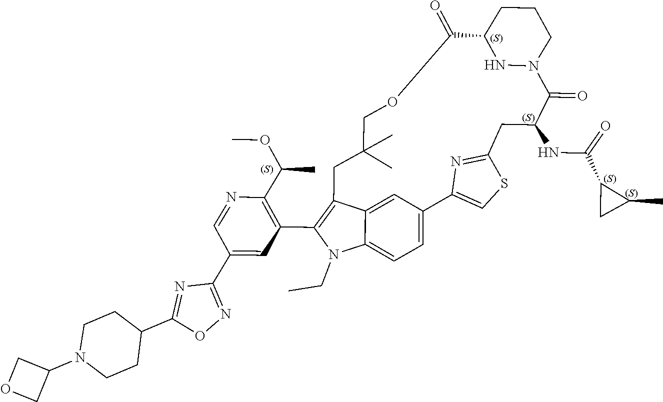 | 920 |
| 17 | 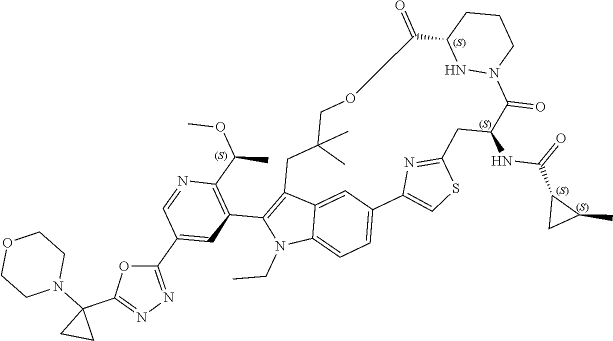 | 906 |
| 19 | 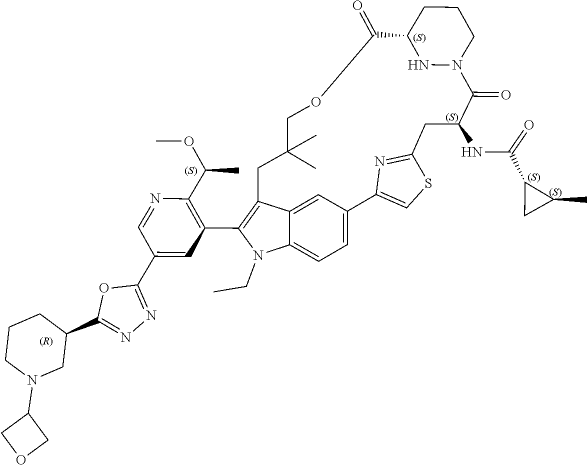 | 920 |
| 20 | 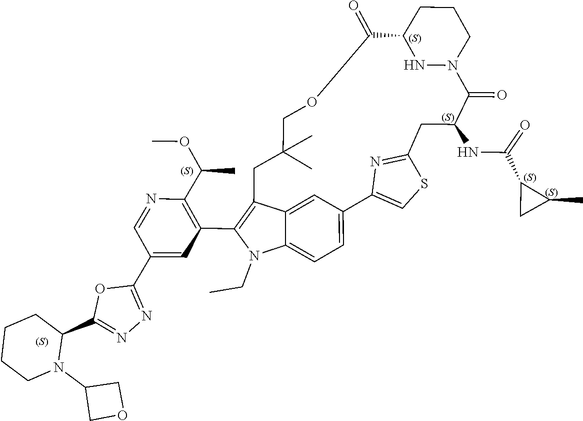 | 920 |
| 21 | 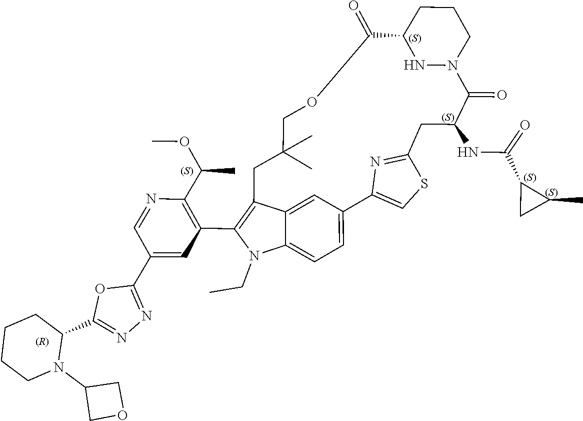 | 920 |
| 22 | 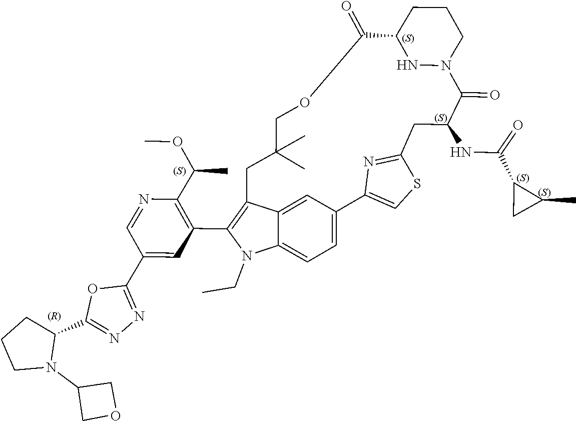 | 906 |
| 23 | 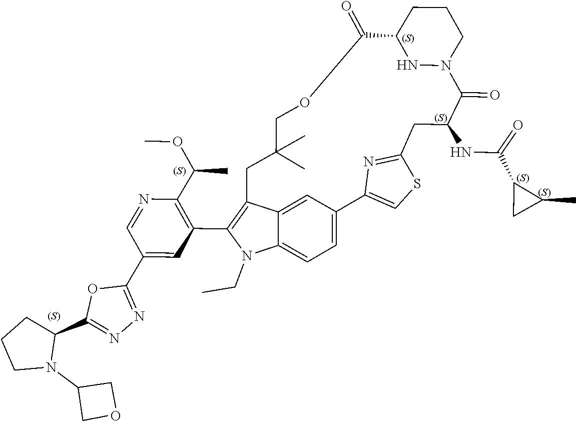 | 906 |
| 24 | 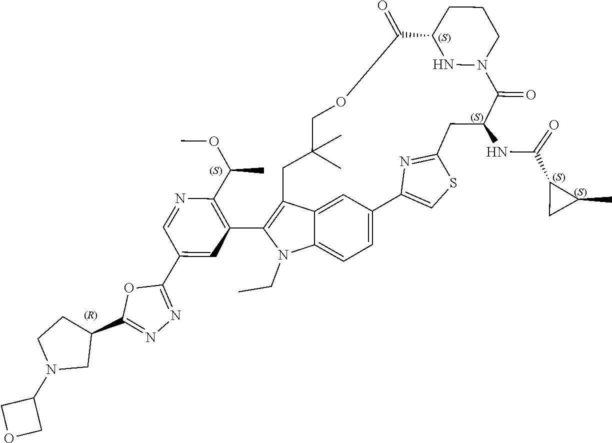 | 906 |
| 25 | 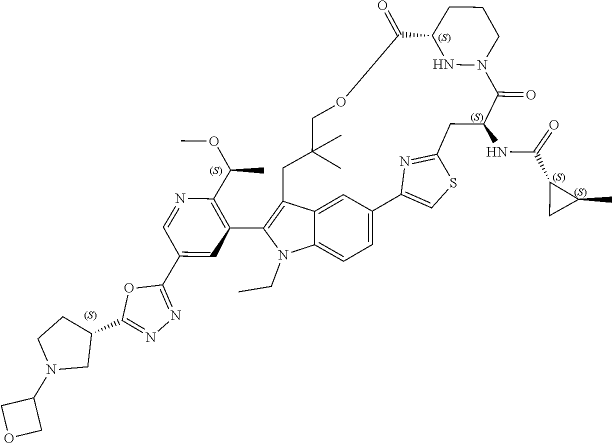 | 906 |
| 36 | 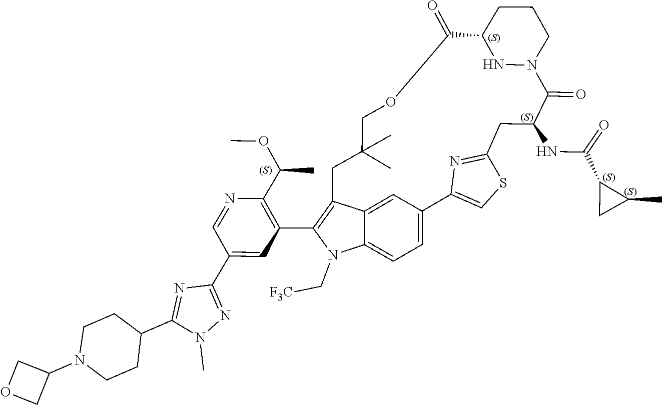 | 987 |
| 37 | 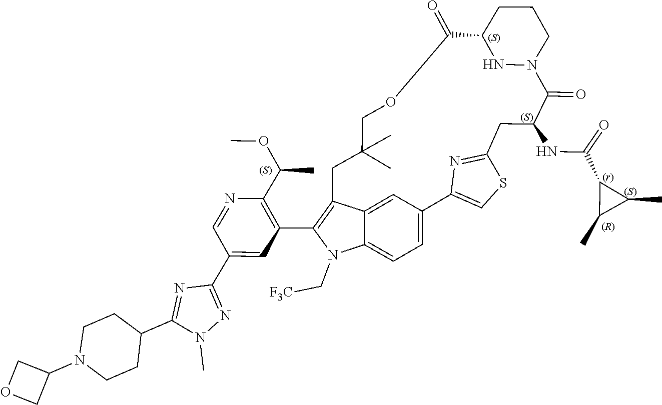 | 1001 |

### Example 38

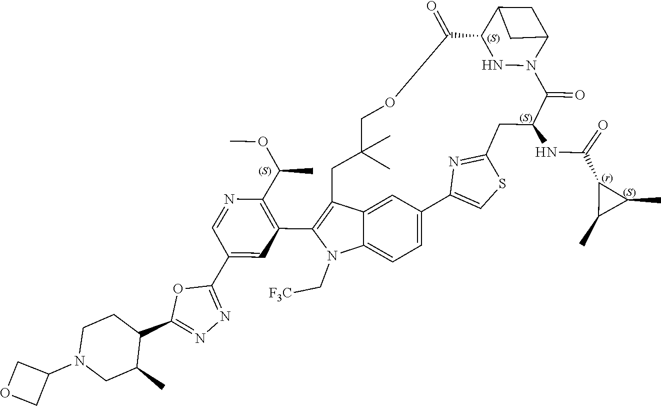

By replacing INT-3 in the synthesis steps of compound 1 with INT-40 and replacing INT-2 with INT-36, 2,2,2-trifluoroethyl trifluoromethanesulfonate, compound 38 can be obtained using similar methods and synthesis steps. ESI-MS (m/z): 1014.8 [M+H]⁺; LC-MS retention time RT=1.80 min.

¹H NMR (500 MHz, DMSO-d₆) δ 9.39-9.29 (m, 1H), 8.52-8.39 (m, 2H), 8.29-8.20 (m, 1H), 7.91 (s, 1H), 7.90-7.84 (m, 1H), 7.83-7.77 (m, 1H), 5.93 (d, J=11.0 Hz, 1H), 5.70-5.60 (m, 1H), 5.37-5.30 (m, 1H), 4.90-4.80 (m, 1H), 4.75-4.69 (m, 1H), 4.58-4.51 (m, 2H), 4.50-4.43 (m, 2H), 4.42-4.32 (m, 2H), 3.58-3.54 (m, 2H), 3.31 (s, 3H), 3.29-3.24 (m, 2H), 3.18-3.12 (m, 1H), 3.07-3.00 (m, 1H), 2.69-2.60 (m, 2H), 2.46-2.29 (m, 4H), 2.25-2.12 (m, 3H), 2.11-2.02 (m, 2H), 1.99-1.92 (m, 1H), 1.66-1.57 (m, 1H), 1.42 (d, J=6.0 Hz, 3H), 1.30-1.22 (m, 2H), 1.20-1.14 (m, 2H), 1.12-1.04 (m, 6H), 0.98-0.88 (m, 6H), 0.31 (s, 3H).

### Example 39

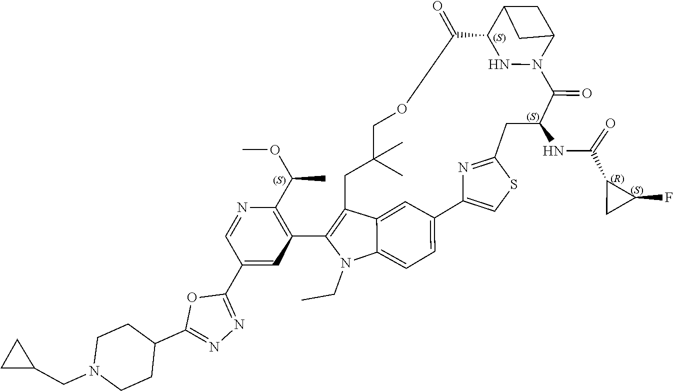

Compound 39 can be obtained by replacing INT-33 in the synthesis steps of compound 8 with INT-38 and replacing INT-4a with (1R,2S)-2-fluorocyclopropanecarboxylic acid using similar methods and synthesis steps. ESI-MS (m/z): 988.8 [M+H]+; LC-MS retention time RT=1.85 min.

¹H NMR (500 MHz, DMSO-d₆) δ 9.34 (d, J=2.0 Hz, 1H), 8.89 (d, J=9.0 Hz, 1H), 8.46 (d, J=1.5 Hz, 1H), 8.27 (d, J=2.0 Hz, 1H), 8.24 (s, 1H), 7.91 (s, 1H), 7.89-7.84 (m, 1H), 7.81 (d, J=9.0 Hz, 1H), 6.00 (d, J=11.0 Hz, 1H), 5.71-5.63 (m, 1H), 4.90-4.81 (m, 1H), 4.72 (d, J=11.0 Hz, 1H), 4.51-4.48 (m, 1H), 4.36-4.32 (m, 1H), 3.55 (s, 3H), 3.15-3.08 (m, 2H), 3.08-2.94 (m, 6H), 2.66-2.63 (m, 1H), 2.38-2.27 (m, 4H), 2.21 (d, J=6.0 Hz, 3H), 2.09-2.03 (m, 3H), 2.03-1.97 (m, 1H), 1.90-1.78 (m, 3H), 1.67-1.62 (m, 1H), 1.43-1.41 (m, 3H), 1.23 (s, 3H), 1.17-1.12 (m, 1H), 0.91 (s, 3H), 0.87-0.80 (m, 2H), 0.48-0.45 (m, 2H), 0.30 (s, 3H), 0.09-0.06 (m, 2H).

### Example 40

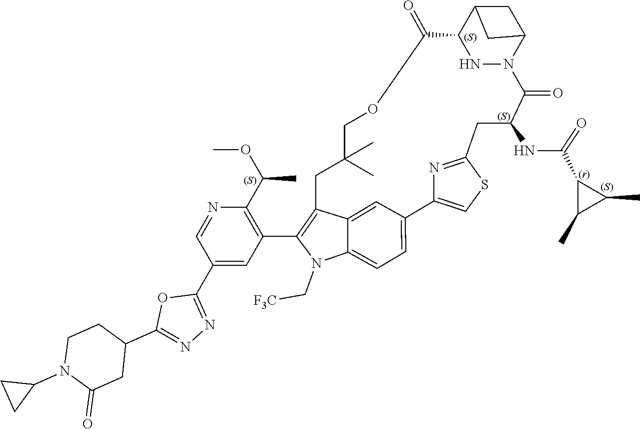

Example 40 was prepared by the following steps:

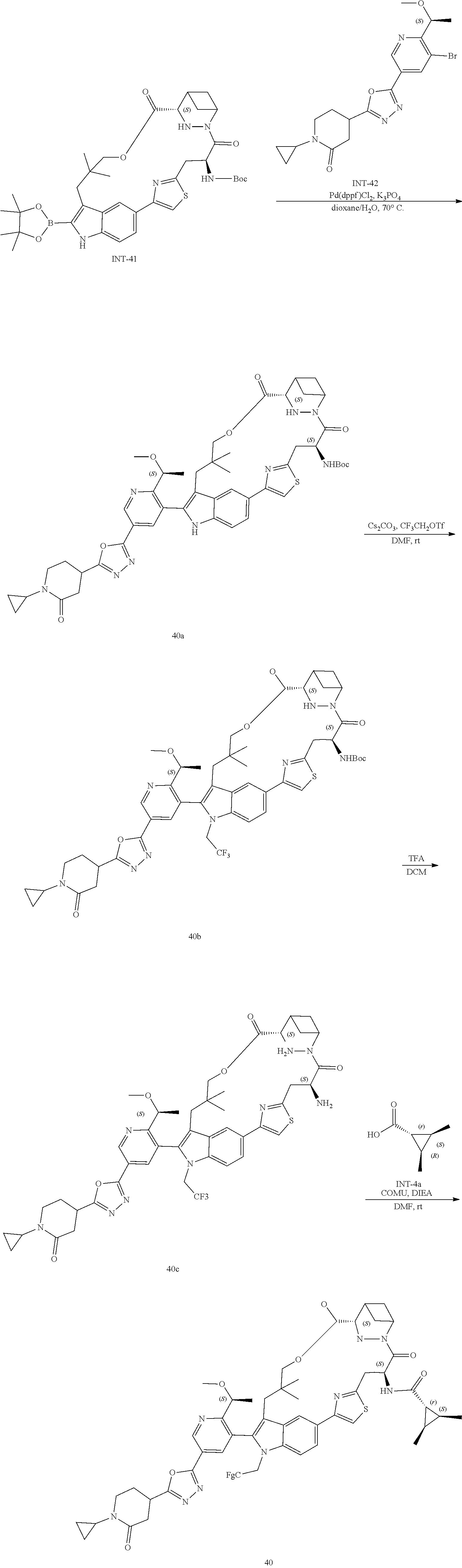

Step 1: Dissolve compound INT-41 (100 mg, 0.14 mmol) in a mixed solution of 1,4-dioxane (4 mL) and water (0.4 mL), and add INT-42 (60 mg, 0.14 mmol), [1,1′-bis(diphenylphosphino)ferrocene]palladium dichloride (10 mg, 0.014 mmol) and potassium phosphate (90 mg, 0.43 mmol) in sequence. The reaction mixture was stirred at 70 °C under nitrogen protection for 16 hours. After the reaction was complete, the reaction solution was concentrated, and the residue was purified by preparative thin-layer chromatography (dichloromethane/methanol=10:1) to obtain compound 40a (80 mg, yield 61.4%) as a light yellow solid. ESI-MS (m/z): 920.2 [M+H]⁺ ∘

Step 2: Compound 40a (80 mg, 0.087 mmol) was dissolved in N,N-dimethylformamide (3 mL), and cesium carbonate (85 mg, 0.261 mmol) and 2,2,2-trifluoroethyl trifluoromethanesulfonate (61 mg, 0.261 mmol) were added thereto. The reaction mixture was stirred at room temperature for 16 hours. After the reaction was complete, water (30 mL) was added to the reaction system, and the mixture was extracted with ethyl acetate (30 mL*2). The organic phases were combined, washed with water and saturated brine, dried over anhydrous sodium sulfate, filtered, and concentrated to obtain crude compound 40b. ESI-MS (m/z): 1002.7 [M+H]⁺ ∘

Step 3: The crude compound 40b was dissolved in dichloromethane (3 mL), and trifluoroacetic acid (1 mL) was added. The mixture was reacted at 0 °C for 3 hours. LCMS monitoring showed that the reaction of the raw material was complete. The mixture was quenched with saturated NaHCO₃ aqueous solution and extracted with dichloromethane (30 mL*3). The organic phases were combined, washed with water and saturated brine, dried over anhydrous sodium sulfate, filtered, and concentrated to obtain the crude compound 40c. ESI-MS (m/z): 902.9 [M+H]⁺ ∘

Step 4: The crude compound 40c and compound INT-4a (14.9 mg, 0.130 mmol) were dissolved in N,N-dimethylformamide (3 mL). N,N-diisopropylethylamine (45 mg, 0.348 mmol) and (1-Cyano-2-ethoxy-2-oxoethylidenaminooxy)dimethylaminomorpholinocarbenium Hexafluorophosphate (48 mg, 0.113 mmol) were added at room temperature. The mixture was reacted at room temperature for 1 hour. The reaction of the raw materials was completed by LCMS monitoring. Water was added to the system, and the mixture was extracted with ethyl acetate (30 mL*3). The organic phases were combined, washed with water and saturated brine, dried over anhydrous sodium sulfate, filtered, and concentrated. The residue was purified by preparative liquid chromatography to obtain compound 40 (15 mg, three-step yield 17.3%) as a white solid. ESI-MS (m/z): 998.4 [M+H]⁺ ∘ LC-MS retention time RT=1.75 min.

¹H NMR (500 MHz, DMSO-d₆) δ 9.34 (s, 1H), 8.54-8.42 (m, 3H), 7.97-7.83 (m, 2H), 7.79-7.68 (m, 1H), 6.01-5.86 (m, 1H), 5.38-5.28 (m, 1H), 5.04-4.89 (m, 2H), 4.80-4.72 (m, 1H), 4.56-4.46 (m, 1H), 4.09-3.96 (m, 1H), 3.68-3.58 (m, 4H), 3.54-3.49 (m, 1H), 3.29-3.24 (m, 2H), 3.19-3.09 (m, 5H), 2.77-2.63 (m, 5H), 2.37-2.26 (m, 2H), 2.20-2.13 (m, 1H), 2.06-1.97 (m, 1H), 1.80-1.74 (m, 1H), 1.68-1.60 (m, 1H), 1.35-1.19 (m, 8H), 1.14-1.06 (m, 6H), 0.74-0.65 (m, 2H), 0.63-0.56 (m, 2H), 0.46 (s, 3H).

### Example 41

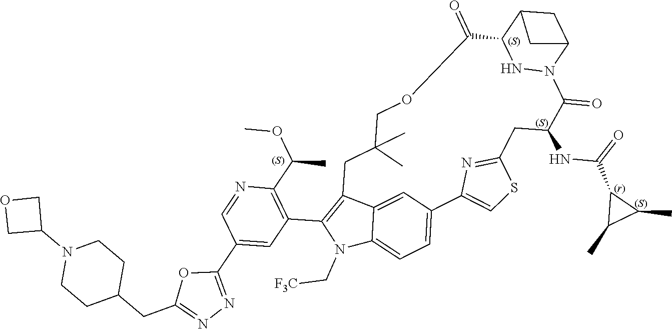

By replacing INT-3 in the synthesis steps of compound 1 with INT-40 and replacing INT-2 with INT-7, compound 41 can be obtained using similar methods and synthesis steps. ESI-MS (m/z): 1014.8 [M+H]+; LC-MS retention time RT=1.72 min.

¹H NMR (500 MHz, DMSO-d₆) δ 9.32 (d, J=2.0 Hz, 1H), 8.46 (d, J=10.0 Hz, 2H), 8.41 (d, J=2.) Hz, 1H), 7.91 (s, 1H), 7.86 (d, J=8.5 Hz, 1H), 7.72 (d, J=8.5 Hz, 1H), 5.97-5.89 (m, 1H), 5.31 (t, J=8.0 Hz, 1H), 5.00-4.91 (m, 2H), 4.78-4.71 (m, 1H), 4.53-4.47 (m, 3H), 4.39 (t, J=6.) Hz, 2H), 4.00-3.94 (m, 1H), 3.65-3.59 (m, 1H), 3.54-3.48 (m, 1H), 3.31-3.23 (m, 1H), 3.19-3.04 (m, 5H), 2.93 (d, J=7.0 Hz, 2H), 2.70-2.64 (m, 3H), 2.48-2.43 (m, 1H), 2.38-2.30 (m, 1H), 2.20-2.12 (m, 1H), 1.89-1.79 (m, 1H), 1.76-1.69 (m, 4H), 1.67-1.59 (m, 1H), 1.36-1.17 (m, 10H), 1.13-1.05 (m, 6H), 0.92 (s, 3H), 0.47 (s, 3H).

### Example 42

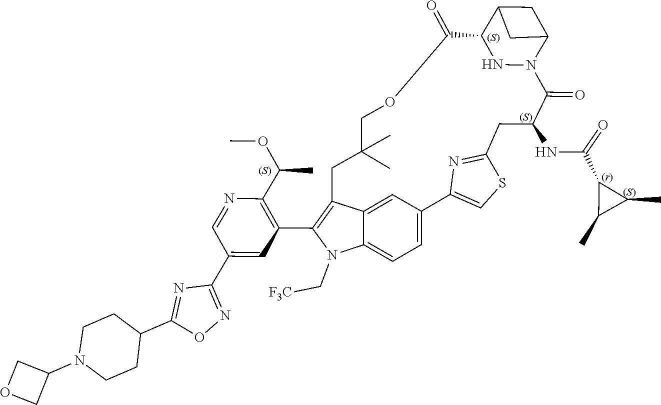

By replacing INT-3 in the synthesis steps of compound 1 with INT-40 and replacing INT-2 with INT-9, compound 42 can be obtained using similar methods and synthesis steps. ESI-MS (m/z): 1000.8 [M+H]+; LC-MS retention time RT=1.85 min.

1H NMR (500 MHz, DMSO-d₆) δ 9.35 (d, J=2.0 Hz, 1H), 8.49-8.43 (m, 2H), 8.24-8.21 (m, 1H), 7.91 (s, 1H), 7.90-7.86 (m, 1H), 7.81 (d, J=8.5 Hz, 1H), 5.98-5.88 (m, 1H), 5.71-5.61 (m, 1H), 5.37-5.31 (m, 1H), 4.91-4.83 (m, 1H), 4.74-4.69 (m, 1H), 4.54 (t, J=6.5 Hz, 2H), 4.51-4.48 (m, 1H), 4.44 (t, J=6.0 Hz, 2H), 4.38-4.32 (m, 1H), 3.57 (s, 2H), 3.44-3.40 (m, 2H), 3.32 (s, 3H), 3.29-3.25 (m, 1H), 3.18-3.13 (m, 2H), 3.08-3.02 (m, 1H), 2.76-2.70 (m, 2H), 2.67-2.62 (m, 1H), 2.49-2.41 (m, 2H), 2.36-2.31 (m, 1H), 2.17-2.08 (m, 3H), 1.99-1.93 (m, 2H), 1.88-1.79 (m, 2H), 1.65-1.59 (m, 1H), 1.43 (d, J=6.0 Hz, 3H), 1.29-1.22 (m, 3H), 1.20-1.17 (m, 2H), 1.12-1.06 (m, 6H), 0.90 (s, 3H).

### Example 43

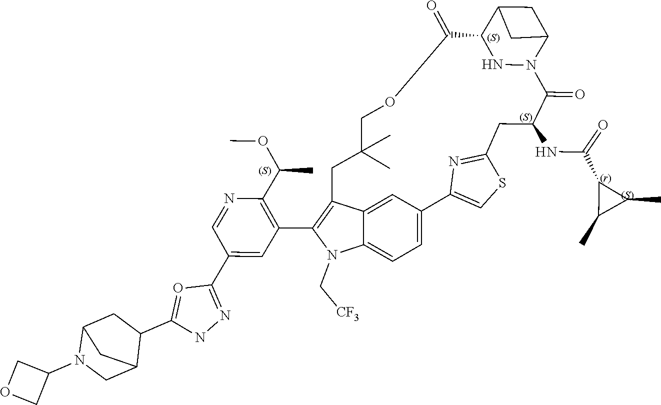

By replacing INT-3 in the synthesis steps of compound 1 with INT-40 and replacing INT-2 with INT-43, compound 43 can be obtained using similar methods and synthesis steps. ESI-MS (m/z): 1012.8 [M+H]+; LC-MS retention time RT=1.73 min.

¹H NMR (500 MHz, DMSO-d₆) δ 9.35-9.30 (m, 1H), 8.50-8.41 (m, 3H), 8.26-7.80 (m, 3H), 7.72 (d, J=8.6 Hz, 1H), 5.97-5.88 (m, 1H), 5.38-5.27 (m, 1H), 5.04-4.82 (m, 2H), 4.78-4.69 (m, 1H), 4.61-4.52 (m, 2H), 4.51-4.47 (m, 1H), 4.42 (t, J=5.8 Hz, 1H), 4.39-4.34 (m, 1H), 4.04-3.95 (m, 1H), 3.87-3.80 (m, 1H), 3.63-3.55 (m, 1H), 3.52-3.48 (m, 1H), 3.31-3.22 (m, 4H), 3.19-3.06 (m, 4H), 2.80-2.74 (m, 1H), 2.68-2.63 (m, 2H), 2.60-2.55 (m, 1H), 2.37-2.30 (m, 1H), 2.19-2.12 (m, 1H), 2.07-1.95 (m, 2H), 1.67-1.56 (m, 2H), 1.56-1.50 (m, 1H), 1.45-1.39 (m, 1H), 1.32-1.17 (m, 7H), 1.12-1.05 (m, 6H), 0.94-0.88 (m, 3H), 0.49-0.28 (m, 3H).

### Example 44

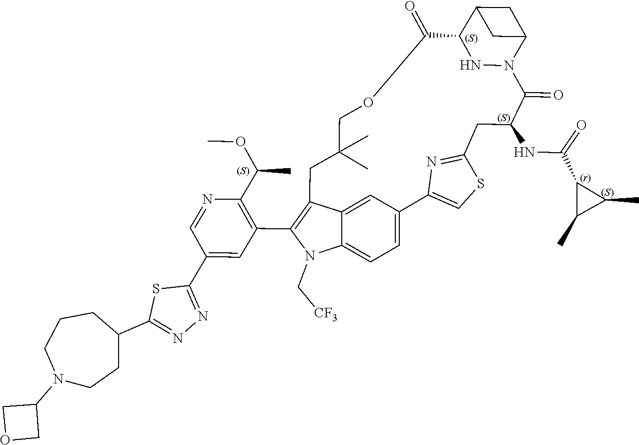

By replacing INT-3 in the synthesis steps of compound 1 with INT-40 and replacing INT-2 with INT-44, compound 44 can be obtained using similar methods and synthesis steps. ESI-MS (m/z): 1030.5 [M+H]+; LC-MS retention time RT=1.84 min.

¹H NMR (500 MHz, DMSO-d₆) δ 9.33-9.28 (m, 1H), 8.49-8.40 (m, 3H), 7.91 (s, 1H), 7.87-7.83 (m, 1H), 7.72 (d, J=8.6 Hz, 1H), 5.95-5.89 (m, 1H), 5.31 (t, J=8.0 Hz, 1H), 4.98-4.91 (m, 2H), 4.74 (d, J=11.0 Hz, 1H), 4.54 (t, J=6.5 Hz, 2H), 4.49 (d, J=5.0 Hz, 1H), 4.40 (t, J=6.0 Hz, 2H), 3.99-3.94 (m, 1H), 3.71-3.65 (m, 1H), 3.63-3.56 (m, 2H), 3.53-3.48 (m, 1H), 3.28-3.25 (m, 1H), 3.18-3.05 (m, 6H), 2.68-2.63 (m, 1H), 2.61-2.55 (m, 1H), 2.48-2.46 (m, 2H), 2.36-2.31 (m, 1H), 2.22-2.12 (m, 3H), 2.01-1.92 (m, 2H), 1.87-1.80 (m, 1H), 1.76-1.68 (m, 1H), 1.66-1.59 (m, 1H), 1.30-1.19 (m, 8H), 1.12-1.06 (m, 6H), 0.91 (s, 3H), 0.47 (s, 3H).

### Example 45

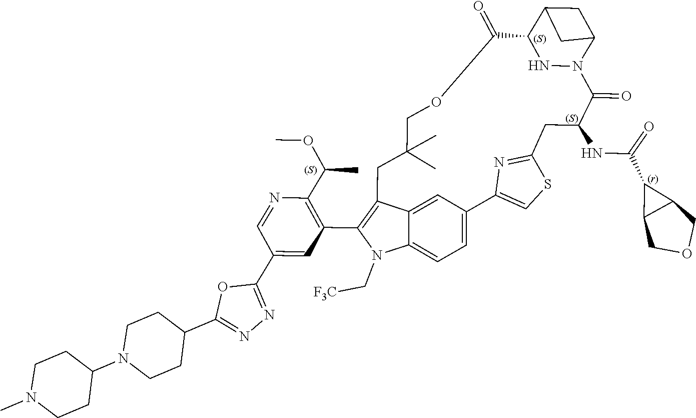

Example 45 was prepared by the following steps:

[chemical structure]

Step 1: Dissolve compound INT-32d (1 g, 1.01 mmol) in dichloromethane (4.5 mL). Add trifluoroacetic acid (1.5 mL) and react at 0 °C for 3 hours. LCMS monitoring indicated that the reaction was complete. Quench with saturated aqueous NaHCO₃ solution and extract with dichloromethane (60 mL*3). The organic phases were combined, washed with water and saturated brine, dried over anhydrous sodium sulfate, filtered, and concentrated to obtain compound 45a (838 mg, 93.2% yield). ESI-MS (m/z): 888.6 [M+H]⁺ ∘

Step 2: Compound 45a (1 g, 1.12 mmol) was dissolved in tetrahydrofuran (5 mL), and N,N-diisopropylethylamine (436 mg, 3.37 mmol) was added thereto. Benzyl chloroformate (288 mg, 1.69 mmol) was then added dropwise to the reaction solution. The reaction solution was stirred at room temperature for 16 hours, and then benzyl chloroformate (288 mg, 1.69 mmol) was added. The reaction solution was stirred at room temperature for another 6 hours. Water was added to the reaction system, and the mixture was extracted with dichloromethane (60 mL*3). The organic phases were combined, washed with saturated brine, dried over anhydrous sodium sulfate, filtered, and concentrated to obtain a pale yellow solid crude compound 45b. ESI-MS (m/z): 1022.6 [M+H]⁺;

Step 3: The crude compound 45b was dissolved in tetrahydrofuran (6 mL), and tetrabutylammonium fluoride (1 M, 3.37 mL) was added thereto. The reaction mixture was stirred at 60 °C for 16 hours. After the reaction was complete, water (30 mL) was added to the reaction system, and the mixture was extracted with ethyl acetate (30 mL*3). The organic phases were combined, washed with saturated brine, dried over anhydrous sodium sulfate, filtered, and concentrated. The residue was purified by silica gel column chromatography (petroleum ether/ethyl acetate=1/3) to obtain a light yellow solid compound 45c (551 mg, yield 62.5%). ESI-MS (m/z): 784.2 [M+H]⁺ ∘

Step 4: Dissolve compound 45c (551 mg, 0.70 mmol) in 1,4-dioxane (10 mL), and add potassium acetate (207 mg, 2.11 mmol), bis(pinacolato)diboron (267 mg, 1.05 mmol) and [1,1′-bis(di-tert-butylphosphino)ferrocene]palladium dichloride (51 mg, 0.070 mmol) in sequence. The mixture was stirred at 80 °C under nitrogen atmosphere for 16 hours. The reaction was complete when monitored by LCMS. Water was added to the system, and the mixture was extracted with ethyl acetate (30 mL*3). The organic phases were combined, washed with saturated brine, dried over anhydrous sodium sulfate, filtered, and concentrated. The residue was purified by silica gel column chromatography (petroleum ether/ethyl acetate=1/3) to give a light yellow solid compound 45d (467 mg, yield 78.0%). ESI-MS (m/z): 832.8 [M+H]⁺ ∘

Step 5: Compound 45d (467 mg, 0.56 mmol) was dissolved in 1,4-dioxane (6 mL) and water (0.6 mL), and INT-31 (261 mg, 0.53 mmol), potassium carbonate (233 mg, 1.68 mmol), and [1,1′-bis(di-tert-butylphosphino)ferrocene]palladium dichloride (36 mg, 0.056 mmol) were added in sequence. The reaction solution was stirred at 50 °C under nitrogen atmosphere for 20 hours. The reaction was completed by LCMS monitoring. Water was added to the system, extracted with ethyl acetate (30 mL*3), the organic phases were combined, washed with saturated brine, dried over anhydrous sodium sulfate, and concentrated. The resulting residue was purified by silica gel column chromatography (petroleum ether/ethyl acetate=1/9) to give compound 45e (529 mg, yield 84.6%) as a pale yellow solid. ESI-MS (m/z): 1114.9 [M+H]⁺ ∘

Step 6: Compound 45e (519 mg, 0.466 mmol) was dissolved in tetrahydrofuran (5 mL) and water (2.5 mL). Lithium hydroxide monohydrate (58 mg, 1.4 mmol) was added at 0 °C and stirred for 1 hour. The reaction was complete as monitored by LCMS. The mixture was diluted with water, adjusted to pH 5 with dilute hydrochloric acid, and extracted with ethyl acetate (30 mL*3). The organic phase was washed with saturated brine, dried over anhydrous sodium sulfate, and concentrated to obtain a pale yellow solid crude compound 45f. ESI-MS (m/z): 1100.8 [M+H]⁺ ∘

Step 7: Add N,N,N′,N′-tetramethylchloroformamidinium hexafluorophosphate (261 mg, 0.93 mmol) and 1-methylimidazole (191 mg, 2.33 mmol) to acetonitrile (10 mL) and stir until dissolved. Add a solution of the crude compound 45f in THF (5 mL) dropwise at room temperature. After the addition is complete, stir and react for 1 hour. LCMS monitoring shows that the reaction of the raw material is complete. Water was added to the system, extracted with ethyl acetate (30 mL*3), the organic phases were combined, washed with saturated brine, dried over anhydrous sodium sulfate, and concentrated. The resulting residue was purified by silica gel column chromatography (petroleum ether/ethyl acetate=1/9) to obtain 45 g (348 mg, two-step yield 69.1%) of a light yellow solid compound. ESI-MS (m/z): 1082.8 [M+H]⁺ ∘

Step 8: Compound 45 g (70 mg, 0.065 mmol), 10% palladium hydroxide (14 mg), 10% palladium on carbon (14 mg) and isopropanol (2 mL) were added to the reaction flask. Under a hydrogen atmosphere, the reaction mixture was stirred at 70 °C for 20 hours. The reaction solution was filtered through celite, and the filtrate was concentrated to obtain crude compound 45h. ESI-MS (m/z): 948.8 [M+H]⁺ ∘

Step 9: The crude compound 45h was dissolved in 1,2-dichloroethane (3 mL), and N-methyl-4-piperidone (22 mg, 0.194 mmol) was added at room temperature. The reaction solution was stirred at room temperature for 10 minutes. Then, sodium acetate borohydride (69 mg, 0.323 mmol) was slowly added to the reaction solution, and the reaction solution was stirred at room temperature for 16 hours. The reaction was complete as monitored by LCMS. Saturated aqueous sodium bicarbonate solution was added to the reaction system to quench the reaction, and the mixture was extracted with ethyl acetate (30 mL*3). The organic phases were combined and washed with saturated brine (20 mL), dried over anhydrous sodium sulfate, and concentrated. The organic phase was purified by silica gel column chromatography (dichloromethane/methanol=8:1) to obtain compound 45i (44 mg, two-step yield 65.1%) as a light yellow solid. ESI-MS (m/z): 1045.8 [M+H]⁺ ∘

Step 10: Compound 45i (44 mg, 0.042 mmol) was dissolved in dichloromethane (3 mL), and trifluoroacetic acid (1 mL) was added under ice-cooling. The reaction mixture was stirred at 0 °C for 3 hours. After the reaction was complete, saturated sodium bicarbonate solution was added to the reaction system under ice bath to adjust the pH to 8. The mixture was extracted with dichloromethane (30 mL×3). The organic phases were combined, washed with saturated brine, dried over anhydrous sodium sulfate, filtered, and concentrated to obtain a light yellow solid compound 45j (38 mg, yield 95.5%). ESI-MS (m/z): 945.8 [M+H]⁺ ∘

Step 11: Compound 45j (38 mg, 0.040 mmol) and compound 45k (7.7 mg, 0.06 mmol) were dissolved in N,N-dimethylformamide (3 mL). N,N-diisopropylethylamine (21 mg, 0.16 mmol) and (1-Cyano-2-ethoxy-2-oxoethylidenaminooxy)dimethylaminomorpholinocarbenium Hexafluorophosphate (22 mg, 0.052 mmol) were added at room temperature. The mixture was reacted at room temperature for 1 hour. The reaction was completed after monitoring by LCMS. Water was added to the system, and the mixture was extracted with ethyl acetate (30 mL*3). The organic phases were combined, washed with water and saturated brine, dried over anhydrous sodium sulfate, filtered, and concentrated. The residue was purified by preparative liquid chromatography to obtain 45 as a white solid (8 mg, yield 18.9%). ESI-MS (m/z): 1055.7 [M+H]⁺ ∘ LC-MS retention time RT=1.50 min.

¹H NMR (500 MHz, DMSO-d₆) δ 9.36-9.31 (m, 1H), 8.67-8.59 (m, 1H), 8.46 (s, 1H), 8.34-8.23 (m, 3H), 7.91 (s, 1H), 7.90-7.85 (m, 1H), 7.83-7.78 (m, 1H), 6.00-5.91 (m, 1H), 5.70-5.59 (m, 1H), 5.41-5.30 (m, 1H), 4.91-4.81 (m, 1H), 4.76-4.69 (m, 1H), 4.52-4.45 (m, 1H), 4.37-4.30 (m, 1H), 3.90-3.85 (m, 2H), 3.67-3.65 (m, 2H), 3.33-3.28 (m, 4H), 3.16-3.11 (m, 1H), 3.07-3.00 (m, 2H), 2.94-2.89 (m, 2H), 2.84-2.80 (m, 2H), 2.66-2.63 (m, 1H), 2.37-2.28 (m, 4H), 2.18-2.14 (m, 4H), 2.08-2.01 (m, 3H), 1.94-1.89 (m, 3H), 1.82-1.75 (m, 2H), 1.70-1.61 (m, 4H), 1.50-1.40 (m, 5H), 0.90 (s, 3H), 0.31 (s, 3H).

### Example 46

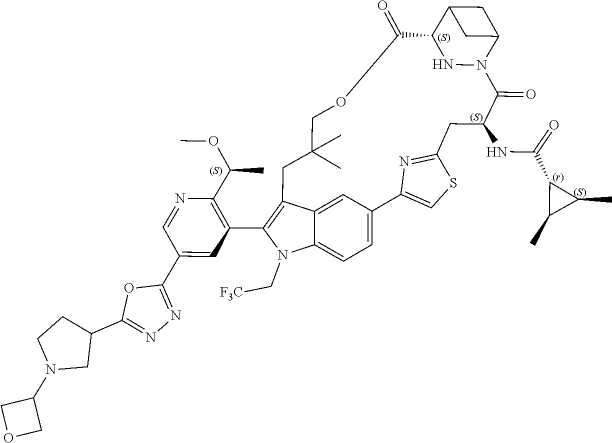

By replacing INT-3 in the synthesis steps of compound 1 with INT-40 and replacing INT-2 with INT-45, compound 46 can be obtained using similar methods and synthesis steps. ESI-MS (m/z): 986.8 [M+H]+; LC-MS retention time RT=1.72 min.

¹H NMR (500 MHz, DMSO-d₆) δ 9.36-9.32 (m, 1H), 8.52-8.42 (m, 3H), 7.93-7.90 (m, 1H), 7.90-7.71 (m, 2H), 5.96-5.90 (m, 1H), 5.37-5.29 (m, 1H), 5.03-4.83 (m, 2H), 4.77-4.71 (m, 1H), 4.62-4.56 (m, 2H), 4.52-4.44 (m, 3H), 4.38-3.98 (m, 1H), 3.81-3.73 (m, 1H), 3.69-3.61 (m, 2H), 3.57-3.49 (m, 1H), 3.33-3.31 (m, 1H), 3.30-3.25 (m, 1H), 3.19-3.04 (m, 4H), 2.99-2.93 (m, 1H), 2.89-2.82 (m, 1H), 2.68-2.59 (m, 3H), 2.49-2.45 (m, 1H), 2.37-2.21 (m, 3H), 2.19-2.13 (m, 1H), 1.67-1.61 (m, 1H), 1.46-1.41 (m, 1H), 1.33-1.22 (m, 4H), 1.21-1.16 (m, 2H), 1.12-1.07 (m, 6H), 0.94-0.90 (m, 3H), 0.49-0.29 (m, 3H).

### Example 47

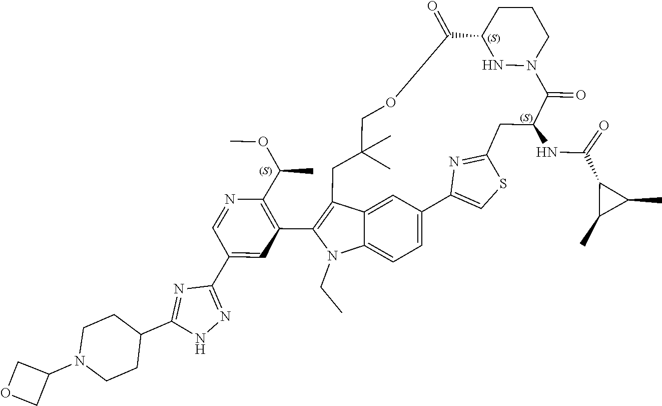

By replacing INT-3 in the synthesis steps of compound 1 with INT-4 and replacing INT-2 with INT-46, compound 47 can be obtained using similar methods and synthesis steps. ESI-MS (m/z): 933.3 [M+H]⁺; LC-MS retention time RT=1.75 min.

### Biological Screening of RAS Inhibitors and Results

### Test Example 1: In Vitro Cell Proliferation Inhibition Assay

Due to the diversity of RAS mutations, and in order to evaluate the activity of the compounds in different RAS mutant cell lines, we selected cell lines with KRAS^{G12D}, KRAS^{G12}V, KRAS^{G12c}, NRAS^{q61L}, KRAS^{G13D}, KRAS^{G12R}, and KRAS^{WT} mutations (see the table below for in vitro activity evaluation and screening of the compounds.

| Cell Line | Histotype | Mutant |
|---|---|---|
| AsPC-1 | Pancreatic | KRAS (p.G12D) |
| | Adenocarcinoma | |
| SW480 | Colon Adenocarcinoma | KRAS (p.G12V) |
| H358 | Lung Adenocarcinoma | KRAS (p.G12C) |
| HepG2 | Hepatoblastoma | NRAS (p.Q61L) |
| HCT116 | Colon Adenocarcinoma | KRAS (p.G13D) |
| PSN1 | Pancreatic | KRAS (p.G12R) |
| | Adenocarcinoma | |
| MV411 | Acute Myeloid Leukemia | KRAS wild type |

### Experimental Protocol: CellTiter-Glo® Cell Luminescent Viability Assay (Promega)

According to the doubling time of different cell lines, different numbers of cells (1000-5000 cells/well) were seeded into 96-well plates containing 180 μL of the corresponding culture medium, and incubated overnight in a cell incubator at 37 °C with 5% C₀₂. On the following day, the test compounds were pre-diluted in the culture medium in a 3-fold serial dilution series, with the highest concentration being 100 PM, resulting in 10 concentration gradients in total. Subsequently, 20 μL of culture medium containing the compounds at different concentrations was added to the cells in the 96-well plates, ensuring a final compound concentration of up to 10 μM, with 10 concentrations in 3-fold dilution. After co-incubation of the cells with the compounds for 72 h, the 96-well plates were removed from the incubator and equilibrated at room temperature for 30 min. Then, 25 μL of CellTiter-Glo® Reagent was added to each well and mixed thoroughly, followed by incubation at room temperature for 10 min. Subsequently, 100 μL of the samples was transferred to white 96-well plates (OptiPlate™-96, PerkinElmer), and fluorescence signal values were read using a multifunctional microplate reader (SpectraMax i3x, Molecular Devices). The signal values were then normalized, and curve fitting was performed using a four-parameter logistic regression equation to calculate the half maximal inhibitory concentration (IC₅₀) of the compounds against the cell lines.

TABLE 1
Antiproliferative activity of the compounds of
the disclosure against RAS mutant cell lines

| | IC₅₀ (nM) | | | | | | |
|---|---|---|---|---|---|---|---|
| Example | AsPC-1 | SW480 | H358 | HepG2 | HCT116 | PSN1 | MV411 |
| 1 | 0.05 | 0.07 | 0.06 | 5.0 | ~10 | 5.1 | 1.7 |
| 2 | 0.06 | 0.09 | 0.002 | 0.01 | 0.07 | 0.01 | 0.16 |
| 4 | 0.21 | 0.52 | 0.001 | 0.06 | 2.8 | 0.07 | 0.35 |
| 5 | 0.08 | 0.11 | 0.034 | 0.66 | 0.02 | 0.03 | 0.66 |
| 6 | 0.02 | 0.03 | 0.007 | 0.04 | 0.44 | 0.04 | 0.12 |
| 7 | 0.03 | 0.14 | 0.004 | 0.05 | 0.70 | 0.05 | 0.49 |
| 8 | 0.01 | 0.01 | 0.01 | 0.02 | 0.03 | 0.01 | 0.07 |
| 9 | 0.09 | 0.06 | 0.041 | 0.16 | 0.23 | 0.01 | 0.76 |
| 10 | 0.005 | 0.01 | 0.021 | 0.10 | 0.14 | 0.07 | 0.42 |
| 18 | 0.03 | 0.13 | 0.39 | 1.6 | 23 | 5.3 | 13 |
| 26 | 0.25 | 0.54 | 0.01 | 0.25 | 3.2 | 0.08 | 0.64 |
| 27 | 0.37 | 0.87 | 0.17 | 0.05 | 11 | 0.16 | 1.1 |
| 45 | 0.06 | 0.37 | 0.01 | 0.022 | 1.1 | 0.04 | 0.42 |
| 43 | 0.12 | 0.13 | 0.05 | 0.04 | 1.1 | 0.05 | 0.97 |
| 46 | 0.05 | 0.06 | 0.01 | 0.05 | 0.58 | 0.09 | 1.0 |
| 38 | 0.01 | 0.04 | 0.04 | 0.11 | 0.02 | 0.01 | 0.45 |
| 42 | 0.01 | 0.02 | 0.03 | 0.01 | 0.03 | 0.01 | 0.56 |
| RMC-6236¹ | 1.2 | 1.5 | 0.08 | 2.8 | 2.6 | 2.0 | 4.2 |
| Compound 6A of | 0.40 | 0.25 | 0.01 | 0.58 | 0.95 | 0.24 | 3.0 |
| WO2024067857¹ | | | | | | | |

¹The reference compound was synthesized in-house and characterized for its structure and biological activity data.
Conclusion: The compounds of the disclosure exhibited significant inhibitory activity against various RAS mutant cell lines.

### Test Example 2: Binding Constant Assay for Complexes

### 2.1 Binary Complex Binding Assay

To evaluate the binding ability of the test compounds to cyclophilin A (CypA), we employed a Bio-layer Interferometry (BLI) method (Octet R₈) to monitor the binding kinetics between the compounds and CypA, and to calculate the affinity of the binary complex. His-tagged CypA protein (3589-CAB, R&D) was first immobilized on an NTA sensor, followed by baseline equilibration for 120 seconds in the assay buffer (20 mM HEPES, pH 7.4, 150 mM NaCl, 5 mM MgCl₂, 1 mM TCEP, 0.01% Tween 20). The sensor was then exposed to buffer solutions containing different concentrations of the compounds to monitor the association process in real time. When the high-concentration compound binding reached a plateau, the sensor was transferred to compound-free buffer to measure the dissociation process. To ensure data accuracy, DMSO normalization was performed, non-specific binding was minimized by buffer optimization, and negative and positive controls were set to verify assay specificity. Data were processed using Octet Analysis Studio 13.0, analyzed using a 1:1 binding model, and the association rate constant (ka), dissociation rate constant (kdis), and equilibrium dissociation constant (K_{D1}) were calculated.

TABLE 2
Determination of the binary complex binding constant (K_{D1})

| Example | K_{D1} (nM) | ka (1/[M*s]) | kdis (1/s) |
|---|---|---|---|
| 4 | 0.81 | 1.5E+05 | 1.2E−04 |
| 5 | 8.6 | 2.0E+05 | 1.7E−03 |
| 6 | 3.0 | 1.3E+05 | 4.0E−04 |
| 7 | 16 | 2.3E+05 | 3.7E−03 |
| 8 | 2.0 | 3.3E+05 | 6.6E−04 |
| 10 | 2.7 | 2.1E+05 | 5.7E−04 |
| RMC-6236¹ | 12 | 2.8E+05 | 3.4E−03 |
| Compound 6A of | 2.0 | 3.4E+05 | 6.8E−04 |
| WO2024067857¹ | | | |

¹The reference compound was synthesized in-house and characterized for its structure and biological activity data.
Conclusion: The compounds of the disclosure exhibited a strong ability to form binary complexes with Cyclophilin A (CypA) protein.

### 2.2 Ternary Complex Binding Assay

To evaluate the ability of the test compounds to induce the formation of a ternary complex comprising RAS and CypA, we employed a Bio-layer Interferometry (BLI) method (Octet R8) to monitor the binding kinetics among the compounds, RAS, and CypA, and to calculate the affinity of the ternary complex. GMPPNP was first incubated with RAS protein to load GMPPNP onto RAS. His-tagged RAS proteins with different mutations were then immobilized on NTA sensors, followed by baseline equilibration for 120 seconds in the assay buffer (20 mM HEPES, pH 7.4, 150 mM NaCl, 5 mM MgCl₂, 1 mM TCEP, 5 μM GMPPNP, 0.01% Tween 20). The sensors were subsequently exposed to buffer containing various concentrations of the compounds and a fixed concentration of CypA, and the association phase of ternary complex formation was monitored in real time. Once binding reached a plateau, the sensors were transferred to compound- and CypA-free buffer to measure the dissociation phase. To ensure accuracy, DMSO normalization was performed, buffer composition was optimized to reduce non-specific binding, and both negative and positive controls were employed to verify assay specificity. Data were processed using Octet Analysis Studio 13.0, analyzed using a 1:1 binding model, and the association rate constant (ka), dissociation rate constant (kdis), and equilibrium dissociation constant (K_{D2}) were calculated.

TABLE 3
Determination of the ternary complex binding constant (K_{D1})

Mutant Ras(on) protein

| | KRAS^{G12D} | KRAS^{G12V} | KRAS^{WT} |
|---|---|---|---|
| Example | K_{D2} (nM) | K_{D2} (nM) | K_{D2} (nM) |
| 4 | 43 | 25 | 27 |
| 5 | 27 | 27 | 17 |
| 6 | 24 | 26 | 16 |
| 7 | 19 | | |
| 8 | 8.8 | 8.3 | 10 |
| 10 | 17 | 12 | 9.3 |
| RMC-6236¹ | 25 | 68 | 53 |
| Compound 6A of | 41 | 56 | 40 |
| WO2024067857¹ | | | |

¹The reference compound was synthesized in-house and characterized for its structure and biological activity data.
Conclusion: The compounds of the disclosure exhibited a strong ability to form ternary complexes with Cyclophilin A (CypA) protein and RAS protein.

### Test Example 3: Pharmacokinetic Study in Mice

To evaluate the pharmacokinetic behavior of the compounds of the disclosure in male CD-1 mice, the test compounds were dissolved in a solvent (5% DMSO/10% solutol/85% water). Twelve male CD-1 mice were divided into two groups, with six animals in each group. Group 1 received a single intravenous bolus (i.v.) administration of the compound, and Group 2 received a single oral (p.o.) administration of the compound. Whole blood samples were collected at 0.083 h (intravenous group only), 0.25, 0.5, 1, 2, 4, 8, and 24 hours after dosing. The concentration of the test compound in whole blood samples was determined using the LC-MS/MS method.

All animals showed good tolerability during the study, and no abnormal clinical signs were observed. The detailed study results are shown in Table 4.

TABLE 4
Pharmacokinetic study results in mice

| | | i.v. | | p.o. | | |
|---|---|---|---|---|---|---|
| | | Cl | Vd | Cmax | AUC | F |
| Compound | dose | (mL/min/kg) | (mL/kg) | (ng/mL) | (ng*h/mL) | (%) |
| 2 | i.v. (1 mpk); | 4.6 | 1950 | 167 | 1787 | 24.5 |
| | p.o. (2 mpk) | | | | | |
| 6 | i.v. (1 mpk); | 2.6 | 1141 | 439 | 5628 | 43.1 |
| | p.o. (2 mpk) | | | | | |
| 8 | i.v. (0.25 mpk); | 1.8 | 1841 | 96 | 2591 | 54.6 |
| | p.o. (0.5 mpk) | | | | | |
| 9 | i.v. (1 mpk); | 3.7 | 2866 | 248 | 5743 | 63.3 |
| | p.o. (2 mpk) | | | | | |

Conclusion: The compounds of the disclosure exhibited slow clearance, high exposure, and favorable pharmacokinetic properties.

### EMBODIMENTS

Embodiment 1. A compound of formula (A), or an isotopic derivative, stereoisomer, or a pharmaceutically acceptable salt thereof:

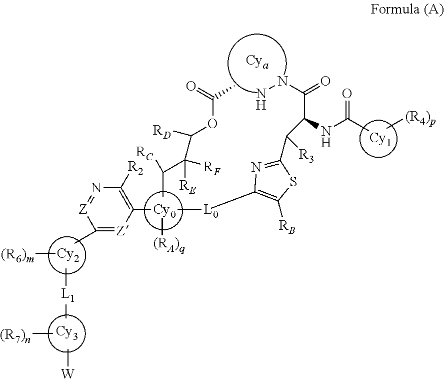

wherein:
Cya represents

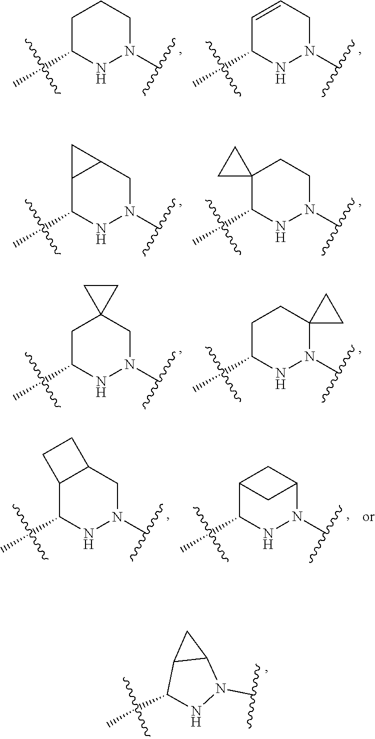

which may optionally be substituted with 0, 1, 2, or 3 substituents selected from halogen or C₁-C₃ alkyl;
R₂ represents halogen, cyano, C₁-C₆ alkyl, —(C₀-C₆ alkylene)-(C₃-C₈ cycloalkyl), or —(C₀-C₆ alkylene)-(4-8-membered heterocycloalkyl), which may optionally be substituted with 0, 1, or 2 substituents selected from: —ORa, —SRa, or —NRaRa′;
R₃ represents hydrogen, hydroxy, —(C₁-C₃ alkylene)-ORa, or C₁-C₃ alkyl;
Cy₁ represents C₃-C₁₀ cycloalkyl or 4-10-membered heterocycloalkyl, and Cy₁ may be mono ring, spiro ring, bridged ring, or fused ring;
R₄ each independently represents hydrogen, halogen, oxo, C₁-C₆ alkyl, —(C₀-C₆ alkylene)(C₃-C₆) cycloalkyl, —(C₀-C₆ alkylene)(4-8-membered) heterocycloalky, —(C₀-C₆ alkylene)ORa, —(C₀-C₆ alkylene)SRa, —(C₀-C₆ alkylene)NRaRa′, —(C₀-C₆ alkylene)CORa, —(C₀-C₆ alkylene)COORa, —(C₀-C₆ alkylene)CONRaRa′, —(C₀-C₆ alkylene) NRaCORa′, —(C₀-C₆ alkylene) OCONRaRa′, —(C₀-C₆ alkylene) NRaCONRaRa′, —(C₀-C₆ alkylene)SORa, —(C₀-C₆ alkylene)S(O)₂Ra, —(C₀-C₆ alkylene)NRaS(O)₂Ra′, —(C₀-C₆ alkylene)CN, —(C₀-C₆ alkylene)(C₆-C₁₀ aryl) or —(C₀-C₆ alkylene)(5-12 membered heteroaryl); wherein the R₄ substituents on two carbon atoms of Cy₁, together with the carbon atoms to which they are attached and the atoms between said two carbon atoms, may form a 3-8 membered ring, which may optionally contain 0, 1, 2, or 3 heteroatoms selected from N, O, or S; or the two R₄ substituents on the same carbon atom of Cy₁, together with the carbon atom to which they are attached, may form a 3-8 membered ring, which may optionally contain 0, 1, 2, or 3 heteroatoms selected from N, O, or S;
optionally, R₃ and R₄ may together form a ring;
Cy₂ represents 5-membered heteroaryl;
Cy₃ represents C₃-C₁₂ cycloalkyl or 4-12-membered heterocycloalkyl, which may be a mono ring, spiro ring, bridged ring, or fused ring;
R₆ and R₇ are each independently selected from: hydrogen, halogen, oxo, —ORa, —NRaRa′, cyano, C₁-C₆ alkyl, C₃-C₈ cycloalkyl, or 4-8-membered heterocycloalkyl; each of the above-mentioned C₁-C₆ alkyl, C₃-C₈ cycloalkyl, and 4-8-membered heterocycloalkyl may be independently substituted with 0, 1, 2, 3, or 4 substituents selected from halogen, oxo, —ORa, —SRa, —NRaRa′, cyano, C₁-C₆ alkyl, C₃-C₈ cycloalkyl, or 4-8-membered heterocycloalkyl;
W is hydrogen or represented by formula (II), wherein * represents the site where W is attached to Cy₃;

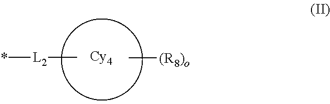

Cy₄ is selected from C₃-C₁₂ cycloalkyl or 4-12-membered heterocycloalkyl, which may be a mono ring, spiro ring, bridged ring, or fused ring;
R₈ is each independently selected from: hydrogen, halogen, oxo, —ORa, —NRaRa′, cyano, or C₁-C₆ alkyl, wherein the C₁-C₆ alkyl may be substituted with 0, 1, 2, 3, or 4 substituents selected from halogen, oxo, —ORa, —NRaRa′, or cyano;
L₁ and L₂ each independently represent single bond or —(C₁-C₆) alkylene-, wherein any methylene of the —(C₁-C₆) alkylene- may be replaced by a carbonyl, NRa, O, or S; the —(C₁-C₆) alkylene- may be substituted with 0, 1, 2, 3, or 4 substituents selected from halogen or C₁-C₃ alkyl; and two substituents on the same carbon atom may together with the carbon atom to which they are attached form a 3-6-membered ring, which optionally contains 0, 1, 2, or 3 heteroatoms selected from N, O, or S;
Cy₀ represents 5-12-membered aryl or heteroaryl;
R_{A} is each independently selected from H, halogen, CN, C₁-C₆ alkyl, —(C₀-C₆ alkylene)-(C₃-C₈ cycloalkyl), —(C₀-C₆ alkylene)-(4-8-membered heterocycloalkyl), —(C₀-C₆ alkylene)-ORa, —(C₀-C₆ alkylene)-SRa, or —(C₀-C₆ alkylene)-NRaRa′; and, optionally, two R_{A} groups on adjacent or non-adjacent atoms of Cy₀ together with the ring atoms of Cy₀ may form a 6-10-membered ring, which may be further substituted with 0, 1, 2, 3, or 4 substituents selected from halogen, C₁-C₃ alkyl, —ORa, —SRa, or —NRaRa′;
L₀ is selected from single bond, —(C₁-C₆) alkylene-, or —(C₂-C₆) alkenylene-, wherein any methylene in the —(C₁-C₆) alkylene- or —(C₂-C₆) alkenylene- may be replaced by a carbonyl, NRa, O, or S; and the —(C₁-C₆) alkylene- or —(C₂-C₆) alkenylene- is optionally substituted with 0, 1, 2, 3, or 4 substituents selected from halogen or C₁-C₃ alkyl;
R_{B} each is independently selected from H, halogen, C₁-C₆ alkyl, —(C₀-C₆ alkylene)-ORa, —(C₀-C₆ alkylene)-SRa, or —(C₀-C₆ alkylene)-NRaRa′; and optionally, R_{B} may form a ring with a R₄ substituent on Cy₁;
R_{C} is selected from H, halogen, C₁-C₆ alkyl, —(C₀-C₆ alkylene)-ORa, —(C₀-C₆ alkylene)-SRa, or —(C₀-C₆ alkylene)-NRaRa′;
R_{D} is selected from H, C₁-C₆ alkyl, —(C₁-C₆ alkylene)-ORa, —(C₁-C₆ alkylene)-SRa, or —(C₁-C₆ alkylene)-NRaRa′;
each of R_{E} and R_{F} is independently selected from H, C₁-C₆ alkyl, halogen, —(C₀-C₆ alkylene)-ORa, —(C₀-C₆ alkylene)-SRa, or —(C₀-C₆ alkylene)-NRaRa′; and optionally, R_{E} and R_{F}, together with the carbon atom to which they are attached, may form a 3-6-membered ring, which may additionally contain 0, 1, or 2 heteroatoms selected from N, O, or S;
Z represents N or CR₅; Z′ represents N or CR₅′; wherein R₅ and R₅′ are each independently selected from hydrogen, halogen, C₁-C₆ alkyl, —(C₀-C₆ alkylene)-(C₃-C₈ cycloalkyl), or —(C₀-C₆ alkylene)-CN;
wherein m, n, o, p, and q are each independently represents 0, 1, 2, 3, or 4;
Ra and Ra′ are each independently selected from hydrogen, C₁-C₆ alkyl, C₃-C₈ cycloalkyl, or a 4-8-membered heterocycloalkyl; when Ra and Ra′ are attached to the same nitrogen atom, Ra and Ra′ together with the nitrogen atom to which they are attached may form a 4-8-membered ring, which may optionally contain 0, 1, 2, or 3 heteroatoms selected from N, O, or S;
the alkyl, cycloalkyl, heterocycloalkyl, and alkylene groups may each independently be substituted by 0, 1, 2, 3, 4, 5, or 6 halogen atoms.

Embodiment 2. The compound according to embodiment 1, or an isotopic derivative, stereoisomer, or a pharmaceutically acceptable salt thereof, having a structure represented by formula (A′):

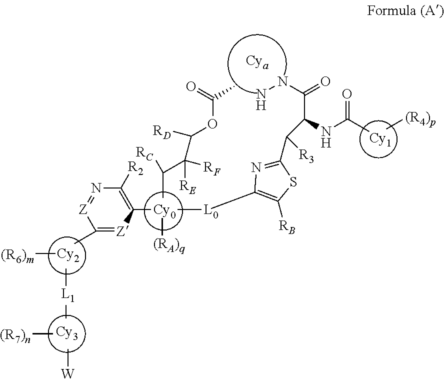

wherein R_{A}, R_{B}, R_{C}, R_{D}, R_{E}, R_{F}, Z, Z′, R₂, R₃, R₄, R₆, R₇, Cya, Cy₀, Cy₁, Cy₂, Cy₃, L₀, L₁, W, m, n, p, q are as defined in embodiment 1.

Embodiment 3. The compound according to any one of the preceding embodiments, or an isotopic derivative, stereoisomer, or a pharmaceutically acceptable salt thereof, wherein R_{B} is H.

Embodiment 4. The compound according to any one of the preceding embodiments, or an isotopic derivative, stereoisomer, or a pharmaceutically acceptable salt thereof, wherein R_{C} and R_{D} are H.

Embodiment 5. The compound according to any one of the preceding embodiments, or an isotopic derivative, stereoisomer, or a pharmaceutically acceptable salt thereof, wherein Z represents CR₅ and Z′ represents CR₅′.

Embodiment 6. The compound according to any one of the preceding embodiments, or an isotopic derivative, stereoisomer, or a pharmaceutically acceptable salt thereof, wherein L₀ is ethenylene, a (C₁-C₃) alkylene, or a single bond; more preferably, L₀ is a single bond.

Embodiment 7. The compound according to any one of the preceding embodiments, or an isotopic derivative, stereoisomer, or a pharmaceutically acceptable salt thereof, wherein R_{E} and R_{F} are each independently selected from H and C₁-C₆ alkyl, and optionally, R_{E} and R_{F} together with the carbon atom to which they are attached form a 3- to 6-membered ring, which may optionally contain 0, 1, or 2 heteroatoms selected from N, O, and S; more preferably, R_{E} and R_{F} are each independently selected from C₁-C₃alkyl.

Embodiment 8. The compound according to any one of the preceding embodiments, or an isotopic derivative, stereoisomer, or a pharmaceutically acceptable salt thereof, having the structure of formula (B):

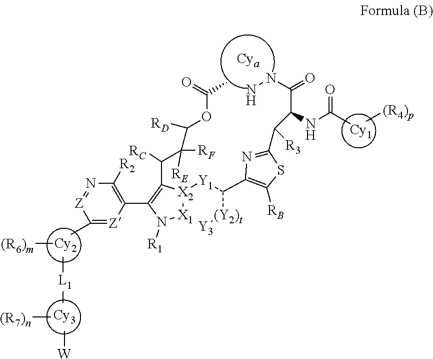

wherein: --- represents single bond or double bond;
X₁, X₂ each independently represents C or N;
Y₁, Y₂, Y₃ each independently represent no bond formed, or a single bond, CR_{A}, N, NR₁′, O, S, wherein, R₁′ represents C₁-C₆ alkyl, —(C₀-C₆ alkylene)-(C₃-C₈ cycloalkyl), —(C₀-C₆ alkylene)-(4-8-membered heterocycloalkyl), —(C₁-C₆ alkylene)-ORa, —(C₁-C₆ alkylene)-SRa or —(C₁-C₆ alkylene)-NRaRa′; wherein, when Y₃ is CR_{A}, R₁ and R_{A} substituent on Y₃, together with the N to which R₁ is connected, X₁, and C atoms, may optionally form a 6- to 10-membered ring, which may further be substituted with 0, 1, 2, 3, or 4 substituents selected from halogen, C₁-C₃ alkyl, —ORa, —SRa, or —NRaRa′;
R₁ represents C₁-C₆ alkyl, —(C₀-C₆ alkylene)-(C₃-C₈ cycloalkyl), —(C₀-C₆ alkylene)-(4-8-membered heterocycloalky), —(C₁-C₆ alkylene)-ORa, —(C₁-C₆ alkylene)-SRa or —(C₁-C₆ alkylene)-NRaRa′;
t is selected from 0, 1, 2, or 3;
R_{A}, R_{B}, R_{C}, R_{D}, R_{E}, R_{F}, Z, Z′, R₂, R₃, R₄, R₆, R₇, Cya, Cy₁, Cy₂, Cy₃, L₁, W, m, n, p are as defined in any of the preceding embodiments.

Embodiment 9. The compound according to any one of the preceding embodiments, or an isotopic derivative, stereoisomer, or a pharmaceutically acceptable salt thereof, having the structure of formula (B′):

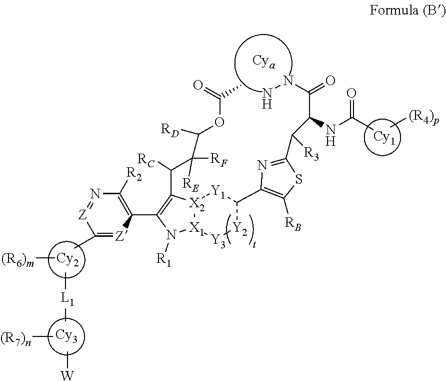

wherein: ---, X₁, X₂, Y₁, Y₂, Y₃, R_{A}, R_{B}, R_{C}, R_{D}, R_{E}, R_{F}, Z, Z′, R₁, R₂, R₃, R₄, R₆, R₇, Cya, Cy₁, Cy₂, Cy₃, L₁, W, m, n, p, t are as defined in embodiment 8.

Embodiment 10. The compound according to any one of embodiments 8 to 9, or an isotopic derivative, stereoisomer, or a pharmaceutically acceptable salt thereof, wherein X₁ and X₂ are each independently C.

Embodiment 11. The compound according to any one of embodiments 8 to 10, or an isotopic derivative, stereoisomer, or a pharmaceutically acceptable salt thereof, wherein

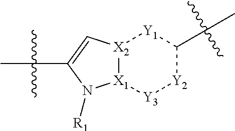

is selected from

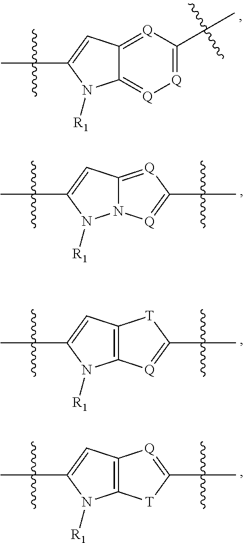

wherein, Q represents CR_{A} or N, T represents NR₁′, O or S; and optionally, when Y₃ is CR_{A}, R₁, together with R_{A} substituent on Y₃ and the ring atoms of Cy₀, may form a 6-10-membered ring, which may be further substituted with 0, 1, 2, 3, or 4 substituents independently selected from halogen, C₁-C₃ alkyl, —ORa, —SRa or —NRaRa′.

Embodiment 12. The compound according to any one of embodiments 8 to 11, or an isotopic derivative, stereoisomer, or a pharmaceutically acceptable salt thereof, wherein

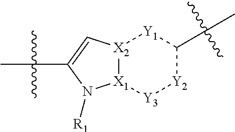

represents

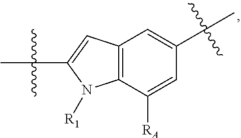

and optionally, R₁ may, together with the R_{A} substituent of Y₃ and the N to which R₁ is connected, X₁, and C atoms, form a 6- to 10-membered ring, which may further be substituted with 0, 1, 2, 3, or 4 substituents selected from halogen, C₁-C₃ alkyl, and —OH.

Embodiment 13. The compound according to any one of embodiments 8 to 10, or an isotopic derivative, stereoisomer, or a pharmaceutically acceptable salt thereof, wherein

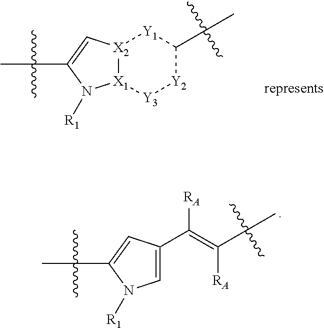

Embodiment 14. The compound according to any one of the preceding embodiments, or an isotopic derivative, stereoisomer, or a pharmaceutically acceptable salt thereof, wherein R_{A} is each independently selected from H, halogen, CN, or C₁-C₃ alkyl; more preferably, R_{A} is each independently H or F.

Embodiment 15. The compound according to any one of the preceding embodiments, or an isotopic derivative, stereoisomer, or a pharmaceutically acceptable salt thereof, having the structure of formula (I):

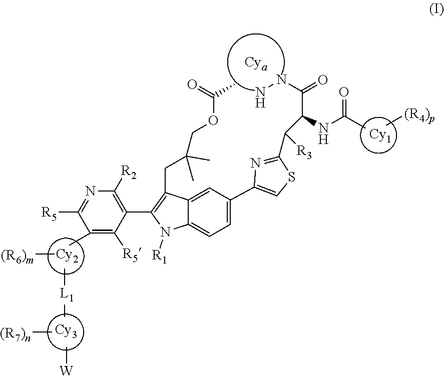

wherein:
Cya represents

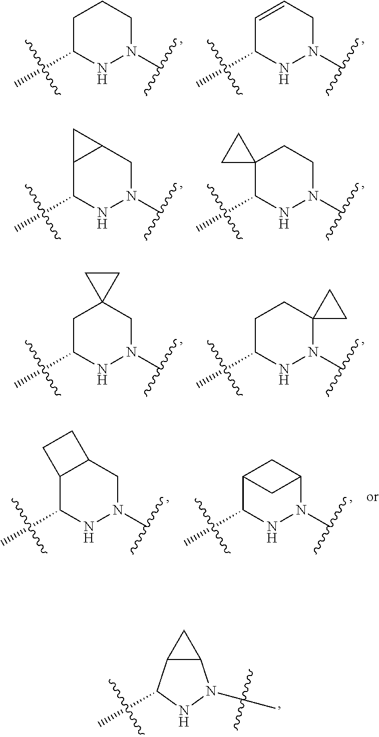

which is optionally substituted with 0, 1, 2, or 3 substituents selected from halogen or C₁-C₃ alkyl;
R₁ represents a C₁-C₆ alkyl, —(C₀-C₆ alkylene)-(C₃-C₈ cycloalkyl), —(C₀-C₆ alkylene)-(4-8-membered heterocycloalky), —(C₁-C₆ alkylene)-ORa, —(C₁-C₆ alkylene)-SRa or —(C₁-C₆ alkylene)-NRaRa′
R₂ represents halogen, cyano, C₁-C₆ alkyl, —(C₀-C₆ alkylene)-(C₃-C₈ cycloalkyl), or —(C₀-C₆ alkylene)-(4-8-membered heterocycloalkyl), which may optionally be substituted with 0, 1, or 2 substituents selected from: —ORa, —SRa, or —NRaRa′;
R₃ represents hydrogen, hydroxyl, —(C₁-C₃ alkylene)-ORa, or C₁-C₃ alkyl;
Cy₁ represents C₃-C₁₀ cycloalkyl or 4-10-membered heterocycloalkyl, and Cy₁ may be a mono ring, spiro ring, bridged ring, or fused ring;
R₄ each independently represents hydrogen, halogen, oxo, C₁-C₆ alkyl, —(C₀-C₆ alkylene)(C₃-C₆) cycloalkyl, —(C₀-C₆ alkylene)(4-8-membered) heterocycloalkyl, —(C₀-C₆ alkylene)ORa, —(C₀-C₆ alkylene)SRa, —(C₀-C₆ alkylene)NRaRa′, —(C₀-C₆ alkylene)CORa, —(C₀-C₆ alkylene)COORa, —(C₀-C₆ alkylene)CONRaRa′, —(C₀-C₆ alkylene) NRaCORa′, —(C₀-C₆ alkylene) OCONRaRa′, —(C₀-C₆ alkylene) NRaCONRaRa′, —(C₀-C₆ alkylene)SORa, —(C₀-C₆ alkylene)S(O)₂Ra, —(C₀-C₆ alkylene)NRaS(O)₂Ra′, —(C₀-C₆ alkylene)CN, —(C₀-C₆ alkylene)(C₆-C₁₀ aryl) or —(C₀-C₆ alkylene)(5-12-membered heteroaryl); wherein the R₄ substituents on two carbon atoms of Cy₁, together with the carbon atoms to which they are attached and the atoms between said two carbon atoms, may form a 3-8 membered ring, which may optionally contain 0, 1, 2, or 3 heteroatoms selected from N, O, or S; or the two R₄ substituents on the same carbon atom of Cy₁, together with the carbon atom to which they are attached, may form a 3-8 membered ring, which may optionally contain 0, 1, 2, or 3 heteroatoms selected from N, O, or S;
R₅, R₅′ are each independently selected from hydrogen, halogen, C₁-C₆ alkyl, —(C₀—C₆ alkylene)(C₃-C₈) cycloalkyl or —(C₀-C₆ alkylene)CN;
Cy₂ represents 5-membered heteroaryl;
Cy₃ represents C₃-C₁₂ cycloalkyl or 4-12-membered heterocycloalkyl, which may be a mono ring, spiro ring, bridged ring, or fused ring;
R₆ and R₇ are each independently selected from: hydrogen, halogen, oxo, —ORa, —NRaRa′, cyano, C₁-C₆ alkyl, C₃-C₈ cycloalkyl, or 4-8-membered heterocycloalkyl; each of the above-mentioned C₁-C₆ alkyl, C₃-C₈ cycloalkyl, and 4-8-membered heterocycloalkyl may be independently substituted with 0, 1, 2, 3, or 4 substituents selected from halogen, oxo, —ORa, —SRa, —NRaRa′, cyano, C₁-C₆ alkyl, C₃-C₈ cycloalkyl, or 4-8-membered heterocycloalkyl;
W is H or represented by formula (II), wherein * represents the site where W is attached to Cy₃:

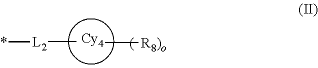

Cy₄ is selected from C₃-C₁₂ cycloalkyl or 4-12-membered heterocycloalkyl, which may be a mono ring, spiro ring, bridged ring, or fused ring;
R₈ is each independently selected from: hydrogen, halogen, oxo, —ORa, —NRaRa′, cyano, or C₁-C₆ alkyl, wherein the C₁-C₆ alkyl may be substituted with 0, 1, 2, 3, or 4 substituents selected from halogen, oxo, —ORa, —NRaRa′, or cyano;
L₁ and L₂ are each independently a single bond or a —(C₁-C₆) alkylene-, wherein any methylene of the —(C₁-C₆) alkylene- may be replaced by a carbonyl, NRa, O, or S; the —(C₁-C₆) alkylene- may be substituted with 0, 1, 2, 3, or 4 substituents selected from halogen or C₁-C₃ alkyl; and two substituents on the same carbon atom may together with the carbon atom to which they are attached form a 3-6-membered ring, which optionally contains 0, 1, 2, or 3 heteroatoms selected from N, O, or S;
wherein m, n, o, p, and q are each independently selected from 0, 1, 2, 3, or 4;
Ra and Ra′ are each independently selected from hydrogen, C₁-C₆ alkyl, C₃-C₈ cycloalkyl, or a 4-8-membered heterocycloalkyl; when Ra and Ra′ are attached to the same nitrogen atom, Ra and Ra′ together with the nitrogen atom to which they are attached may form a 4-8-membered ring, which may optionally contain 0, 1, 2, or 3 heteroatoms selected from N, O, or S;
the alkyl, cycloalkyl, heterocycloalkyl, and alkylene groups may each independently be substituted by 0, 1, 2, 3, 4, 5, or 6 halogen atoms.

Embodiment 16. The compound according to any one of the preceding embodiments, or an isotopic derivative, stereoisomer, or a pharmaceutically acceptable salt thereof, having the structure of formula (I′):

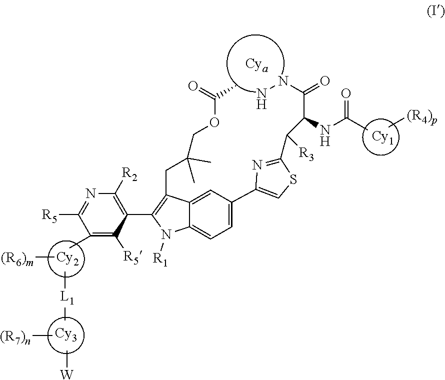

wherein: R₁, R₂, R₃, R₄, R₅, R₅′, R₆, R₇, Cya, Cy₁, Cy₂, Cy₃, L₁, W, m, n, p are as defined in embodiment 15.

Embodiment 17. The compound according to any one of the preceding embodiments, or an isotopic derivative, stereoisomer, or a pharmaceutically acceptable salt thereof, wherein Cya represents

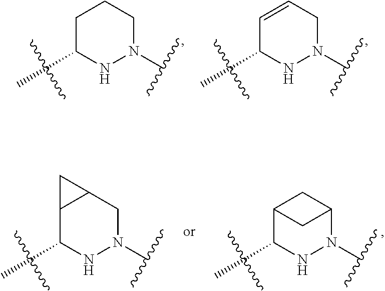

which is optionally substituted with 0, 1, 2, or 3 substituents selected from halogen or C₁-C₃ alkyl.

Embodiment 18. The compound according to any one of the preceding embodiments, or an isotopic derivative, stereoisomer, or a pharmaceutically acceptable salt thereof, wherein Cya represents

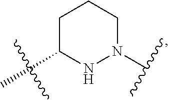 [SMILES: *N1CCC[C@@H](C)N1]

which is optionally substituted with 0, 1, 2, or 3 substituents selected from halogen or C₁-C₃ alkyl; preferably, Cya represents

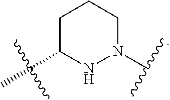 [SMILES: *N1CCC[C@@H](C)N1]

Embodiment 19. The compound according to any one of the preceding embodiments, or an isotopic derivative, stereoisomer, or a pharmaceutically acceptable salt thereof, wherein Cya represents

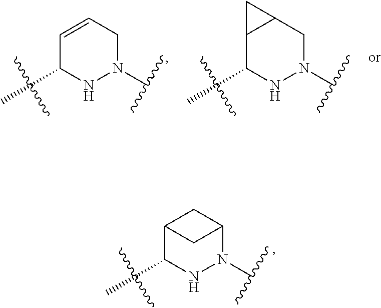

which is optionally substituted with 0, 1, 2, or 3 substituents selected from halogen or C₁-C₃ alkyl; preferably, Cya represents

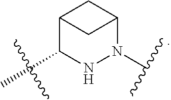 [SMILES: *N1N[C@H](C)C2CC1C2]

Embodiment 20. The compound according to any one of the preceding embodiments, or an isotopic derivative, stereoisomer, or a pharmaceutically acceptable salt thereof, wherein W is represented by formula (II).

Embodiment 21. The compound according to any one of the preceding embodiments, or an isotopic derivative, stereoisomer, or a pharmaceutically acceptable salt thereof, wherein L₁ and L₂ each independently represent a single bond or a —(C₁-C₆)alkylene- group, wherein any methylene of the —(C₁-C₆)alkylene- may optionally be replaced by a carbonyl, NRa, O, or S, and the —(C₁-C₆)alkylene- group may be substituted with 0, 1, 2, 3, or 4 C₁-C₃ alkyl substituents, and two substituents on the same carbon atom together with the carbon atom to which they are attached may form a 3- to 6-membered ring, wherein the 3- to 6-membered ring optionally contains 0, 1, 2, or 3 heteroatoms selected from N, O, or S.

Embodiment 22. The compound according to any one of the preceding embodiments, or an isotopic derivative, stereoisomer, or a pharmaceutically acceptable salt thereof, wherein R₁ represents C₁-C₆ alkyl group, —(C₀-C₆ alkylene)-(C₃-C₈ cycloalkyl) group, —(C₀-C₆ alkylene)-(4-8-membered heterocycloalkyl) group, halogen-substituted C₁-C₆ alkyl group, halogen-substituted —(C₀-C₆ alkylene)-(C₃-C₈ cycloalkyl) group, or halogen-substituted —(C₀-C₆ alkylene)-(4-8-membered heterocycloalkyl) group; preferably, R₁ represents C₁-C₆ alkyl group or halogen-substituted C₁-C₆ alkyl group; more preferably, R₁ represents ethyl or —CH₂CF₃.

Embodiment 23. The compound according to any one of the preceding embodiments, or an isotopic derivative, stereoisomer, or a pharmaceutically acceptable salt thereof, wherein R₂ represents C₁-C₆ alkyl group which may be substituted with 0 or 1 C₁-C₃ alkoxy group; preferably, R₂ represents 1-methoxyethyl; more preferably, R₂ represents

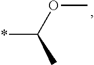

wherein, * represents the site where R₂ is attached to the corresponding position in formula.

Embodiment 24. The compound according to any one of the preceding embodiments, or an isotopic derivative, stereoisomer, or a pharmaceutically acceptable salt thereof, wherein R₃ is preferably H.

Embodiment 25. The compound according to any one of the preceding embodiments, or an isotopic derivative, stereoisomer, or a pharmaceutically acceptable salt thereof, wherein Cy₁ represents C₃-C₈ cycloalkyl group or 4- to 8-membered heterocycloalkyl group; preferably, Cy₁ represents C₃-C₈ cycloalkyl group; more preferably, Cy₁ represents cyclopropyl group.

Embodiment 26. The compound according to any one of the preceding embodiments, or an isotopic derivative, stereoisomer, or a pharmaceutically acceptable salt thereof, wherein R₄ each independently represents hydrogen, halogen, or C₁-C₃ alkyl group.

Embodiment 27. The compound according to any one of the preceding embodiments, or a pharmaceutically acceptable salt, isotopic derivative, or stereoisomer thereof, wherein the structure of -Cy₁-(R₄)p is selected from the following:

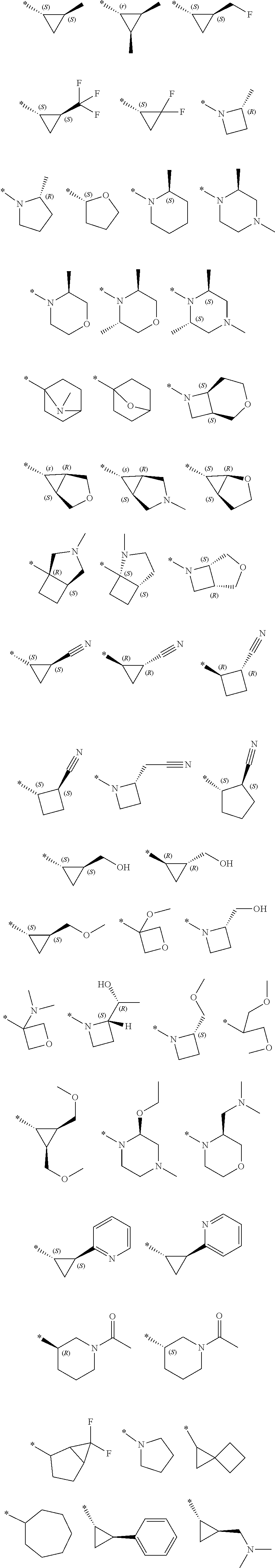

wherein, * represents the site where -Cy₁-(R₄)p is attached to the corresponding position in formula.

Embodiment 28. The compound according to any one of the preceding embodiments, or an isotopic derivative, stereoisomer, or a pharmaceutically acceptable salt thereof, wherein R₅ and R₅′ are each independently selected from hydrogen, halogen, or C₁-C₆ alkyl; preferably, R₅ and R₅′ are hydrogen.

Embodiment 29. The compound according to any one of the preceding embodiments, or an isotopic derivative, stereoisomer, or a pharmaceutically acceptable salt thereof, wherein

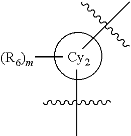

is selected from

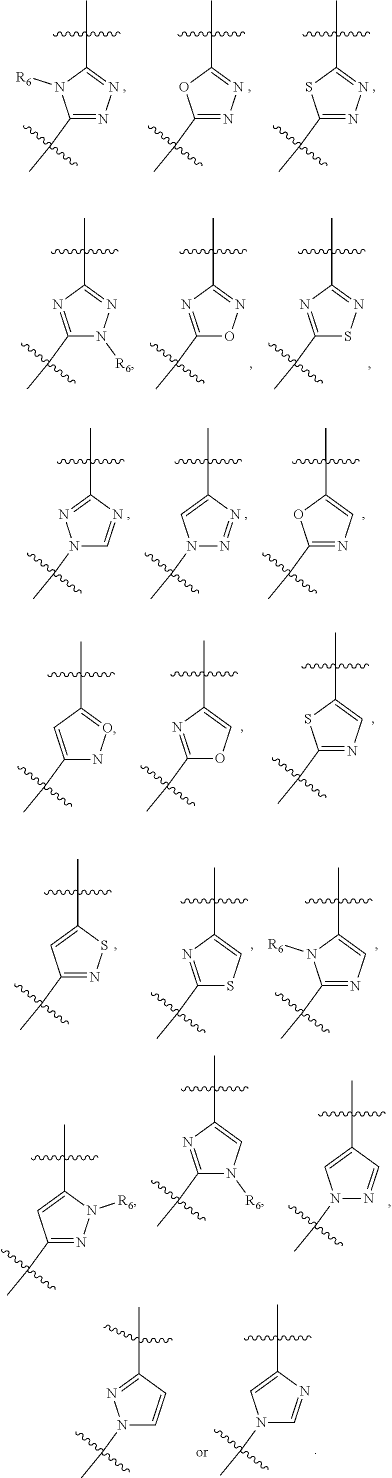

Embodiment 30. The compound according to any one of the preceding embodiments, or an isotopic derivative, stereoisomer, or a pharmaceutically acceptable salt thereof, wherein Cy₂ is not pyrazole or isoxazole.

Embodiment 31. The compound according to any one of the preceding embodiments, or an isotopic derivative, stereoisomer, or a pharmaceutically acceptable salt thereof, wherein Cy₂ is not

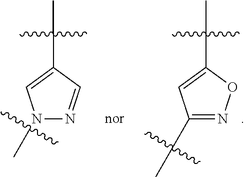

Embodiment 32. The compound according to any one of the preceding embodiments, or an isotopic derivative, stereoisomer, or a pharmaceutically acceptable salt thereof, wherein

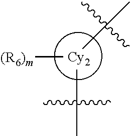

is not

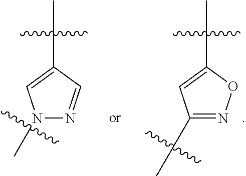

Embodiment 33. The compound according to any one of the preceding embodiments, or an isotopic derivative, stereoisomer, or a pharmaceutically acceptable salt thereof, wherein

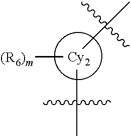

is selected from

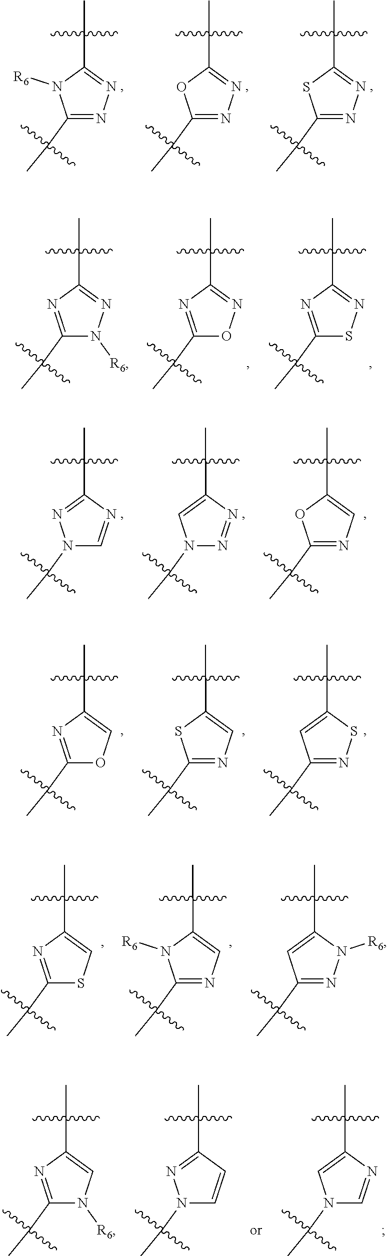

preferably,

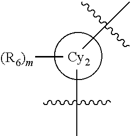

is selected from

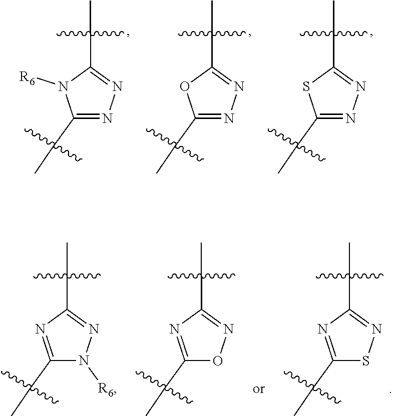

Embodiment 34. The compound according to any one of the preceding embodiments, or an isotopic derivative, stereoisomer, or a pharmaceutically acceptable salt thereof, wherein Cy₃ represents a 4- to 12-membered heterocycloalkyl, which may be a mono ring, spiro ring, bridged ring, or fused ring; preferably, Cy₃ represents a 4- to 8-membered heterocycloalkyl, which may be a mono ring, spiro ring, bridged ring, or fused ring.

Embodiment 35. The compound according to any one of the preceding embodiments, or an isotopic derivative, stereoisomer, or a pharmaceutically acceptable salt thereof, wherein Cy₃ represents

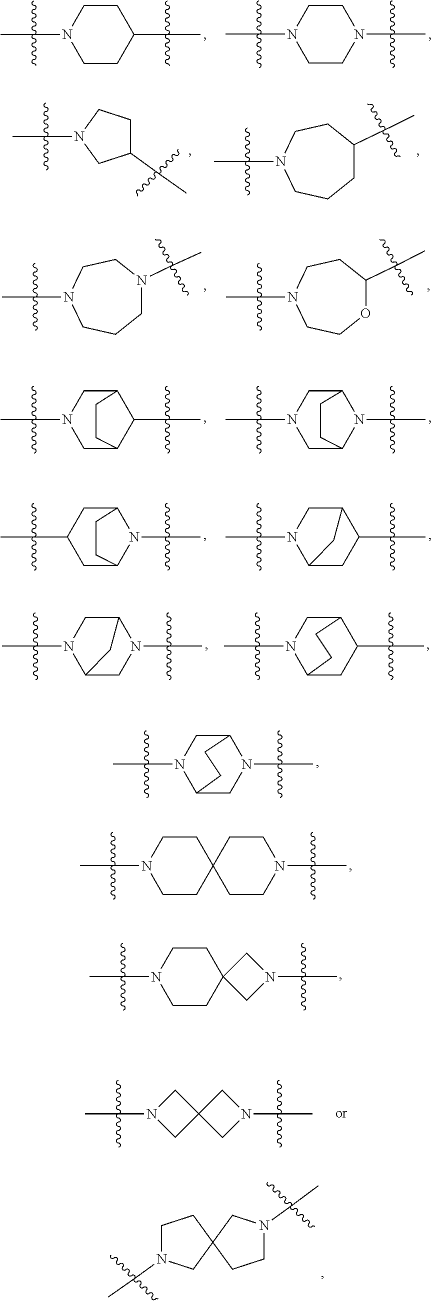

and Cy₃ may have n substituents selected from R₇; more preferably, Cy₃ represents

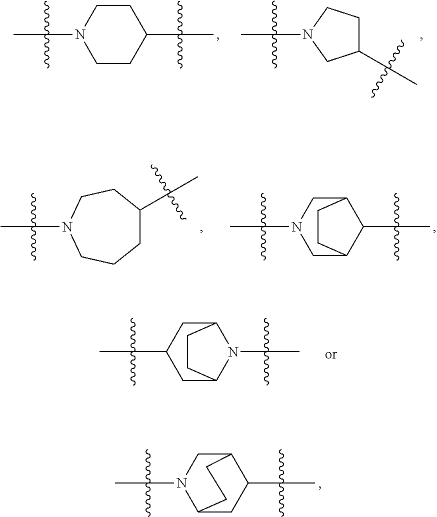

and Cy₃ may have n substituents selected from R₇.

Embodiment 36. The compound according to any one of the preceding embodiments, or an isotopic derivative, stereoisomer, or a pharmaceutically acceptable salt thereof, wherein R₆ is each independently selected from hydrogen, C₁-C₆ alkyl, C₃-C₈ cycloalkyl, or 4- to 8-membered heterocycloalkyl, each of said C₁-C₆ alkyl, C₃-C₈ cycloalkyl, and 4- to 8-membered heterocycloalkyl may independently be optionally substituted with 0, 1 or 2 substituents selected from halogen, oxo, —ORa, —NRaRa′, cyano, C₁-C₆ alkyl, C₃-C₈ cycloalkyl, or 4- to 8-membered heterocycloalkyl; preferably, R₆ is each independently selected from hydrogen, C₁-C₆ alkyl or C₃-C₈ cycloalkyl, each of said C₁-C₆ alkyl and C₃-C₈ cycloalkyl may independently be optionally substituted with 0, 1 or 2 substituents selected from halogen, C₁-C₆ alkyl or C₃-C₆ cycloalkyl; more preferably, R₆ is each independently selected from hydrogen or methyl.

Embodiment 37. The compound according to any one of the preceding embodiments, or an isotopic derivative, stereoisomer, or a pharmaceutically acceptable salt thereof, wherein R₇ is each independently selected from hydrogen, halogen, oxo, —ORa, —N RaRa′, cyano, or C₁-C₆ alkyl, wherein each of said C₁-C₆ alkyl is independently optionally substituted with 0, 1, or 2 substituents selected from halogen, oxo, —ORa, —NRaRa′, or cyano; preferably, R₇ is each independently selected from hydrogen, halogen, oxo, hydroxyl, cyano, or C₁-C₆ alkyl.

Embodiment 38. The compound according to any one of the preceding embodiments, or an isotopic derivative, stereoisomer, or a pharmaceutically acceptable salt thereof, wherein L₁ represents single bond, —CH₂—, —O—, or —NH—.

Embodiment 39. The compound according to any one of the preceding embodiments, or an isotopic derivative, stereoisomer, or a pharmaceutically acceptable salt thereof, wherein

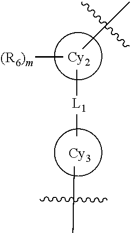

represents

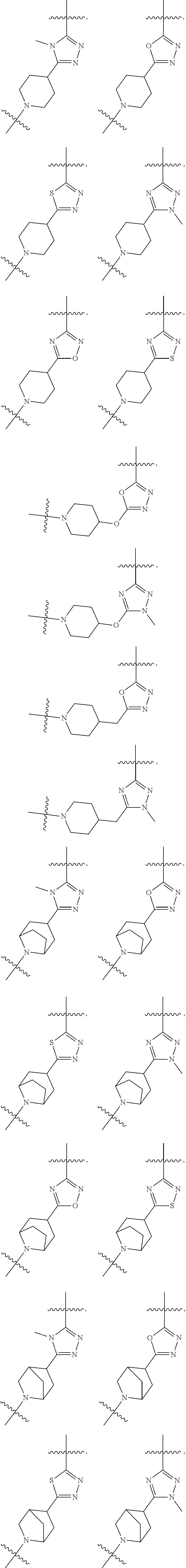

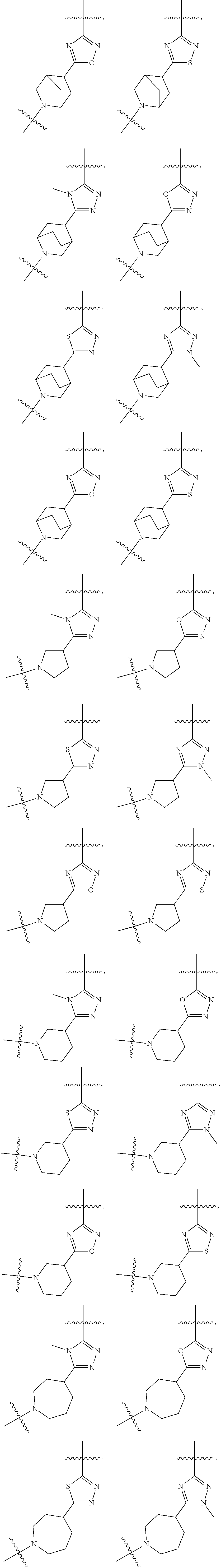

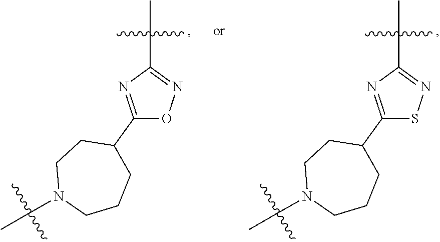 [SMILES: *c1noc(C2CCCN(*)CC2)n1 ; *c1nsc(C2CCCN(*)CC2)n1]

and Cy₃ may have n substituents selected from R₇.

Embodiment 40. The compound according to any one of the preceding embodiments, or an isotopic derivative, stereoisomer, or a pharmaceutically acceptable salt thereof, wherein Cy₄ is C₅-C₆ cycloalkyl or 5-6 membered heterocycloalkyl, o is ≥1, and R₈ is not hydrogen.

Embodiment 41. The compound according to any one of the preceding embodiments, or an isotopic derivative, stereoisomer, or a pharmaceutically acceptable salt thereof, wherein Cy₄ is selected from C₃-C₄ cycloalkyl or 4-membered heterocycloalkyl.

Embodiment 42. The compound according to any one of the preceding embodiments, or an isotopic derivative, stereoisomer, or a pharmaceutically acceptable salt thereof, wherein Cy₄ is selected from

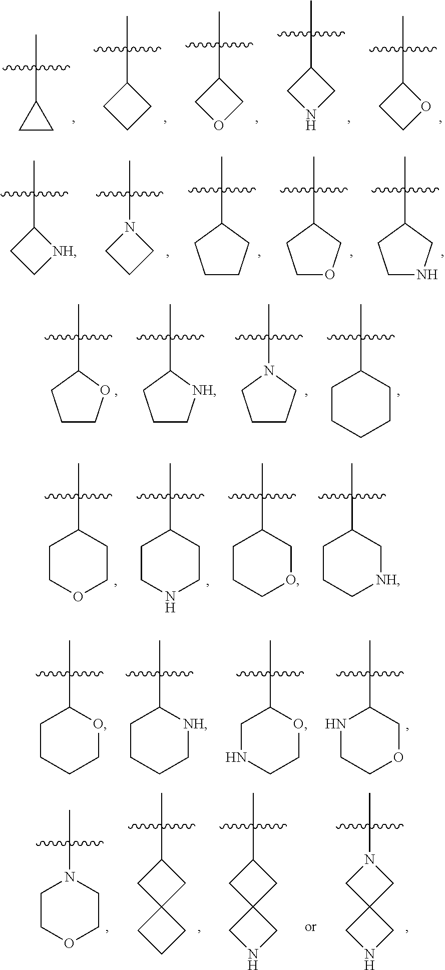

and Cy₄ may have o substituents selected from R₈; preferably, Cy₄ is selected from

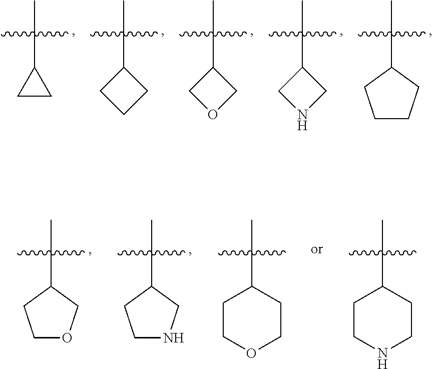

and Cy₄ may have o substituents selected from R₈; more preferably, Cy₄ is selected from

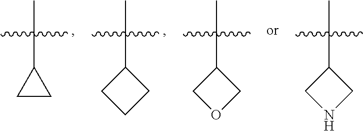

and Cy₄ may have o substituents selected from R₈.

Embodiment 43. The compound according to any one of the preceding embodiments, or an isotopic derivative, stereoisomer, or a pharmaceutically acceptable salt thereof, wherein L₂ represents a single bond, —CH₂—, or —O—; preferably, L₂ represents a single bond.

Embodiment 44. The compound according to any one of the preceding embodiments, or an isotopic derivative, stereoisomer, or a pharmaceutically acceptable salt thereof, wherein R₈ is each independently selected from hydrogen, halogen, or C₁-C₆ alkyl.

Embodiment 45. The compound according to any one of the preceding embodiments, or an isotopic derivative, stereoisomer, or a pharmaceutically acceptable salt thereof, wherein

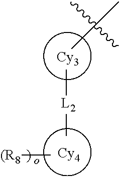

represents

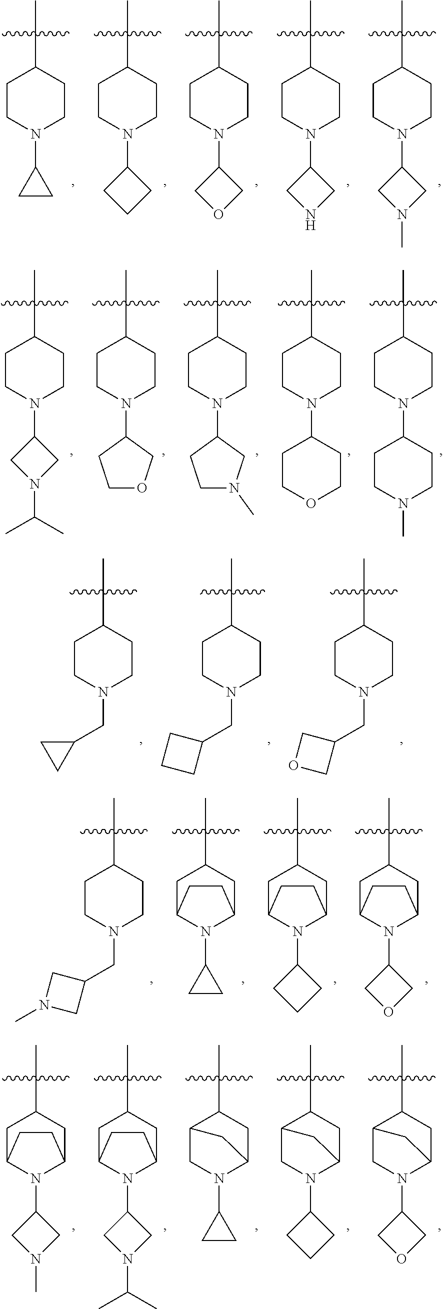

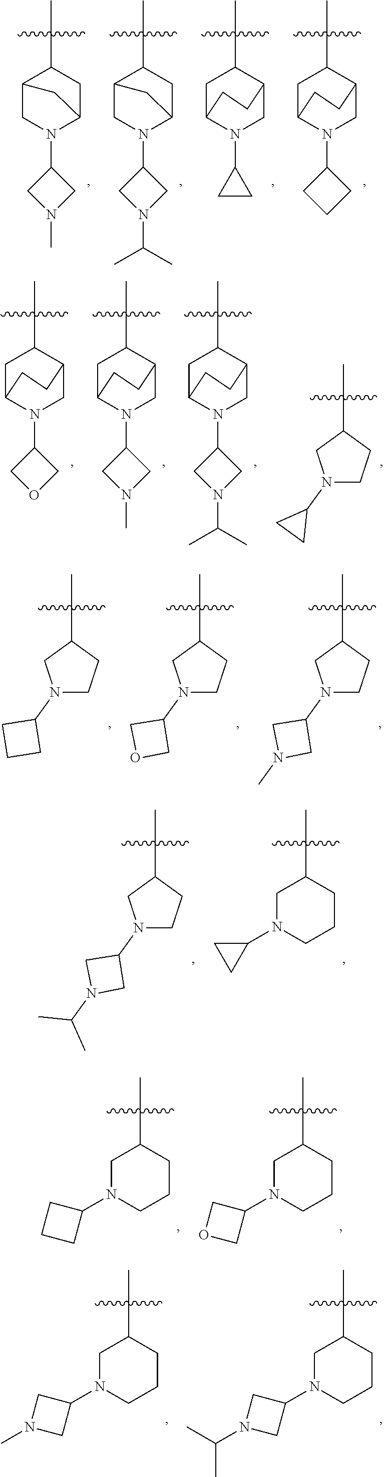

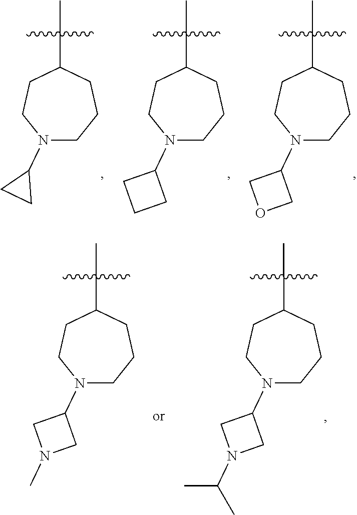 [SMILES: *C1CCCN(C2CC2)CC1 ; *C1CCCN(C2CCC2)CC1 ; *C1CCCN(C2COC2)CC1 ; *C1CCCN(C2CN(C)C2)CC1 ; *C1CCCN(C2CN(C(C)C)C2)CC1]

and Cy₃ may have n substituents selected from R₇.

Embodiment 46. The compound according to any one of the preceding embodiments, or an isotopic derivative, stereoisomer, or a pharmaceutically acceptable salt thereof, wherein

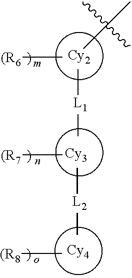

represents

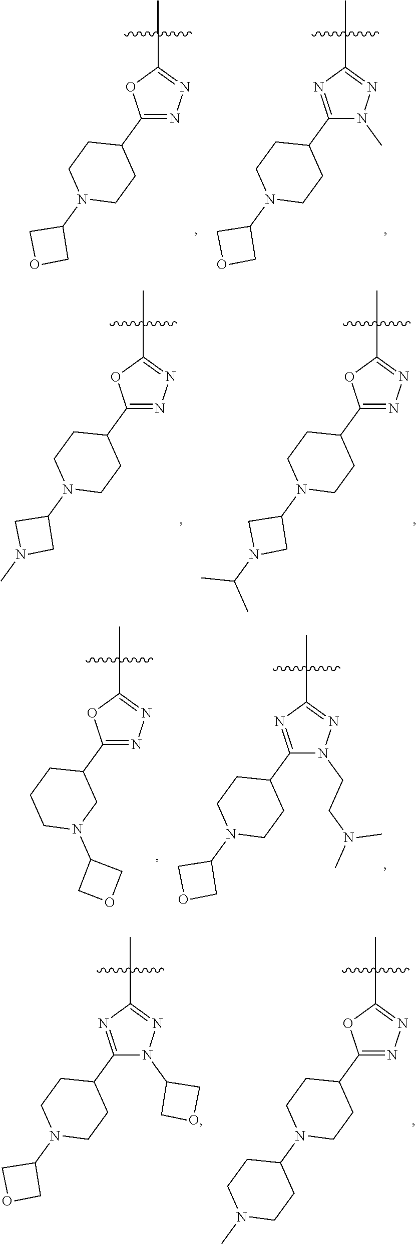

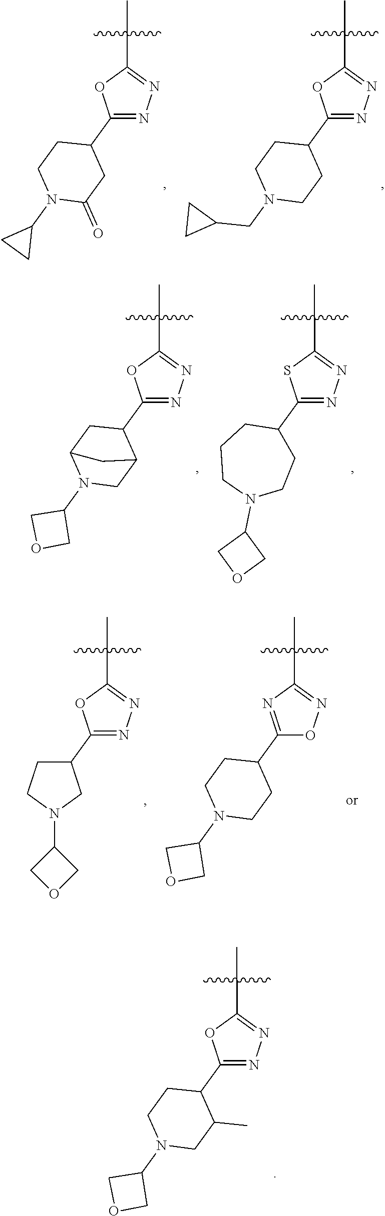

Embodiment 47. The compound according to any one of the preceding embodiments, or an isotopic derivative, stereoisomer, or a pharmaceutically acceptable salt thereof, wherein m, n, o, p, q, and t are each independently 0, 1, or 2.

Embodiment 48. The compound according to any one of the preceding embodiments, or an isotopic derivative, stereoisomer, or a pharmaceutically acceptable salt thereof, wherein Ra and Ra′ are each independently selected from hydrogen, C₁-C₆ alkyl, C₃-C₈ cycloalkyl, or 4- to 8-membered heterocycloalkyl; preferably, Ra and Ra′ are each independently selected from hydrogen or C₁-C₆ alkyl; more preferably, Ra and Ra′ are each independently selected from hydrogen or C₁-C₃ alkyl.

Embodiment 49. The compound according to any one of the preceding embodiments, or an isotopic derivative, stereoisomer, or a pharmaceutically acceptable salt thereof, having a structure represented by formula (III):

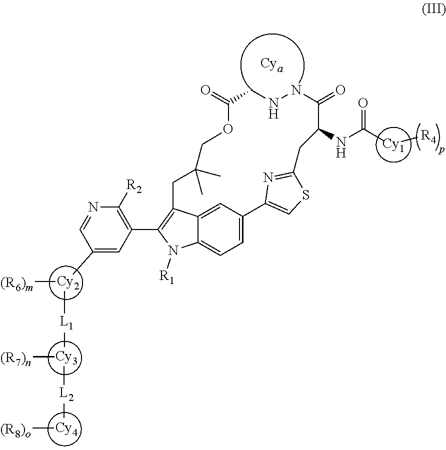

wherein:
Cya represents

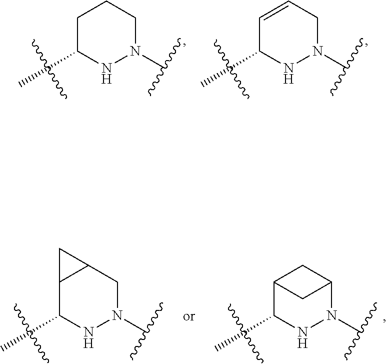

which is optionally substituted with 0, 1, 2, or 3 substituents selected from halogen or C₁-C₃ alkyl;
R₁ represents ethyl or —CH₂CF₃;
R₂ represents C₁-C₆ alkyl, which may be substituted with 0 or 1 C₁-C₃ alkoxy;
Cy₁ represents a C₃-C₈ cycloalkyl group or a 4-8 membered heterocycloalkyl group;
R₄ each independently represents hydrogen, halogen, or C₁-C₃ alkyl;
Cy₂ represents a 5-membered heteroaryl group, and Cy₂ is not

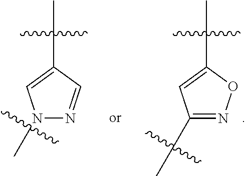

Cy₃ represents a 4-12-membered heterocycloalkyl;
R₆ is each independently selected from hydrogen, C₁-C₆ alkyl, C₃-C₈ cycloalkyl, or 4-8-membered heterocycloalkyl, wherein each of the C₁-C₆ alkyl, C₃-C₈ cycloalkyl, and 4-8-membered heterocycloalkyl is independently optionally substituted with 0, 1, or 2 substituents selected from halogen, oxo, —ORa, —NRaRa′, cyano, C₁-C₆ alkyl, C₃-C₈ cycloalkyl, or 4-8-membered heterocycloalkyl;
R₇ is each independently selected from hydrogen, halogen, oxo, —ORa, —NRaRa′, cyano, and C₁-C₆ alkyl, wherein the C₁-C₆ alkyl is independently optionally substituted with 0, 1, or 2 substituents selected from halogen, oxo, —ORa, —NRaRa′, and cyano;
L₁ represents a single bond or —(C₁-C₆)alkylene-, wherein any methylene in the —(C₁-C₆)alkylene- may be replaced by a carbonyl, NRa, O, or S;
Cy₄ is selected from C₃-C₁₂ cycloalkyl or 4-12-membered heterocycloalkyl, which may be a mono ring, spiro ring, bridged ring, or fused ring; and when Cy₄ is a C₅-C₆ cycloalkyl or a 5-6-membered heterocycloalkyl, o≥1 and R₈ is not hydrogen;
L₂ represents a single bond or —(C₁-C₆)alkylene-, wherein any methylene in the —(C₁-C₆)alkylene- may optionally be replaced by a carbonyl, NRa, O or S;
R₈ is each independently selected from: hydrogen, halogen, oxo, —ORa, —NRaRa′, cyano, and C₁-C₆ alkyl, wherein the C₁-C₆ alkyl may be substituted with 0, 1, 2, 3, or 4 substituents selected from halogen, oxo, —ORa, —NRaRa′, and cyano;
m, n, o, p each independently represent 0, 1, or 2;
Ra, Ra′ each independently represent hydrogen, C₁-C₆ alkyl, C₃-C₈ cycloalkyl, or 4-8-membered heterocycloalkyl;
the alkyl, cycloalkyl, heterocycloalkyl, and alkylene groups are each independently optionally substituted with 0, 1, 2, 3, 4, 5, or 6 halogen atoms.

Embodiment 50. The compound according to any one of the preceding embodiments, or an isotopic derivative, stereoisomer, or a pharmaceutically acceptable salt thereof, having a structure represented by formula (III′):

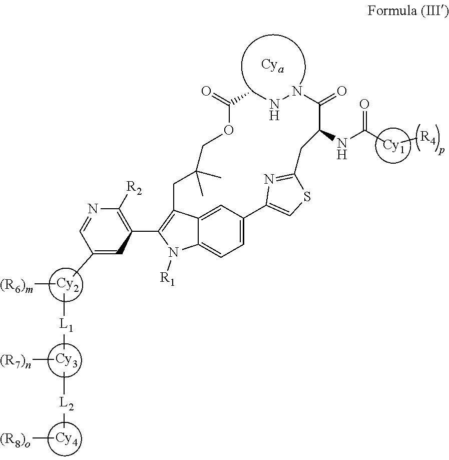

R₁, R₂, R₄, R₆, R₇, R₈, Cya, Cy₁, Cy₂, Cy₃, Cy₄, L₁, L₂, m, n, o, p are as defined in embodiment 49.

Embodiment 51. The compound according to any one of embodiments 49 to 50, or an isotopic derivative, stereoisomer, or a pharmaceutically acceptable salt thereof, wherein Cya represents

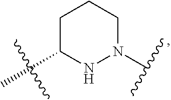 [SMILES: *N1CCC[C@@H](C)N1]

which is optionally substituted with 0, 1, 2, or 3 substituents selected from halogen or C₁-C₃ alkyl; preferably, Cya represents

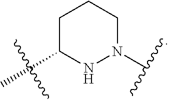 [SMILES: *N1CCC[C@@H](C)N1]

Embodiment 52. The compound according to any one of embodiments 49 to 50, or an isotopic derivative, stereoisomer, or a pharmaceutically acceptable salt thereof, wherein Cya represents

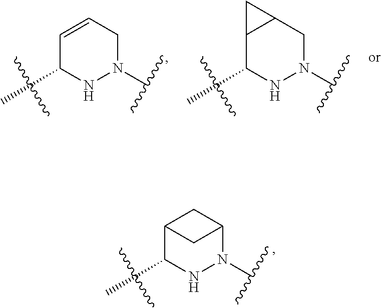

which is optionally substituted with 0, 1, 2, or 3 substituents selected from halogen or C₁-C₃ alkyl; preferably, Cya represents

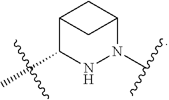 [SMILES: *N1N[C@H](C)C2CC1C2]

Embodiment 53. The compound according to any one of embodiments 49 to 52, or an isotopic derivative, stereoisomer, or a pharmaceutically acceptable salt thereof, wherein R₂ represents 1-methoxyethyl; preferably, R₂ represents

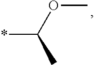 [SMILES: *[C@H](C)OC]

wherein * represents the site where R₂ is attached to the corresponding position in formula.

Embodiment 54. The compound according to any one of embodiments 49 to 53, or an isotopic derivative, stereoisomer, or a pharmaceutically acceptable salt thereof, wherein Cy₁ represents a C₃-C₈ cycloalkyl group; preferably, Cy₁ represents a cyclopropyl group.

Embodiment 55. The compound according to any one of embodiments 49 to 54, or an isotopic derivative, stereoisomer, or a pharmaceutically acceptable salt thereof, wherein

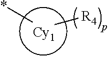

represents

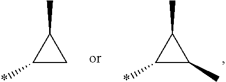

wherein * represents the site where Cy₁ is attached to the corresponding position in formula.

Embodiment 56. The compound according to any one of embodiments 49 to 55, or an isotopic derivative, stereoisomer, or a pharmaceutically acceptable salt thereof, wherein

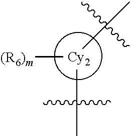

is selected from

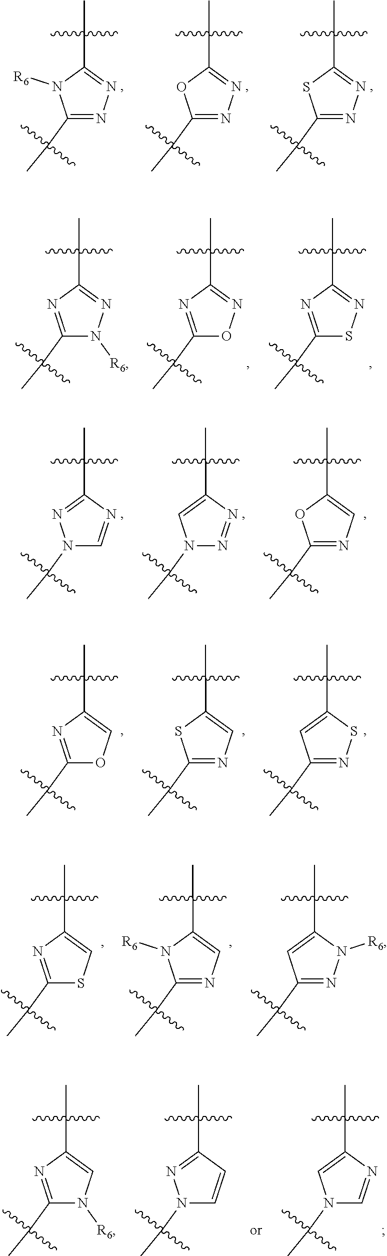

preferably,

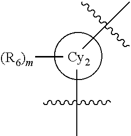

is selected from

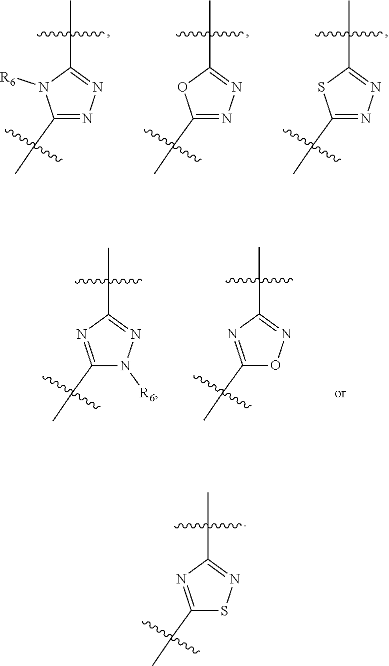

Embodiment 57. The compound according to any one of embodiments 49 to 56, or an isotopic derivative, stereoisomer, or a pharmaceutically acceptable salt thereof, wherein R₆ is each independently selected from hydrogen, C₁-C₆ alkyl, or C₃-C₈ cycloalkyl, each of the C₁-C₆ alkyl and C₃-C₈ cycloalkyl is independently optionally substituted with 0, 1, or 2 substituents selected from halogen, C₁-C₆ alkyl, or C₃-C₆ cycloalkyl; more preferably, R₆ is each independently selected from hydrogen or methyl.

Embodiment 58. The compound according to any one of embodiments 49 to 57, or an isotopic derivative, stereoisomer, or a pharmaceutically acceptable salt thereof, wherein R₇ is each independently selected from hydrogen, halogen, oxo, hydroxyl, cyano, or C₁-C₆ alkyl.

Embodiment 59. The compound according to any one of embodiments 49 to 58, or an isotopic derivative, stereoisomer, or a pharmaceutically acceptable salt thereof, wherein L₁ represents a single bond, —CH₂—, —O—, or —NH—.

Embodiment 60. The compound according to any one of embodiments 49 to 59, or an isotopic derivative, stereoisomer, or a pharmaceutically acceptable salt thereof, wherein Cy₄ is a C₅-C₆ cycloalkyl group or a 5- to 6-membered heterocycloalkyl group, o≥1, and R₈ is not hydrogen.

Embodiment 61. The compound according to any one of embodiments 49 to 60, or an isotopic derivative, stereoisomer, or a pharmaceutically acceptable salt thereof, wherein Cy₄ is selected from a C₃-C₄ cycloalkyl group or a 4-membered heterocycloalkyl group.

Embodiment 62. The compound according to any one of embodiments 49 to 61, or an isotopic derivative, stereoisomer, or a pharmaceutically acceptable salt thereof, wherein L₂ represents a single bond, —CH₂—, or —O—; preferably, L₂ represents a single bond.

Embodiment 63. The compound according to any one of embodiments 49 to 62, or an isotopic derivative, stereoisomer, or a pharmaceutically acceptable salt thereof, wherein R₈ is each independently selected from hydrogen, halogen, or C₁-C₆ alkyl.

Embodiment 64. The compound according to any one of embodiments 49 to 63, or an isotopic derivative, stereoisomer, or a pharmaceutically acceptable salt thereof, wherein Ra and Ra′ are each independently hydrogen or C₁-C₆ alkyl; preferably, Ra and Ra′ are each independently hydrogen or C₁-C₃ alkyl.

Embodiment 65. The compound according to any one of the preceding embodiments, or an isotopic derivative, stereoisomer, or a pharmaceutically acceptable salt thereof, having the structure represented by Formula (IV):

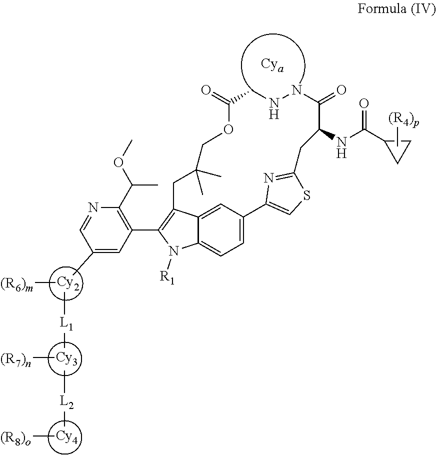

wherein:

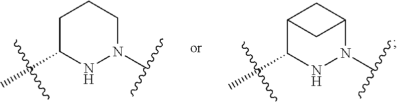

Cya represents H or H
R₁ represents ethyl or —CH₂CF₃;
R₄ each independently represents hydrogen, halogen, or C₁-C₃ alkyl;

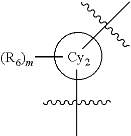

is selected from

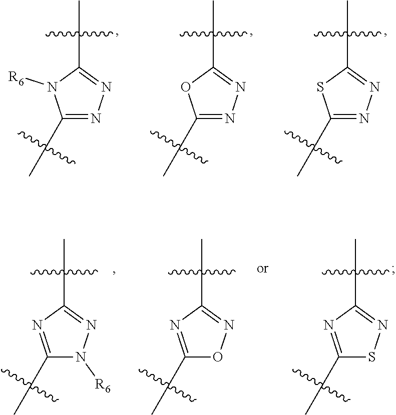

Cy₃ is selected from 4- to 8-membered heterocycloalkyl;
Cy₄ is selected from C₃-C₄ cycloalkyl or 4-membered heterocycloalkyl;
R₆ is each independently selected from hydrogen and methyl;
R₇ is each independently selected from hydrogen, halogen, oxo, or C₁-C₆ alkyl;
R₈ is each independently selected from hydrogen, halogen, or C₁-C₆ alkyl;
L₁ represents a single bond, —CH₂—, or —O—;
L₂ represents a single bond, —CH₂—, or —O—;
wherein n, o, and p are each independently 0, 1, or 2;
Ra, Ra′ are each independently hydrogen or C₁-C₆ alkyl; preferably, Ra, Ra′ are each independently hydrogen or C₁-C₃ alkyl;
the alkyl, cycloalkyl, heterocycloalkyl, and alkylene may each independently be substituted with 0, 1, 2, 3, 4, 5, or 6 halogen atoms.

Embodiment 66. The compound according to any one of the preceding embodiments, or an isotopic derivative, stereoisomer, or a pharmaceutically acceptable salt thereof, having the structure represented by Formula (IV′):

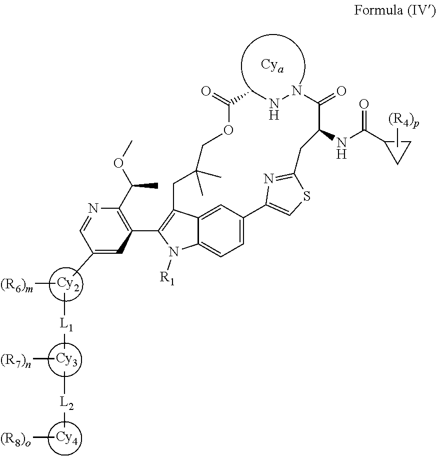

R₁, R₄, R₆, R₇, R₈, Cya, Cy₂, Cy₃, Cy₄, L₁, L₂, m, n, o, p are as defined in embodiment 65.

Embodiment 67. The compound according to any one of embodiments 65 to 66, or an isotopic derivative, stereoisomer, or a pharmaceutically acceptable salt thereof, wherein

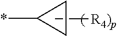

represents

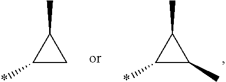

wherein, * represents the site where Cy₁ is attached to the corresponding position in the formula.

Embodiment 68. The compound according to any one of embodiments 65 to 67, or an isotopic derivative, stereoisomer, or a pharmaceutically acceptable salt thereof, wherein Cya represents

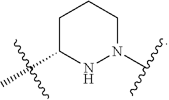 [SMILES: *N1CCC[C@@H](C)N1]

Embodiment 69. The compound according to any one of embodiments 65 to 67, or an isotopic derivative, stereoisomer, or a pharmaceutically acceptable salt thereof, wherein Cya represents

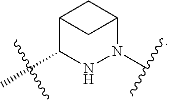 [SMILES: *N1N[C@H](C)C2CC1C2]

Embodiment 70. A compound having the following structure, or an isotope derivative, stereoisomer thereof, or a pharmaceutically acceptable salt thereof:

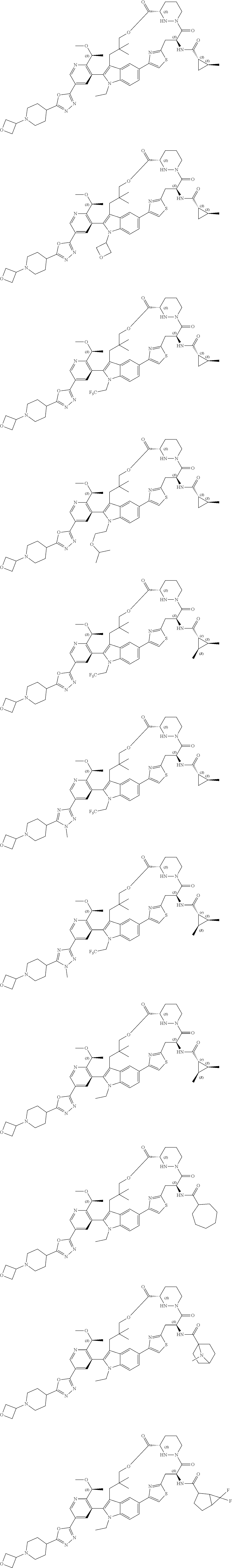

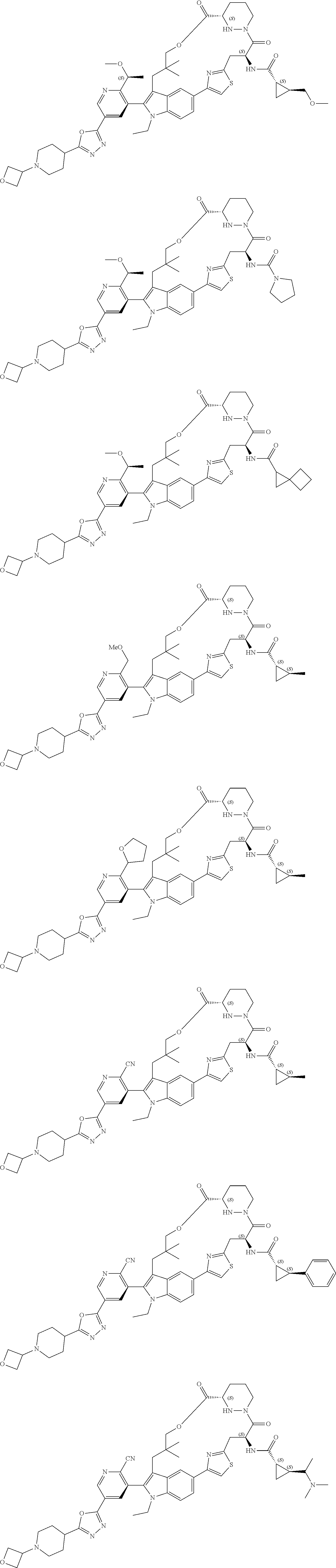

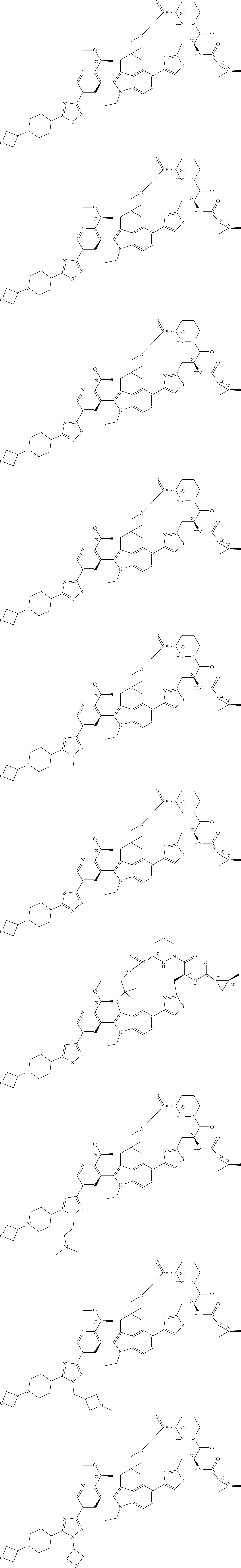

[chemical structure]

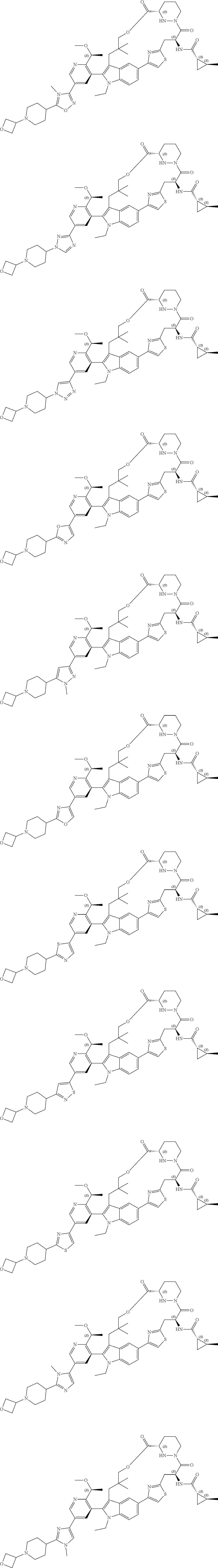

[chemical structure]

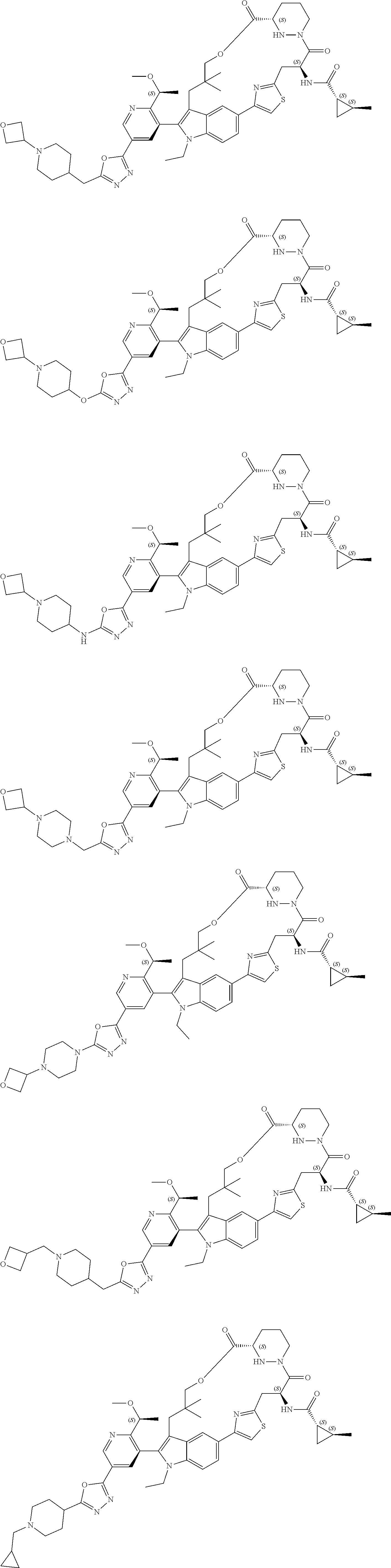

[chemical structure]

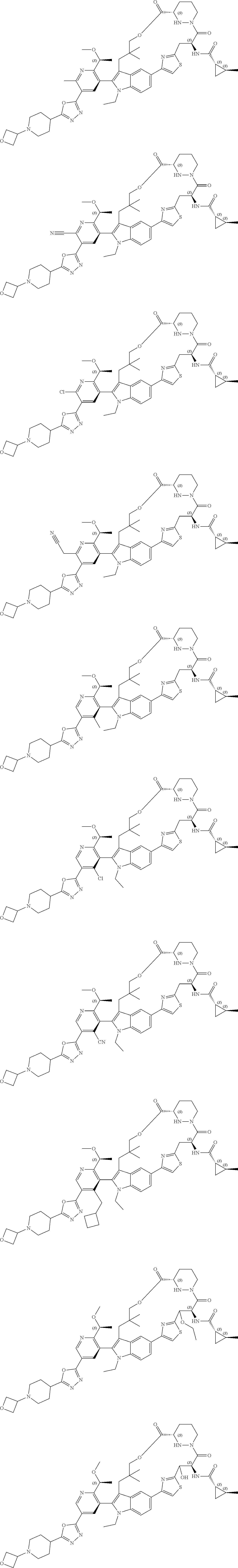

[chemical structure]

[chemical structure]

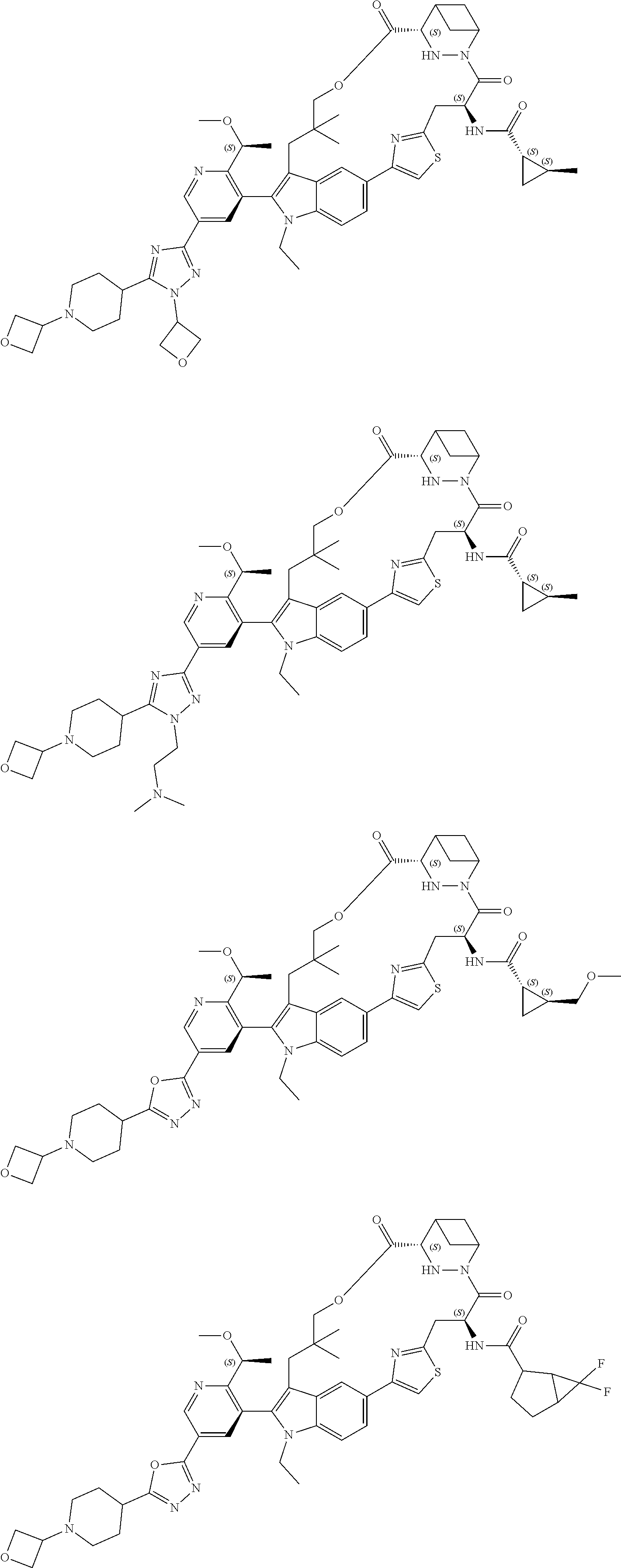

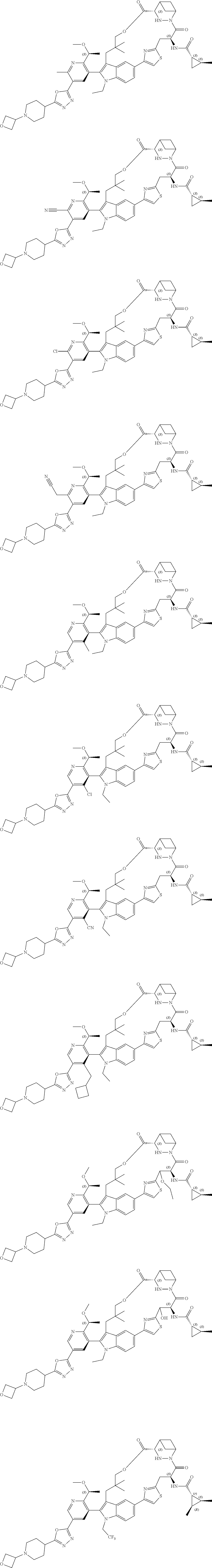

[chemical structure]

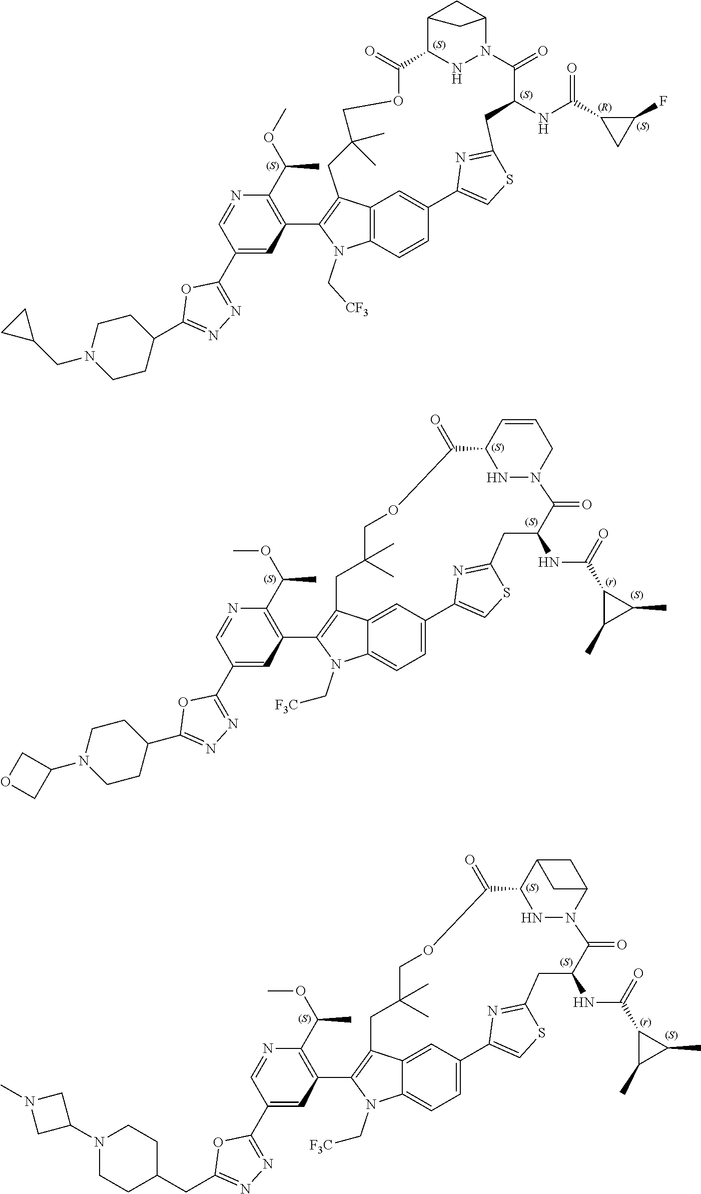

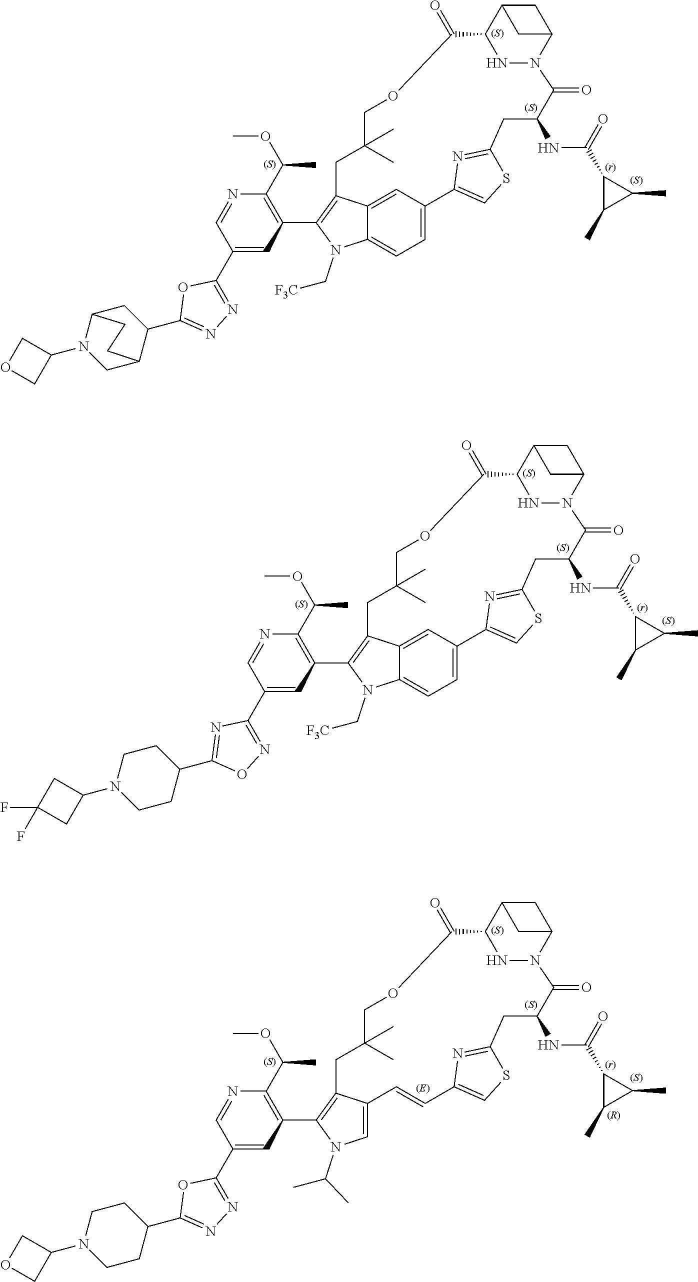

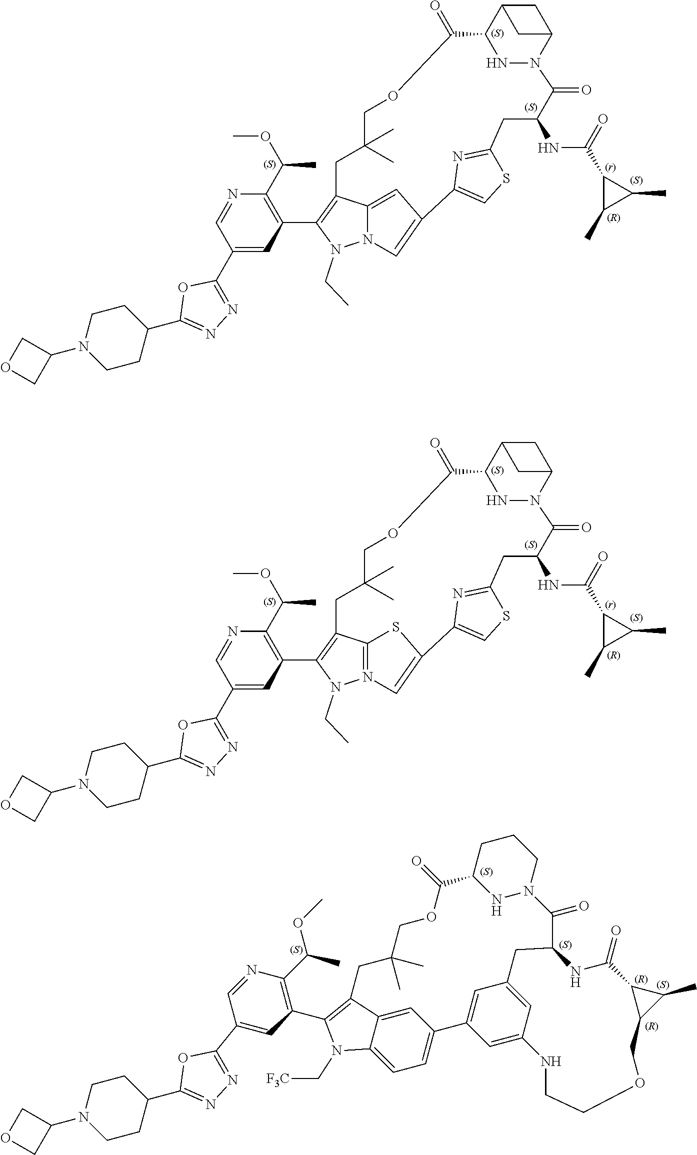

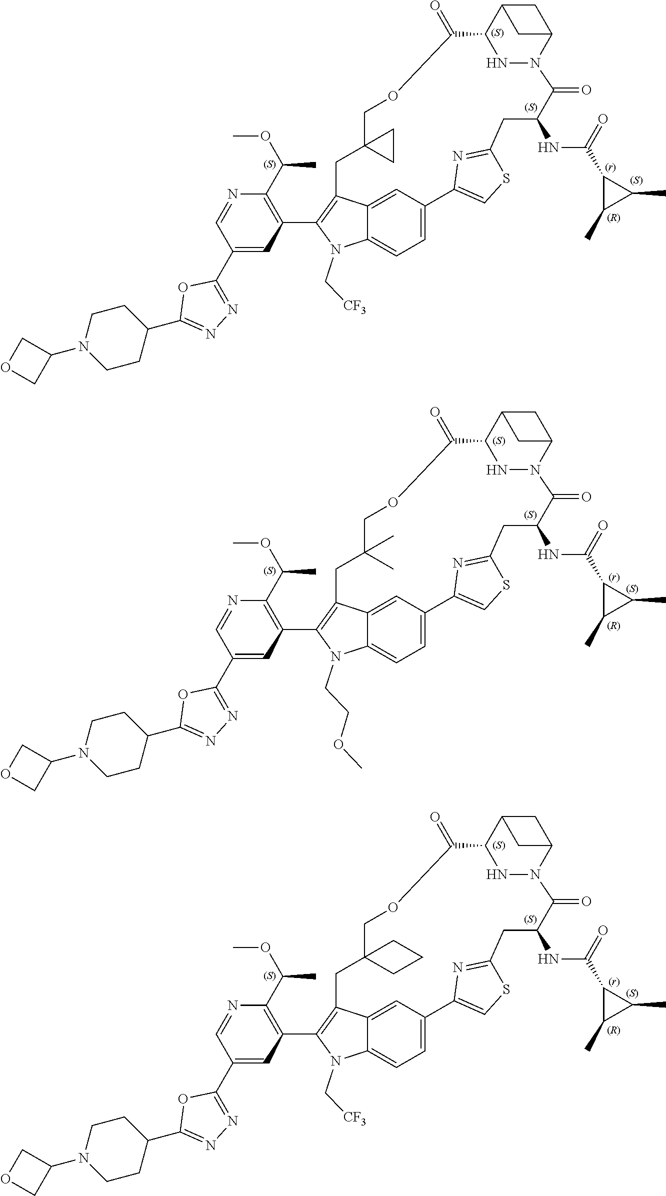

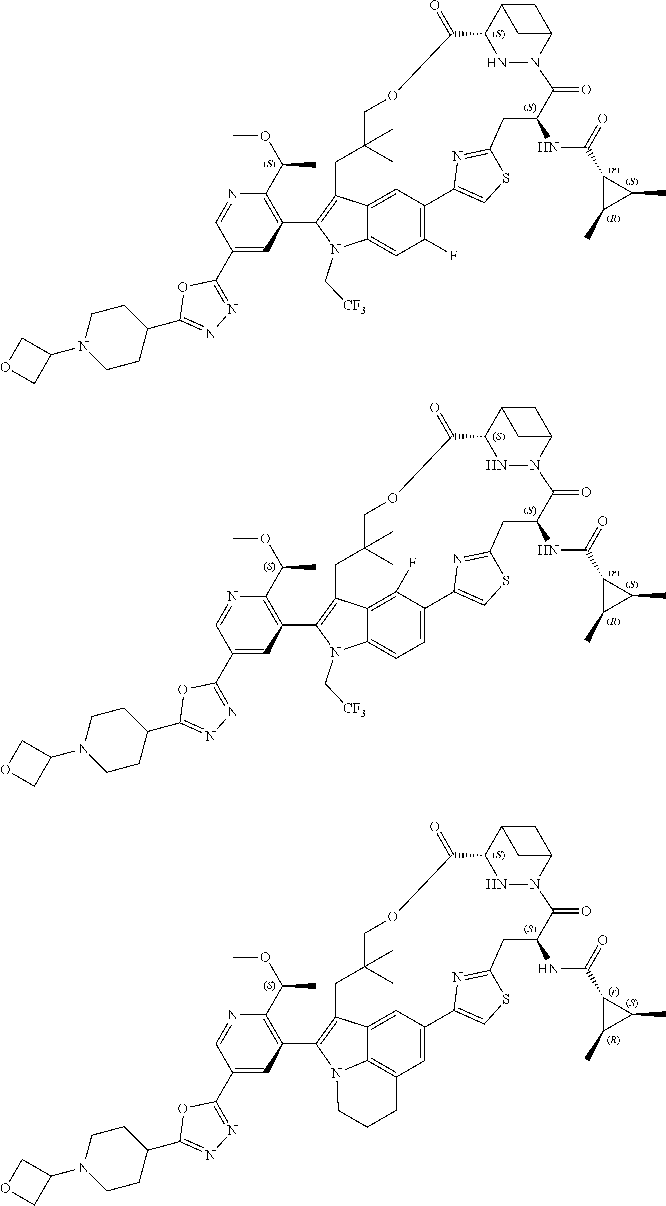

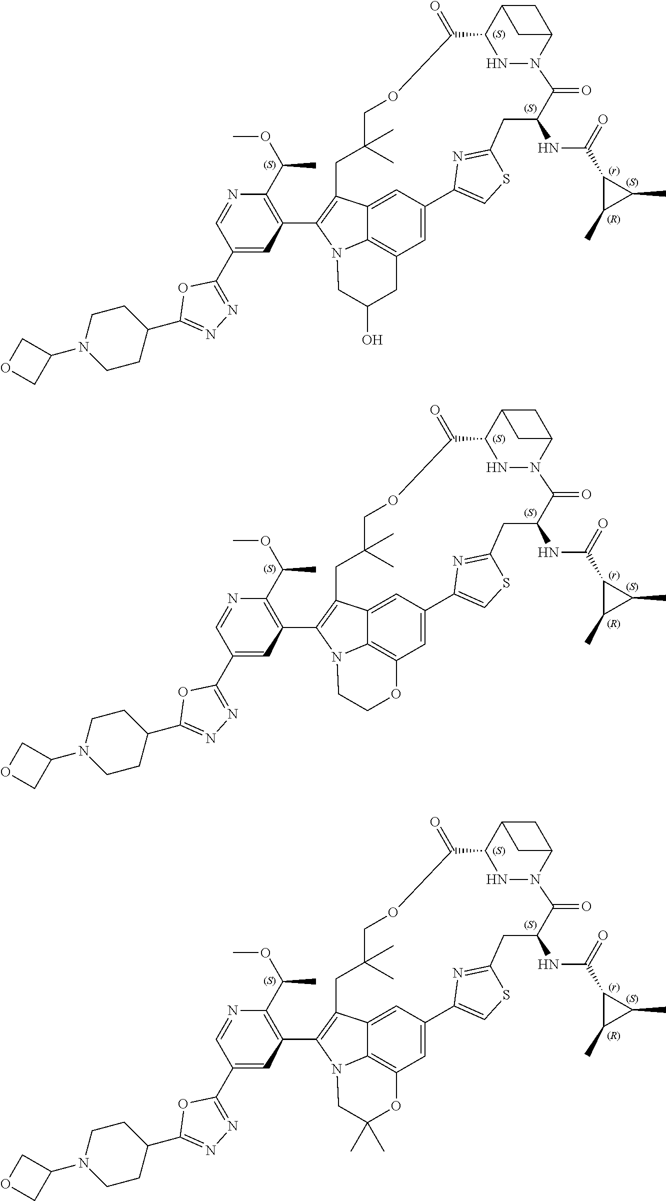

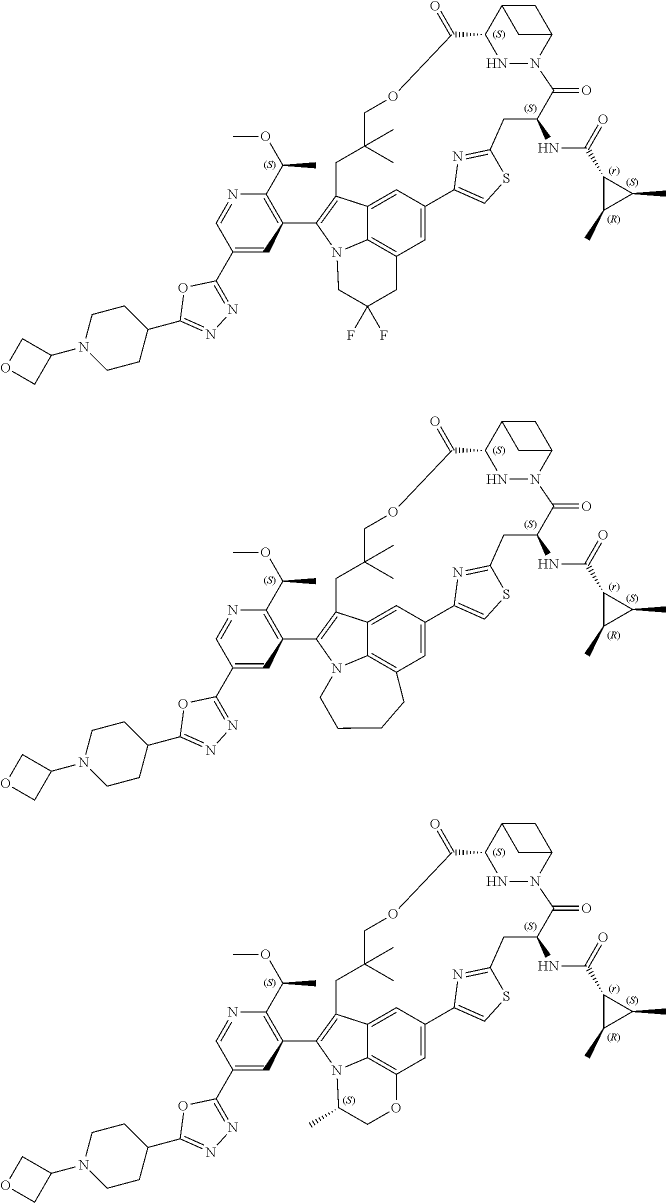

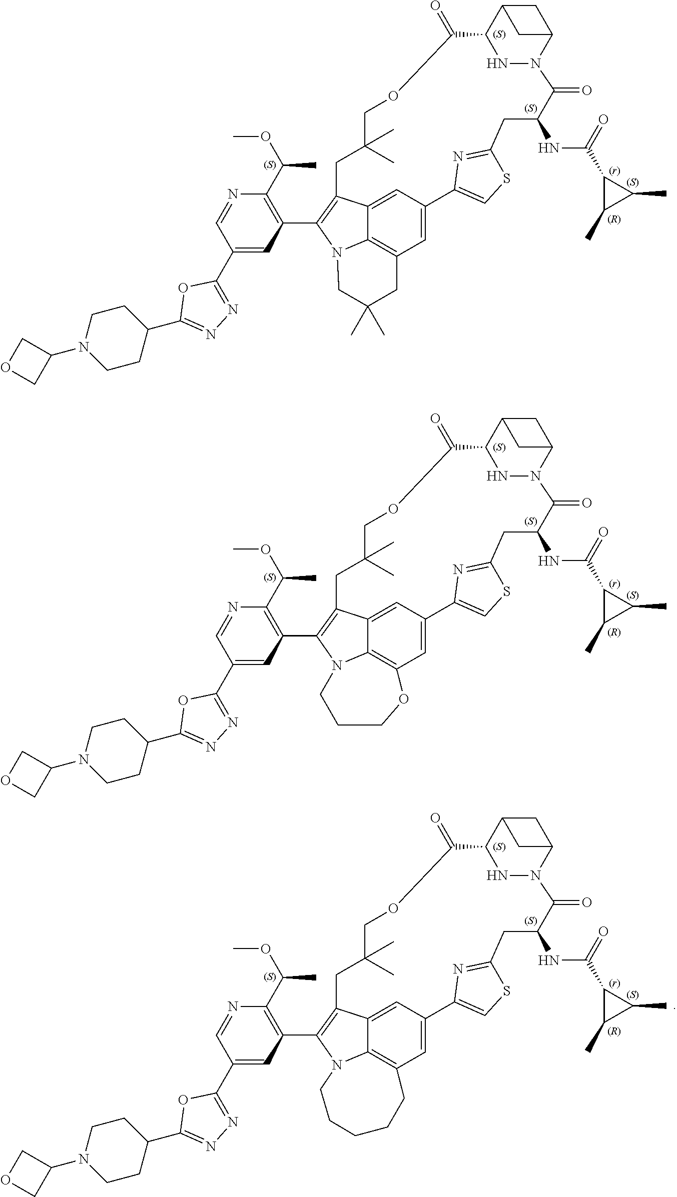

Embodiment 71. A pharmaceutical composition comprising a compound according to any one of embodiments 1 to 70, or an isotopic derivative, stereoisomer, or a pharmaceutically acceptable salt thereof, and at least one pharmaceutically acceptable carrier.

Embodiment 72. Use of a compound according to any one of embodiments 1 to 70, or an isotopic derivative, stereoisomer, or a pharmaceutically acceptable salt thereof, or the pharmaceutical composition according to embodiment 71, in the manufacture of a medicament for the prevention and/or treatment of cancer, tumor, inflammatory disease, autoimmune disease, or immune-mediated disease.

Embodiment 73. Use of a compound according to any one of embodiments 1 to 70, or an isotopic derivative, stereoisomer, or a pharmaceutically acceptable salt thereof, or the pharmaceutical composition according to embodiment 71, for the prevention and/or treatment of cancer, tumor, inflammatory disease, autoimmune disease, or immune-mediated disease.

Embodiment 74. A method for the prevention and/or treatment of cancer, tumor, inflammatory disease, autoimmune disease, or immune-mediated disease, comprising administering to a subject in need thereof a therapeutically effective amount of a compound according to any one of embodiments 1 to 70, or an isotopic derivative, stereoisomer, or a pharmaceutically acceptable salt thereof, or the pharmaceutical composition according to embodiment 71.

Embodiment 75. The use according to embodiment 72 or 73, or the method according to embodiment 74, wherein the cancer, tumor, inflammatory disease, autoimmune disease, or immune-mediated disease is a RAS protein-associated disease.

Embodiment 76. The use or method according to embodiment 75, wherein the RAS protein is one or more selected from KRAS protein, NRAS protein, or HRAS protein.

Embodiment 77. The use or method according to embodiment 75 or 76, wherein the cancer, tumor, inflammatory disease, autoimmune disease or immune-mediated disease includes a RAS mutation.

Embodiment 78. The use or method according to embodiment 77, wherein the RAS mutation comprises one or more selected from KRAS mutation, NRAS mutation, or HRAS mutation.

Embodiment 79. The use or method according to embodiment 77 or 78, wherein the RAS mutation is located at position 12, 13, and/or 61.

Embodiment 80. The use or method according to any one of embodiments 77 to 79, wherein the RAS mutation includes one or more selected from KRAS G12C, KRAS G12D, KRAS G12V, KRAS G12R, KRAS G13D, or NRAS Q61L.

1. A compound having the following structure selected from:

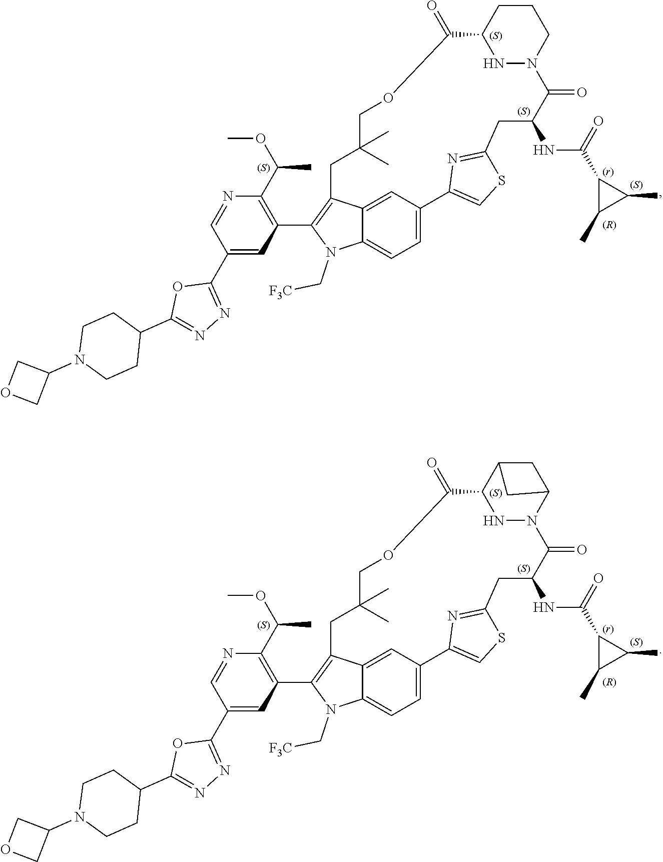

2. A compound having the following structure selected from: 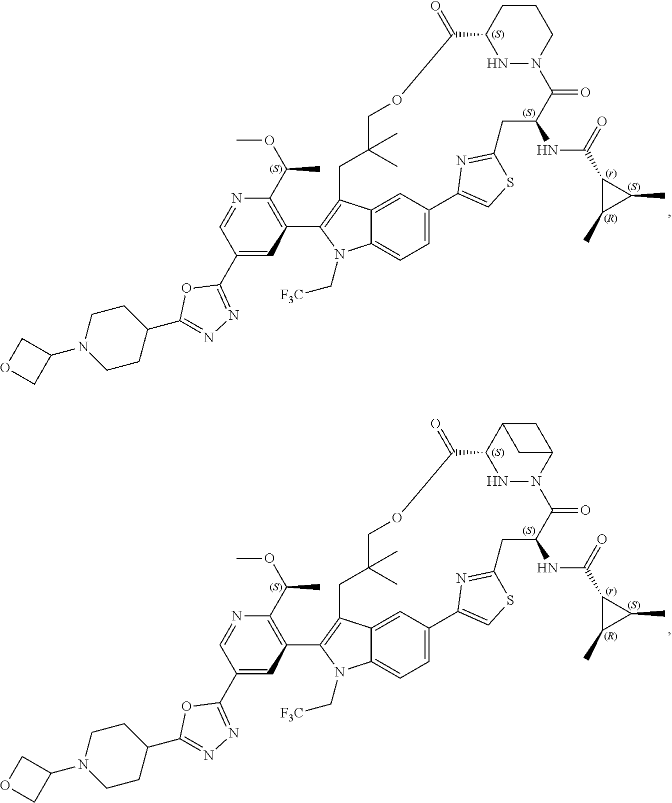
or a pharmaceutically acceptable salt thereof.

3. A pharmaceutical composition comprising a compound according to claim 1, and at least one pharmaceutically acceptable carrier.

4. A pharmaceutical composition comprising a compound or a pharmaceutically acceptable salt thereof according to claim 2, and at least one pharmaceutically acceptable carrier.
